(19) United States
(12) Patent Application Publication
(10) Pub. No.: US 2022/0283175 A1
(43) Pub. Date: Sep. 8, 2022

(54) METALLOENZYMES FOR BIOMOLECULAR RECOGNITION OF N-TERMINAL MODIFIED PEPTIDES

(71) Applicant: Encodia, Inc., San Diego, CA (US)

(72) Inventors: Eric OKERBERG, San Diego, CA (US); Stephen VERESPY, III, San Diego, CA (US); Jason C. KLIMA, San Diego, CA (US); Soumya GANGULY, San Diego, CA (US); Zachary MILES, San Diego, CA (US); Jason Duarte JACINTHO, San Diego, CA (US); Aaron WISE, San Diego, CA (US)

(73) Assignee: Encodia, Inc., San Diego, CA (US)

(21) Appl. No.: 17/727,677

(22) Filed: Apr. 22, 2022

Related U.S. Application Data
(63) Continuation of application No. PCT/US2021/065798, filed on Dec. 30, 2021.
(60) Provisional application No. 63/250,199, filed on Sep. 29, 2021.
(60) Provisional application No. 63/133,166, filed on Dec. 31, 2020.

(51) Int. Cl. G01N 33/68; G01N 33/573

(52) U.S. Cl. CPC G01N 33/6818; G01N 33/573

(57) ABSTRACT

The present disclosure relates to a metalloprotein binder that specifically binds to a N-terminally modified peptide. Also provided herein is a method and related kits for treating or analyzing a peptide using the metalloprotein binder and/or modified cleavase. In some embodiments, the method provided herein comprises binding metalloprotein binder-coding tag conjugates to a modified N-terminal amino acid residue of an immobilized peptide associated with a recording tag, transferring identifying information from the coding tag to the recording tag using a ligation or primer extension, and cleaving the modified N-terminal amino acid residue. The method and metalloprotein binders provided herein are useful for de novo peptide identification or sequencing.

### CROSS-REFERENCE TO RELATED APPLICATIONS

The present application is a continuation application of International Patent Application Serial No. PCT/US2021/065798, filed on Dec. 30, 2021, entitled “METALLOENZYMES FOR BIOMOLECULAR RECOGNITION OF N-TERMINAL MODIFIED PEPTIDES,” which claims priority to U.S. provisional patent application No. 63/133,166, filed on Dec. 31, 2020, and No. 63/250,199, filed on Sep. 29, 2021. The disclosures and contents of the above-referenced applications are incorporated herein by reference in their entireties for all purposes.

### STATEMENT REGARDING FEDERALLY SPONSORED RESEARCH

This invention was made with Government support awarded by National Institute of General Medical Sciences of the National Institutes of Health under Grant Number R44GM123836. The United States Government has certain rights in this invention pursuant to this grant.

### SEQUENCE LISTING ON ASCII TEXT

This patent application file contains a Sequence Listing submitted in computer readable ASCII text format (file name: 4614-2002930_SeqList_ST25.txt, date recorded: Apr. 15, 2022, size: 197,880 bytes). The content of the Sequence Listing file is incorporated herein by reference in its entirety.

### TECHNICAL FIELD

The present disclosure generally relates to biotechnology, in particular to methods for analysis or sequencing of peptides employing N-terminal modifying reagents and N-terminal binding agents engineered from metalloenzymes. The disclosure finds utility at least in a variety of methods and related kits for high-throughput peptide sequencing.

### BACKGROUND

High-throughput nucleic acid sequencing has transformed life science research through improved sensitivity and lower costs, and consequently has found multiple applications in medicine and personal genomics. Similar high-throughput approaches to protein sequencing are not currently available, yet knowledge about protein identity in a sample can be crucial for better understanding of proteome dynamics in health and disease. This information can enable precision medicine and can be used in multiple diagnostic applications. Despite advances in mass spectroscopy (MS), corresponding innovation in proteomics is needed to have a similar broad-ranging impact on biomedical research. MS suffers from several drawbacks including high instrument cost, requirement for a sophisticated user, poor quantification ability, and limited ability to make measurements spanning the dynamic range of the proteome. For example, since proteins ionize at different levels of efficiencies, absolute quantitation and even relative quantitation between sample is challenging. Also, MS typically only analyzes the more abundant species, making characterization of low abundance proteins challenging.

Several approaches to high-throughput protein sequencing have been published, including U.S. Pat. No. 9,435,810 B2, WO2010/065531A1, US 2019/0145982 A1, US 2020/0348308 A1, which utilize N-terminal amino acid (NTAA) recognition as a critical step during a protein sequencing assay. A number of methods to evolve specific NTAA binders from different scaffolds have been disclosed, including directed evolution approaches to derive variant amino acyl tRNA synthetases, N-recognins such as ClpS and ClpS2, anticalins, and aminopeptidases (disclosed in US 2019/0145982 A1, U.S. Pat. No. 9,435,810 B2). However, identifying binding agents that afford amino acid specificity with sufficiently strong affinity has proven challenging. There remains a need in the art for improved techniques relating to macromolecule recognition and/or analysis. There is a need for proteomics technology that is highly-parallelized, accurate, sensitive, and high-throughput.

The present disclosure describes the development of peptide sequencing reagents including specific NTAA binders, and methods that fulfill this and other needs. These and other embodiments of the invention will be apparent upon reference to the following detailed description. To this end, various references are set forth herein which describe in more detail certain background information, procedures, compounds and/or compositions, and are each hereby incorporated by reference in their entireties.

### BRIEF SUMMARY

The summary is not intended to be used to limit the scope of the claimed subject matter. Other features, details, utilities, and advantages of the claimed subject matter will be apparent from the detailed description including those embodiments disclosed in the accompanying drawings and in the appended claims.

The present disclosure relates to an engineered metalloprotein binder that specifically binds to an N-terminally modified peptide via interaction with modified N-terminal amino acid (NTAA) residue of the peptide. Also provided herein is a method and related kits for treating a peptide using or comprising the binder and/or the modified or engineered cleavase. In some embodiments, also provided herein is a method and related kits for transferring information using a plurality of enzymes, including for performing a ligation, extension, and cleavage reaction with nucleic acid molecules associated with the peptide for analysis.

In one embodiment, provided herein is an engineered metalloprotein binder that specifically binds to an N-terminally modified target peptide modified by an N-terminal modifier agent, wherein:

(a) the N-terminally modified target peptide has a formula: Z-P1-P2-peptide, wherein Z is an N-terminal modification capable of coordinating or chelating a zinc metal cation M, P1-P2-peptide is a target peptide before modification with the N-terminal modifier agent, Z-P1 is a modified N-terminal amino acid (NTAA) residue of the target peptide, and P2 is a penultimate terminal amino acid residue of the target peptide;
(b) the engineered metalloprotein binder specifically binds to the N-terminally modified target peptide through interaction between the engineered metalloprotein binder and the Z-P1 of the N-terminally modified target peptide; and
(c) the engineered metalloprotein binder comprises an amino acid sequence X1-C/H/D/E-X2-C/H/D/E-X3-C/H/D/E-X4, wherein C/H/D/E is any single amino acid residue independently selected from the group consisting of amino acid residues C (Cys), H (His), D (Asp) and E (Glu); X1, X2, X3 and X4 are each any amino acid sequence comprising between 0 and 200 amino acid residues in length, and wherein the amino acid sequence X1-C/H/D/E-X2-C/H/D/E-X3-C/H/D/E-X4 chelates a zinc metal cation M with a thermodynamic dissociation constant of 0.5 nM or less. In preferred embodiments, X1, X2, X3 and X4 together comprise at least 30 amino acid residues in length.

In another embodiment, provided herein is an isolated nucleic acid molecule comprising a polynucleotide having a sequence encoding the engineered metalloprotein binder described in the previous paragraph.

In yet another embodiment, provided herein is a method of treating a target peptide, the method comprises:

(a) contacting the target peptide with an N-terminal modifier agent to form an N-terminally modified peptide having a formula: Z-P1-P2-peptide, wherein Z is an N-terminal modification capable of coordinating or chelating a zinc metal cation M, P1-P2-peptide is a target peptide before modification with the N-terminal modifier agent, Z-P1 is a modified N-terminal amino acid (NTAA) residue of the target peptide, and P2 is a penultimate terminal amino acid residue of the target peptide; and
(b) contacting an engineered metalloprotein binder with the N-terminally modified target peptide to allow the engineered binder to specifically bind to the N-terminally modified target peptide through interaction between the engineered binder and the modified NTAA residue of the N-terminally modified target peptide, wherein the engineered binder comprises an amino acid sequence X1-C/H/D/E-X2-C/H/D/E-X3-C/H/D/E-X4, wherein C/H/D/E is any single amino acid residue independently selected from the group consisting of amino acid residues C (Cys), H (His), D (Asp) and E (Glu); X1, X2, X3 and X4 are each any amino acid sequence comprising between 0 and 200 amino acid residues in length, and wherein the amino acid sequence X1-C/H/D/E-X2-C/H/D/E-X3-C/H/D/E-X4 chelates a zinc metal cation M with a thermodynamic dissociation constant of 0.5 nM or less. In preferred embodiments, X1, X2, X3 and X4 together comprise at least 30 amino acid residues in length.

In yet another embodiment, provided herein is a kit for treating a target peptide, the kit comprises:

(a) an engineered metalloprotein binder as described above; and
(b) one of more of the following:
1) an N-terminal modifier agent to form an N-terminally modified peptide having a formula: Z-P1-P2-peptide, wherein Z is an N-terminal modification capable of coordinating or chelating a zinc metal cation M, P1-P2-peptide is a target peptide before modification with the N-terminal modifier agent, Z-P1 is a modified N-terminal amino acid (NTAA) residue of the target peptide, and P2 is a penultimate terminal amino acid residue of the target peptide;
2) an agent configured for removing the modified NTAA residue from the N-terminally modified target peptide, thereby exposing a new NTAA residue;
3) an agent configured for immobilizing the target peptide on a solid support;
4) a solid support;
5) a nucleic acid recording tag;
6) a nucleic acid tag or a nucleic acid coding tag;
7) a detectable label; and/or
8) a peptide coupling reagent.

### BRIEF DESCRIPTION OF THE DRAWINGS

Non-limiting embodiments of the present invention will be described by way of example with reference to the accompanying figures, which are schematic and are not intended to be drawn to scale. For purposes of illustration, not every component is labeled in every figure, nor is every component of each embodiment of the invention shown where illustration is not necessary to allow those of ordinary skill in the art to understand the invention.

FIG. 1 depicts an exemplary design of the NGPS peptide sequencing assay utilizing N-terminal amino acid (NTAA)-specific binding agents. (1) Peptide molecules are each associated with a DNA recording tag (RT) and attached to beads at a low molecular density, a sparsity that permits only intramolecular information transfer to occur. The N-terminal amino acid (NTAA) residue (F) of the peptide is labeled with an N-terminal modification (NTM). (2) Next, immobilized and labeled peptides are contacted with binding agents specific for labeled NTAA (labeled F (F*)-specific binding agent is shown). Each binding agent comprises a DNA coding tag (CT) that comprises identifying information regarding the binding agent. After binding and washing, the coding tag identifying information is transferred enzymatically to the recording tag (via extension or ligation), generating an extended RT. (3) The labeled NTAA is removed by using mild Edman-like elimination chemistry or by a Cleavase enzyme. The cycle 1-2-3 is repeated n times. After n cycles, the extended RT representing the n amino acids of the peptide sequence is formed and can be sequenced by NGS. A representative structure of the extended RT after 7 cycles is shown.

FIG. 2A. Exemplary active site architecture common in zinc binding metalloenzymes. FIG. 2B. Potential zinc binding N-terminal modifications: sulfamoylbenzene-NHS ester and -isothiocyanate. FIG. 2C. Proposed sulfamoylbenzene, “PMI” and aminoguanidine zinc coordination by the modified N-termini of a peptide.

FIG. 3. Examples of zinc-binding NTMs.

FIG. 4. Examples of zinc-binding NTMs.

FIG. 5. Exemplary metal-binding isosteres of picolinic acid (1) for use as NTMs.

FIG. 6A-FIG. 6C. Structures of zinc-binding NTMs experimentally tested in this study. The tested NTMs are designated as M64-M98.

FIG. 7A. Structures of the SABA-modified XAAAE peptides. FIG. 7B. Inhibition of hCAII activity by the SABA-modified XAAAE peptides (IC50 values were determined).

FIG. 8A-FIG. 8D. Exemplary design of N-terminal modifications (NTMs) to enable NTM-NTAA (NTM-P1) binding with minimal P2 bias. The size and shape of the NTM is designed to fill the metalloprotein binder substrate pocket such that only the P1 residue of the peptide makes substantial contact with the substrate pocket, but not P2 residue. FIG. 8A. Structure of a bipartite NTM (NTMa) comprised of “binding” region (“N”) and a separate metal-binding group (MBG) connected with an amide bond. N could be a natural amino acid residue. NTMb has a composite metal-binding region (both groups involve in metal binding). FIG. 8B. The NTMs are activated using standard methods (activated ester) and are coupled to the N-terminal amine on the P1 residue. FIG. 8C. An engineered metalloprotein binder binds to the modified NTAA of the peptide by interacting with the P1 residue and the NTM. Metal ion present in the metalloprotein binding pocket interacts with the MBG. “N” can interact with amino acid residues distant from the metal-coordinating residues. FIG. 8D. An engineered metalloprotein binder binds to the modified NTAA of the peptide by interacting with P1 and NTMb. Metal ion present in the metalloprotein binding pocket interacts with both groups of NTMb.

FIG. 9A-B. FIG. 9A. Derivatives of NTM M64 were evaluated using colorimetric IC50 assay to determine relative binding affinity of NTMs to wild-type hCAII protein based on NTM inhibition capacity. The slopes versus the concentration of NTM were put into a non-linear regression equation to determine the IC50 of the selected NTMs to the wild-type hCAII. FIG. 9B. Derivatives of NTM M64 were installed on the N-terminus of a model peptide AAEIR. The N-terminally modified peptides were then evaluated using colorimetric IC50 assay to determine relative binding affinity of NTM-AAEIR to wild-type hCAII protein based on NTM-AAEIR inhibition capacity. The slopes versus the concentration of NTM-AAEIR were put into a non-linear regression equation to determine the IC50 of the selected NTM-AAEIR peptides to the wild-type hCAII.

FIG. 10 illustrates heatmap data showing encoding efficiency (calculated as fraction of recording tags encoded) for the engineered M64-D binder (SEQ ID NO: 48) in the multiplex encoding assay on immobilized set of 288 peptides (17×17 combination of different P1 and P2 residues). Arrow on the left shows primary binder's specificity. The more intense white color of a cell representing a particular P1-P2 combination indicates higher encoding efficiency, and the number in the cell indicates encoding yield for peptide with the P1-P2 combination (fraction of recording tags encoded).

FIG. 11 illustrates heatmap data showing encoding efficiency (calculated as fraction of recording tags encoded) for the engineered M64-F binder (SEQ ID NO: 51) in the multiplex encoding assay on immobilized set of 288 peptides (17×17 combination of different P1 and P2 residues). Arrow on the left shows primary binder's specificity. The more intense white color of a cell representing a particular P1-P2 combination indicates higher encoding efficiency, and the number in the cell indicates encoding yield for peptide with the P1-P2 combination (fraction of recording tags encoded).

FIG. 12. illustrates heatmap data showing encoding efficiency (calculated as fraction of recording tags encoded) for the engineered M64-E binder (SEQ ID NO: 55) in the multiplex encoding assay on immobilized set of 288 peptides (17×17 combination of different P1 and P2 residues). Arrow on the left shows primary binder's specificity. The more intense white color of a cell representing a particular P1-P2 combination indicates higher encoding efficiency, and the number in the cell indicates encoding yield for peptide with the P1-P2 combination (fraction of recording tags encoded).

FIG. 13. illustrates heatmap data showing encoding efficiency (calculated as fraction of recording tags encoded) for the engineered M64-T binder (SEQ ID NO: 57) in the multiplex encoding assay on immobilized set of 288 peptides (17×17 combination of different P1 and P2 residues). Arrow on the left shows primary binder's specificity. The more intense white color of a cell representing a particular P1-P2 combination indicates higher encoding efficiency, and the number in the cell indicates encoding yield for peptide with the P1-P2 combination (fraction of recording tags encoded).

### DETAILED DESCRIPTION

The present disclosure relates to a metalloprotein binder that specifically binds to an N-terminally modified amino acid residue of a peptide. Also provided herein is a method and related kits for modifying N-terminal amino acid residue of a peptide with a N-terminal modifier agent, as well as for treating a peptide using the metalloprotein binder. In some embodiments, also provided herein is a method and related kits for transferring information regarding the metalloprotein binder that specifically binds to the N-terminally modified amino acid residue of a peptide and identifying the N-terminal amino acid residue of the peptide based on this information. Transferring information involves one or more enzymes, including for performing a nucleic acid ligation, nucleic acid extension and/or a N-terminal amino acid cleavage reaction. In some embodiments, a plurality of peptides obtained from a sample is analyzed. In some embodiments, the sample is obtained from a subject. In some embodiments, the peptide sequencing or analysis method includes using a plurality of binding agents associated with coding tags to detect a plurality of peptides to be analyzed. Also provided are kits containing components and/or reagents for performing the provided methods for peptide sequencing and/or analysis. In some embodiments, the kits also include instructions for using the kit to perform any of the methods provided herein.

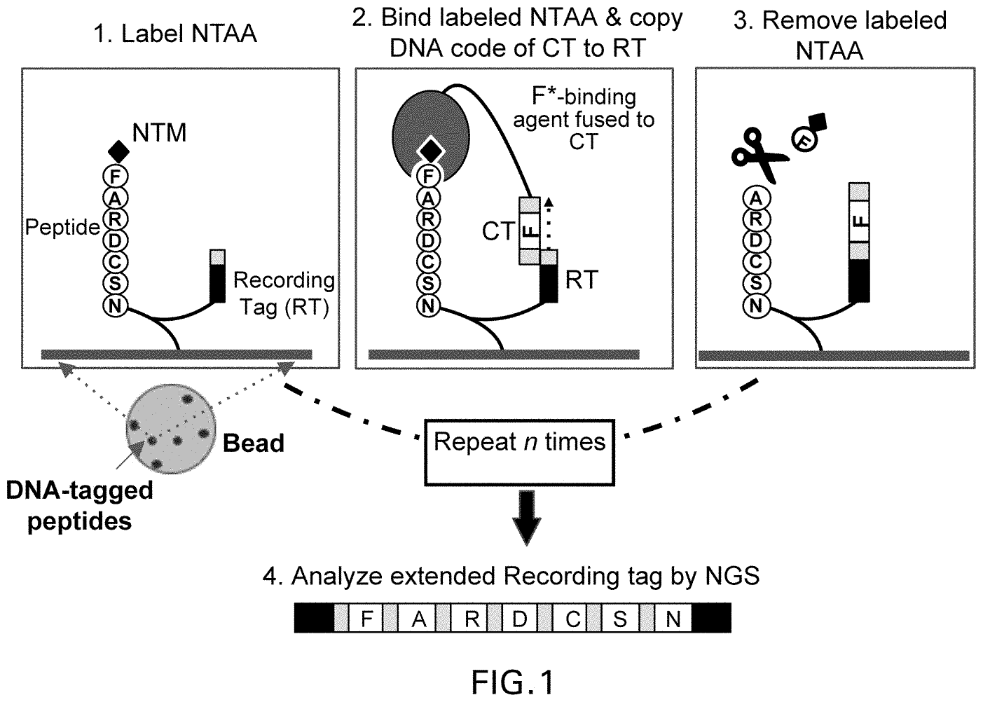

Highly-parallel characterization and recognition of macromolecules such as peptides remains a challenge. In proteomics, one goal is to identify and quantitate numerous proteins in a sample, which is a formidable task to accomplish in a high-throughput way. One approach for peptide sequencing disclosed in, for example, U.S. Pat. No. 9,435,810 B2, US 2019/0145982 A1, US 2020/0348308 A1, comprises contacting a peptide immobilized on a support with one or more N-terminal amino acid (NTAA) binding agents, obtaining and/or transferring information regarding the NTAA binding agent bound to the NTAA of the peptide, and identifying the NTAA of the peptide based on the obtained information. To identify penultimate terminal amino acid residue of the peptide, the NTAA of the peptide is removed after obtaining and/or transferring information step, thus exposing the penultimate terminal amino acid residue of the peptide as a new NTAA of the peptide. After that, the described above steps of contacting the peptide with one or more NTAA binding agents and obtaining and/or transferring information regarding the NTAA binding agent bound to the NTAA of the peptide are repeated (see, for example, FIG. 1). The information regarding specific NTAA binding agents for each cycle are collected and either processed immediately or stored until later or the end of the cycles (when all or most amino acid residues of the peptide are cleaved). The described method requires a set of specific binding agents (binders), wherein each binder from the set binds with high affinity to a particular NTAA and does not bind to other NTAAs. U.S. Pat. No. 9,435,810 B2 discloses an approach to make specific NTAA binders by introducing mutations into E. coli methionine aminopeptidase and different tRNA synthetases, since these enzymes have intrinsic specificity for free amino acids and can be utilized as a scaffold for specific binders. However, this approach resulted in binders with μM or higher affinity constants, and approximately 2:1 ratio of specific to non-specific binding (U.S. Pat. No. 9,435,810, Examples 1 and 10). Accordingly, there remains a need for more accurate, sensitive and high-throughput techniques relating to protein sequencing and/or analysis, as well as to products, methods and kits for accomplishing the same.

The disclosed methods herein are aimed to obtain specific binders to NTAAs with a high binding affinity (preferably, equilibrium dissociation constant K_{d} is less than 200 nM). Weak binding affinity (K_{d}>200 nM) imparts some constraints on utility for methods (material production, high protein concentration, etc.). Chemical modification of N-terminus of a peptide can be used to improve binder affinity through additional hydrogen bonding and hydrophobic interactions. One approach to impart NTAA/binder affinity is to modify N-termini with established small molecule inhibitors of specific macromolecule targets (such as metalloenzymes) and employ those targets as binders. Medicinal chemistry programs have provided countless high affinity N-terminal modification (NTM)/binder pairs as starting points. However, synthetic tractability, facile installation, and prediction of appropriate binder/NTAA interactions make identification of ideal NTM/binder pairs a complicated proposition. Once appropriate reagents are identified, the P1, P2, etc. specificity must be evaluated and potentially tuned through genetic modification of the binder protein sequence (herein, P1 is a N-terminal amino acid residue and P2 is a penultimate terminal amino acid residue of the peptide to be analyzed). The capacity for altered NTAA specificity is strongly dependent on the tertiary structure of the initial protein scaffold and the NTM binding site, as well as the NTM chemical structure(s). Preferably, a single N-terminal modifier agent can be used for all NTAAs during binding, and also can be utilized for removal of the NTAA after binding and collecting information regarding the binder during multi-cycle approach for peptide sequencing.

Disclosed herein are metal chelating pharmacophores as high affinity, universal N-terminal modifications (NTMs) recognized by structurally diverse metalloenzymes that serve as binder scaffolds. Disclosed herein are N-terminal modifier agents that interact with and modify (or functionalize) N-terminal amino acid residues (P1 residues) of peptides to be analyzed. Such an N-terminal modifier agent modifies a peptide to form NTM-P1 group at the N-terminus of the peptide, wherein NTM is a chemical group that incorporate a metal binding group (MBG) in order to coordinate or chelate a metal ion. This approach employs metal ions as dual action affinity reagents, simultaneously recognized by both the binder scaffold and the NTM. This facilitates high affinity binder/NTM interactions and is used as a mechanism for protein tertiary structure to impart NTAA specificity. Metalloenzymes offer nM or sub-nM affinity towards their substrates, and an enhanced affinity in the disclosed methods is derived from an ability of the NTAA modification to coordinate an active site metal ion. Common structural elements in metal binding proteins (such as the conserved HEXGHXXGXXH zinc binding sequence) enable multiple orthogonal protein scaffolds to serve as binders, with the aim of attaining the NTAA specificity required for the disclosed protein sequencing assay. Numerous high affinity metal chelating pharmacophores identified in medicinal chemistry programs provide a wealth of potential metal binding NTM's. The scope of known metal binding NTMs include those with simple installation and potential compatibility with both chemical and enzymatic N-terminal elimination (NTE) of peptide's NTAA. The approach described herein provides the opportunity to derive multiple binders, with varied NTAA specificity, against a single, high affinity metal binding NTM.

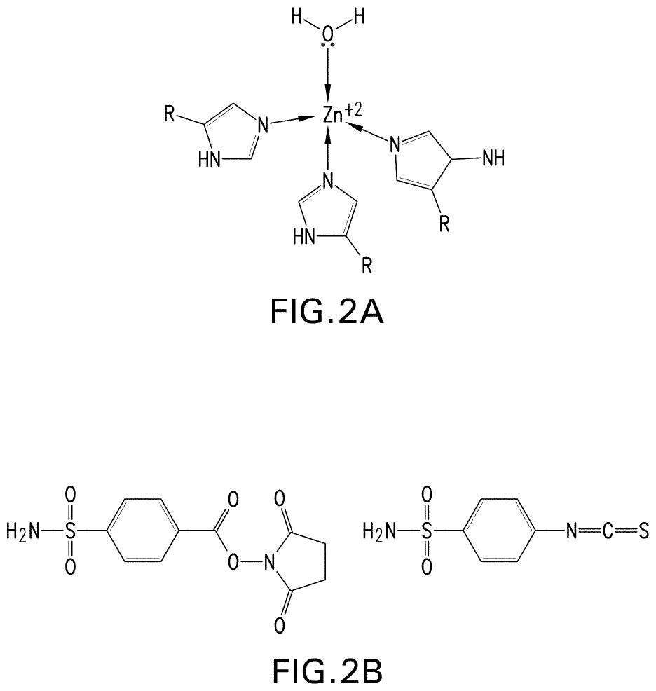

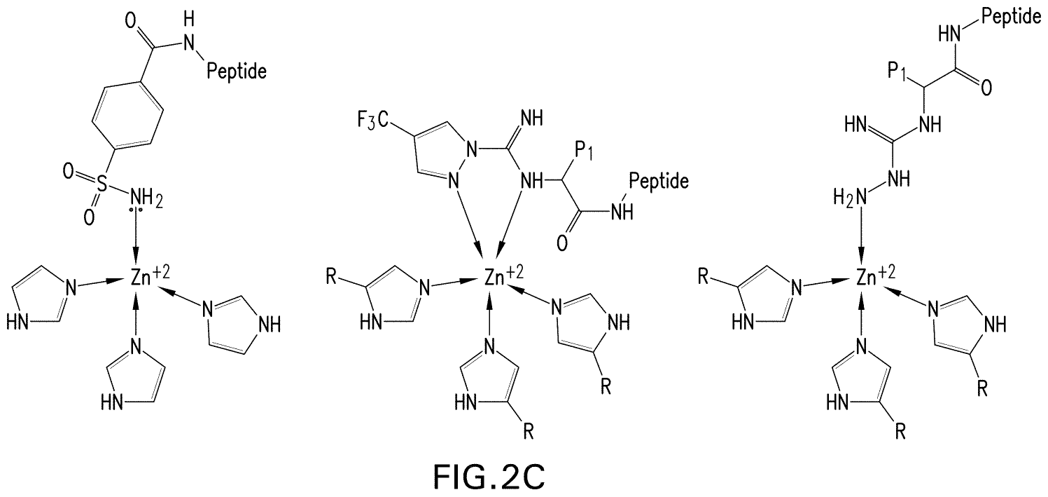

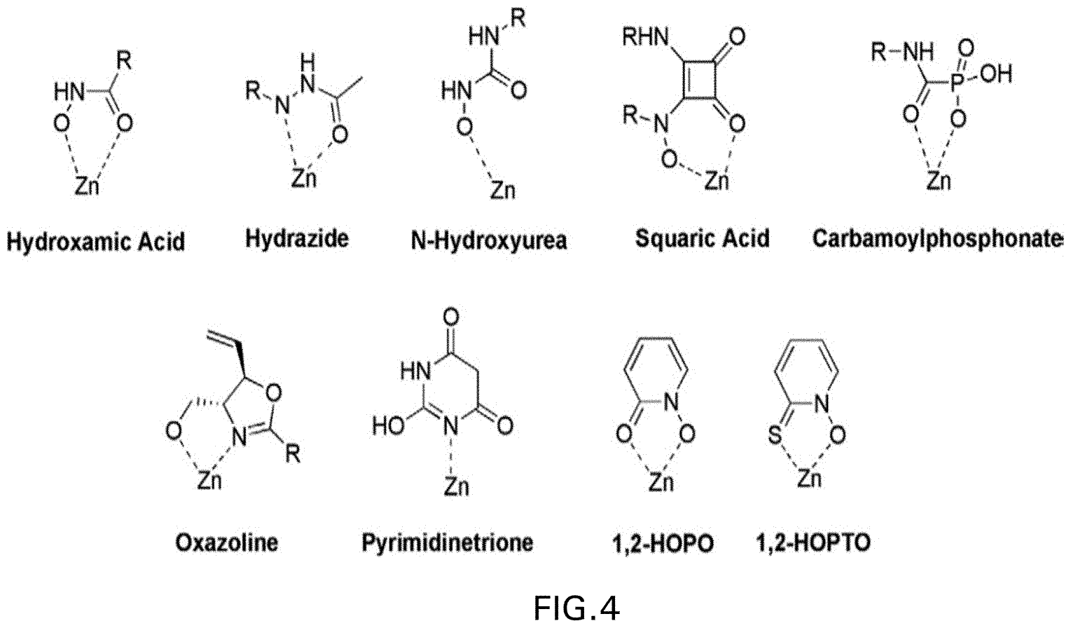

In metalloenzymes, active site histidines (and/or cysteines, glutamates, aspartates) coordinate metal ions in a multidentate fashion to yield a high affinity metal binding site. An “activated” water molecule is often coordinated to the protein bound metal ion to affect catalysis (FIG. 2A). This water molecule is generally displaced by metal binding pharmacophores, resulting in high affinity but non-target specific interactions. Medicinal chemistry efforts therefore focus on defining additional substituents to impart selectivity for a particular target. Common metal binding pharmacophores include sulfonamides, hydroxamates, carboxylates, thiols, phosphonates, pyrazoles, etc. with typical Kd values ranging from pM to nM (see, for example, FIG. 2-FIG. 5). The fact that many metalloprotease inhibitors are derived from peptide substrates supports the notion that NTM-peptides can serve as effective ligands.

Several metal binding groups are evaluated as metal binding NTMs. Preferred NTMs are those that can be installed on NTAA of a peptide, provide high affinity and specificity during binding reactions with metalloprotein binders that recognize NTM-modified NTAAs (including a proper size of NTMs that fit a binding pocket of the binding metalloprotein binder), and also are compatible with removal of the NTM-modified NTAAs after binding. Removal of a modified terminal amino acid can be accomplished by a number of known techniques, including chemical cleavage and enzymatic cleavage. Methods and reagents for chemical cleavage, such as mild Edman-like degradation, are disclosed in, for example, in US 2020/0348307 A1 or WO2020/223133 A1. Mild conditions are preferably used during cleavage, since they are compatible with transferring information regarding the binding agent during the encoding assay. Most preferably, utilized mild conditions are compatible with DNA (do not compromise integrity of DNA or DNA-related assays). Alternatively, instead of chemical cleavage, an engineered enzyme (cleavase) is used for removal of a modified terminal amino acid. Enzymatic cleavage can be accomplished by an engineered cleavase, such as aminopeptidase, a carboxypeptidase, dipeptidyl peptidase, dipeptidyl aminopeptidase, or variant thereof. Some engineered cleavases are disclosed in the published patents and patent applications U.S. Pat. No. 9,435,810 B2, WO2010/065322 or US 2021/0214701 A1.

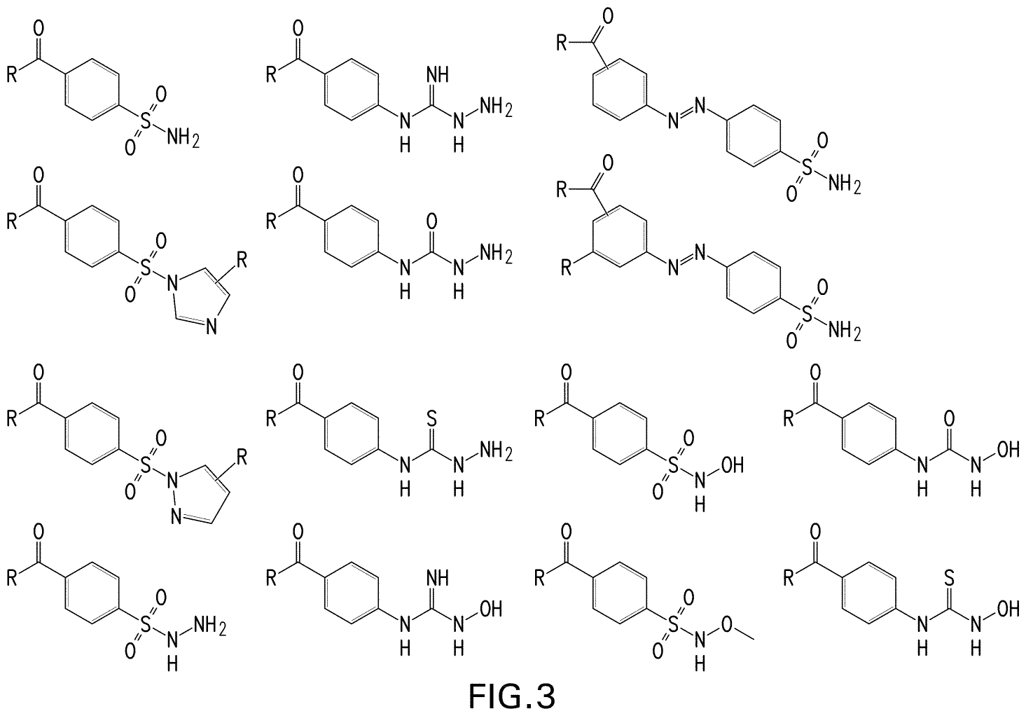

In some embodiments, the provided N-terminal modifier agents and/or NTMs comprise chemical moieties that are known functional inhibitors of metalloenzymes, or structural variants thereof. Some examples of NTMs include phenylsulfonamide substituents that afford strong affinity, ease of installation, broad specificity, and structural similarity to cleavase substrates. Some variants of sulfonamides include aryl (benzene, pyrazole, imidazole), amino acid, alkyl sulfonamides. Terminal sulfonamides and N-substituted sulfonamides can be utilized. Arylsulfonamides are very well established inhibitors of carbonic anhydrase (CA). Other derivatives of sulfonamides can also impart high affinity metal binding. Further, isothiocyanate activated phenylsulfonamides enable efficient N-terminal installation and Edman-like degradation of NTM-NTAAs. Alternatively, hydrazides, semicarbazides, imidazoles, and pyrrazoles are established metal binding groups and are structurally related to reagents implemented in mild chemical cleavage of modified NTAAs described in WO2020/223133 A1. For example, aryl-4,5-dihydro-1H-pyrazole-1-carboxamide derivatives bearing a sulfonamide moiety show nanomolar inhibition constants against carbonic anhydrases (Hargunani P, et al., Aryl-4,5-dihydro-1H-pyrazole-1-carboxamide Derivatives Bearing a Sulfonamide Moiety Show Single-digit Nanomolar-to-Subnanomolar Inhibition Constants against the Tumor-associated Human Carbonic Anhydrases IX and XII. Int J Mol Sci. 2020 Apr. 9; 21(7):2621). In other embodiments, hydroxamates, compounds bearing the functional group RC(O)N(OH)R′, with R and R′ are organic residues and CO is a carbonyl group can be utilized in NTMs. Many hydroxamates are used as metal chelators and display nanomolar affinities against metalloenzymes (established as inhibitors for matrix metalloenzymeases (MMPs), aminopeptidases, histone deacetylases (HDACs), peptide deformylases, carboxypeptidases, and carbonic anhydrases). In other embodiments, thiol groups or carboxylates can be included in NTMs, since these groups are common in Fe²⁺ and in Mg²⁺ binding motifs, respectively. In other embodiments, benzoxaborole derivatives can be utilized in NTMs as they were shown to potently inhibit carbonic anhydrases (Langella E, et al, Exploring benzoxaborole derivatives as carbonic anhydrase inhibitors: a structural and computational analysis reveals their conformational variability as a tool to increase enzyme selectivity. J Enzyme Inhib Med Chem. 2019 December; 34(1):1498-1505). In other embodiments, NTMs as shown in FIGS. 3-5 are provided and utilized in the methods disclosed herein.

In some embodiments, N-terminal modifier agent or an NTM group comprises a compound of Formula (1):

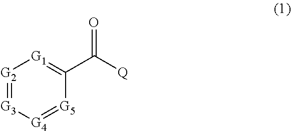

wherein Q is OH, OR^{Q} or OM,
each R^{Q} is independently aryl, heteroaryl, or heterocycloalkyl each of which is optionally substituted with one or more groups selected from halo, nitro, cyano, sulfonate, carboxylate, alkylsulfonyl, and N of heteroaryl is optionally oxidized; or R^{Q} can be —C(═O)R or —C(═O)—OR; M is a cationic counterion;
G¹-G⁵ are each independently selected from CH, CJ, and N, provided not more than 3 of G¹-G⁵ are N; J at each occurrence is independently selected from H, C₁-C₂ alkyl, NO₂, C₁-C₂ haloalkyl, C₁-C₂ haloalkoxy, halo, —OR¹, —N(R¹)₂, —SR¹, —S(O)ₙR¹, —NR¹SO₂R¹, —SO₂N(R¹)₂, —SO₃R¹, —B(OR¹)₂, —C(═O)R¹, —CN, —N═N—R¹, —C(N)R¹, —CON(R¹)₂, —CSN(R¹)₂—COOR¹, —C(O)Ar, and tetrazole, where Ar represents a phenyl or 5-6 membered heteroaryl ring that is optionally substituted with one or two groups selected from halo, CN, R¹ and OR¹;
R¹ is independently selected at each occurrence from H, OR², N(R²)₂, C₁-C₂ alkyl, C₁-C₂ haloalkyl, aryl, heteroaryl, that is optionally substituted with one or two groups selected from halo, N(R²)ₙ, COOH, —S(O)ₙR², —S(O)₂N(R²)ₙ;
R² is independently selected from H, OH, NH₂ or C₁-C₂ alkyl; and
n at each occurrence is independently 1 or 2.

In some embodiments, N-terminal modifier agent or an NTM group comprises a compound of the following Formulas:

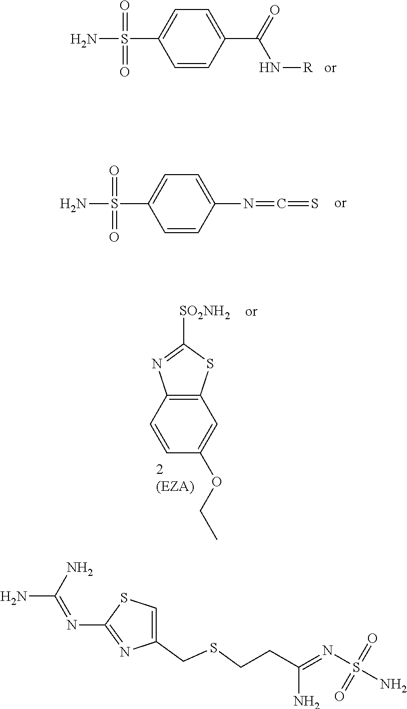

In some embodiments, N-terminal modifier agent comprises:

either
(b1) a metal-binding compound of Formula (AA):

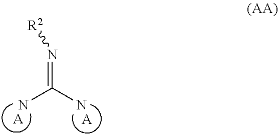

wherein:
R² is H or R⁴;
R⁴ is C₁₋₆ alkyl, which is optionally substituted with one or two members selected from halo, C₁₋₃ alkyl, C₁₋₃ alkoxy, C₁₋₃ haloalkyl, phenyl, 5-membered heteroaryl, and 6-membered heteroaryl, wherein the phenyl, 5-membered heteroaryl, and 6-membered heteroaryl are optionally substituted with one or two members selected from halo, —OH, C₁₋₃ alkyl, C₁₋₃ alkoxy, C₁₋₃ haloalkyl, NO₂, CN, COOR″, and CON(R″)₂,
where each R″ is independently H or C₁₋₃ alkyl;
each ring A is a 5-membered heteroaryl ring containing up to three N atoms as ring members and is optionally fused to an additional phenyl or a 5-6 membered heteroaryl ring, and wherein the 5-membered heteroaryl ring and optional fused phenyl or 5-6 membered heteroaryl ring are each optionally substituted with one or two groups selected from C₁₋₄ alkyl, C₁₋₄ alkoxy, —OH, halo, C₁₋₄ haloalkyl, NO₂, COOR, CONR₂, —SO₂R*, —NR₂, phenyl, and 5-6 membered heteroaryl;
wherein each R is independently selected from H and C₁₋₃ alkyl optionally substituted with OH, OR*, —NH₂, —NHR*, or —NR*₂; and
each R* is C₁₋₃ alkyl, optionally substituted with OH, oxo, C₁₋₂ alkoxy, or CN;
wherein two R, or two R″, or two R* on the same N can optionally be taken together to form a 4-7 membered heterocyclic ring, optionally containing an additional heteroatom selected from N, O and S as a ring member, and optionally substituted with one or two groups selected from halo, C₁₋₂ alkyl, OH, oxo, C₁₋₂ alkoxy, or CN;
   or
(b2) a metal-binding compound of the formula R³—NCS;
wherein R³ is H or an optionally substituted group selected from phenyl, 5-membered heteroaryl, 6-membered heteroaryl, C₁₋₃ haloalkyl, and C₁₋₆ alkyl,
wherein the optional substituents are one to three members selected from halo, —OH, C₁₋₃ alkyl, C₁₋₃ alkoxy, C₁₋₃ haloalkyl, NO₂, CN, COOR′, —N(R′)₂, CON(R′)₂, phenyl, 5-membered heteroaryl, 6-membered heteroaryl, and C₁₋₆ alkyl, wherein the phenyl, 5-membered heteroaryl, 6-membered heteroaryl, and C₁₋₆ alkyl are each optionally substituted with one or two members selected from halo, —OH, C₁₋₃ alkyl, C₁₋₃ alkoxy, C₁₋₃ haloalkyl, NO₂, CN, COOR′, —N(R′)₂, and CON(R′)₂;
where each R′ is independently H or C₁₋₃ alkyl;
wherein two R′ on the same N can optionally be taken together to form a 4-7 membered heterocyclic ring, optionally containing an additional heteroatom selected from N, O and S as a ring member, and optionally substituted with one or two groups selected from halo, C₁₋₂ alkyl, OH, oxo, C₁₋₂ alkoxy, or CN.

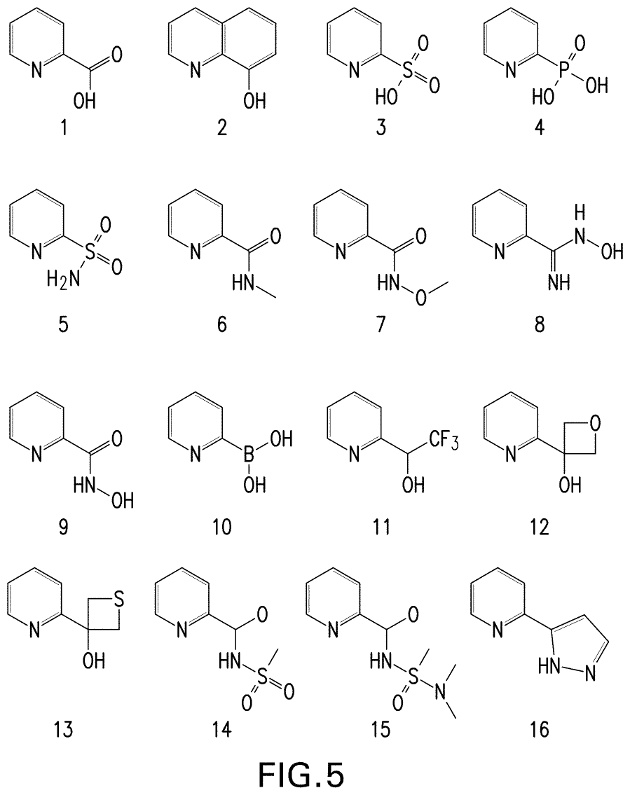

Some non-limiting examples of metal-binding NTMs that can be installed on N-terminus of a peptide are shown in FIGS. 2-4. Other examples include metal-binding isosteres of picolinic acid as provided in Dick B L, Cohen S M. Metal-Binding Isosteres as New Scaffolds for Metalloenzyme Inhibitors. Inorg Chem. 2018 Aug. 6; 57(15):9538-9543 (see FIG. 5), and derivatives of Famotidine, potent inhibitor of carbonic anhydrase II (Angeli A, Ferraroni M, Supuran C T. Famotidine, an Antiulcer Agent, Strongly Inhibits Helicobacter pylori and Human Carbonic Anhydrases. ACS Med Chem Lett. 2018 Sep. 4; 9(10):1035-1038).

In some embodiments, an N-terminal modifier agent used to modify the NTAA of a peptide, or an NTM group, comprises a chemical moiety that is a potent inhibitor of a metalloenzyme used as a binding agent that specifically binds to the modified NTAA of the peptide. In other embodiments, the N-terminal modifier agent or the NTM group comprises a chemical moiety that is a derivative of the metalloenzyme inhibitor. A metalloprotein binder provided herein would preferably have several of the following characteristics. In a preferred embodiment, it recognizes and binds to the modified NTAA residue (NTM-P1 residue) with a high affinity and specificity. In some embodiments, instead of binding to a single specific amino acid residue, a metalloprotein binder specifically binds independently to structurally similar modified NTAA residues, for example, to small hydrophobic amino acid residues modified with a N-terminal modifier agent or to negatively charged residues modified with a N-terminal modifier agent. At the same time, interaction with P2 amino acid of the peptide is limited, so that the binding affinity of the binder to the NTM-P1 residue does not depend significantly on P2 residue. In some embodiments, binding affinity and/or specificity between a metalloprotein binder and a NTM-P1 residue of the peptide is predominantly or substantially determined by interaction between the metalloprotein binder and the NTM-P1 residue of the peptide. In some embodiments, binding affinity and/or specificity between a metalloprotein binder and a NTM-P1 residue of the peptide differs no more than 3 fold, no more than 2 fold or no more than 1.5 fold depending on identity of the P2 residue of the peptide. In some preferred embodiments, a metalloprotein binder possesses additional characteristics, such as monomeric structure, ease of production, limited number of cysteines (preferably less than two Cys residues), high stability (thermal or in the presence of a detergent), limited post-translational modifications (e.g., glycosylation, phosphorylation), stable tertiary structure upon genetic manipulation, and compatibility with phage display or other protein engineering platforms that enable selection of preferred variants. Many classes of metalloenzymes can be evolved to be utilized in the methods disclosed herein. Importantly, high affinity and specificity towards NTM-P1 residue of the peptide are to be achieved by selecting a combination of a metalloenzyme and specific NTM.

Several high-throughput screening methods known in the art can be used to select metalloenzyme variants with desired specificity by utilizing a panel of metalloenzyme mutants and, optionally, a panel of structurally-related NTMs. To start the maturation process, an appropriate metalloenzyme scaffold may be chosen based on size of the binding pocket that should accommodate NTM-P1. Another important consideration is knowledge about potential evolvability of P1/P2 specificity based on natural substrates or known inhibitors of metalloenzymes. Based on the knowledge about natural substrates or known inhibitors, several classes of metalloenzymes can be considered as desired candidates for specific binders. First, metalloproteases, such as dipeptidyl peptidases or aminopeptidases, are good candidates, since they are known to have peptides as substrates, possess substrates specificity, but at the same time structurally-related variants of these enzymes have diverse specificity for substrates. Aminopeptidases catalyze the cleavage of specific amino acids from the N-terminus of peptides, so their binding pocket can be evolved to recognize specific NTM-P1 groups. Dipeptidyl peptidases catalyze the cleavage of specific dipeptides from the N-terminus of peptides, so they can also be evolved to recognize specific NTM-P1 groups if the size of NTM is similar to the size of an amino acid. Examples of suitable aminopeptidase scaffolds include M1 aminopeptidases, such as aminopeptidase N, leucyl-, arginine-, methionyl-, aspartyl-, alanyl-, glutamyl-, prolyl-, and cystinyl-aminopeptidases. Some of the suitable dipeptidyl peptidase scaffolds include Cathepsin C (dipeptidyl peptidase-1), Dipeptidyl-peptidase II, dipeptidyl peptidase-3, dipeptidyl peptidase-4, dipeptidyl peptidase-6, dipeptidyl peptidase-7, dipeptidyl peptidase-8, dipeptidyl peptidase-9, dipeptidyl peptidase-10. Other suitable metalloprotease scaffolds include metzincins (astacins, serralysins, snapalysins, leishmanolysins, pappalysins, archaemetzincins, fragilysins, cholerilysins, toxilysins, igalysins, matrix metalloproteases (MMPs), collagenases, stromelysins, gelatinases, ADAM proteases), gluzincins, thermolysins, minigluzincins, cowrins, M48/M56 integral membrane MMPs, leukotriene A-4 hydrolases, anthrax lethal factor, clostridial neurotoxins, neprilysins, inverzincins, aspzincins, funnelins, carboxypeptidases. Other suitable metalloenzyme scaffolds include peptide deformylases (zinc, nickel, cobalt, and iron), histone deacetylases, carbonic anhydrases, phospholipases, oxidoreductases (iron), cytochromes, prostaglandin-endoperoxide synthases (COX1/2), alcohol dehydrogenases, sorbitol dehydrogenases, transcription factors with zinc finger domains or ring finger domains, metal responsive transcription factor-1, metal transporters (such as ZnuA-Syn, PsaA, TroA, ZinT, MntC), metallo-beta lactamase.

In some embodiments, suitable scaffolds include synthetic or artificial metalloenzymes, where known metal-binding motifs are introduced into “naive” scaffolds. There are numerous known metal binding motifs that can be used for incorporation into “naive” scaffolds, such as HEXXH or HEXGHXXGXXH for zinc ion. Other non-limiting examples include Zn²⁺ binding motifs provided in Andreini C, et al., Zinc through the three domains of life. J Proteome Res. 2006 November; 5(11):3173-8. Several public databases are known in the art that provide information on metal-binding sites detected in the three-dimensional (3D) structures of biological macromolecules. Examples include the MetalPDB database presented in Putignano V, et al., MetalPDB in 2018: a database of metal sites in biological macromolecular structures. Nucleic Acids Res. 2018 Jan. 4; 46(D1):D459-D464, or the MetalMine database (Kensuke Nakamura et al., MetalMine: a database of functional metal-binding sites in proteins; Plant Biotechnology 26, 517-521 (2009)). There are a number of approaches known in the art for making artificial metalloenzymes, for example, Schwizer F, Okamoto Y, Heinisch T, Gu Y, Pellizzoni M M, Lebrun V, Reuter R, Köhler V, Lewis J C, Ward T R. Artificial Metalloenzymes: Reaction Scope and Optimization Strategies. Chem Rev. 2018 Jan. 10; 118(1):142-231; Reetz M T. Directed Evolution of Artificial Metalloenzymes: A Universal Means to Tune the Selectivity of Transition Metal Catalysts? Acc Chem Res. 2019 Feb. 19; 52(2):336-344; Liang A D, Serrano-Plana J, Peterson R L, Ward T R. Artificial Metalloenzymes Based on the Biotin-Streptavidin Technology: Enzymatic Cascades and Directed Evolution. Acc Chem Res. 2019 Mar. 19; 52(3):585-595, incorporated by reference herein. For example, lipocalins or streptavidin can be used as scaffolds for artificial metalloenzymes. In some embodiments, DNA/RNA scaffolds can be used for metalloenzymes, such as zinc binding ribozymes or zinc/peptide binding aptamers.

Various metal ions can be utilized in the methods disclosed herein. In some embodiments, one of divalent metal ions, such as Mn(II), Fe(II), Co(II), Ni(II) or Zn(II) is used together with engineered metalloenzymes and NTMs that bind such divalent metal ion with a high affinity. Numerous examples of natural metalloenzymes with intrinsic specificity to these divalent metal ions are described in the art. For some metalloenzyme scaffolds, for example for metallo-aminopeptidases, several different divalent metal ions can be used interchangeably, because such metalloenzymes were shown to be active when reconstituted with any of these different divalent metal ions (Rouffet M, Cohen S M. Emerging trends in metalloenzyme inhibition. Dalton Trans. 2011 Apr. 14; 40(14):3445-54).

During binding reaction between a metalloprotein binder and an NTM-P1 group of a peptide to be analyzed, the corresponding metal ion can be added to the reaction or can be comprised in the metalloprotein binder (as a part of the metalloenzyme holoprotein).

Numerous specific details are set forth in the following description in order to provide a thorough understanding of the present disclosure. These details are provided for the purpose of example and the claimed subject matter may be practiced according to the claims without some or all of these specific details. It is to be understood that other embodiments can be used and structural changes can be made without departing from the scope of the claimed subject matter. It should be understood that the various features and functionality described in one or more of the individual embodiments are not limited in their applicability to the particular embodiment with which they are described. They instead can be applied, alone or in some combination, to one or more of the other embodiments of the disclosure, whether or not such embodiments are described, and whether or not such features are presented as being a part of a described embodiment. For the purpose of clarity, technical material that is known in the technical fields related to the claimed subject matter has not been described in detail so that the claimed subject matter is not unnecessarily obscured.

All publications, including patent documents, scientific articles and databases, referred to in this application are incorporated by reference in their entireties for all purposes to the same extent as if each individual publication were individually incorporated by reference. Citation of the publications or documents is not intended as an admission that any of them is pertinent prior art, nor does it constitute any admission as to the contents or date of these publications or documents.

All headings are for the convenience of the reader and should not be used to limit the meaning of the text that follows the heading, unless so specified.

### Definitions

Unless defined otherwise, all technical and scientific terms used herein have the same meaning as is commonly understood by one of ordinary skill in the art to which the present disclosure belongs. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, the singular forms “a,” “an” and “the” include plural referents unless the context clearly dictates otherwise. Thus, for example, reference to “a peptide” includes one or more peptides, or mixtures of peptides. Also, and unless specifically stated or obvious from context, as used herein, the term “or” is understood to be inclusive and covers both “or” and “and”.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X.

As used herein, the term “sample” refers to anything which may contain an analyte for which an analyte assay is desired. As used herein, a “sample” can be a solution, a suspension, liquid, powder, a paste, aqueous, non-aqueous or any combination thereof. The sample may be a biological sample, such as a biological fluid or a biological tissue. Examples of biological fluids include urine, blood, plasma, serum, saliva, semen, stool, sputum, cerebral spinal fluid, tears, mucus, amniotic fluid or the like. Biological tissues are aggregate of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s).

In some embodiments, the sample is a biological sample. A biological sample of the present disclosure encompasses a sample in the form of a solution, a suspension, a liquid, a powder, a paste, an aqueous sample, or a non-aqueous sample. As used herein, a “biological sample” includes any sample obtained from a living or viral (or prion) source or other source of macromolecules and biomolecules, and includes any cell type or tissue of a subject from which nucleic acid, protein and/or other macromolecule can be obtained. The term “subject” includes a mammal. The biological sample can be a sample obtained directly from a biological source or a sample that is processed. For example, isolated nucleic acids that are amplified constitute a biological sample. Biological samples include, but are not limited to, body fluids, such as blood, plasma, serum, cerebrospinal fluid, synovial fluid, urine and sweat, tissue and organ samples from animals and plants and processed samples derived therefrom. In some embodiments, the sample can be derived from a tissue or a body fluid, for example, a connective, epithelium, muscle or nerve tissue; a tissue selected from the group consisting of brain, lung, liver, spleen, bone marrow, thymus, heart, lymph, blood, bone, cartilage, pancreas, kidney, gall bladder, stomach, intestine, testis, ovary, uterus, rectum, nervous system, gland, and internal blood vessels; or a body fluid selected from the group consisting of blood, urine, saliva, bone marrow, sperm, an ascitic fluid, and subfractions thereof, e.g., serum or plasma.

The terms “level” or “levels” are used to refer to the presence and/or amount of a target, e.g., a substance or an organism that is part of the etiology of a disease or disorder, and can be determined qualitatively or quantitatively. A “qualitative” change in the target level refers to the appearance or disappearance of a target that is not detectable or is present in samples obtained from normal controls. A “quantitative” change in the levels of one or more targets refers to a measurable increase or decrease in the target levels when compared to a healthy control.

As used herein, the term “macromolecule” encompasses large molecules composed of smaller subunits. Examples of macromolecules include, but are not limited to peptides, nucleic acids, carbohydrates, lipids, macrocycles, or a combination or complex thereof. A macromolecule also includes a chimeric macromolecule composed of a combination of two or more types of macromolecules, covalently linked together (e.g., a peptide linked to a nucleic acid). A macromolecule assembly may be composed of the same type of macromolecule (e.g., protein-protein) or of two or more different types of macromolecules (e.g., protein-DNA).

The term “peptide” is used interchangeably with the term “peptide” and encompasses peptides and proteins, and refers to a molecule comprising a chain of two or more amino acids joined by peptide bonds. In some embodiments, a peptide comprises 2 to 50 amino acids. In some embodiments, a peptide does not comprise a secondary, tertiary, or higher structure. In some embodiments, the peptide is a protein. In some embodiments, a protein comprises 30 or more amino acids, e.g. having more than 50 amino acids. In some embodiments, in addition to a primary structure, a protein comprises a secondary, tertiary, or higher structure. The amino acids of the peptides are most typically L-amino acids, but may also be D-amino acids, modified amino acids, amino acid analogs, amino acid mimetics, or any combination thereof. Peptides may be naturally occurring, synthetically produced, or recombinantly expressed. Peptides may be synthetically produced, isolated, recombinantly expressed, or be produced by a combination of methodologies as described above. Peptides may also comprise additional groups modifying the amino acid chain, for example, functional groups added via post-translational modification. The polymer may be linear or branched, it may comprise modified amino acids, and it may be interrupted by non-amino acids. The term also encompasses an amino acid polymer that has been modified naturally or by intervention; for example, disulfide bond formation, glycosylation, lipidation, acetylation, phosphorylation, or any other manipulation or modification, such as conjugation with a labeling component.

As used herein, the term “amino acid” refers to an organic compound comprising an amine group, a carboxylic acid group, and a side-chain specific to each amino acid, which serve as a monomeric subunit of a peptide. An amino acid includes the 20 standard, naturally occurring or canonical amino acids as well as non-standard amino acids. The standard, naturally-occurring amino acids include Alanine (A or Ala), Cysteine (C or Cys), Aspartic Acid (D or Asp), Glutamic Acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr). An amino acid may be an L-amino acid or a D-amino acid. Non-standard amino acids may be modified amino acids, amino acid analogs, amino acid mimetics, non-standard proteinogenic amino acids, or non-proteinogenic amino acids that occur naturally or are chemically synthesized. Examples of non-standard amino acids include, but are not limited to, selenocysteine, pyrrolysine, and N-formylmethionine, β-amino acids, Homo-amino acids, Proline and Pyruvic acid derivatives, 3-substituted alanine derivatives, glycine derivatives, ring-substituted phenylalanine and tyrosine derivatives, N-methyl amino acids.

As used herein, the term “metalloenzyme” refers to a macromolecule containing a binding pocket that incorporates a metal ion, which plays a crucial role in recognition of a metalloenzyme's substrate and is directly bound to the macromolecule or to a macromolecule-bound prosthetic group. Non-limiting examples of macromolecular scaffolds for metalloenzymes include peptides or polynucleotides. There are natural metalloenzymes (such as various metalloproteins, including metalloproteases), or artificial metalloenzymes. Artificial metalloenzymes result from anchoring a metal-containing moiety within a macromolecular scaffold (preferably, peptide or polynucleotide). Metal ions in metalloenzymes are usually coordinated by nitrogen, oxygen or sulfur centers with very high association constants (Kₐ>10¹⁰ M⁻¹, and often Kₐ>10¹⁵ M⁻¹).

As used herein, the term “metalloprotein binder” refers to an engineered (non-natural) protein-based binder derived from a metalloenzyme by mutating a substrate-binding pocket of the metalloenzyme to accommodate a modified N-terminal amino acid of a peptide substrate (Z-P1).

As used herein, the term “N-terminal modifier agent” refers to a small molecule that interacts with a peptide to be analyzed and modifies (or functionalizes) the N-terminal amino acid residue (P1 residue) of the peptide. The interaction between N-terminal modifier agent and peptide creates an N-terminal modification (NTM) of the P1 residue, forming NTM-P1 group at the N-terminus of the peptide. The disclosed herein N-terminal modifier agents and/or NTMs incorporate at least one metal binding group in order to coordinate a metal ion.

As used herein, the term “post-translational modification” refers to modifications that occur on a peptide after its translation, e.g., translation by ribosomes, is complete. A post-translational modification may be a covalent chemical modification or enzymatic modification. A post-translational modification includes modifications of the amino terminus and/or the carboxyl terminus of a peptide. Modifications of the terminal amino group include, but are not limited to, des-amino, N-lower alkyl, N-di-lower alkyl, and N-acyl modifications. The term post-translational modification can also include peptide modifications that include one or more detectable labels.

As used herein, the term “binding agent” or “binder” refers to a nucleic acid molecule, a peptide, a protein, carbohydrate, or a small molecule that binds to, associates, unites with, recognizes, or combines with a binding target, e.g., a peptide or a component or feature of a peptide. A binding agent may form a covalent association or non-covalent association with the peptide or component or feature of a peptide. A binding agent may also be a chimeric binding agent, composed of two or more types of molecules, such as a nucleic acid molecule-peptide chimeric binding agent or a carbohydrate-peptide chimeric binding agent. A binding agent may be a naturally occurring, synthetically produced, or recombinantly expressed molecule. A binding agent may bind to a single monomer or subunit of a peptide (e.g., a single amino acid of a peptide) or bind to a plurality of linked subunits of a peptide (e.g., a di-peptide, tri-peptide, or higher order peptide of a longer peptide, peptide, or protein molecule). A binding agent may bind to a linear molecule or a molecule having a three-dimensional structure (also referred to as conformation). For example, an antibody binding agent may bind to linear peptide, peptide, or protein, or bind to a conformational peptide, peptide, or protein. A binding agent may preferably bind to a chemically modified or labeled amino acid (e.g., an amino acid that has been labeled by a chemical reagent) over a non-modified or unlabeled amino acid, such as terminal amino acid. For example, a binding agent may preferably bind to an N-terminal amino acid (NTAA) residue of a peptide that has been labeled or modified over an N-terminal amino acid (NTAA) residue that is unlabeled or unmodified. A binding agent may bind to a post-translational modification of a peptide molecule. A binding agent may exhibit selective binding to a component or feature of a peptide (e.g., a binding agent may selectively bind to one of the 20 possible natural amino acid residues and bind with very low affinity or not at all to the other 19 natural amino acid residues). A binding agent may exhibit less selective binding, where the binding agent is capable of binding or configured to bind to a plurality of components or features of a peptide (e.g., a binding agent may bind with similar affinity to two or more different amino acid residues). A binding agent may comprise a coding tag, which may be joined to the binding agent by a linker.

As used herein, the term “linker” refers to one or more of a nucleotide, a nucleotide analog, an amino acid, a peptide, a peptide, a polymer, or a non-nucleotide chemical moiety that is used to join two molecules. A linker may be used to join a binding agent with a coding tag, a recording tag with a peptide, a peptide with a support, a recording tag with a solid support, etc. In certain embodiments, a linker joins two molecules via enzymatic reaction or chemistry reaction (e.g., click chemistry).

The term “ligand” as used herein refers to any molecule or moiety connected to the compounds described herein. “Ligand” may refer to one or more ligands attached to a compound. In some embodiments, the ligand is a pendant group or binding site (e.g., the site to which the binding agent binds).

The terminal amino acid at one end of a peptide or peptide chain that has a free amino group is referred to herein as the “N-terminal amino acid” (NTAA). The terminal amino acid at the other end of the chain that has a free carboxyl group is referred to herein as the “C-terminal amino acid” (CTAA). The amino acids making up a peptide may be numbered in order, with the peptide being “n” amino acids in length. As used herein, NTAA is considered the n^{th} amino acid (also referred to herein as the “n NTAA”). Using this nomenclature, the next amino acid is the n−1 amino acid, then the n−2 amino acid, and so on down the length of the peptide from the N-terminal end to C-terminal end. In certain embodiments, an NTAA, CTAA, or both may be modified or labeled with a chemical moiety.

As used herein, the term “barcode” refers to a nucleic acid molecule of about 2 to about 30 bases (e.g., 2, 3, 4, 5, 6, 7, 8, 9, 10, 11, 12, 13, 14, 15, 16, 17, 18, 19, 20, 21, 22, 23, 24, 25, 26, 27, 28, 29 or 30 bases) providing a unique identifier tag or origin information for a peptide, a binding agent, a set of binding agents from a binding cycle, a sample peptides, a set of samples, peptides within a compartment (e.g., droplet, bead, or separated location), peptides within a set of compartments, a fraction of peptides, a library of peptides, or a library of binding agents. A barcode can be an artificial sequence or a naturally occurring sequence. In certain embodiments, each barcode within a population of barcodes is different. In other embodiments, a portion of barcodes in a population of barcodes is different, e.g., at least about 10%, 15%, 20%, 25%, 30%, 35%, 40%, 45%, 50%, 55%, 60%, 65%, 70%, 75%, 80%, 85%, 90%, 95%, 97%, or 99% of the barcodes in a population of barcodes is different. A population of barcodes may be randomly generated or non-randomly generated. In certain embodiments, a population of barcodes are error-correcting or error-tolerant barcodes. Barcodes can be used to computationally deconvolute the multiplexed sequencing data and identify sequence reads derived from an individual peptide, sample, library, etc. A barcode can also be used for deconvolution of a collection of peptides that have been distributed into small compartments for enhanced mapping. For example, rather than mapping a peptide back to the proteome, the peptide is mapped back to its originating protein molecule or protein complex.

As used herein, the term “coding tag” refers to a polynucleotide with any suitable length, e.g., a nucleic acid molecule of about 2 bases to about 100 bases, including any integer including 2 and 100 and in between, that comprises identifying information for its associated binding agent. A “coding tag” may also be made from a “sequenceable polymer” (see, e.g., Niu et al., 2013, Nat. Chem. 5:282-292; Roy et al., 2015, Nat. Commun. 6:7237). A coding tag may comprise a barcode sequence, which is optionally flanked by one spacer on one side or optionally flanked by a spacer on each side. A coding tag may also be comprised of an optional UMI and/or an optional binding cycle-specific barcode. A coding tag may be single stranded or double stranded. A double stranded coding tag may comprise blunt ends, overhanging ends, or both. A coding tag may refer to the coding tag that is directly attached to a binding agent, to a complementary sequence hybridized to the coding tag directly attached to a binding agent (e.g., for double stranded coding tags), or to coding tag information present in an extended recording tag. In certain embodiments, a coding tag may further comprise a binding cycle specific spacer or barcode, a unique molecular identifier, a universal priming site, or any combination thereof.

As used herein, the term “spacer” (Sp) refers to a nucleic acid molecule of about 1 base to about 20 bases (e.g., 1, 2, 3, 4, 5, 6, 7, 8, 9, 10, 11, 12, 13, 14, 15, 16, 17, 18, 19, or 20 bases) in length that is present on a terminus of a recording tag or coding tag. In certain embodiments, a spacer sequence flanks a barcode sequence of a coding tag on one end or both ends. Following binding of a binding agent to a peptide, annealing between complementary spacer sequences on their associated coding tag and recording tag, respectively, allows transfer of binding information through a primer extension reaction or ligation to the recording tag. Sp′ refers to spacer sequence complementary to Sp. Preferably, spacer sequences within a library of binding agents possess the same number of bases. A common (shared or identical) spacer may be used in a library of binding agents. A spacer sequence may have a “cycle specific” sequence in order to track binding agents used in a particular binding cycle. The spacer sequence (Sp) can be constant across all binding cycles, be specific for a particular class of peptides, or be binding cycle number specific. In some embodiments, only the sequential binding of correct cognate pairs results in interacting spacer elements and effective primer extension. A spacer sequence may comprise sufficient number of bases to anneal to a complementary spacer sequence in a recording tag to initiate a primer extension (also referred to as polymerase extension) reaction, or provide a “splint” for a ligation reaction, or mediate a “sticky end” ligation reaction.

As used herein, the term “recording tag” refers to a nucleic acid molecule, or a sequenceable polymer molecule (see, e.g., Niu et al., 2013, Nat. Chem. 5:282-292; Roy et al., 2015, Nat. Commun. 6:7237; Lutz, 2015, Macromolecules 48:4759-4767; each of which are incorporated by reference in its entirety) to which identifying information of a coding tag can be transferred. Identifying information can comprise any information characterizing a molecule such as information pertaining to sample, fraction, partition, spatial location, interacting neighboring molecule(s), cycle number, etc. Additionally, the presence of UMI information can also be classified as identifying information. In certain embodiments, after a binding agent binds to a peptide, information from a coding tag linked to a binding agent can be transferred to the recording tag associated with the peptide while the binding agent is bound to the peptide. In other embodiments, after a binding agent binds to a peptide, information from a recording tag associated with the peptide can be transferred to the coding tag linked to the binding agent while the binding agent is bound to the peptide. A recording tag may be directly linked to a peptide, linked to a peptide via a multifunctional linker, or associated with a peptide by virtue of its proximity (or co-localization) on a support. A recording tag may be linked via its 5′ end or 3′ end or at an internal site, as long as the linkage is compatible with the method used to transfer coding tag information to the recording tag or vice versa. A recording tag may further comprise other functional components, e.g., a universal priming site, unique molecular identifier, a barcode (e.g., a sample barcode, a fraction barcode, spatial barcode, a compartment tag, etc.), a spacer sequence that is complementary to a spacer sequence of a coding tag, or any combination thereof. The spacer sequence of a recording tag is preferably at the 3′-end of the recording tag in embodiments where polymerase extension is used to transfer coding tag information to the recording tag.

As used herein, the term “primer extension”, also referred to as “polymerase extension”, refers to a reaction catalyzed by a nucleic acid polymerase (e.g., DNA polymerase) whereby a nucleic acid molecule (e.g., oligonucleotide primer, spacer sequence) that anneals to a complementary strand is extended by the polymerase, using the complementary strand as template.

As used herein, the term “unique molecular identifier” or “UMI” refers to a nucleic acid molecule of about 3 to about 40 bases (3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 bases) in length providing a unique identifier tag for each macromolecule, peptide or binding agent to which the UMI is linked. A peptide UMI can be used to computationally deconvolute sequencing data from a plurality of extended recording tags to identify extended recording tags that originated from an individual peptide. A peptide UMI can be used to accurately count originating peptide molecules by collapsing NGS reads to unique UMIs. A binding agent UMI can be used to identify each individual molecular binding agent that binds to a particular peptide.

As used herein, the term “universal priming site” or “universal primer” or “universal priming sequence” refers to a nucleic acid molecule, which may be used for library amplification and/or for sequencing reactions. A universal priming site may include, but is not limited to, a priming site (primer sequence) for PCR amplification, flow cell adaptor sequences that anneal to complementary oligonucleotides on flow cell surfaces enabling bridge amplification in some next generation sequencing platforms, a sequencing priming site, or a combination thereof. The term “forward” when used in context with a “priming site” or “primer” may also be referred to as “5′” or “sense”. The term “reverse” when used in context with a “priming site” or “primer” may also be referred to as “3′” or “antisense”.

As used herein, the term “extended recording tag” refers to a recording tag to which information of at least one binding agent's coding tag (or its complementary sequence) has been transferred following binding of the binding agent to a peptide. Information of the coding tag may be transferred to the recording tag directly (e.g., ligation) or indirectly (e.g., primer extension). Information of a coding tag may be transferred to the recording tag enzymatically or chemically. An extended recording tag may comprise binding agent information of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more coding tags. The base sequence of an extended recording tag may reflect the temporal and sequential order of binding of the binding agents identified by their coding tags, may reflect a partial sequential order of binding of the binding agents identified by the coding tags, or may not reflect any order of binding of the binding agents identified by the coding tags. In certain embodiments where the extended recording tag does not represent the peptide sequence being analyzed with 100% identity, errors may be due to off-target binding by a binding agent, or to a “missed” binding cycle.

As used herein, the term “solid support”, “solid surface”, or “solid substrate”, or “sequencing substrate”, or “substrate” refers to any solid material, including porous and non-porous materials, to which a peptide can be associated directly or indirectly, by any means known in the art, including covalent and non-covalent interactions, or any combination thereof. A solid support may be two-dimensional (e.g., planar surface) or three-dimensional (e.g., gel matrix or bead). A solid support can be any support surface including, but not limited to, a bead, a microbead, an array, a glass surface, a silicon surface, a plastic surface, a filter, a membrane, a PTFE membrane, a PTFE membrane, a nitrocellulose membrane, a nitrocellulose-based polymer surface, nylon, a silicon wafer chip, a flow through chip, a flow cell, a biochip including signal transducing electronics, a channel, a microtiter well, an ELISA plate, a spinning interferometry disc, a nitrocellulose membrane, a nitrocellulose-based polymer surface, a polymer matrix, a nanoparticle, or a microsphere. Materials for a solid support include but are not limited to acrylamide, agarose, cellulose, dextran, nitrocellulose, glass, gold, quartz, polystyrene, polyethylene vinyl acetate, polypropylene, polyester, polymethacrylate, polyacrylate, polyethylene, polyethylene oxide, polysilicates, polycarbonates, poly vinyl alcohol (PVA), Teflon, fluorocarbons, nylon, silicon rubber, polyanhydrides, polyglycolic acid, polyvinylchloride, polylactic acid, polyorthoesters, functionalized silane, polypropylfumerate, collagen, glycosaminoglycans, polyamino acids, dextran, or any combination thereof. Solid supports further include thin film, membrane, fibers, shaped polymers such as tubes, particles, beads, microspheres, microparticles, or any combination thereof. For example, when solid surface is a bead, the bead can include, but is not limited to, a ceramic bead, a polystyrene bead, a polymer bead, a polyacrylate bead, a methylstyrene bead, an agarose bead, a cellulose bead, a dextran bead, an acrylamide bead, a solid core bead, a porous bead, a paramagnetic bead, a glass bead, a controlled pore bead, a silica-based bead, or any combinations thereof. A bead may be spherical or an irregularly shaped. A bead or support may be porous. In certain embodiments, beads range in size from about 0.2 micron to about 200 microns, or from about 0.5 micron to about 5 micron. In some embodiments, beads can be about 1, 1.5, 2, 2.5, 5, 7, 10, 15, or 20 μm in diameter. In certain embodiments, “a bead” solid support may refer to an individual bead or a plurality of beads. In some embodiments, the solid surface is a nanoparticle. In certain embodiments, the nanoparticles range in size from about 1 nm to about 500 nm in diameter, for example, between about 1 nm and about 50 nm, between about 10 nm and about 50 nm, between about 10 nm and about 200 nm, between about 50 nm and about 100 nm, between about 50 nm and about 200 nm, between about 100 nm and about 200 nm, or between about 200 nm and about 500 nm in diameter.

As used herein, the term “nucleic acid molecule” or “polynucleotide” refers to a single- or double-stranded polynucleotide containing deoxyribonucleotides or ribonucleotides that are linked by 3′-5′ phosphodiester bonds, as well as polynucleotide analogs. A nucleic acid molecule includes, but is not limited to, DNA, RNA, and cDNA. A polynucleotide analog may possess a backbone other than a standard phosphodiester linkage found in natural polynucleotides and, optionally, a modified sugar moiety or moieties other than ribose or deoxyribose. Polynucleotide analogs contain bases capable of hydrogen bonding by Watson-Crick base pairing to standard polynucleotide bases, where the analog backbone presents the bases in a manner to permit such hydrogen bonding in a sequence-specific fashion between the oligonucleotide analog molecule and bases in a standard polynucleotide. Examples of polynucleotide analogs include, but are not limited to xeno nucleic acid (XNA), bridged nucleic acid (BNA), glycol nucleic acid (GNA), peptide nucleic acids (PNAs), γPNAs, morpholino polynucleotides, locked nucleic acids (LNAs), threose nucleic acid (TNA), 2′-O-Methyl polynucleotides, and boronophosphate polynucleotides. A polynucleotide analog may possess purine or pyrimidine analogs, including for example, 7-deaza purine analogs, 8-halopurine analogs, 5-halopyrimidine analogs, or universal base analogs that can pair with any base, including hypoxanthine, nitroazoles, isocarbostyril analogues, azole carboxamides, and aromatic triazole analogues, or base analogs with additional functionality, such as a biotin moiety for affinity binding. In some embodiments, the nucleic acid molecule or oligonucleotide is a modified oligonucleotide. In some embodiments, the nucleic acid molecule or oligonucleotide is a DNA with pseudo-complementary bases, a DNA with protected bases, an RNA molecule, a BNA molecule, an XNA molecule, a LNA molecule, a PNA molecule, a γPNA molecule, or a morpholino DNA, or a combination thereof. In some embodiments, the nucleic acid molecule or oligonucleotide is backbone modified, sugar modified, or nucleobase modified. In some embodiments, the nucleic acid molecule or oligonucleotide has nucleobase protecting groups such as Alloc, electrophilic protecting groups such as thiranes, acetyl protecting groups, nitrobenzyl protecting groups, sulfonate protecting groups, or traditional base-labile protecting groups.

As used herein, “nucleic acid sequencing” means the determination of the order of nucleotides in a nucleic acid molecule or a sample of nucleic acid molecules, and “peptide sequencing” refers to the determination of the order of amino acids in a peptide molecule or a sample of peptide molecules.

As used herein, “next generation sequencing” refers to high-throughput sequencing methods that allow the sequencing of millions to billions of molecules in parallel. Examples of next generation sequencing methods include sequencing by synthesis, sequencing by ligation, sequencing by hybridization, polony sequencing, ion semiconductor sequencing, and pyrosequencing. By attaching primers to a solid substrate and a complementary sequence to a nucleic acid molecule, a nucleic acid molecule can be hybridized to the solid substrate via the primer and then multiple copies can be generated in a discrete area on the solid substrate by using polymerase to amplify (these groupings are sometimes referred to as polymerase colonies or polonies). Consequently, during the sequencing process, a nucleotide at a particular position can be sequenced multiple times (e.g., hundreds or thousands of times)—this depth of coverage is referred to as “deep sequencing.” Examples of high throughput nucleic acid sequencing technology include platforms provided by Illumina, BGI, Qiagen, Thermo-Fisher, and Roche, including formats such as parallel bead arrays, sequencing by synthesis, sequencing by ligation, capillary electrophoresis, electronic microchips, “biochips,” microarrays, parallel microchips, and single-molecule arrays (See e.g., Service, Science (2006) 311:1544-1546).

As used herein, “analyzing” the peptide means to identify, detect, quantify, characterize, distinguish, or a combination thereof, all or a portion of the components of the peptide. For example, analyzing a peptide includes determining all or a portion of the amino acid sequence (contiguous or non-continuous) of the peptide. Analyzing a peptide also includes partial identification of a component of the peptide. Analyzing the peptide also includes obtaining an information regarding at least one amino acid residue of the peptide. As used herein, “obtaining an information regarding at least one amino acid residue” refers to identifying, detecting, quantifying, characterizing, distinguishing, or a combination thereof, at least one amino acid residue of the peptide. Obtaining an information regarding at least one amino acid residue also includes partial identification of the amino acid residue of the peptide. For example, partial identification of amino acids in the peptide sequence can identify an amino acid in the peptide sequence as belonging to a subset of possible amino acids. Analysis typically begins with analysis of the n NTAA, and then proceeds to the next amino acid of the peptide (i.e., n−1, n−2, n−3, and so forth). This is accomplished by elimination of the n NTAA, thereby converting the n−1 amino acid of the peptide to an N-terminal amino acid (referred to herein as the “n−1 NTAA”). Analyzing the peptide may include combining different types of analysis, for example obtaining epitope information, amino acid sequence information, post-translational modification information, or any combination thereof.

As used herein, the term “detectable label” refers to a substance which can indicate the presence of another substance when associated with it. The detectable label can be a substance that is linked to or incorporated into the substance to be detected. In some embodiments, a detectable label is suitable for allowing for detection and also quantification, for example, a detectable label that emitting a detectable and measurable signal. Detectable labels include any labels that can be utilized and are compatible with the provided peptide analysis assay format and include, but not limited to, a bioluminescent label, a biotin/avidin label, a chemiluminescent label, a chromophore, a coenzyme, a dye, an electro-active group, an electrochemiluminescent label, an enzymatic label (e.g. alkaline phosphatase, luciferase or horseradish peroxidase), a fluorescent label, a latex particle, a magnetic particle, a metal, a metal chelate, a phosphorescent dye, a protein label, a radioactive element or moiety, and a stable radical.

The term “unmodified” (also “wild-type” or “native”) as used herein is used in connection with biological materials such as nucleic acid molecules and proteins (e.g., metalloprotein binders), refers to those which are found in nature and not modified by human intervention.

The term “modified” or “engineered” (or “variant”, or “mutant”) as used in reference to nucleic acid molecules and protein molecules, e.g., an engineered metalloprotein binder, implies that such molecules are created by human intervention and/or they are non-naturally occurring. The variant, mutant or engineered metalloprotein binder is a polypeptide or peptide having an altered amino acid sequence, relative to an unmodified or wild-type protein, such as starting metalloenzyme scaffold, or a portion thereof. An engineered metalloprotein binder is a polypeptide or peptide which differs from a wild-type metalloenzyme scaffold sequence, or a portion thereof, by one or more amino acid substitutions, deletions, additions, or combinations thereof. Sequence of an engineered metalloprotein binder can contain 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more amino acid differences (e.g., mutations) compared to the sequence of starting metalloenzyme scaffold. An engineered metalloprotein binder generally exhibits at least 70%, 80%, 85%, 86%, 87%, 88%, 89%, 90%, 91%, 92%, 93%, 94%, 95%, 96%, 97%, 98%, 99% or more sequence identity to a corresponding wild-type starting metalloenzyme scaffold. An engineered metalloprotein binder can exhibit at least 70%, 80%, 85%, 86%, 87%, 88%, 89%, 90%, 91%, 92%, 93%, 94%, 95%, 96%, 97%, 98%, 99% or more sequence homology to a corresponding wild-type starting metalloenzyme scaffold. Non-naturally occurring amino acids as well as naturally occurring amino acids are included within the scope of permissible substitutions or additions. An engineered metalloprotein binder is not limited to any engineered binders made or generated by a particular method of making and includes, for example, an engineered metalloprotein binder made or generated by genetic selection, protein engineering, directed evolution, de novo recombinant DNA techniques, or combinations thereof. The term “variant” in the context of variant or engineered metalloprotein binder is not to be construed as imposing any condition for any particular starting composition or method by which the variant or engineered metalloprotein binder is created. Thus, variant or engineered metalloprotein binder denotes a composition and not necessarily a product produced by any given process. A variety of techniques including genetic selection, protein engineering, recombinant methods, chemical synthesis, or combinations thereof, may be employed.

In some embodiments, variants of a metalloprotein binder displaying only non-substantial or negligible differences in structure can be generated by making conservative amino acid substitutions in the engineered metalloprotein binder. By doing this, engineered metalloprotein binder variants that comprise a sequence having at least 90% (90%, 91%, 92%, 93%, 94%, 95%, 96%, 97%, 98%, and 99%) sequence identity with the engineered metalloprotein binder sequences can be generated, retaining at least one functional activity of the engineered metalloprotein binder, e.g., ability to specifically bind to the N-terminally modified target peptide. Examples of conservative amino acid changes are known in the art. Examples of non-conservative amino acid changes that are likely to cause major changes in protein structure are those that cause substitution of (a) a hydrophilic residue, e.g., serine or threonine, for (or by) a hydrophobic residue, e.g., leucine, isoleucine, phenylalanine, valine or alanine; (b) a cysteine or proline for (or by) any other residue; (c) a residue having an electropositive side chain, e.g., lysine, arginine, or histidine, for (or by) an electronegative residue, e.g., glutamic acid or aspartic acid; or (d) a residue having a bulky side chain, e.g., phenylalanine, for (or by) one not having a side chain, e g., glycine. Methods of making targeted amino acid substitutions, deletions, truncations, and insertions are generally known in the art. For example, amino acid sequence variants can be prepared by mutations in the DNA. Methods for polynucleotide alterations are well known in the art, for example, Kunkel et al. (1987) Methods in Enzymol. 154:367-382; U.S. Pat. No. 4,873,192 and the references cited therein.

The term “sequence identity” as used herein refers to the sequence identity between genes or proteins at the nucleotide or amino acid level, respectively. “Sequence identity” is a measure of identity between proteins at the amino acid level and a measure of identity between nucleic acids at nucleotide level. The protein sequence identity may be determined by comparing the amino acid sequence in a given position in each sequence when the sequences are aligned. Similarly, the nucleic acid sequence identity may be determined by comparing the nucleotide sequence in a given position in each sequence when the sequences are aligned. “Sequence identity” means the percentage of identical subunits at corresponding positions in two sequences when the two sequences are aligned to maximize subunit matching, i.e., taking into account gaps and insertions. Sequence identity is present when a subunit position in both of the two sequences is occupied by the same nucleotide or amino acid, e.g., if a given position is occupied by an adenine in each of two DNA molecules, then the molecules are identical at that position. For example, if 7 positions in a sequence of 10 nucleotides in length are identical to the corresponding positions in a second 10-nucleotide sequence, then the two sequences have 70% sequence identity. Methods for the alignment of sequences for comparison are well known in the art, such methods include the BLAST algorithm, which calculates percent sequence identity and performs a statistical analysis of the similarity between the two sequences. The software for performing BLAST analysis is publicly available through the National Center for Biotechnology Information (NCBI) website.

The term “sequence homology” as used herein refers to the sequence similarity between proteins at the amino acid level. “Sequence homology” is a measure of similarity between proteins at the amino acid level. The protein sequence homology may be determined by comparing the amino acid sequence in a given position in each sequence when the sequences are aligned. “Sequence homology” means the percentage of homologous subunits (i.e., amino acids) at corresponding positions in two sequences when the two sequences are aligned to maximize subunit matching, i.e., taking into account gaps which factor in insertions and deletions in the aligned sequences. Sequence homology is present when a subunit position in each of the two or more sequences is occupied by the identical amino acid or functionally similar amino acids (e.g., isosteric or isoelectric amino acid identities; amino acid residues that belong to the same functional class, such as e.g. positively charged residues, or small hydrophobic residues). Sequence homology is absent when a subunit position in each of the two or more sequences is occupied by a functionally different amino acid (i.e., lacking structural similarity). Methods for the alignment of sequences for comparison are well known in the art, such methods include the BLAST algorithm, which calculates percent sequence homology and performs a statistical analysis of the homology between the two sequences. The software for performing BLAST analysis is publicly available through the National Center for Biotechnology Information (NCBI) website.

The terms “corresponding to position(s)” or “position(s) . . . with reference to position(s)” of or within a peptide or a polynucleotide, such as recitation that nucleotides or amino acid positions “correspond to” nucleotides or amino acid positions of a disclosed sequence, such sequence set forth in the Sequence Listing, refers to nucleotides or amino acid positions identified in the polynucleotide or in the peptide upon alignment with the disclosed sequence using a standard alignment algorithm, such as the BLAST algorithm (NCBI). One skilled in the art can identify any given amino acid residue in a given peptide at a position corresponding to a particular position of a reference sequence, such as set forth in the Sequence Listing, by performing alignment of the peptide sequence with the reference sequence (for example, by using BLASTP publicly available through the NCBI website), matching the corresponding position of the reference sequence with the position in peptide sequence and thus identifying the amino acid residue within the peptide. Amino acid positions corresponding to the recited residues can be also determined by structural alignment to the experimentally-determined template structure in the PDB (as given by the PDB accession code after making structural truncations corresponding to the SEQ ID NO of interest), such as for each of the SEQ ID NOs: 7-59. The reference structures used in the structural alignment can be experimentally determined or generated by homology modeling using state of the art homology modeling methods such as Rosetta or PyRosetta macromolecular software suites, machine learning models such as AlphaFold2, or the like. Other useful structural alignment methods and/or programs include, but are not limited to, TM-align, PyMOL (superalign, cealign, and align methods), LSQMAN, Fr-TM-align, DALI, DaliLite, CE, CE-MC, and the like.

The term “peptide bond” as used herein refers to a chemical bond formed between two molecules (such as two amino acids) when the carboxyl group of one molecule reacts with the amino group of the other molecule, releasing a water molecule (H₂O).

The term “modified amino acid residue” as used herein refers to an amino acid residue within a peptide that comprises a modification that distinguish it from the corresponding original, or unmodified, amino acid residue. In some embodiments, the modification can be a naturally occurring post-translational modification of the amino acid residue. In other embodiments, the modification is a non-naturally occurring modification of the amino acid residue; such modified amino acid residue is not naturally present in peptides of living organisms (represents an unnatural amino acid residue). Such modified amino acid residue can be made by modifying a natural amino acid residue within the peptide by a modifying reagent, or can be chemically synthesized and incorporated into the peptide during peptide synthesis.

The terms “specifically binding” and “specifically recognizing” are used interchangeably herein and generally refer to an engineered metalloprotein binder that binds to a cognate target peptide or a portion thereof more readily than it would bind to a random, non-cognate peptide. The term “specificity” is used herein to qualify the relative affinity by which an engineered metalloprotein binder binds to a cognate target peptide. Specific binding typically means that an engineered metalloprotein binder binds to a cognate target peptide at least twice more likely that to a random, non-cognate peptide (a 2:1 ratio of specific to non-specific binding). Non-specific binding refers to background binding, and is the amount of signal that is produced in a binding assay between an engineered metalloprotein binder and an N-terminally modified target peptide when the modified NTAA residue cognate for the engineered metalloprotein binder is not present at the N-terminus of the target peptide. In some embodiments, specific binding refers to binding between an engineered metalloprotein binder and an N-terminally modified target peptide with a dissociation constant (Kd) of 200 nM or less.

In some embodiments, binding specificity between an engineered metalloprotein binder and an N-terminally modified target peptide is predominantly or substantially determined by interaction between the engineered metalloprotein binder and the modified NTAA residue of the N-terminally modified target peptide, which means that there is only minimal or no interaction between the engineered metalloprotein binder and the penultimate terminal amino acid residue (P2) of the target peptide, as well as other residues of the target peptide. In some embodiments, the engineered metalloprotein binder binds with at least 5 fold higher binding affinity to the modified NTAA residue of the target peptide than to any other region of the target peptide. In some embodiments, the engineered metalloprotein binder has a substrate binding pocket with certain size and/or geometry matching the size and/or geometry of the modified NTAA residue of the N-terminally modified target peptide, to which the engineered metalloprotein binder specifically binds to. In such embodiments, the modified NTAA residue occupies a volume encompassing a substrate binding pocket of the engineered metalloprotein binder that effectively precludes the P2 residue of the target peptide from entering into the substrate binding pocket or interacting with affinity-determining residues of the engineered metalloprotein binder. In some embodiments, the engineered metalloprotein binder specifically binds to N-terminally modified target peptides, wherein the target peptides share the same modified NTAA residue that interacts with the engineered metalloprotein binder, but have different P2 residues. In some embodiments, the engineered metalloprotein binder is capable of specifically binding to each N-terminally modified target peptide from a plurality of N-terminally modified target peptides, wherein the plurality of N-terminally modified target peptides contains at least 3, at least 5, or at least 10 N-terminally modified target peptides that were modified with the same N-terminal modifier agent, have the same modified NTAA residue, and have different P2 residues. Thus, in preferred embodiments, the engineered metalloprotein binder possesses binding affinity towards the modified NTAA residue of the N-terminally modified target peptide, but has little or no affinity towards P2 or other residues of the target peptide.

As used herein, the term “heterocycle”, “heterocyclic”, or “heterocyclyl” refers to a saturated or an unsaturated non-aromatic group having from 1 to 10 annular carbon atoms and from 1 to 4 annular heteroatoms, such as nitrogen, sulfur or oxygen, and the like, wherein the nitrogen and sulfur atoms are optionally oxidized, and the nitrogen atom(s) are optionally quaternized. A heterocyclyl group may have a single ring or multiple condensed rings, but excludes heteroaryl groups. A heterocycle comprising more than one ring may be fused, spiro or bridged, or any combination thereof. In fused ring systems, one or more of the fused rings can be aryl or heteroaryl. Examples of heterocyclyl groups include, but are not limited to, tetrahydropyranyl, dihydropyranyl, piperidinyl, piperazinyl, pyrrolidinyl, thiazolinyl, thiazolidinyl, tetrahydrofuranyl, tetrahydrothiophenyl, 2,3-dihydrobenzo[b]thiophen-2-yl, 4-amino-2-oxopyrimidin-1(2H)-yl, and the like.

The term “substituted” means that the specified group or moiety bears one or more substituents in place of a hydrogen atom of the unsubstituted group, including, but not limited to, substituents such as alkoxy, acyl, acyloxy, carbonylalkoxy, acylamino, amino, aminoacyl, aminocarbonylamino, aminocarbonyloxy, cycloalkyl, cycloalkenyl, aryl, heteroaryl, aryloxy, cyano, azido, halo, hydroxyl, nitro, carboxyl, thiol, thioalkyl, cycloalkyl, cycloalkenyl, alkyl, alkenyl, alkynyl, heterocyclyl, aralkyl, aminosulfonyl, sulfonylamino, sulfonyl, oxo, carbonylalkylenealkoxy and the like. The term “unsubstituted” means that the specified group bears no substituents. The term “optionally substituted” means that the specified group is unsubstituted or substituted by one or more substituents and thus includes both substituted and unsubstituted versions of the group. Where the term “substituted” is used to describe a structural system, the substitution is meant to occur at any valency-allowed position on the system.

It is understood that aspects and embodiments of the invention described herein include “consisting of” and/or “consisting essentially of” aspects and embodiments.

Throughout this disclosure, various embodiments of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Other objects, advantages and features of the present invention will become apparent from the following specification taken in conjunction with the accompanying drawings.

### I. Binders for N-Terminally Modified Peptides

In one embodiment, provided herein is a metalloprotein binder that specifically binds to a N-terminally modified target peptide, wherein: said N-terminally modified target peptide is derived from a target peptide and said N-terminally modified target peptide has a formula: Z-P1-P2-peptide, said Z being a metal-binding N-terminal modification, said P1 being the N-terminal amino acid residue of said target peptide, and P2 being a penultimate terminal amino acid residue of said target peptide; and said binder specifically binds to said N-terminally modified target peptide through interaction between said binder and said Z and P1 of said N-terminally modified target peptide, wherein the binding specificity between said binder and said N-terminally modified target peptide is predominantly or substantially determined by said interaction between said binder and said P1 of said N-terminally modified target peptide.

The present binders can specifically bind to any suitable N-terminally modified target peptide. For example, the length of the target peptide and/or the N-terminally modified target peptide can be greater than 4 amino acids, greater than 5 amino acids, greater than 6 amino acids, greater than 7 amino acids, greater than 8 amino acids, greater than 9 amino acids, greater than 10 amino acids, greater than 11 amino acids, greater than 12 amino acids, greater than 13 amino acids, greater than 14 amino acids, greater than 15 amino acids, greater than 20 amino acids, greater than 25 amino acids, or greater than 30 amino acids.

The P1 or the N-terminal amino acid residue of a target peptide can be any suitable amino acid residue. In some embodiments, the P1 can comprise a naturally-occurring amino acid residue. In some embodiments, the P1 can comprise a modification, e.g., a naturally-occurring or a non-natural modification. In some embodiments, the P1 can comprise an amino acid with a post-translational modification. The P2 or the penultimate terminal amino acid residue of a target peptide can be any suitable amino acid residue. In some embodiments, the P2 can comprise a naturally-occurring amino acid residue. In some embodiments, the P2 can comprise a modification, e.g., a naturally-occurring or a non-natural modification. In some embodiments, the P2 can comprise an amino acid with a post-translational modification.

The Z can comprise any suitable metal-binding N-terminal modification. For example, the Z can comprise a synthetic N-terminal modification. In another example, the Z can comprise an amino acid moiety and/or has a size, e.g., length axis or volume, shape, and/or configuration similar to or exceeding a natural amino acid. In some embodiments, the Z can be a bipartite N-terminal modification (NTM) that comprises a natural or unnatural amino acid portion (AA) and a metal-binding group. The amino acid portion (AA) and the N-terminal metal-binding group can be connected or linked by any suitable bond or linkage. For example, the amino acid portion (AA) and the N-terminal metal-binding group can be connected with an amide bond. In some embodiments, the Z does not comprise an amino acid moiety. The Z can be a bipartite N-terminal modification (NTM) that comprises a small (or small molecule) chemical entity having a size, e.g., length axis or volume, shape, and/or configuration similar to or exceeding a natural amino acid, and a N-terminal metal-binding group. The small (or small molecule) chemical entity and the N-terminal metal-binding group can be connected or linked by any suitable bond or linkage, for example, an amide bond. Preferably, the Z can have a size, e.g., length axis of about 5-10 Å and volume of about 100-1000 Å³. In some embodiments, the small (or small molecule) chemical entity has a length axis of about 5, 6, 7, 8, 9 or 10 Å, or any range thereof. In some embodiments, the small (or small molecule) chemical entity has a volume of about 100, 200, 300, 400, 500, 600, 700, 800, 900, 1000 Å³ or any range thereof.

In some embodiments, the interaction between the binder and the Z has minimal or no impact or influence on the binding specificity between the binder and the N-terminally modified target peptide. In some embodiments, the interaction between the binder and the Z at least partially determines the binding strength or binding efficiency between the binder and the N-terminally modified target peptide.

In some embodiments, there is minimal or no interaction between the binder and the P2. In some embodiments, there is minimal interaction between the binder and the P2, and the minimal interaction between the binder and the P2 has minimal or no impact or influence on the binding specificity, strength and/or efficiency between the binder and the N-terminally modified target peptide; and/or P1-P2 occupies a volume or shape encompassing a cavity or pocket of the binder that effectively precludes the P2 from entering into or interacting with an affinity determining region of the binder. In some embodiments, the volume of the cavity or pocket is greater than the volume occupied by a glycine residue. In some embodiments, the volume of the pocket or cavity is less than about 1,000 Å³.

In some embodiments, the present metalloprotein binders can specifically bind to N-terminally modified target peptide that contains a particular or specific N-terminal amino acid residue and have the ability to distinguish N-terminally modified target peptides that contain different N-terminal amino acid residues. In some embodiments, the present binders can also specifically bind to N-terminally modified target peptides that contain a group of N-terminal amino acid residues, and have the ability to distinguish such N-terminally modified target peptides from other N-terminally modified target peptides that contain N-terminal amino acid residue(s) outside the recognized group of the N-terminal amino acid residues. In some embodiments, the present binder specifically binds to multiple N-terminally modified target peptides that comprise the same P1 residue. In some embodiments, the present binder specifically binds to multiple N-terminally modified target peptides that comprise different P1 residues. For example, the present binder can specifically bind to multiple N-terminally modified target peptides that comprise 2, 3, 4, 5, 6, 7, 8, 9 or 10 different P1 residues.

The engineered metalloprotein binder can be derived or evolved from any suitable metalloenzyme. The engineered metalloprotein binder can have any suitable binding region, core or substrate pocket. For example, the engineered metalloprotein binder can comprise a b-barrel substrate pocket. In some embodiments, upon binding to a N-terminally modified target peptide, the Z-P1 group of the N-terminally modified target peptide occupy the metalloprotein binder substrate pocket. The pocket volume of the metalloenzyme from which the metalloprotein binder is derived can span volumes ranging from 200 Å³-3.000 Å³ encompassing a range of Z-P1 sizes. For example, the pocket volume of the metalloenzyme from which the metalloprotein binder is derived can span volumes ranging from 200 Å³-500 Å³, 500 Å³-1,000 Å³, 1,000 Å³-2,000 Å³, 2,000 Å³-3,000 Å³, or any subrange thereof, encompassing a range of Z-P1 sizes. The engineered metalloprotein binder can specifically binds to a N-terminally modified target peptide with any suitable P1 residue.

In some embodiments, the present metalloprotein binders can have a binding signal and/or affinity towards a modified target peptide comprising a specific P1 residue that is at least 2-fold or higher as compared to the binder's binding signal and/or affinity towards an otherwise identical modified target peptide but comprising a different P1 residue. In some embodiments, the present metalloprotein binders can have a binding signal and/or affinity towards a modified target peptide comprising a specific P1 residue that is at least 2-fold, 3-fold, 4-fold, 5-fold, 6-fold, 7-fold, 8-fold, 9-fold, 10-fold, 15-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1,000-fold, 1,500-fold, 2,000-fold, or higher, as compared to the binder's binding signal and/or affinity towards an otherwise identical modified target peptide but comprising a different P1 residue.

A nucleic acid encoding the above engineered metalloprotein binder is also provided herein. A vector, e.g., an expression vector, comprising the nucleic acid encoding the above engineered metalloprotein binder is also provided herein. A host cell comprising the above nucleic acid or the vector is further provided herein. The host cell can be any suitable type of cell. For example, the host cell can be a mammalian or human host cell.

In yet another embodiment, provided herein is a kit for obtaining an information regarding at least one amino acid residue of a peptide, the kit comprises:
a) a N-terminal modifier agent that is configured to contact a peptide to form a N-terminally modified peptide having a formula: Z-P1-P2-peptide, wherein P1 is a N-terminal amino acid residue of the peptide, P2 is a penultimate terminal amino acid residue of the peptide, and Z is an N-terminal modification capable of coordinating or chelating a metal ion M; and/or
b) a metalloenzyme that binds to the metal ion M that is configured to specifically bind to the N-terminally modified peptide through interaction between the metalloenzyme, the metal ion M and the Z-P1-P2-peptide, wherein the binding specificity between the metalloenzyme and the Z1-P1-P2-peptide is predominantly or substantially determined by interaction between the metalloenzyme and a Z1-P1 group of the Z1-P1-P2-peptide.

### II. Methods of Treating Target Peptides

In another embodiment, provided herein is a method of treating a target peptide, which method comprises: a) contacting a target peptide with a N-terminal modifier agent to form a N-terminally modified target peptide having a formula: Z-P1-P2-peptide, said Z being a metal-binding N-terminal modification, said P1 being the N-terminal amino acid residue of said target peptide, and P2 being a penultimate terminal amino acid residue of said target peptide; and b) contacting a metalloprotein binder with said N-terminally modified target peptide to allow said binder to specifically bind to said N-terminally modified target peptide through interaction between said binder and said Z and P1 of said N-terminally modified target peptide, wherein the binding specificity between said binder and said N-terminally modified target peptide is predominantly or substantially determined by said interaction between said binder and said P1 of said N-terminally modified target peptide.

In yet another embodiment, provided herein is a method for obtaining an information regarding at least one amino acid residue of a peptide, comprising the steps of:
a) contacting a peptide with a first N-terminal modifier agent to form a N-terminally modified peptide having a formula:
Z1-P1-P2-peptide, wherein P1 is a N-terminal amino acid residue of the peptide, P2 is a penultimate terminal amino acid residue of the peptide, and Z1 is an N-terminal modification capable of coordinating or chelating a metal ion M1; b) providing a first metalloenzyme that binds to the metal ion M1 and allowing specific binding between the Z1-P1-P2-peptide, the first metalloenzyme and the metal ion M1, wherein the binding specificity between the first metalloenzyme and the Z1-P1-P2-peptide is predominantly or substantially determined by interaction between the first metalloenzyme and a Z1-P1 group of the Z1-P1-P2-peptide; c) obtaining an information regarding the first metalloenzyme; and d) obtaining an information regarding the P1 amino acid residue of the peptide based on the obtained information regarding the first metalloenzyme.

In another embodiment, at step (b) of the method, a first set of metalloenzymes comprising the first metalloenzyme is provided, and each metalloenzyme from the first set of metalloenzymes binds to the metal ion M1.

In yet another embodiment, the method further comprises the following steps:
i) cleaving a peptide bond between P1 and P2 of the Z-P1-P2-peptide to form a second peptide having P2 as a new N-terminal amino acid residue;
ii) contacting the peptide with a second N-terminal modifier agent to form a N-terminally modified peptide having a formula: Z2-P2-peptide, wherein Z2 is an N-terminal modification capable of coordinating or chelating a metal ion M2;
iii) providing a second metalloenzyme that binds to the metal ion M2 and allowing specific binding between the Z2-P2-peptide, the second metalloenzyme and the metal ion M2, wherein the binding specificity between the second metalloenzyme and the Z2-P2-peptide is predominantly or substantially determined by interaction between the second metalloenzyme and a Z2-P2 group of the Z2-P2-peptide;
iv) obtaining an information regarding the second metalloenzyme; and
v) obtaining an information regarding the P2 amino acid residue of the peptide based on the obtained information regarding the second metalloenzyme.

The present methods can be used to treat any suitable target peptide or a target peptide with suitable length. For example, the length of the target peptide and/or the N-terminally modified target peptide can be greater than 4 amino acids, greater than 5 amino acids, greater than 6 amino acids, greater than 10 amino acids, greater than 15 amino acids, greater than 20 amino acids, or greater than 30 amino acids.

In some embodiments, the interaction between the binder and the Z has minimal or no impact or influence on the binding specificity between the binder and the N-terminally modified target peptide. In some embodiments, the interaction between the binder and the Z at least partially determines the binding strength or binding efficiency between the binder and the N-terminally modified target peptide.

In some embodiments, there is minimal or no interaction between the binder and the P2. In some embodiments, there is minimal interaction between the binder and the P2, and the minimal interaction between the binder and the P2 has minimal or no impact or influence on the binding specificity, strength and/or efficiency between the binder and the N-terminally modified target peptide; and/or P1-P2 occupies a volume or shape encompassing a cavity or pocket of the binder that effectively precludes the P2 from entering into or interacting with an affinity determining region of the binder.

The present binders used in the present methods can specifically bind to N-terminally modified target peptides that contain a particular or specific N-terminal amino acid residue, and they have the ability to distinguish N-terminally modified target peptides that contain different N-terminal amino acid residues. In other embodiments, the binders disclosed herein can specifically bind to N-terminally modified target peptides that contain a group of N-terminal amino acid residues, and they have the ability to distinguish such N-terminally modified target peptides from other N-terminally modified target peptides that contain N-terminal amino acid residue(s) outside the recognized group of the N-terminal amino acid residues. In some embodiments, the present binder used in the present methods specifically binds to multiple N-terminally modified target peptides that comprise the same P1 residue. In some embodiments, the present binder used in the present methods specifically binds to multiple N-terminally modified target peptides that comprise different P1 residues. For example, the present binder used in the present methods can specifically bind to multiple N-terminally modified target peptides that comprise 2, 3, 4, 5, 6, 7, 8, 9 or 10 different P1 residues.

The present methods can further comprise a step c) cleaving the peptide bond between the P1 and P2 to form a peptide wherein the P2 becomes N-terminal amino acid residue of the nascent peptide. The peptide bond between the P1 and P2 can be cleaved using any agent or reaction. For example, the peptide bond between the P1 and P2 can be cleaved using a chemical agent or reaction. In another example, the peptide bond between the P1 and P2 can be cleaved using a modified cleavase. In some embodiments, the peptide bond between the P1 and P2 is cleaved using an above descried modified or engineered cleavase described in U.S. published patent application US 2021/0214701 A1.

In some embodiments, the cleavage is conducted while the binder is bound with the N-terminally modified target peptide. In some embodiments, the cleavage is conducted after the binder is released and/or removed from the N-terminally modified target peptide.

In some embodiments, steps a)-c) can be repeated one or more times to form a peptide having a newly exposed N-terminal amino acid residue at the beginning of each cycle.

In the present methods, any suitable number of binder(s) can be used. In some embodiments, the binding step can comprise contacting a single binder with a collection of N-terminally modified target peptides to allow the binder to bind specifically to a subset of the N-terminally modified target peptides. In some embodiments, the binding step can comprise contacting a plurality of binders with N-terminally modified target peptides to allow the binders to specifically bind to at least one of the N-terminally modified target peptides.

In some embodiments, the binder used in the present methods can comprise a coding tag with identifying information regarding the binder. The coding tag can comprise any suitable type of molecule or composition. For example, the coding tag can comprise or can be a DNA molecule, an RNA molecule, a PNA molecule, a BNA molecule, an XNA, molecule, an LNA molecule, a γPNA molecule, or a combination thereof. In another example, the coding tag can comprise a unique molecular identifier (UMI) and/or a universal priming site. The binding agent and the coding tag can be joined or linked directly, or indirectly, e.g., via a linker.

The present methods can further comprise step d) transferring the identifying information of the coding tag to a recording tag attached to the N-terminally modified target peptide, thereby generating an extended recording tag on the N-terminally modified target peptide. Transferring the identifying information of the coding tag to the recording tag (or vice versa) can be effected using any agent or reaction. For example, transferring the identifying information of the coding tag to the recording tag (or vice versa) can be effected by primer extension or ligation.

In some embodiments, the steps of: a) contacting a target peptide with a N-terminal modifier agent; b) contacting a binder with the N-terminally modified target peptide; d) transferring the identifying information of the coding tag to a recording tag attached to the N-terminally modified target peptide; and c) cleaving the peptide bond between the P1 and P2 to form a peptide wherein the P2 becomes N-terminal amino acid residue of the nascent peptide, can be repeated in sequential order to generate one or more additional extended recording tags.

In some embodiments, the present methods can further comprise releasing the binder from the N-terminally modified target peptide and/or removing the released binder after step b) and before step c) or d). In some embodiments, the present methods can further comprise releasing the binder from the N-terminally modified target peptide and/or removing the released binder after step d) and before step c).

In some embodiments, the present methods can further comprise analyzing the one or more extended recording tag(s). The one or more extended recording tags can be amplified prior to analysis. The one or more extended recording tags can be analyzed using any suitable agent or reaction. For example, the one or more extended recording tags can be analyzed using a nucleic acid sequencing method. Any suitable nucleic acid sequencing method can be used. In some embodiments, the nucleic acid sequencing method can be sequencing by synthesis, sequencing by ligation, sequencing by hybridization, polony sequencing, ion semiconductor sequencing, or pyrosequencing. In some embodiments, the nucleic acid sequencing method can be single molecule real-time sequencing, nanopore-based sequencing, or direct imaging of DNA using advanced microscopy.

### III. Modified or Engineered Cleavases

In another embodiment, provided herein is a modified or an engineered cleavase comprising a mutation, e.g., one or more amino acid modification(s), deletion(s), addition(s) or substitution(s), in an unmodified cleavase, wherein: said modified or engineered cleavase is derived from a dipeptidyl peptidase of Thermomonas hydrothermalis or Caldithrix abyssii and removes or is configured to remove a single N-terminally modified amino acid from a target peptide. In some embodiments, the present modified or engineered cleavase is configured to cleave the peptide bond between an N-terminally modified amino acid residue and a penultimate terminal amino acid residue of the target peptide.

The present modified or engineered cleavase can comprise any suitable active site. For example, the present modified or engineered cleavase can comprise an active site that interacts with the amide bond between the N-terminally modified amino acid residue and a penultimate terminal amino acid residue of the target peptide. The present modified or engineered cleavase can remove or can be configured to remove any suitable single N-terminally modified amino acid from a target peptide containing any suitable N-terminal modification.

The present modified or engineered cleavase can comprise any suitable amino acid sequence variation(s) as compared with the amino acid sequence of the unmodified cleavase. For example, the present modified or engineered cleavase can comprise an amino acid sequence that exhibits at least 50% identity, at least 60% identity, at least 70% identity, at least 80% identity, or at least 90%, or at least 95%, or more identity with the unmodified cleavase.

The present or engineered modified cleavase can comprise any suitable type of mutation(s). For example, wherein the mutation can comprise an amino acid substitution, deletion, addition, or a combination thereof.

In some embodiments, the present modified or engineered cleavase is derived from a dipeptidyl peptidase of Thermomonas hydrothermalis comprising an amino acid sequence set forth in SEQ ID NO:3 (WT sequence with the signal peptide) or SEQ ID NO:4 (WT sequence without the signal peptide).

The present modified or engineered cleavase can comprise any suitable amino acid sequence variations as compared with the amino acid sequence of the unmodified cleavase. For example, the present modified or engineered cleavase can comprise an amino acid sequence that exhibits at least 20% identity, at least 30% identity, at least 40% identity, at least 50% identity, at least 60% identity, at least 70% identity, at least 80% identity, at least 90% or more identity or at least 95% or more identity to the amino acid sequence set forth in SEQ ID NO:3 or SEQ ID NO:4, or a specific binding fragment thereof.

In some embodiments, the present modified or engineered cleavase has a mutation, with reference to numbering of SEQ ID NO: 3 or SEQ ID NO: 4, selected from the group consisting of N214X, W215X, R219X, N329X, N333X, A671X, D673X, G674X, N682X, M692X, I651X, and a combination thereof, X being one of the 20 naturally occurring amino acids other than the amino acid residue of the unmodified dipeptidyl peptidase at the mutated position. In some embodiments, the present modified or engineered cleavase has one or more amino acid modification(s) of N214M, W215G, R219T, N329R, D673A, and/or G674V.

In some embodiments, the present modified or engineered cleavase is derived from a dipeptidyl peptidase of Caldithrix abyssii comprising an amino acid sequence set forth in SEQ ID NO: 5 (WT sequence with the signal peptide) or SEQ ID NO: 6 (WT sequence without the signal peptide).

The present modified or engineered cleavase can comprise any suitable amino acid sequence variations as compared with the amino acid sequence of the unmodified cleavase. For example, the present modified or engineered cleavase can comprise an amino acid sequence that exhibits at least 20% identity, at least 30% identity, at least 40% identity, at least 50% identity, at least 60% identity, at least 70% identity, at least 80% identity, at least 90% or more identity or at least 95% or more identity to the amino acid sequence set forth in SEQ ID NO: 5 or SEQ ID NO: 6, or a specific binding fragment thereof.

In some embodiments, the present modified or engineered cleavase has a mutation, with reference to numbering of SEQ ID NO: 5 or SEQ ID NO: 6, selected from the group consisting of N207M, W208X, R212X, N322X, D663X, and a combination thereof, X being one of the 20 naturally occurring amino acids other than the amino acid residue of the unmodified dipeptidyl peptidase at the mutated position. In some embodiments, the present modified or engineered cleavase has one or more amino acid modification(s) of N207M, W208G, R212V, N322I, D663A, or a combination thereof.

In some embodiments, disclosed herein is a modified cleavase comprising a dipeptidyl aminopeptidase comprising at least two mutations in a substrate binding site, wherein the modified cleavase removes or is configured to remove a single labeled terminal amino acid from a peptide. In some embodiments, the single labeled terminal amino acid is an N-terminal labeled amino acid of the peptide, and the modified cleavase comprises at least two amino acid substitutions in an amine binding site.

In some embodiments, the modified cleavase does not remove an unlabeled terminal dipeptide from the peptide.

In some embodiments, a method of treating a peptide is provided, the method comprising the steps of:
(a) contacting the peptide with a reagent for labeling a terminal amino acid of the peptide to produce a labeled peptide; and
(b) contacting the labeled peptide with a modified cleavase, the modified cleavase comprising a dipeptidyl aminopeptidase comprising at least two mutations in a substrate binding site, wherein the modified cleavase removes or is configured to remove a single labeled terminal amino acid from a peptide.

In some embodiments, the substrate binding site of the modified cleavase is a Z-P1 binding site, wherein Z-P1 is a modified N-terminal amino acid (NTAA) residue of the target peptide.

### IV. Kits of Treating Target Peptides

In yet another embodiment, provided herein is a kit of treating a target peptide, which kit comprises: a) a N-terminal modifier agent that is configured to contact a target peptide to form a N-terminally modified target peptide having a formula: Z-P1-P2-peptide, said Z being a N-terminal modification, said P1 being the N-terminal amino acid residue of said target peptide, and P2 being a penultimate terminal amino acid residue of said target peptide; and/or b) a binder that is configured to specifically bind to said N-terminally modified target peptide through interaction between said binder and said Z and P1 of said N-terminally modified target peptide, wherein the binding specificity between said binder and said N-terminally modified target peptide is predominantly or substantially determined by said interaction between said binder and said P1 of said N-terminally modified target peptide.

In some embodiments, the interaction between the binder and the Z has minimal or no impact or influence on the binding specificity between the binder and the N-terminally modified target peptide. In some embodiments, the interaction between the binder and the Z at least partially determines the binding strength or binding efficiency between the binder and the N-terminally modified target peptide.

The present binders used in the present kits can specifically bind to N-terminally modified target peptide that contains a particular or specific N-terminal amino acid residue and have the ability to distinguish N-terminally modified target peptides that contain different N-terminal amino acid residues. The present binders used in the present kits can also specifically bind to N-terminally modified target peptides that contain a group of N-terminal amino acid residues, and have the ability to distinguish such N-terminally modified target peptides from other N-terminally modified target peptides that contain N-terminal amino acid residue(s) outside the recognized group of N-terminal amino acid residues. In some embodiments, the present binder used in the present kits specifically binds to multiple N-terminally modified target peptides that comprise the same P1 residue. In some embodiments, the present binder used in the present kits specifically binds to multiple N-terminally modified target peptides that comprise different P1 residues. For example, the present binder used in the present kits can specifically bind to multiple N-terminally modified target peptides that comprise 2, 3, 4, 5, 6, 7, 8, 9 or 10 different P1 residues.

In some embodiments, the present kits further comprise: c) an agent that is configured to cleave the peptide bond between the P1 and P2 to form a peptide wherein after cleavage, the P2 becomes N-terminal amino acid residue of the nascent peptide. The peptide bond between the P1 and P2 can be cleaved using any agent or reaction. In some embodiments, the peptide bond between the P1 and P2 is cleaved using a chemical agent or reaction. In another example, the present kits can comprise an enzyme for cleaving the peptide bond between the P1 and P2. In some embodiments, the present kits can comprise a modified or an engineered cleavase described in U.S. published patent application US 2021/0214701 A1.

In another embodiment, the present kits further comprise: c) a modified cleavase comprising a dipeptidyl aminopeptidase comprising at least two mutations in a substrate binding site, wherein the modified cleavase removes or is configured to remove a single labeled terminal amino acid from a peptide.

In some embodiments, the present modified cleavase provided herein is a modified or an engineered cleavase comprising a mutation, e.g., one or more amino acid modification(s), deletion(s), addition(s) or substitution(s), in an unmodified cleavase, wherein: said modified or engineered cleavase is derived from a dipeptidyl peptidase of Thermomonas hydrothermalis or Caldithrix abyssii and removes or is configured to remove a single N-terminally modified amino acid from a target peptide. The present modified cleavase is configured to cleave the peptide bond between an N-terminally modified amino acid residue and a penultimate terminal amino acid residue of the target peptide.

In some embodiments, the present kits can comprise a plurality of binders that are configured to specifically bind to the N-terminally modified target peptide.

In some embodiments, the binder used in the present kits can comprise a coding tag with identifying information regarding the binder. For example, the coding tag can comprise or can be a DNA molecule, an RNA molecule, a PNA molecule, a BNA molecule, an XNA, molecule, an LNA molecule, a γPNA molecule, or a combination thereof.

In some other embodiments, the engineered binder further comprises a detectable label.

In some embodiments, the present kits can further comprise: d) a reagent for transferring the identifying information of the coding tag to a recording tag attached to the N-terminally modified target peptide, thereby generating an extended recording tag on the N-terminally modified target peptide. For example, the present kits can further comprise a chemical ligation reagent or a biological ligation reagent for transferring the identifying information. In some embodiments, the present kits can further comprise a reagent for primer extension of single-stranded nucleic acid or double-stranded nucleic acid for transferring the identifying information.

In some embodiments, the present kits can further comprise a reagent for releasing the binder from the N-terminally modified target peptide and/or for removing the released binder.

In some embodiments, the present kits can further comprise an amplification reagent for amplifying the one or more extended recording tag(s). In some embodiments, the present kits can further comprise a solid support.

### V. Target Peptide Assays

In some embodiments, the methods provided include using macromolecules, especially target peptide(s) associated with a recording tag, in a macromolecule analysis assay. In some particular embodiments, the macromolecules with associated and/or attached recording tags are subjected to a peptide analysis assay. In some embodiments, the macromolecule analysis assay is performed to assess the macromolecule, or to identify or determine at least a portion of the sequence of the peptide macromolecule, such as disclosed in earlier published applications US 2019/0145982 A1, US 2020/0348308 A1, US 2020/0348307 A1, US 2021/0208150 A1. In some embodiments, a plurality of macromolecules is analyzed using the described methods.

In some embodiments, the provided methods are for generating a nucleic acid encoded library representation of the binding history of the macromolecule. This nucleic acid encoded library can be amplified, and analyzed using high-throughput next generation digital sequencing methods, enabling millions to billions of molecules to be analyzed per run. The creation of a nucleic acid encoded library of binding information is useful in another way in that it enables enrichment, subtraction, and normalization by DNA-based techniques that make use of hybridization. These DNA-based methods are easily and rapidly scalable and customizable, and more cost-effective than those available for direct manipulation of other types of macromolecule libraries, such as protein libraries. Thus, nucleic acid encoded libraries of binding information can be processed prior to sequencing by one or more techniques to enrich and/or subtract and/or normalize the representation of sequences. This enables information of maximum interest to be extracted much more efficiently, rapidly and cost-effectively from very large libraries whose individual members may initially vary in abundance over many orders of magnitude.

In an exemplary workflow for analyzing peptides or peptides, the method generally includes contacting and binding of a binding agent comprising a coding tag to terminal amino acid (e.g., NTAA) of a peptide and transferring the binding agent's coding tag information to the recording tag associated with the peptide, thereby generating a first order extended recording tag. The terminal amino acid bound by the binding agent may be a chemically labeled or modified terminal amino acid. In some embodiments, the terminal amino acid (e.g., NTAA) is eliminated after the information from the coding tag is transferred. The terminal amino acid eliminated may be a chemically labeled or modified terminal amino acid. Removal of the NTAA by contacting with an enzyme or chemical reagents converts the penultimate amino acid of the peptide to a terminal amino acid. The peptide analysis may include one or more cycles of binding with additional binding agents to the terminal amino acid, transferring information from the additional binding agents to the extended nucleic acid thereby generating a higher order extended recording tag containing information from two or more coding tags, and eliminating the terminal amino acid in a cyclic manner. Additional binding, transfer, labeling, and removal, can occur as described above up to n amino acids to generate an n^{th} order extended nucleic acid, which collectively represent the peptide. In some embodiments, the order of the steps in the process for a degradation-based peptide or peptide sequencing assay can be reversed or be performed in various orders. For example, in some embodiments, the terminal amino acid labeling can be conducted before and/or after the peptide is bound to the binding agent. In some embodiments, the workflow may include one or more wash steps before and/or after binding of the binding agents, transfer of information, labeling or modifying of the terminal amino acid, and/or removal of the terminal amino acid.

In some embodiments, the disclosed binders are used in the NGPS (next generation peptide sequencing) assay. The NGPS peptide sequencing assay comprises several chemical and enzymatic steps in a cyclical progression. The fact that NGPS sequencing is single molecule confers several key advantages to the process, including robustness to inefficiencies in the various cyclical chemical/enzymatic steps.

An exemplary NGPS method for analyzing a macromolecule (e.g., peptide) analyte comprises the following steps:
(a) providing the peptide analyte and an associated recording tag joined to a solid support;
(b) contacting the peptide analyte with a first binding agent capable of binding to the peptide analyte, wherein the first binding agent comprises a first coding tag that comprises identifying information regarding the first binding agent;
(c) following binding of the first binding agent to the peptide analyte, transferring the identifying information regarding the first binding agent from the first coding tag to the recording tag to generate a first order extended recording tag;
(d) contacting the peptide analyte with a second binding agent capable of binding to the peptide analyte, wherein the second binding agent comprises a second coding tag that comprises identifying information regarding the second binding agent;
(e) following binding of the second binding agent to the peptide analyte, transferring the identifying information regarding the second binding agent from the second coding tag to the first order extended recording tag to generate a second order extended recording tag; and
(f) analyzing the second order extended recording tag, wherein analyzing comprises a sequencing method, and obtaining the identifying information regarding the first binding agent and the identifying information regarding the second binding agent to provide information regarding the peptide analyte, thereby analyzing the peptide analyte.

In preferred embodiments of the NGPS assay, binding agents are configured to recognize a modified NTAA on the immobilized peptide (NTAA-specific binding agents, FIG. 1). The steps of NGPS also include cleavage of the modified NTAA after binding and encoding steps. Then, the steps of binding, encoding, NTAA functionalization and cleavage are repeated n times to generate a DNA-encoded library on the recording tag associated with the immobilized peptide, representing identifying information at least for some amino acid residues of the immobilized peptide. Sequencing of the recording tag after completion of the n cycles provides the identifying information for these amino acid residues (both identities and order of the amino acid residues can be decoded from the sequence of the recording tag), which results in identification of the immobilized peptide.

Typically, for successful encoding (which comprises transferring the identifying information regarding the binding agent bound to the peptide from the coding tag of the binding agent to the recording tag), binding agents have affinity (Kd) to a component of the peptide of less than 500 nM, and preferably less than 100 nM; sometimes in the range of 10-100 nM, or in the range of 1-10 nM.

The described approach can be used to characterize and/or identify thousands, tens of thousands, or millions peptide analytes in parallel (in a single assay).

FIG. 1 depicts an exemplary degradation-like approach using a cyclic process including coding tag information transfer to a recording tag attached to the peptide, terminal amino acid elimination (e.g., NTAA elimination), and repeating the process in a cyclic manner. The peptide is attached, directly or indirectly, on a solid support. For example, the peptide can be immobilized on a solid support via a capture agent. Either the protein or capture agent may co-localize or be labeled with a recording tag, and proteins with associated recording tags are directly immobilized on a solid support. Information can be transferred from the coding tag on the bound binding agent to a proximal recording tag using any suitable means including by ligation or primer extension. In one embodiment as depicted, the coding tag includes spacer that is complementary to the spacer in the recording tag and can be used to initiate a primer extension reaction to transfer recording tag information to the coding tag. The final extended recording tag is optionally flanked by universal priming sites to facilitate downstream amplification and/or DNA sequencing. The forward universal priming site (e.g., Illumina's P5-S1 sequence) can be part of the original recording tag design and the reverse universal priming site (e.g., Illumina's P7-S2′ sequence) can be added (e.g., by extension) to the final extended recording tag. This final step may be done independently of a binding agent.

In the workflow as depicted in FIG. 1, the first step includes labeling or modifying the N-terminal amino acid (NTAA) with a functionalization reagent to enable removal of the NTAA in a later step; the functionalizing reagent generates an NTAA residue containing a functionalization moiety (e.g., a modification or label). A second step includes contacting the peptide with a binding agent that is attached to a DNA coding tag. In some embodiments, the labeling or modification of the NTAA may be performed prior to or after contacting the peptide with a binding agent. Upon binding of the binding agent to the NTAA of the peptide, information of the coding tag is transferred to the recording tag (e.g., via primer extension or ligation) to generate an extended recording tag. Lastly, the functionalized NTAA is eliminated via chemical or biological (e.g., enzymatic) means to expose a new NTAA.

As illustrated, the cycle is repeated “n” times to generate a final extended recording tag. In some embodiments, the order in the steps in the process for a degradation-based peptide sequencing assay can be reversed or moved around. In some embodiments, the terminal amino acid functionalization can be conducted after the peptide is bound to a support. In some embodiments, the analysis assay may include one or more additional steps, such as a wash step and/or treatment with other reagents. In some embodiments, the provided methods may be performed such that the C-terminal amino acid is modified, labeled, contacted by a binding agent, and/or eliminated from the peptide.

In some embodiments, the method includes obtaining and preparing macromolecules (e.g., peptides and proteins) from a single cell type or multiple cell types. In some embodiments, the sample comprises a population of cells. In some embodiments, the macromolecules (e.g., proteins, peptides, or peptides) are from a cellular or subcellular component, an extracellular vesicle, an organelle, or an organized subcomponent thereof. The macromolecules (e.g., proteins, peptides, or peptides) may be from organelles, for example, mitochondria, nuclei, or cellular vesicles.

In certain embodiments, a peptide, peptide, or protein can be fragmented before analyzing by the NGPS assay. For example, the fragmented peptide can be obtained by fragmenting a protein from a sample, such as a biological sample. The peptide, peptide, or protein can be fragmented by any means known in the art, including fragmentation by a protease or endopeptidase. In certain embodiments, a peptide, peptide, or protein is fragmented by proteinase K, or optionally, a thermolabile version of proteinase K to enable rapid inactivation. Protein and peptide fragmentation into peptides can be performed before or after attachment of a DNA recording tag. In certain embodiments, following enzymatic or chemical cleavage, the resulting peptide fragments are approximately the same desired length, e.g., from about 10 amino acids to about 70 amino acids, A cleavage reaction may be monitored, preferably in real time, by spiking the protein or peptide sample with a short test FRET (fluorescence resonance energy transfer) peptide comprising a peptide sequence containing a proteinase or endopeptidase cleavage site.

Various reactions may be used to attach the peptides to a solid support, or attach binders to corresponding coding tags. The peptides may be attached directly or indirectly to the solid support. In some cases, the peptide is attached to the solid support via a capture nucleic acid (capture DNA). Exemplary reactions include the copper catalyzed reaction of an azide and alkyne to form a triazole (Huisgen 1, 3-dipolar cycloaddition), strain-promoted azide alkyne cycloaddition (SPAAC), reaction of a diene and dienophile (Diels-Alder), strain-promoted alkyne-nitrone cycloaddition, reaction of a strained alkene with an azide, tetrazine or tetrazole, alkene and azide [3+2] cycloaddition, alkene and tetrazine inverse electron demand Diels-Alder (IEDDA) reaction (e.g., m-tetrazine (mTet) or phenyl tetrazine (pTet) and trans-cyclooctene (TCO)); or pTet and an alkene), alkene and tetrazole photoreaction, Staudinger ligation of azides and phosphines, and various displacement reactions, such as displacement of a leaving group by nucleophilic attack on an electrophilic atom (Horisawa 2014, Knall, Hollauf et al. 2014). Exemplary displacement reactions include reaction of an amine with: an activated ester; an N-hydroxysuccinimide ester; an isocyanate; an isothioscyanate, an aldehyde, an epoxide, or the like. In some embodiments, iEDDA click chemistry is used for immobilizing peptides to a solid support since it is rapid and delivers high yields at low input concentrations. In another embodiment, m-tetrazine rather than tetrazine is used in an iEDDA click chemistry reaction, as m-tetrazine has improved bond stability. In another embodiment, phenyl tetrazine (pTet) is used in an iEDDA click chemistry reaction. In one case, a peptide is labeled with a bifunctional click chemistry reagent, such as alkyne-NHS ester (acetylene-PEG-NHS ester) reagent or alkyne-benzophenone to generate an alkyne-labeled peptide. In some embodiments, an alkyne can also be a strained alkyne, such as cyclooctynes including Dibenzocyclooctyl (DBCO), etc.

In certain embodiments where multiple proteins are immobilized on the same solid support, the proteins can be spaced appropriately to accommodate methods of analysis to be used to assess the proteins. For example, it may be advantageous to space the proteins that optimally to allow a nucleic acid-based method for assessing and sequencing the proteins to be performed. In some embodiments, the method for assessing and sequencing the proteins involve a binding agent which binds to the protein and the binding agent comprises a coding tag with information that is transferred to a nucleic acid attached to the proteins (e.g., recording tag). In some cases, information transfer from a coding tag of a binding agent bound to one protein may reach a neighboring protein.

In some embodiments, the surface of the solid support is passivated (blocked). A “passivated” surface refers to a surface that has been treated with outer layer of material. Methods of passivating surfaces include standard methods from the fluorescent single molecule analysis literature, including passivating surfaces with polymer like polyethylene glycol (PEG) (Pan et al., 2015, Phys. Biol. 12:045006), polysiloxane (e.g., Pluronic F-127), star polymers (e.g., star PEG) (Groll et al., 2010, Methods Enzymol. 472:1-18), hydrophobic dichlorodimethylsilane (DDS)+self-assembled Tween-20 (Hua et al., 2014, Nat. Methods 11:1233-1236), diamond-like carbon (DLC), DLC+PEG (Stavis et al., 2011, Proc. Natl. Acad. Sci. USA 108:983-988), and zwitterionic moiety (e.g., U.S. Patent Application Publication US 2006/0183863). In addition to covalent surface modifications, a number of passivating agents can be employed as well including surfactants like Tween-20, polysiloxane in solution (Pluronic series), poly vinyl alcohol (PVA), and proteins like BSA and casein. Alternatively, density of macromolecules (e.g., proteins, peptide, or peptides) can be titrated on the surface or within the volume of a solid substrate by spiking a competitor or “dummy” reactive molecule when immobilizing the proteins, peptides or peptides to the solid substrate.

To control protein spacing on the solid support, the density of functional coupling groups for attaching the protein (e.g., TCO or carboxyl groups (COOH)) may be titrated on the substrate surface. In some embodiments, multiple proteins are spaced apart on the surface or within the volume (e.g., porous supports) of a solid support such that adjacent proteins are spaced apart at a distance of about 50 nm to about 500 nm. In some embodiments, multiple a proteins are spaced apart on the surface of a solid support with an average distance of at least 50 nm, at least 60 nm, at least 70 nm, at least 80 nm, at least 90 nm, at least 100 nm, or at least 500 nm. In some embodiments, multiple a proteins are spaced apart on the surface of a solid support with an average distance of at least 50 nm. In some embodiments, proteins are spaced apart on the surface or within the volume of a solid support such that, empirically, the relative frequency of inter- to intra-molecular events (e.g. transfer of information) is <1:10; <1:100; <1:1,000; or <1:10,000.

In some embodiments, the provided methods includes an oligonucleotides that comprise hairpin structure and a restriction enzyme site (or portion thereof). In some embodiments, the methods include the use of a reaction system wherein mixed enzymes are provided to the reaction. For example, the activities of the polymerase, the nucleic acid joining reagent and the double strand nucleic acid cleaving reagent, are provided with suitable conditions, transferring information from a coding tag to the recording tag to generate an extended recording tag. In the provided methods, the recording tag used comprises at least a partially double stranded DNA structure. Some advantages using the described methods include high information transfer (encoding) success, simple design for a step-wise reaction, option to perform in a single step/as a single pot reaction, reducing the need for spacers or reducing spacer length, and/or minimizing DNA-DNA interactions in the system.

In one embodiment, the macromolecule (e.g., protein or peptide) is labeled with a DNA recording tag. In some embodiments, the sample is provided with a plurality of recording tags. In some embodiments, a plurality of macromolecules in the sample is provided with recording tags. The recording tags may be associated or attached, directly or indirectly to the macromolecules using any suitable means. In some embodiments, a macromolecule may be associated with one or more recording tags. In some embodiments, the recording tag may be any suitable sequenceable moiety to which identifying information can be transferred (e.g., information from one or more coding tags).

In some embodiments, the recording tag can include a sample identifying barcode. A sample barcode is useful in the multiplexed analysis of a set of samples in a single reaction vessel or immobilized to a single solid substrate or collection of solid substrates (e.g., a planar slide, population of beads contained in a single tube or vessel, etc.). For example, macromolecules from many different samples can be labeled with recording tags with sample-specific barcodes, and then all the samples pooled together prior to immobilization to a solid support, cyclic binding of the binding agent, and recording tag analysis. Alternatively, the samples can be kept separate until after creation of a DNA-encoded library, and sample barcodes attached during PCR amplification of the DNA-encoded library, and then mixed together prior to sequencing. This approach could be useful when assaying analytes (e.g., proteins) of different abundance classes.

In some embodiments, the recording tags associated with a library of peptides share a common spacer sequence. In other embodiments, the recording tags associated with a library of peptides have binding cycle specific spacer sequences that are complementary to the binding cycle specific spacer sequences of their cognate binding agents. In some embodiments, the spacer sequence in the recording tag is designed to have minimal complementarity to other regions in the recording tag; likewise, the spacer sequence in the coding tag should have minimal complementarity to other regions in the coding tag. In some cases, the spacer sequence of the recording tags and coding tags should have minimal sequence complementarity to components such unique molecular identifiers, barcodes (e.g., compartment, partition, sample, spatial location), universal primer sequences, encoder sequences, cycle specific sequences, etc. present in the recording tags or coding tags.

In certain embodiments, a recording tag comprises a universal priming site, e.g., a forward or 5′ universal priming site. A universal priming site is a nucleic acid sequence that may be used for priming a library amplification reaction and/or for sequencing. A universal priming site may include, but is not limited to, a priming site for PCR amplification, flow cell adaptor sequences that anneal to complementary oligonucleotides on flow cell surfaces (e.g., Illumina next generation sequencing), a sequencing priming site, or a combination thereof. A universal priming site can be about 10 bases to about 60 bases. In some embodiments, a universal priming site comprises an Illumina P5 primer (5′-AATGATACGGCGACCACCGA-3′—SEQ ID NO:1) or an Illumina P7 primer (5′-CAAGCAGAAGACGGCATACGAGAT-3′—SEQ ID NO:2).

In some embodiments, the one or more tags or information of the one or more tags are transferred to the recording tag (e.g., via primer extension or ligation) to extend the recording tag. In some embodiments, one or more of the tags (e.g., compartment tag, a partition barcode, sample barcode, a fraction barcode, etc.) further comprise a functional moiety capable of reacting with an internal amino acid, the peptide backbone, or N-terminal amino acid on the plurality of protein complexes, proteins, or peptides. In some embodiments, the functional moiety is a click chemistry moiety, an aldehyde, an azide/alkyne, or a malemide/thiol, or an epoxide/nucleophile, an inverse electron demain Diels-Alder (iEDDA) group, or a moiety for a Staudinger reaction. In some specific embodiments, a plurality of compartment tags is formed by printing, spotting, ink jetting the compartment tags into the compartment, or a combination thereof. In some embodiments, the tag is attached to a peptide to link the tag to the macromolecule via a peptide-peptide linkage. In some embodiments, the tag-attached peptide comprises a protein ligase recognition sequence.

In some embodiments, before providing the peptide analyte and the associated nucleic acid recording tag joined to the solid support, the provided methods further comprise attaching the peptide analyte to the nucleic acid recording tag optionally joined to the solid support. Various alternatives can be used during the attachment step. For example, the peptide analyte can first be attached to the nucleic acid recording tag forming a conjugate, and then the conjugate is attached to the solid support. Alternatively, the nucleic acid recording tag can be attached (immobilized) to the solid support, and then the peptide analyte is attached to the immobilized nucleic acid recording tag.

In certain embodiments, a peptide or peptide macromolecule can be immobilized to a solid support by an affinity capture reagent (and optionally covalently crosslinked), wherein the recording tag is associated with the affinity capture reagent directly, or alternatively, the macromolecule can be directly immobilized to the solid support with a recording tag. In one embodiment, the macromolecule is attached to a bait nucleic acid which hybridizes to a capture nucleic acid and is ligated to a capture nucleic acid which comprises a reactive coupling moiety for attaching to the solid support. In some embodiments, the bait or capture nucleic acid may serve as a recording tag to which information regarding the peptide can be transferred. In some embodiments, the macromolecule is attached to a bait nucleic acid to form a nucleic acid-macromolecule conjugate. In some embodiments, the immobilization methods comprise bringing the nucleic acid-macromolecule conjugate into proximity with a solid support by hybridizing the bait nucleic acid to a capture nucleic acid (e.g. capture hairpin DNA) attached to the solid support, and covalently coupling the nucleic acid-macromolecule conjugate to the solid support. In some cases, the nucleic acid-macromolecule conjugate is coupled indirectly to the solid support, such as via a linker. In some embodiments, a plurality of the nucleic acid-macromolecule conjugates is coupled on the solid support and any adjacently coupled nucleic acid-macromolecule conjugates are spaced apart from each other at an average distance of about 50 nm or greater.

In some embodiments, providing the peptide and an associated recording tag joined to a solid support comprises the following steps: attaching the peptide to the recording tag to generate a nucleic acid-peptide conjugate; bringing the nucleic acid-peptide conjugate into proximity with a solid support by hybridizing the recording tag in the nucleic acid-peptide conjugate to a capture nucleic acid attached to the solid support; and covalently coupling the nucleic acid-peptide conjugate to the solid support.

In some embodiments, providing the peptide and an associated recording tag joined to a solid support further comprises attaching the peptide analyte to the nucleic acid recording tag optionally joined to the solid support.

In some embodiments, the nucleic acid recording tag is associated directly or indirectly to the peptide analyte via a non-nucleotide chemical moiety.

In some embodiments, providing conditions to allow transfer of identifying information from a coding tag of the binding agent to a recording tag associated with the peptide comprises addition of an enzyme (such as DNA polymerase or DNA ligase) to the immobilized peptide, as well as an appropriate buffer for this enzyme (such as a buffer for DNA polymerase or DNA ligase). Standard buffers that provide functionality of DNA polymerase or DNA ligase are known in the art.

In preferred embodiments, to provide encoding reaction specificity, transfer of identifying information regarding a binding agent from a coding tag of the binding agent to a recording tag associated with an immobilized peptide occurs only following (or after) binding of the binding agent to the immobilized peptide. The binding agent binds specifically to a component of the immobilized peptide (in various embodiments, binds to a single NTAA residue, to a modified amino acid residue, such as post-translationally-modified residue, to an epitope, or to more than one epitopes simultaneously); and binding of the binding agent to the immobilized peptide does not depend on the presence of the recording tag associated with the immobilized peptide.

In the present invention, the nucleic acid recording tag associated with the peptide is an element of the disclosed analytical assay and is not a component of the peptide. Thus, binding agents of the present invention do not bind to the nucleic acid recording tag.

In some embodiments, the conjugation of the macromolecule with a recording tag is performed using standard amine coupling chemistries. For example, the e-amino group (e.g., of lysine residues) and the N-terminal amino group may be susceptible to labeling with amine-reactive coupling agents, depending on the pH of the reaction (Mendoza et al., Mass Spectrom Rev (2009) 28(5): 785-815).

In some embodiments, the recording tags may comprise a reactive moiety for a cognate reactive moiety present on the target macromolecule, e.g., the target protein, (e.g., click chemistry labeling, photoaffinity labeling). For example, recording tags may comprise an azide moiety for interacting with alkyne-derivatized proteins, or recording tags may comprise a benzophenone for interacting with native proteins, etc. Upon binding of the target protein by the target protein specific binding agent, the recording tag and target protein are coupled via their corresponding reactive moieties. After the target protein is labeled with the recording tag, the target-protein specific binding agent may be removed by digestion of the DNA capture probe linked to the target-protein specific binding agent. For example, the DNA capture probe may be designed to contain uracil bases, which are then targeted for digestion with a uracil-specific excision reagent (e.g., USER™), and the target-protein specific binding agent may be dissociated from the target protein. In some embodiments, other types of linkages besides hybridization can be used to link the recording tag to a macromolecule.

Coding tag information associated with a specific binding agent may be transferred to a recording tag using a variety of methods. In any of the preceding embodiments, the transfer of identifying information (e.g., from a coding tag to a recording tag) can be accomplished by ligation (e.g., an enzymatic or chemical ligation, a splint ligation, a sticky end ligation, a single-strand (ss) ligation such as a ssDNA ligation, or any combination thereof), a polymerase-mediated reaction (e.g., primer extension of single-stranded nucleic acid or double-stranded nucleic acid), or any combination thereof.

In some embodiments, a DNA polymerase that is used for primer extension possesses strand-displacement activity and has limited or is devoid of 3′-5 exonuclease activity. Several of many examples of such polymerases include Klenow exo-(Klenow fragment of DNA Pol 1), T4 DNA polymerase exo-, T7 DNA polymerase exo (Sequenase 2.0), Pfu exo-, Vent exo-, Deep Vent exo-, Bst DNA polymerase large fragment exo-, Bca Pol, and Phi29 Pol exo-. In a preferred embodiment, the DNA polymerase is active at room temperature and up to 45° C. In another embodiment, a “warm start” version of a thermophilic polymerase is employed such that the polymerase is activated and is used at about 40° C.-50° C. An exemplary warm start polymerase is Bst 2.0 Warm Start DNA Polymerase (New England Biolabs).

In some embodiments, various conditions for one or more steps of the method may be modified by one skilled in the art. For example, the temperature for contacting of the binding agents to the macromolecules or for hybridization of the spacer sequences on the recording tag and coding tag can be increased or decreased to modify specificity or stringency of the interactions. In some embodiments, to minimize non-specific interaction of the coding tag labeled binding agents in solution with the nucleic acids of immobilized proteins, competitor (also referred to as blocking) oligonucleotides complementary to nucleic acids containing spacer sequences (e.g., on the recording tag) can be added to binding reactions to minimize non-specific interactions. In some embodiments, the blocking oligonucleotides contain a sequence that is complementary to the coding tag or a portion thereof attached to the binding agent. In some embodiments, the coding tag comprises a hairpin nucleic acid, and the hairpin includes a sequence that is complementary to a spacer and/or barcode of the coding tag. Excess competitor oligonucleotides are washed from the binding reaction prior to primer extension, which effectively dissociates the annealed competitor oligonucleotides from the nucleic acids on the recording tag, especially when exposed to slightly elevated temperatures (e.g., 30-50° C.).

Coding tag information associated with a specific binding agent may be transferred to a nucleic acid on the recording tag associated with the immobilized peptide via ligation. Ligation may be a blunt end ligation or sticky end ligation. Ligation may be an enzymatic ligation reaction. Examples of ligases include, but are not limited to CV DNA ligase, T4 DNA ligase, T7 DNA ligase, T3 DNA ligase, Taq DNA ligase, E. coli DNA ligase. Alternatively, a ligation may be a chemical ligation reaction. In one embodiment, a spacer-less ligation is accomplished by using hybridization of a “recording helper” sequence with an arm on the coding tag. The annealed complement sequences are chemically ligated using standard chemical ligation or “click chemistry” (Gunderson et al., Genome Res (1998) 8(11): 1142-1153; Litovchick et al., Artif DNA PNA XNA (2014) 5(1): e27896; Roloff et al., Methods Mol Biol (2014) 1050:131-141).

Various aspects of coding tag and recording tag compositions, as well as aspects of transferring identifying information from a coding tag to a recording tag are disclosed in the earlier published application US 2019/0145982 A1, incorporated herein.

In another embodiment, transfer of PNAs can be accomplished with chemical ligation using published techniques. The structure of PNA is such that it has a 5′ N-terminal amine group and an unreactive 3′ C-terminal amide. Chemical ligation of PNA requires that the termini be modified to be chemically active. This is typically done by derivatizing the 5′ N-terminus with a cysteinyl moiety and the 3′ C-terminus with a thioester moiety. Such modified PNAs easily couple using standard native chemical ligation conditions (Roloff et al., (2013) Bioorgan. Med. Chem. 21:3458-3464).

In certain embodiments, an extended recording tag associated with the immobilized peptide may comprise information from multiple coding tags representing multiple, successive binding events. In these embodiments, a single, concatenated extended recording tag associated with the immobilized peptide can be representative of a single peptide. As referred to herein, transfer of coding tag information to the recording tag associated with the immobilized peptide also includes transfer to an extended recording tag as would occur in methods involving multiple, successive binding events. In certain embodiments, the binding event information is transferred from a coding tag to the recording tag associated with the immobilized peptide in a cyclic fashion. Cross-reactive binding events can be informatically filtered out after sequencing by requiring that at least two different coding tags, identifying two or more independent binding events, map to the same class of binding agents (cognate to a particular protein). The coding tag may contain an optional UMI sequence in addition to one or more spacer sequences.

In certain embodiments, a binding agent may be a selective binding agent. As used herein, selective binding refers to the ability of the binding agent to preferentially bind to a specific ligand (e.g., amino acid or class of amino acids) relative to binding to a different ligand (e.g., another amino acid or class of amino acids). Selectivity is commonly referred to as the equilibrium constant for the reaction of displacement of one ligand by another ligand in a complex with a binding agent. Typically, such selectivity is associated with the spatial geometry of the ligand and/or the manner and degree by which the ligand binds to a binding agent, such as by hydrogen bonding or Van der Waals forces (non-covalent interactions) or by reversible or non-reversible covalent attachment to the binding agent. It should also be understood that selectivity may be relative, and as opposed to absolute, and that different factors can affect the same, including ligand concentration. Thus, in one example, a binding agent selectively binds one of the twenty standard amino acids. In some embodiments, a binding agent may exhibit flexibility and variability in target binding preference in some or all of the positions of the targets. In some embodiments, a binding agent may have a preference for one or more specific target terminal amino acids and have a flexible preference for a target at the penultimate position. In some other examples, a binding agent may have a preference for one or more specific target amino acids in the penultimate amino acid position and have a flexible preference for a target at the terminal amino acid position. In some embodiments, a binding agent is selective for a target comprising a terminal amino acid and other components of a macromolecule. In some particular examples, a binding agent is selective for a target comprising a terminal amino acid and an amide peptide backbone.

In the practice of the methods disclosed herein, the ability of a binding agent to selectively bind a feature or component of a macromolecule, e.g., a peptide, need only be sufficient to allow transfer of its coding tag information to the recording tag associated with the peptide. Thus, selectively need only be relative to the other binding agents to which the peptide is exposed. It should also be understood that selectivity of a binding agent need not be absolute to a specific amino acid, but could be selective to a class of amino acids, such as amino acids with polar or non-polar side chains, or with electrically (positively or negatively) charged side chains, or with aromatic side chains, or some specific class or size of side chains, and the like. In some embodiments, the ability of a binding agent to selectively bind a feature or component of a macromolecule is characterized by comparing binding abilities of binding agents. For example, the binding ability of a binding agent to the target can be compared to the binding ability of a binding agent which binds to a different target, for example, comparing a binding agent selective for a class of amino acids to a binding agent selective for a different class of amino acids. In some embodiments, a binding agent selective for non-polar side chains is compared to a binding agent selective for polar side chains. In some embodiments, a binding agent selective for a feature, component of a peptide, or one or more amino acid exhibits at least 1×, at least 2×, at least 5×, at least 10×, at least 50×, at least 100×, or at least 500× more binding compared to a binding agent selective for a different feature, component of a peptide, or one or more amino acid.

In a particular embodiment, the binding agent has a high affinity and high selectivity for the macromolecule, e.g., the peptide, of interest. In particular, a high binding affinity with a low off-rate may be efficacious for information transfer between the coding tag and recording tag. In certain embodiments, a binding agent has a Kd of about <200 nM, <100 nM, <50 nM, <10 nM, <5 nM, <1 nM, <0.5 nM, or <0.1 nM. In a particular embodiment, the binding agent is added to the peptide at a concentration >1×, >5×, >10×, >100×, or >1000× its Kd to drive binding to completion. A binding agent may be engineered for high affinity for a modified NTAA, high specificity for a modified NTAA, or both. In some embodiments, binding agents can be developed through directed evolution of promising affinity scaffolds using phage display.

In certain embodiments, the binding agent further comprises one or more detectable labels such as fluorescent labels, in addition to the binding moiety. In some embodiments, the binding agent does not comprise a polynucleotide such as a coding tag. In some embodiments, the binding agent comprises an aptamer. In one embodiment, the binding agent comprises a peptide and a detectable label. In one embodiment, the detectable label is optically detectable. In some embodiments, the detectable label comprises a fluorescently moiety, a color-coded nanoparticle, a quantum dot or any combination thereof. In one embodiment the label comprises a polystyrene dye encompassing a core dye molecule such as a FluoSphere™, Nile Red, fluorescein, rhodamine, derivatized rhodamine dyes, such as TAMRA, phosphor, polymethadine dye, fluorescent phosphoramidite, TEXAS RED, green fluorescent protein, acridine, cyanine, cyanine 5 dye, cyanine 3 dye, 5-(2′-aminoethyl)-aminonaphthalene-1-sulfonic acid (EDANS), BODIPY, 120 ALEXA or a derivative or modification of any of the foregoing. In one embodiment, the detectable label is resistant to photobleaching while producing lots of signal (such as photons) at a unique and easily detectable wavelength, with high signal-to-noise ratio.

In certain embodiments, a macromolecule, e.g., a peptide, is also contacted with a non-cognate binding agent. As used herein, a non-cognate binding agent is referring to a binding agent that is selective for a different peptide feature or component than the particular peptide being considered. For example, if the n NTAA is phenylalanine, and the peptide is contacted with three binding agents selective for phenylalanine, tyrosine, and asparagine, respectively, the binding agent selective for phenylalanine would be first binding agent capable of selectively binding to the n^{th} NTAA (i.e., phenylalanine), while the other two binding agents would be non-cognate binding agents for that peptide (since they are selective for NTAAs other than phenylalanine). The tyrosine and asparagine binding agents may, however, be cognate binding agents for other peptides in the sample. Thus, it should be understood that whether an agent is a binding agent or a non-cognate binding agent will depend on the nature of the particular peptide feature or component currently available for binding. Also, if multiple peptides are analyzed in a multiplexed reaction, a binding agent for one peptide may be a non-cognate binding agent for another, and vice versa.

In some embodiments, each unique binding agent within a library of binding agents has a unique barcode sequence. For example, 20 unique barcode sequences may be used for a library of 20 binding agents that bind to the 20 modified NTAA residues of immobilized peptides. In other embodiments, two or more different binding agents may share the same barcode sequence.

A coding tag may include a terminator nucleotide incorporated at the 3′ end of the 3′ spacer sequence. After a binding agent binds to a peptide and their corresponding coding tag and recording tags anneal via complementary spacer sequences, it is possible for primer extension to transfer information from the coding tag to the recording tag, or to transfer information from the recording tag to the coding tag. Addition of a terminator nucleotide on the 3′ end of the coding tag prevents transfer of recording tag information to the coding tag. It is understood that for embodiments described herein involving generation of extended coding tags, it may be preferable to include a terminator nucleotide at the 3′ end of the recording tag to prevent transfer of coding tag information to the recording tag.

A coding tag may be a single stranded molecule, a double stranded molecule, or a partially double stranded. A coding tag may comprise blunt ends, overhanging ends, or one of each. In some embodiments, a coding tag is partially double stranded, which prevents annealing of the coding tag to internal encoder and spacer sequences in a growing extended recording tag. In some embodiments, the coding tag comprises a hairpin. In certain embodiments, the hairpin comprises mutually complementary nucleic acid regions are connected through a nucleic acid strand. In some embodiments, the nucleic acid hairpin can also further comprise 3′ and/or 5′ single-stranded region(s) extending from the double-stranded stem segment. In some embodiments, the hairpin comprises a single strand of nucleic acid. In some embodiments, the coding tag sequence can be optimized for the particular sequencing analysis platform. For example, if the extended nucleic acid is to be analyzed using a nanopore sequencing instrument, the barcode sequences (e.g., sequences comprising identifying information from the coding tag) can be designed to be optimally electrically distinguishable in transit through a nanopore.

A coding tag is joined to a binding agent directly or indirectly, by any means known in the art, including covalent and non-covalent interactions. In some embodiments, a coding tag may be joined to binding agent enzymatically or chemically. In some embodiments, a coding tag may be joined to a binding agent via ligation. In other embodiments, a coding tag is joined to a binding agent via affinity binding pairs (e.g., biotin and streptavidin). In some cases, a coding tag may be joined to a binding agent to an unnatural amino acid, such as via a covalent interaction with an unnatural amino acid. In some particular embodiments, a binding agent is joined to a coding tag via a covalent linkage.

In some embodiments, a binding agent is joined to a coding tag via SpyCatcher-SpyTag interaction. The SpyTag peptide forms an irreversible covalent bond to the SpyCatcher protein via a spontaneous isopeptide linkage, thereby offering a genetically encoded way to create peptide interactions that resist force and harsh conditions (Zakeri et al., 2012, Proc. Natl. Acad. Sci. 109:E690-697; Li et al., 2014, J. Mol. Biol. 426:309-317). A binding agent may be expressed as a fusion protein comprising the SpyCatcher protein. In some embodiments, the SpyCatcher protein is appended on the N-terminus or C-terminus of the binding agent. The SpyTag peptide can be coupled to the coding tag using standard conjugation chemistries (Bioconjugate Techniques, G. T. Hermanson, Academic Press (2013)). In some particular embodiments, a binding agent is joined to a coding tag via methods, disclosed in the following published U.S. patents and patent applications: U.S. Pat. Nos. 9,547,003, 10,247,727, 10,527,609, 10,526,379, US 2016/272543 A1.

In some embodiments, an enzyme-based strategy is used to join the binding agent to a coding tag. For example, the binding agent may be joined to a coding tag using a formylglycine (FGly)-generating enzyme (FGE). In one example, a protein, e.g., SpyLigase, is used to join the binding agent to the coding tag (Fierer et al., Proc Natl Acad Sci USA. 2014 Apr. 1; 111(13): E1176-E1181).

In other embodiments, a binding agent is joined to a coding tag via SnoopTag-SnoopCatcher peptide-protein interaction. The SnoopTag peptide forms an isopeptide bond with the SnoopCatcher protein (Veggiani et al., Proc. Natl. Acad. Sci. USA, 2016, 113:1202-1207). A binding agent may be expressed as a fusion protein comprising the SnoopCatcher protein. In some embodiments, the SnoopCatcher protein is appended on the N-terminus or C-terminus of the binding agent. The SnoopTag peptide can be coupled to the coding tag using standard conjugation chemistries.

In yet other embodiments, a binding agent is joined to a coding tag via the HaloTag protein fusion tag and its chemical ligand. HaloTag is a modified haloalkane dehalogenase designed to covalently bind to synthetic ligands (HaloTag ligands) (Los et al., 2008, ACS Chem. Biol. 3:373-382). The synthetic ligands comprise a chloroalkane linker attached to a variety of useful molecules. A covalent bond forms between the HaloTag and the chloroalkane linker that is highly specific, occurs rapidly under physiological conditions, and is essentially irreversible.

In some embodiments, contacting of the first binding agent and second binding agent to the peptide, and optionally any further binding agents (e.g., third binding agent, fourth binding agent, fifth binding agent, and so on), are performed at the same time. For example, the first binding agent and second binding agent, and optionally any further order binding agents, can be pooled together, for example to form a library of binding agents. In another example, the first binding agent and second binding agent, and optionally any further order binding agents, rather than being pooled together, are added simultaneously to the peptide. In one embodiment, a library of binding agents comprises at least 20 binding agents that selectively bind to the 20 modified NTAA residues of immobilized peptides. In some embodiments, a library of binding agents comprises binding agents that selectively bind to the modified NTAA residues.

In other embodiments, the first binding agent and second binding agent, and optionally any further order binding agents, are each contacted with the peptide in separate binding cycles, added in sequential order. In certain embodiments, multiple binding agents are used at the same time in parallel. This parallel approach saves time and reduces non-specific binding by non-cognate binding agents to a site that is bound by a cognate binding agent (because the binding agents are in competition).

In certain embodiments, the concentration of the binding agents in a solution is controlled to reduce background and/or false positive results of the assay. In some embodiments, the concentration of a binding agent can be at any suitable concentration, e.g., at about 0.0001 nM, about 0.001 nM, about 0.01 nM, about 0.1 nM, about 1 nM, about 10 nM, about 100 nM, about 200 nM, about 500 nM, or about 1,000 nM.

In some embodiments, the ratio between the soluble binding agent molecules and the immobilized macromolecule, e.g., peptides, can be at any suitable range, e.g., at about 0.00001:1, about 0.0001:1, about 0.001:1, about 0.01:1, about 0.1:1, about 1:1, about 2:1, about 5:1, about 10:1, about 30:1, about 40:1, about 50:1, about 60:1, about 80:1, about 90:1, about 100:1, about 10⁴:1, about 10⁵:1, about 10⁶:1, or higher, or any ratio in between the above listed ratios. Higher ratios between the soluble binding agent molecules and the immobilized peptide(s) and/or the nucleic acids can be used to drive the binding and/or the coding tag information transfer to completion. This may be particularly useful for detecting and/or analyzing low abundance peptides in a sample.

In some embodiments, following the transfer of identifying information from a coding tag to a recording tag, at least one terminal amino acid is removed, cleaved, or eliminated from the peptide. In some embodiments, the at least one removed terminal amino acid comprises a modified amino acid using any of the methods or reagents provided herein. In embodiments relating to methods of analyzing peptides or peptides using a degradation based approach, following contacting and binding of a first binding agent to an n terminal amino acid (e.g., NTAA) of a peptide of n amino acids and transfer of the first binding agent's coding tag information to a nucleic acid associated with the peptide, thereby generating a first order extended nucleic acid (e.g., on the recording tag), the n NTAA is eliminated as described herein. Removal of the n labeled NTAA by contacting with an enzyme or chemical reagents converts the n−1 amino acid of the peptide to an N-terminal amino acid, which is referred to herein as an n−1 NTAA. A second binding agent is contacted with the peptide and binds to the n−1 NTAA, and the second binding agent's coding tag information is transferred to the first order extended nucleic acid thereby generating a second order extended nucleic acid (e.g., for generating a concatenated n^{th} order extended nucleic acid representing the peptide). Elimination of the n−1 labeled NTAA converts the n−2 amino acid of the peptide to an N-terminal amino acid, which is referred to herein as n−2 NTAA. Additional binding, transfer, labeling, and removal, can occur as described above up to n amino acids to generate an n^{th} order extended nucleic acid or n separate extended nucleic acids, which collectively represent the peptide. As used herein, an n “order” when used in reference to a binding agent, coding tag, or extended nucleic acid, refers to the n binding cycle, wherein the binding agent and its associated coding tag is used or the n binding cycle where the extended nucleic acid recording tag is created.

In some embodiments, chemical methods to cleave a modified NTAA residue of an immobilized peptide are disclosed in the published applications WO 2020/223133 and U.S. 2020/0348307 A1.

In some embodiments, enzymatic methods to cleave a modified NTAA residue of an immobilized peptide are disclosed in the published applications US 2021/0214701 A1. In some particular embodiments, enzymatic methods include use of the modified or engineered cleavase that is derived from a dipeptidyl peptidase of Thermomonas hydrothermalis comprising an amino acid sequence set forth in SEQ ID NO:3 (WT sequence with the signal peptide) or SEQ ID NO:4 (WT sequence without the signal peptide). Some embodiments of enzymatic methods to cleave a modified NTAA residue of immobilized peptides are disclosed above in the section III (Modified or engineered cleavases).

The length of the final extended recording tags generated by the methods described herein is dependent upon multiple factors, including the length of the coding tag (e.g., barcode sequence and spacer) and the length of the starting recording tags (e.g., the recording tag may optionally include a unique molecular identifier, spacer, universal priming site, barcode(s), or combinations thereof), the number of transfer cycles performed, and whether coding tags from each binding cycle are transferred to the same extended recording tag or to multiple extended recording tags.

After the transfer of the final identifying information to the extended recording tag from a coding tag, the recording tag can be capped by addition of a universal reverse priming site via ligation, primer extension or other methods known in the art. In some embodiments, the universal forward priming site in the recording tag (e.g., on the recording tag) is compatible with the universal reverse priming site that is appended to the final extended recording tag. In some embodiments, a universal reverse priming site is an Illumina P7 primer (5′-CAAGCAGAAGACGGCATACGAGAT-3′—SEQ ID NO:2) or an Illumina P5 primer (5′-AATGATACGGCGACCACCGA-3′—SEQ ID NO:1). The sense or antisense P7 may be appended, depending on strand sense of the recording tag to which the identifying information from the coding tag is transferred to. An extended nucleic acid library can be cleaved or amplified directly from the solid support (e.g., beads) and used in traditional next generation sequencing assays and protocols.

In some embodiments, a primer extension reaction is performed on a library of single stranded extended recording tags to copy complementary strands thereof. Extended recording tags can be processed and analyzed using a variety of nucleic acid sequencing methods. In some embodiments, the collection of extended recording can be concatenated. In some embodiments, the extended recording tag can be amplified prior to determining the sequence. A library of recording tags may be amplified in a variety of ways. A library of recording tags (e.g., recording tags comprising identifying information from one or more coding tags) may undergo exponential amplification, e.g., via PCR or emulsion PCR. Emulsion PCR is known to produce more uniform amplification (Hori, Fukano et al., Biochem Biophys Res Commun (2007) 352(2): 323-328). Alternatively, a library of recording tags may undergo linear amplification, e.g., via in vitro transcription of template DNA using T7 RNA polymerase. The library of recording tags (e.g., extended nucleic acids) can be amplified using primers compatible with the universal forward priming site and universal reverse priming site contained therein. A library of recording tags can also be amplified using tailed primers to add sequence to either the 5′-end, 3′-end or both ends of the extended nucleic acids. Sequences that can be added to the termini of the extended nucleic acids include library specific index sequences to allow multiplexing of multiple libraries in a single sequencing run, adaptor sequences, read primer sequences, or any other sequences for making the library of extended nucleic acids compatible for a sequencing platform. An example of a library amplification in preparation for next generation sequencing is as follows: a 20 μl PCR reaction volume is set up using an extended nucleic acid library eluted from ˜1 mg of beads (˜10 ng), 200 μM dNTP, 1 μM of each forward and reverse amplification primers, 0.5 μl (1 U) of Phusion Hot Start enzyme (New England Biolabs) and subjected to the following cycling conditions: 98° C. for 30 sec followed by 20 cycles of 98° C. for 10 sec, 60° C. for 30 sec, 72° C. for 30 sec, followed by 72° C. for 7 min, then hold at 4° C.

Examples of next generation sequencing methods that can be used for sequencing of the extended recording tags include sequencing by synthesis, sequencing by ligation, sequencing by hybridization, polony sequencing, ion semiconductor sequencing, and pyrosequencing. By attaching primers to a solid substrate and a complementary sequence to a nucleic acid molecule, a nucleic acid molecule can be hybridized to the solid substrate via the primer and then multiple copies can be generated in a discrete area on the solid substrate by using polymerase to amplify (these groupings are sometimes referred to as polymerase colonies or polonies). Consequently, during the sequencing process, a nucleotide at a particular position can be sequenced multiple times (e.g., hundreds or thousands of times)—this depth of coverage is referred to as “deep sequencing.” Examples of high throughput nucleic acid sequencing technology include platforms provided by Illumina, BGI, Qiagen, Thermo-Fisher, and Roche, including formats such as parallel bead arrays, sequencing by synthesis, sequencing by ligation, capillary electrophoresis, electronic microchips, “biochips,” microarrays, parallel microchips, and single-molecule arrays, as reviewed by Service (Science (2006) 311:1544-1546). Other approaches to sequencing of the extended recording tags can be used, such as described in U.S. Pat. Nos. 6,969,488, 6,172,218, and 6,306,597, incorporated herein.

The sequencing methods described herein can be advantageously carried out in multiplex formats such that multiple different recording tags are manipulated simultaneously. In particular embodiments, different recording tags can be treated in a common reaction vessel or on a surface of a particular substrate. This allows convenient delivery of sequencing reagents, removal of unreacted reagents and detection of incorporation events in a multiplex manner.

In some embodiments, the information from analysis (e.g., sequencing) of at least a portion of the extended recording tag can be used to associate the sequences determined to corresponding a peptide and align to the proteome. In some cases, following sequencing of the extended recording tags, the resulting sequences can be collapsed by their UMIs and then associated to their corresponding peptides and aligned to the totality of the proteome. In some cases, resulting sequences can also be collapsed by their compartment tags and associated to their corresponding compartmental proteome, which in a particular embodiment contains only a single or a very limited number of protein molecules. In some embodiments, both protein identification and quantification can be derived from this digital peptide information.

The methods disclosed herein can be used for analysis, including detection, quantitation and/or sequencing, of a plurality of macromolecules simultaneously (multiplexing). Multiplexing as used herein refers to analysis of a plurality of macromolecules (e.g. peptides) in the same assay. The plurality of macromolecules can be derived from the same sample or different samples. The plurality of macromolecules can be derived from the same subject or different subjects. The plurality of macromolecules that are analyzed can be different macromolecules, or the same macromolecule derived from different samples. A plurality of macromolecules includes 2 or more macromolecules, 5 or more macromolecules, 10 or more macromolecules, 50 or more macromolecules, 100 or more macromolecules, 500 or more macromolecules, 1000 or more macromolecules, 5,000 or more macromolecules, 10,000 or more macromolecules, 50,000 or more macromolecules, 100,000 or more macromolecules, 500,000 or more macromolecules, or 1,000,000 or more macromolecules.

### VI. Kits and Articles of Manufacture

Provided herein are kits and articles of manufacture comprising components for preparing and analyzing macromolecules (e.g., proteins, peptides, or peptides). The kits and articles of manufacture may include any one or more of the reagents and components used in the methods described above. In some embodiments, the kits optionally include instructions for use. In some embodiments, the kits comprise one or more of the following components: recoding tag(s), reagent(s) for attaching the recording tag, reagent(s) for transferring information from the probe tag to the recording tag, binding agent(s), reagent(s) for transferring identifying information from the coding tag to the recording tag, sequencing reagent(s), solid support(s), enzyme(s), buffer(s), and/or sample processing reagent(s) (e.g. fixation and permeabilization reagent(s).

In another embodiment, provided herein is a kit for obtaining an information regarding at least one amino acid residue of a peptide, the kit comprises:
a) a N-terminal modifier agent that is configured to contact a peptide to form a N-terminally modified peptide having a formula: Z-P1-P2-peptide, wherein P1 is a N-terminal amino acid residue of the peptide, P2 is a penultimate terminal amino acid residue of the peptide, and Z is an N-terminal modification capable of coordinating or chelating a metal ion M; and/or
b) a metalloprotein binder that binds to the metal ion M that is configured to specifically bind to the N-terminally modified peptide through interaction between the metalloprotein binder, the metal ion M and the Z-P1-P2-peptide, wherein the binding specificity between the metalloprotein binder and the Z1-P1-P2-peptide is predominantly or substantially determined by interaction between the metalloprotein binder and a Z1-P1 group of the Z1-P1-P2-peptide.

In some embodiments, the kit comprises a plurality of metalloprotein binders.

In some embodiments, the kit further comprises reagents for treating the peptides. Any combination of fractionation, enrichment, and subtraction methods, of the macromolecules, e.g., the proteins, may be performed. For example, the reagent may be used to fragment or digest the macromolecules, e.g., the proteins. In some cases, the kit comprises reagents and components to fractionate, isolate, subtract, enrich the macromolecules, e.g., the proteins. In some embodiments, the kits further comprises a protease such as trypsin, LysN, or LysC.

In some embodiments, the kit also comprises one or more buffers or reaction fluids necessary for any of the desired reaction to occur. Buffers including wash buffers, reaction buffers, and binding buffers, elution buffers and the like are known to those or ordinary skill in the arts. In some embodiments, the kits further include buffers and other components to accompany other reagents described herein.

Reagents and kit components may be provided in any suitable container. In some embodiments, the kits further include buffers and other components to accompany other reagents described herein. The reagents, buffers, and other components may be provided in vials (such as sealed vials), vessels, ampules, bottles, jars, flexible packaging (e.g., sealed Mylar or plastic bags), and the like. Any of the components of the kits may be sterilized and/or sealed. For example, a kit may provide one or more reaction or storage buffers. Reagents may be provided in a form that is usable in a particular assay, or in a form that requires addition of one or more other components before use (e.g. in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from about 7 to about 10.

In some embodiments, the kits or articles of manufacture may further comprise instruction(s) on the methods and uses described herein. In some embodiments, the instructions are directed to methods of analyzing the macromolecules (e.g., proteins, peptides, or peptides). The kits described herein may also include other materials desirable from a commercial and user standpoint, including other buffers, diluents, filters, syringes, and package inserts with instructions for performing any methods described herein. Any of the components of the kits may be sterilized and/or sealed.

Any of the above-mentioned kit and components, and any molecule, molecular complex or conjugate, reagent (e.g., chemical or biological reagents), agent, structure (e.g., support, surface, particle, or bead), reaction intermediate, reaction product, binding complex, or any other article of manufacture disclosed and/or used in the exemplary kits and methods, may be provided separately or in any suitable combination in order to form a kit.

Further aspects of the invention are discussed below.

In one embodiment, provided herein is an engineered metalloprotein binder that specifically binds to an N-terminally modified target peptide modified by an N-terminal modifier agent, wherein:
(a) the N-terminally modified target peptide has a formula: Z-P1-P2-peptide, wherein Z is an N-terminal modification capable of coordinating or chelating a zinc metal cation M, P1-P2-peptide is a target peptide before modification with the N-terminal modifier agent, Z-P1 is a modified N-terminal amino acid (NTAA) residue of the target peptide, and P2 is a penultimate terminal amino acid residue of the target peptide;
(b) the engineered metalloprotein binder specifically binds to the N-terminally modified target peptide through interaction between the engineered metalloprotein binder and the Z-P1 of the N-terminally modified target peptide; and
(c) the engineered metalloprotein binder comprises an amino acid sequence X1-C/H/D/E-X2-C/H/D/E-X3-C/H/D/E-X4, wherein C/H/D/E is any single amino acid residue independently selected from the group consisting of amino acid residues C (Cys), H (His), D (Asp) and E (Glu); X1, X2, X3 and X4 are each any amino acid sequence comprising between 0 and 200 amino acid residues in length, and wherein the amino acid sequence X1-C/H/D/E-X2-C/H/D/E-X3-C/H/D/E-X4 chelates a zinc metal cation M with a thermodynamic dissociation constant of 0.5 nM or less.

In another embodiment, provided herein is method of treating a target peptide, the method comprises the following steps:
(a) contacting the target peptide with an N-terminal modifier agent to form an N-terminally modified peptide having a formula: Z-P1-P2-peptide, wherein Z is an N-terminal modification capable of coordinating or chelating a zinc metal cation M, P1-P2-peptide is a target peptide before modification with the N-terminal modifier agent, Z-P1 is a modified N-terminal amino acid (NTAA) residue of the target peptide, and P2 is a penultimate terminal amino acid residue of the target peptide; and
(b) contacting an engineered metalloprotein binder with the N-terminally modified target peptide to allow the engineered binder to specifically bind to the N-terminally modified target peptide through interaction between the engineered binder and the modified NTAA residue of the N-terminally modified target peptide, wherein the engineered binder comprises an amino acid sequence X1-C/H/D/E-X2-C/H/D/E-X3-C/H/D/E-X4, wherein C/H/D/E is any single amino acid residue independently selected from the group consisting of amino acid residues C (Cys), H (His), D (Asp) and E (Glu); X1, X2, X3 and X4 are each any amino acid sequence comprising between 0 and 200 amino acid residues in length, and wherein the amino acid sequence X1-C/H/D/E-X2-C/H/D/E-X3-C/H/D/E-X4 chelates a zinc metal cation M with a thermodynamic dissociation constant of 0.5 nM or less.

In preferred embodiments, X1, X2, X3 and X4 together comprise at least 30 amino acid residues in length. It is because X1-C/H/D/E-X2-C/H/D/E-X3-C/H/D/E-X4 should form a 3D structure that chelates a zinc metal cation M and accommodates a modified N-terminal amino acid (NTAA) residue of the target peptide. Three C/H/D/E residues form an active Zn(II) binding site within this 3D structure and each forms separate coordination bonds with the metal cation. The forth coordination bond is formed between the metal cation and the NTM of the N-terminally modified peptide upon binding of the N-terminally modified peptide to the engineered metalloprotein binder.

In some embodiments, X1, X2, X3 and X4 together comprise at least 40, 50, 60, 70, 80, 90, 100, 150, or 200 amino acid residues in length. In some embodiments, X1, X2, X3 and X4 are each any amino acid sequence comprising between 1 and 200, 1 and 100, 1 and 50, 5 and 200, 5 and 100, 10 and 200, or 10 and 100 amino acid residues in length.

In preferred embodiments, the engineered metalloprotein binder chelates a zinc metal cation Zn(II) with a thermodynamic dissociation constant of less than 0.5 nM, less than 0.1 nM or less than 0.001 nM. For example, the wild-type hCAII metalloenzyme binds Zn(II) with thermodynamic dissociation constant (Kd) of ˜4 pM (Ippolito J A, et al., Structure-assisted redesign of a protein-zinc-binding site with femtomolar affinity. Proc Natl Acad Sci USA. 1995 May 23; 92(11):5017-21). Other natural and designed metalloproteins have zinc binding constants (Kd) ranging from fM to nM (Petros A K, et al., Femtomolar Zn(II) affinity in a peptide-based ligand designed to model thiolate-rich metalloprotein active sites. Inorg Chem. 2006 Dec. 11; 45(25):9941-58; Chan K L, et al., Characterization of the Zn(II) binding properties of the human Wilms' tumor suppressor protein C-terminal zinc finger peptide. Inorg Chem. 2014 Jun. 16; 53(12):6309-20).

In some embodiments, the engineered metalloprotein binder binds to the N-terminally modified target peptide with at least a 100-fold greater binding affinity than a model peptide that has at least 90% homology to the engineered binder over the entire sequence length, wherein the model peptide does not comprise the amino acid sequence X1-C/H/D/E-X2-C/H/D/E-X3-C/H/D/E-X4. For example, when at least one of the C/H/D/E residues of the engineered metalloprotein binder is mutated, the resulting motif is no longer capable of chelating a zinc metal cation M with a thermodynamic dissociation constant (Kd) of 0.5 nM or less. Such binder has a significantly reduced (such as at least 2, 5, 10, 100 or 1000 fold reduced) binding affinity towards the N-terminally modified target peptide. These reductions were calculated for exemplary binder scaffolds having sequences set forth in SEQ ID NO: 7-27 as shown in Tables 4-7 (the “Native Binder ΔKd” parameter).

In some embodiments, the engineered metalloprotein binder comprises an amino acid sequence having at least about 90% sequence homology to any one of the amino acid sequences selected from the group consisting of SEQ ID NO: 7-SEQ ID NO: 59. In some embodiments, the engineered metalloprotein binder comprises an amino acid sequence having at least about 70, 75, 80, 85, 90, 95, 97, 98 or 99% sequence homology to any one of the amino acid sequences selected from the group consisting of SEQ ID NO: 7-SEQ ID NO: 59.

In some embodiments, the engineered metalloprotein binder comprises an amino acid sequence having at least about 90% sequence identity to any one of the amino acid sequences selected from the group consisting of SEQ ID NO: 7-SEQ ID NO: 59. In some embodiments, the engineered metalloprotein binder comprises an amino acid sequence having at least about 70, 75, 80, 85, 90, 95, 97, 98 or 99% sequence identity to any one of the amino acid sequences selected from the group consisting of SEQ ID NO: 7-SEQ ID NO: 59.

In some embodiments, the engineered metalloprotein binder comprises an amino acid sequence, which differs from one of the amino acid sequences set forth in SEQ ID NO: 7, SEQ ID NO: 8, SEQ ID NO: 9, SEQ ID NO: 14, SEQ ID NO: 15, SEQ ID NO: 16, SEQ ID NO: 17, SEQ ID NO: 21, SEQ ID NO: 25, SEQ ID NO: 27, SEQ ID NO: 30, SEQ ID NO: 31, SEQ ID NO: 39 or SEQ ID NO: 43 by at least one amino acid residue in a Z-P1 binding site, or within 6 Å of the Z-P1 binding site of the engineered metalloprotein binder, wherein the Z-P1 binding site comprises amino acids corresponding to amino acid positions 59, 64, 66, 88, 89, 91, 93, 103, 116, 118, 127, 128, 131, 137, 139, 193, 194, 195, 196, 198, 203, and 205 of SEQ ID NO: 7; or the Z-P1 binding site comprises amino acids corresponding to amino acid positions 60, 63, 65, 68, 87, 88, 90, 92, 102, 115, 117, 127, 137, 139, 193, 194, 195, 196, 197, 198, 203, and 205 of SEQ ID NO: 8; or the Z-P1 binding site comprises amino acids corresponding to amino acid positions 1, 2, 17, 59, 61, 64, 89, 91, 93, 103, 116, 118, 127, 131, 139, 193, 194, 195, 196, 197, 198, 199, 203, and 205 of SEQ ID NO: 9; or the Z-P1 binding site comprises amino acids corresponding to amino acid positions 99, 100, 132-136, 190, 191, 194, 200, and 222 of SEQ ID NO: 14; or the Z-P1 binding site comprises amino acids corresponding to amino acid positions 63-66, 83, 84, 86, 89, 92, 93, 96, 102, 149, 152-155, 158, and 175-177 of SEQ ID NO: 15; or the Z-P1 binding site comprises amino acids corresponding to amino acid positions 117, 255, 256, 257, 258-260, 268, 270, 271, 290, 293, 294, 297, 315, 316, 377, 779, and 821 of SEQ ID NO: 16; or the Z-P1 binding site comprises amino acids corresponding to amino acid positions 41, 58, 59, 60, 61, 62, 65, 77, 105, 107, 108, 109, 110-112, 147, 150, 151, 154, 155, 158, and 185 of SEQ ID NO: 17; or the Z-P1 binding site comprises amino acids corresponding to amino acid positions 133-137, 153, 158, 160, 161, 169, 173, 176, 177, 180, 186, 191, 192, 209, 216, and 230 of SEQ ID NO: 21; or the Z-P1 binding site comprises amino acids corresponding to amino acid positions 4-7, 9, 10, 14, 58, 90, 113, 134-138, 162, 181, 183-185, and 212 of SEQ ID NO: 25; or the Z-P1 binding site comprises amino acids corresponding to amino acid positions 7, 9, 20, 104, 108, 139, 141, 164, 167, 169, 170, 171, 202, 204-210, 242, 245, and 248 of SEQ ID NO: 27; or the Z-P1 binding site comprises amino acids corresponding to amino acid positions 90, 93, 96-99, 132, 133, 136, 142, 152, and 153 of SEQ ID NO: 30; or the Z-P1 binding site comprises amino acids corresponding to amino acid positions 165, 166, 169, 227-230, 231, 235, 248, and 352 of SEQ ID NO: 31; or the Z-P1 binding site comprises amino acids corresponding to amino acid positions 106-109, 141, 142, 145, 151, and 166-168 of SEQ ID NO: 39; or the Z-P1 binding site comprises amino acids corresponding to amino acid positions 106, 107, 313-317, 325, 327, 379, 382-389, 448, 453, 506, and 564-566 of SEQ ID NO: 43. To better accommodate Z-P1-P2-peptide in a substrate-binding pocket, the engineered metalloprotein binder can be mutated at amino acid residues of the Z-P1 binding site, or at amino acid residues within 6 Å of the Z-P1 binding site, which roughly corresponds to amino acid residues adjacent to the Z-P1 binding site. For example, any amino acid residues within the Z-P1 binding site or having a Ca atom within 6 Å of the Z-P1 binding site could be mutated to any of the 20 amino acid residues. The Z-P1 binding site of the binder comprises amino acid residues that are involved in binding of the modified N-terminal amino acid (NTAA) residue of the target peptide.

In some embodiments, the N-terminal modifier agent is a compound of the following formula:

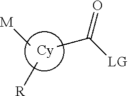

wherein:
M is a metal binding group that comprises sulfonamide, hydroxamic acid, sulfamate, or sulfamide;
the group

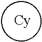

is a 5 or 6 membered aromatic ring which may contain up to three heteroatoms selected from N, O and S as ring members, and is optionally substituted by R;
R represents one or two optional substituents selected from the group consisting of F, Cl, CH₃, CF₂H, CF₃, OH, OCH₃, OCF₃, NH₂, N(CH₃)₂, NO₂, SCH₃, SO₂CH₃, CH₂OH, B(OH)₂, CN, CONH₂, and CONHCH₃; and
LG is a leaving group.

In some embodiments, LG is selected from the group consisting of N-succinimidyloxy, sulfo-N-succinimidyloxy, pentafluorophenoxy, tetrafluorophenoxy, 4-sulfo-phenoxy, and pyridinyl-2-oxy N-oxide.

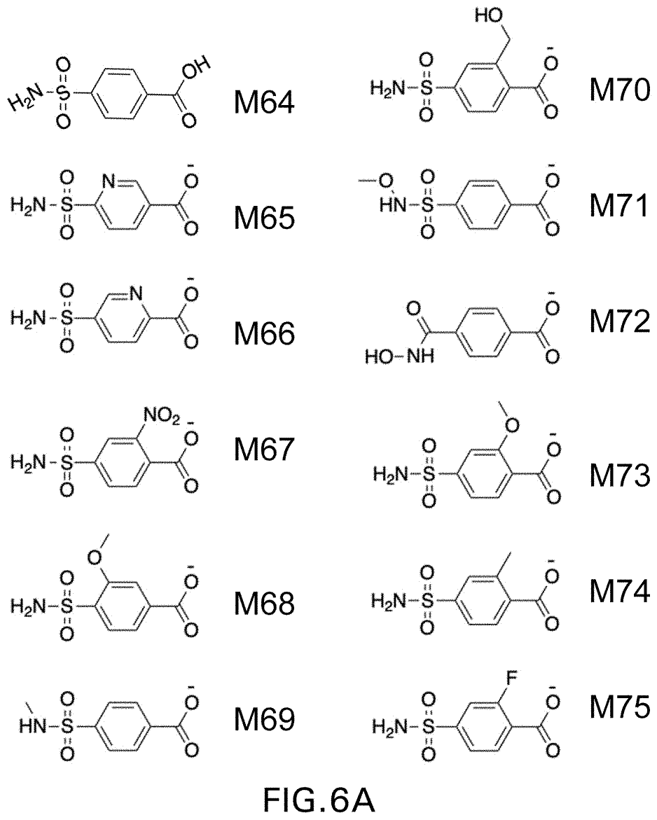

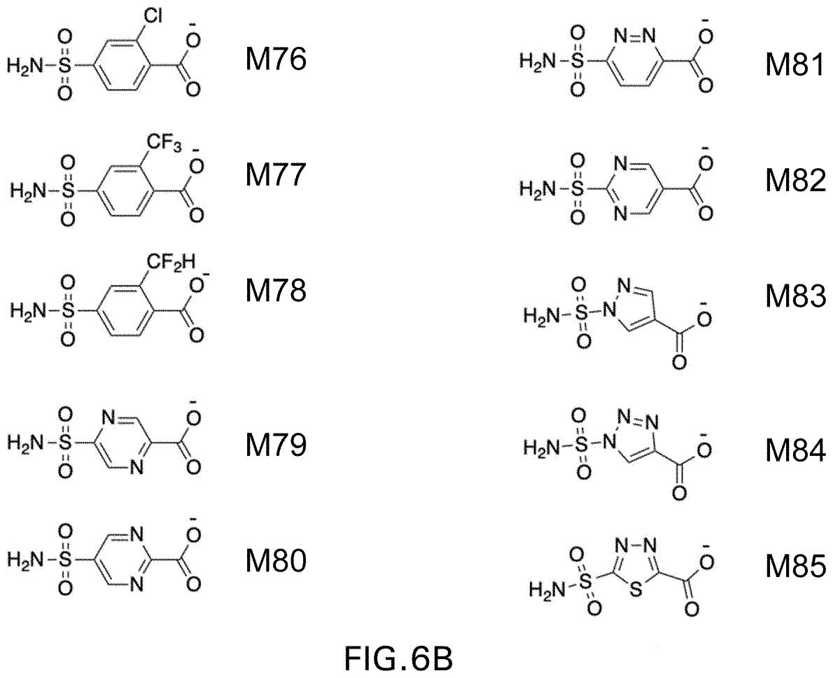

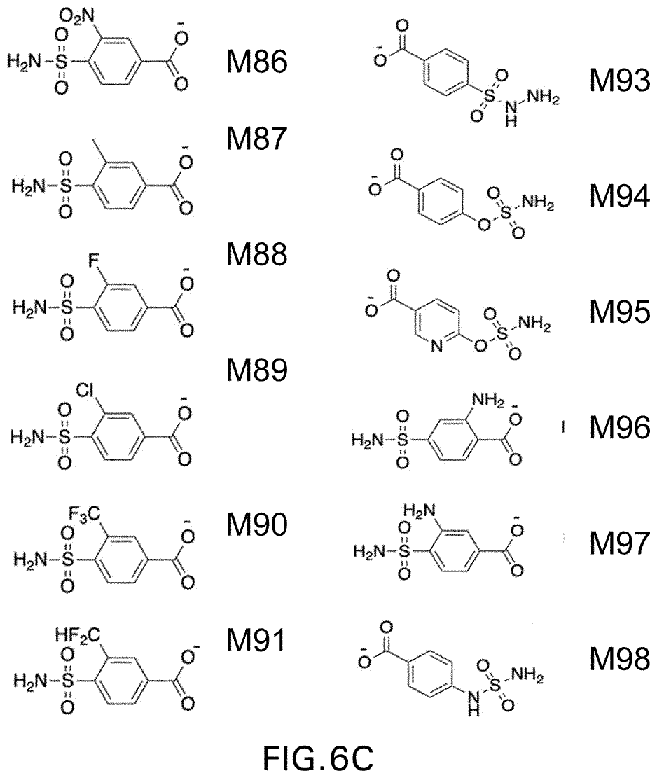

In some embodiments, the N-terminal modifier agent is one selected from the group consisting of NTM M64-NTM M98, the structures of which are shown in FIG. 6A-FIG. 6C.

In some embodiments, the engineered metalloprotein binder binds to the N-terminally modified target peptide with a thermodynamic dissociation constant (Kd) of 500 nM or less. In preferred embodiments, the engineered metalloprotein binder binds to the N-terminally modified target peptide with a thermodynamic dissociation constant (Kd) of less than 300 nM, less than 200 nM, less than 100 nM, less than 10 nM or less than 5 nM.

In some embodiments, the methods disclosed herein further comprise step (c): removing the modified NTAA residue from the N-terminally modified target peptide, thereby exposing a new NTAA residue.

In some embodiments, steps (a), (b) and (c) of the methods disclosed herein are repeated sequentially at least one time. In each modifying-binding-cleaving cycle, information regarding the (current) N-terminal amino acid of the target peptide can be obtained. Repeating this cycle more than one time can provide information regarding the amino acid sequence of the target peptide (both identity and order of the amino acid residues of the target peptide can be obtained).

In some embodiments, the methods disclosed herein further comprise immobilizing the target peptide on a solid support before step (a).

In some embodiments, the engineered binder comprises a detectable label or a nucleic acid tag, or a nucleic acid coding tag.

In some embodiments, the target peptide immobilized on a solid support is associated with a nucleic acid recording tag. In these embodiments, the engineered binder can comprise a nucleic acid coding tag that comprises identifying information regarding the engineered binder. Methods of encoding a history of binding events into nucleic acid sequence are disclosed in US published application US 2019/0145982 A1, and can be utilized with the methods disclosed herein.

In some embodiments, the N-terminal modifier agent further comprises a peptide coupling reagent.

Suitable reagents that are known in the art for performing the coupling reaction (amide bond formation) between the NTM and the NTAA include conventional peptide coupling reagents such as carbodiimides (e.g., dicyclohexyl carbodiimide (DCC), diisopropyl carbodiimide (DIPC), 1-ethyl-3-(3-dimethylaminopropyl)carbodiimide (EDC), 1-cyclohexyl-(2-morpholinoethyl)carbodiimide tosylate (CMCT), and the like), aminium/uronium salts (e.g., COMU, HATU, HBTU, TBTU, HCTU, and TSTU), phosphonium coupling reagents including PyBOP, PyAOP, PyOxim, and BOP, and phosphonate coupling reagents such as (3-(diethoxyphosphoryloxy)-1,2,3-benzotriazin-4(3H)-one) (DEPBT), and propylphosphonic anhydride (T3P). Suitable carbodiimide reagents include compounds of Formula (1) described below. Suitable aminium/uronium coupling reagents include compounds of Formula (2) described below.

In some embodiments, coupling conditions are used to minimize racemization of the NTMaa moiety of the N-terminal modifier agent during installation onto target peptides (Ramu, Vasanthakumar G., et al., “DEPBT as Coupling Reagent To Avoid Racemization in a Solution-Phase Synthesis of a Kyotorphin Derivative.” 2014, Synthesis 46 (11): 1481-86).

In some embodiments, the chemical reagent comprises compound of Formula (1):

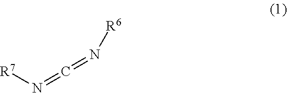

or a salt or conjugate thereof,
wherein
R⁶ and R⁷ are each independently C₁₋₆ alkyl, —CO₂C₁₋₄ alkyl, —OR^{k}, aryl, heteroaryl, cycloalkyl or heterocyclyl, wherein the C₁₋₆ alkyl, —CO₂C₁₋₄ alkyl, —OR^{k}, aryl, and cycloalkyl are each unsubstituted or substituted; and
R^{k} is H, C₁₋₆ alkyl, or heterocyclyl, wherein the C₁₋₆ alkyl and heterocyclyl are each unsubstituted or substituted; wherein heterocyclyl can be 5-8 membered ring comprising one or two heteroatoms selected from N, O and S as ring members, where the heteroaryl can be a 5-6 membered single ring or 8-10 membered bicyclic ring, each of which comprises one to three heteroatoms selected from N, O and S as ring members. Cycloalkyls include 3-7 membered carbocyclic rings, optionally substituted. Heterocyclyl can be 5-8 membered ring comprising one or two heteroatoms selected from N, O and S as ring members, and include tetrahydrofuranyl, piperidinyl, piperazinyl, dihydropyranyl, dioxanyl, and the like. Heteroaryl can be a 5-6 membered single ring or 8-10 membered bicyclic ring, each of which comprises one to three heteroatoms selected from N, O and S as ring members. Aryl includes phenyl, which can be substituted or unsubstituted. Heteroaryl includes pyridinyl, pyrimidinyl, or pyrazinyl; oxazolyl, isoxazolyl, thiazolyl, isothiazolyl, furanyl, thienyl, pyrrolidinyl, imidazolyl, pyrazolyl, and triazolyl, as well as a bicyclic group comprising any one of these fused to phenyl. Suitable substituents for the alkyl, cycloalkyl, heterocyclyl, aryl and heteroaryl groups include halo, hydroxy, amino, C₁-C₂ alkylamino, di-(C₁-C₂ alkyl)amino, C₁-C₂ alkyl, C₁-C₂ alkoxy, NO₂, CN, C₁-C₂ haloalkyl, C₁-C₂ haloalkoxy, and halo, and for non-aromatic groups also oxo.

In some embodiments of Formula (1), R⁶ and R⁷ are each independently C₁₋₆ alkyl, 3-7 membered cycloalkyl, —CO₂C₁₋₄ alkyl, or aryl, especially phenyl. In some embodiments, R⁶ and R⁷ are each independently H, C₁₋₆ alkyl, phenyl, or cycloalkyl. In some embodiments, R⁶ and R⁷ are the same. In some embodiments, R⁶ and R⁷ are different.

In some embodiments, one of R⁶ and R⁷ is C₁₋₆ alkyl and the other is selected from the group consisting of C₁₋₆ alkyl, —CO₂C₁₋₄ alkyl, and —OR^{k}, wherein the C₁₋₆ alkyl, —CO₂C₁₋₄ alkyl, and —OR^{k} are each unsubstituted or substituted. In some embodiments, one or both of R⁶ and R⁷ is C₁₋₆ alkyl, optionally substituted with aryl, such as phenyl. In some embodiments, one or both of R⁶ and R⁷ is C₁₋₆ alkyl, optionally substituted with heterocyclyl. In some embodiments, one of R⁶ and R⁷ is —CO₂C₁₋₄ alkyl and the other is selected from the group consisting of C₁₋₆ alkyl, —CO₂C₁₋₄ alkyl, and —OR^{k}, wherein the C₁₋₆ alkyl, —CO₂C₁₋₄ alkyl, and —OR^{k} are each unsubstituted or substituted. In some embodiments, one of R⁶ and R⁷ is optionally substituted aryl and the other is selected from the group consisting of C₁₋₆ alkyl, —CO₂C₁₋₄ alkyl, —OR^{k}, aryl, heteroaryl, cycloalkyl and heterocyclyl, wherein the C₁₋₆ alkyl, —CO₂C₁₋₄ alkyl, —OR^{k}, aryl, and cycloalkyl are each unsubstituted or substituted. In some embodiments, one or both of R⁶ and R⁷ is aryl, optionally substituted with up to three groups selected from C₁₋₆ alkyl, halo, and NO₂.

In yet another embodiment, provided herein is a method of treating a target peptide, the method comprises:
(a) contacting the target peptide with an N-terminal modifier agent to form an N-terminally modified peptide having a formula: Z-P1-P2-peptide, wherein Z is an N-terminal modification capable of coordinating or chelating a zinc metal cation M, P1-P2-peptide is a target peptide before modification with the N-terminal modifier agent, Z-P1 is a modified N-terminal amino acid (NTAA) residue of the target peptide, and P2 is a penultimate terminal amino acid residue of the target peptide; and
(b) contacting an engineered metalloprotein binder with the N-terminally modified target peptide to allow the engineered binder to specifically bind to the N-terminally modified target peptide through interaction between the engineered binder and the modified NTAA residue of the N-terminally modified target peptide, wherein the engineered binder comprises an amino acid sequence X1-C/H/D/E-X2-C/H/D/E-X3-C/H/D/E-X4, wherein C/H/D/E is any single amino acid residue independently selected from the group consisting of amino acid residues C (Cys), H (His), D (Asp) and E (Glu); X1, X2, X3 and X4 are each any amino acid sequence comprising between 0 and 200 amino acid residues in length, and wherein the amino acid sequence X1-C/H/D/E-X2-C/H/D/E-X3-C/H/D/E-X4 chelates a zinc metal cation M with a thermodynamic dissociation constant of 0.5 nM or less.

In some embodiments, the engineered metalloprotein binder binds to the N-terminally modified target peptide with at least a 100-fold greater binding affinity than a model peptide that has at least 90% homology to the engineered binder over the entire sequence length, wherein the model peptide does not comprise the amino acid sequence X1-C/H/D/E-X2-C/H/D/E-X3-C/H/D/E-X4.

In some embodiments, the engineered metalloprotein binder comprises an amino acid sequence having at least about 90% sequence homology to any one of the amino acid sequences selected from the group consisting of SEQ ID NO: 7-SEQ ID NO: 59.

In some embodiments, the engineered metalloprotein binder comprises an amino acid sequence, which differs from one of the amino acid sequences set forth in SEQ ID NO: 7, SEQ ID NO: 8, SEQ ID NO: 9, SEQ ID NO: 14, SEQ ID NO: 15, SEQ ID NO: 16, SEQ ID NO: 17, SEQ ID NO: 21, SEQ ID NO: 25, SEQ ID NO: 27, SEQ ID NO: 30, SEQ ID NO: 31, SEQ ID NO: 39 or SEQ ID NO: 43 by at least one amino acid residue in a Z-P1 binding site, or within 6 Å of the Z-P1 binding site of the engineered metalloprotein binder, wherein the Z-P1 binding site comprises amino acids corresponding to amino acid positions 59, 64, 66, 88, 89, 91, 93, 103, 116, 118, 127, 128, 131, 137, 139, 193, 194, 195, 196, 198, 203, and 205 of SEQ ID NO: 7; or the Z-P1 binding site comprises amino acids corresponding to amino acid positions 60, 63, 65, 68, 87, 88, 90, 92, 102, 115, 117, 127, 137, 139, 193, 194, 195, 196, 197, 198, 203, and 205 of SEQ ID NO: 8; or the Z-P1 binding site comprises amino acids corresponding to amino acid positions 1, 2, 17, 59, 61, 64, 89, 91, 93, 103, 116, 118, 127, 131, 139, 193, 194, 195, 196, 197, 198, 199, 203, and 205 of SEQ ID NO: 9; or the Z-P1 binding site comprises amino acids corresponding to amino acid positions 99, 100, 132-136, 190, 191, 194, 200, and 222 of SEQ ID NO: 14; or the Z-P1 binding site comprises amino acids corresponding to amino acid positions 63-66, 83, 84, 86, 89, 92, 93, 96, 102, 149, 152-155, 158, and 175-177 of SEQ ID NO: 15; or the Z-P1 binding site comprises amino acids corresponding to amino acid positions 117, 255, 256, 257, 258-260, 268, 270, 271, 290, 293, 294, 297, 315, 316, 377, 779, and 821 of SEQ ID NO: 16; or the Z-P1 binding site comprises amino acids corresponding to amino acid positions 41, 58, 59, 60, 61, 62, 65, 77, 105, 107, 108, 109, 110-112, 147, 150, 151, 154, 155, 158, and 185 of SEQ ID NO: 17; or the Z-P1 binding site comprises amino acids corresponding to amino acid positions 133-137, 153, 158, 160, 161, 169, 173, 176, 177, 180, 186, 191, 192, 209, 216, and 230 of SEQ ID NO: 21; or the Z-P1 binding site comprises amino acids corresponding to amino acid positions 4-7, 9, 10, 14, 58, 90, 113, 134-138, 162, 181, 183-185, and 212 of SEQ ID NO: 25; or the Z-P1 binding site comprises amino acids corresponding to amino acid positions 7, 9, 20, 104, 108, 139, 141, 164, 167, 169, 170, 171, 202, 204-210, 242, 245, and 248 of SEQ ID NO: 27; or the Z-P1 binding site comprises amino acids corresponding to amino acid positions 90, 93, 96-99, 132, 133, 136, 142, 152, and 153 of SEQ ID NO: 30; or the Z-P1 binding site comprises amino acids corresponding to amino acid positions 165, 166, 169, 227-230, 231, 235, 248, and 352 of SEQ ID NO: 31; or the Z-P1 binding site comprises amino acids corresponding to amino acid positions 106-109, 141, 142, 145, 151, and 166-168 of SEQ ID NO: 39; or the Z-P1 binding site comprises amino acids corresponding to amino acid positions 106, 107, 313-317, 325, 327, 379, 382-389, 448, 453, 506, and 564-566 of SEQ ID NO: 43.

In some embodiments, the engineered metalloprotein binder binds to the N-terminally modified target peptide with a thermodynamic dissociation constant (Kd) of 500 nM or less.

In some embodiments, the engineered metalloprotein binder comprises a detectable label or a nucleic acid tag.

### EXAMPLARY EMBODIMENTS

The following enumerated embodiments represent certain embodiments and examples of the invention:
1. A method for obtaining an information regarding at least one amino acid residue of a peptide, comprising the steps of:
a) contacting a peptide with a first N-terminal modifier agent to form a N-terminally modified peptide having a formula:
Z1-P1-P2-peptide, wherein P1 is a N-terminal amino acid residue of the peptide, P2 is a penultimate terminal amino acid residue of the peptide, and Z1 is an N-terminal modification capable of coordinating or chelating a metal ion M1;
b) providing a first metalloprotein binder that binds to the metal ion M1 and allowing specific binding between the Z1-P1-P2-peptide, the first metalloprotein binder and the metal ion M1, wherein the binding specificity between the first metalloprotein binder and the Z1-P1-P2-peptide is predominantly or substantially determined by interaction between the first metalloprotein binder and a Z1-P1 group of the Z1-P1-P2-peptide;
c) obtaining an information regarding the first metalloprotein binder; and
d) obtaining an information regarding the P1 amino acid residue of the peptide based on the obtained information regarding the first metalloprotein binder.
2. The method of embodiment 1, wherein at step (b) a first set of metalloprotein binders comprising the first metalloprotein binder is provided, and each metalloprotein binder from the first set of metalloprotein binders binds to the metal ion M1.
3. The method of embodiment 1, further comprising the following steps:
i) cleaving a peptide bond between P1 and P2 of the Z-P1-P2-peptide to form a second peptide having P2 as a new N-terminal amino acid residue;
ii) contacting the peptide with a second N-terminal modifier agent to form a N-terminally modified peptide having a formula: Z2-P2-peptide, wherein Z2 is an N-terminal modification capable of coordinating or chelating a metal ion M2;
iii) providing a second metalloprotein binder that binds to the metal ion M2 and allowing specific binding between the Z2-P2-peptide, the second metalloprotein binder and the metal ion M2, wherein the binding specificity between the second metalloprotein binder and the Z2-P2-peptide is predominantly or substantially determined by interaction between the second metalloprotein binder and a Z2-P2 group of the Z2-P2-peptide;
iv) obtaining an information regarding the second metalloprotein binder; and
v) obtaining an information regarding the P2 amino acid residue of the peptide based on the obtained information regarding the second metalloprotein binder.
4. The method of embodiment 2, further comprising the following steps:
i) cleaving a peptide bond between P1 and P2 of the Z-P1-P2-peptide to form a second peptide having P2 as a new N-terminal amino acid residue;
ii) contacting the peptide with a second N-terminal modifier agent to form a N-terminally modified peptide having a formula: Z2-P2-peptide, wherein Z2 is an N-terminal modification capable of coordinating or chelating a metal ion M2;
iii) providing a second set of metalloprotein binders comprising a second metalloprotein binder, wherein each metalloprotein binder from the second set of metalloprotein binders binds to the metal ion M2, and allowing specific binding between the Z2-P2-peptide, the second metalloprotein binder and the metal ion M2, wherein the binding specificity between the second metalloprotein binder and the Z2-P2-peptide is predominantly or substantially determined by interaction between the second metalloprotein binder and a Z2-P2 group of the Z2-P2-peptide;
iv) obtaining an information regarding the second metalloprotein binder; and
v) obtaining an information regarding P2 amino acid residue of the peptide based on the obtained information regarding the second metalloprotein binder.
5. The method of embodiment 3 or 4, wherein the first N-terminal modifier agent is the same as the second N-terminal modifier agent, and Z1 is the same as Z2.
6. The method of any one of embodiments 3-5, wherein the first set of metalloprotein binders is the same as the second set of metalloprotein binders, and M1 is the same as M2.
7. The method of any one of embodiments 1-6, wherein the peptide is immobilized to a solid support.
8. The method of any one of embodiments 3-7, wherein the peptide bond between the P1 and

P2 is cleaved using a chemical agent or an enzyme.
9. The method of any one of embodiments 1-6, wherein the metal ion M is Zn(II).
10. The method of any one of embodiments 3-9, wherein the first metalloprotein binder has an affinity for the Z1-P1 group of the Z1-P1-P2-peptide with a Kd of less than 200 nM and the second metalloprotein binder has an affinity for the Z2-P2 group of the Z2-P2-peptide with a Kd of less than 200 nM.
11. The method of any one of embodiments 1-10, wherein obtaining an information regarding P1 amino acid residue comprises identifying P1 amino acid residue.
12. The method of any one of embodiments 1-11, wherein
providing the peptide comprises providing the peptide associated with a recording tag immobilized on a solid support;
the first metalloprotein binder is associated with a coding tag comprising identifying information regarding the first metalloprotein binder;
obtaining an information regarding the first metalloprotein binder comprises, upon binding of the first metalloprotein binder to the first N-terminally modified peptide, transferring identifying information from the coding tag to the recording tag associated with the immobilized peptide to generate an extended recording tag; and
obtaining an information regarding P1 amino acid residue of the peptide comprises analyzing the extended recording tag by a sequencing method.
13. The method of any one of embodiments 1-17, wherein the first metalloprotein binder is fluorescently labeled; and identifying P1 amino acid residue of the peptide comprises detecting the fluorescence from the first metalloprotein binder.
14. A method of treating a target peptide, the method comprises the following steps:
(a) contacting the target peptide with an N-terminal modifier agent to form an N-terminally modified peptide having a formula: Z-P1-P2-peptide, wherein Z is an N-terminal modification capable of coordinating or chelating a zinc metal cation M, P1-P2-peptide is a target peptide before modification with the N-terminal modifier agent, Z-P1 is a modified N-terminal amino acid (NTAA) residue of the target peptide, and P2 is a penultimate terminal amino acid residue of the target peptide; and
(b) contacting an engineered metalloprotein binder with the N-terminally modified target peptide to allow the engineered binder to specifically bind to the N-terminally modified target peptide through interaction between the engineered binder and the modified NTAA residue of the N-terminally modified target peptide, wherein the engineered binder comprises an amino acid sequence X1-C/H/D/E-X2-C/H/D/E-X3-C/H/D/E-X4, wherein C/H/D/E is any single amino acid residue independently selected from the group consisting of amino acid residues C (Cys), H (His), D (Asp) and E (Glu); X1, X2, X3 and X4 are each any amino acid sequence comprising between 0 and 200 amino acid residues in length, and wherein the amino acid sequence X1-C/H/D/E-X2-C/H/D/E-X3-C/H/D/E-X4 chelates a zinc metal cation M with a thermodynamic dissociation constant of 0.5 nM or less. In preferred embodiments, X1, X2, X3 and X4 together comprise at least 30 amino acid residues in length.
15. The method of embodiment 14, wherein the engineered metalloprotein binder binds to the N-terminally modified target peptide with at least a 100-fold greater binding affinity than a model peptide that has at least 90% homology to the engineered binder over the entire sequence length, wherein the model peptide does not comprise the amino acid sequence X1-C/H/D/E-X2-C/H/D/E-X3-C/H/D/E-X4.
16. The method of embodiment 14 or embodiment 15, wherein the engineered metalloprotein binder comprises an amino acid sequence having at least about 90% sequence homology to any one of the amino acid sequences selected from the group consisting of SEQ ID NO: 7-SEQ ID NO: 59.
17. The method of any one of embodiments 14-16, wherein the engineered metalloprotein binder comprises an amino acid sequence, which differs from one of the amino acid sequences set forth in SEQ ID NO: 7, SEQ ID NO: 8, SEQ ID NO: 9, SEQ ID NO: 14, SEQ ID NO: 15, SEQ ID NO: 16, SEQ ID NO: 17, SEQ ID NO: 21, SEQ ID NO: 25, SEQ ID NO: 27, SEQ ID NO: 30, SEQ ID NO: 31, SEQ ID NO: 39 or SEQ ID NO: 43 by at least one amino acid residue in a Z-P1 binding site, or within 6 Å of the Z-P1 binding site of the engineered metalloprotein binder, wherein the Z-P1 binding site comprises amino acids corresponding to amino acid positions 59, 64, 66, 88, 89, 91, 93, 103, 116, 118, 127, 128, 131, 137, 139, 193, 194, 195, 196, 198, 203, and 205 of SEQ ID NO: 7; or the Z-P1 binding site comprises amino acids corresponding to amino acid positions 60, 63, 65, 68, 87, 88, 90, 92, 102, 115, 117, 127, 137, 139, 193, 194, 195, 196, 197, 198, 203, and 205 of SEQ ID NO: 8; or the Z-P1 binding site comprises amino acids corresponding to amino acid positions 1, 2, 17, 59, 61, 64, 89, 91, 93, 103, 116, 118, 127, 131, 139, 193, 194, 195, 196, 197, 198, 199, 203, and 205 of SEQ ID NO: 9; or the Z-P1 binding site comprises amino acids corresponding to amino acid positions 99, 100, 132-136, 190, 191, 194, 200, and 222 of SEQ ID NO: 14; or the Z-P1 binding site comprises amino acids corresponding to amino acid positions 63-66, 83, 84, 86, 89, 92, 93, 96, 102, 149, 152-155, 158, and 175-177 of SEQ ID NO: 15; or the Z-P1 binding site comprises amino acids corresponding to amino acid positions 117, 255, 256, 257, 258-260, 268, 270, 271, 290, 293, 294, 297, 315, 316, 377, 779, and 821 of SEQ ID NO: 16; or the Z-P1 binding site comprises amino acids corresponding to amino acid positions 41, 58, 59, 60, 61, 62, 65, 77, 105, 107, 108, 109, 110-112, 147, 150, 151, 154, 155, 158, and 185 of SEQ ID NO: 17; or the Z-P1 binding site comprises amino acids corresponding to amino acid positions 133-137, 153, 158, 160, 161, 169, 173, 176, 177, 180, 186, 191, 192, 209, 216, and 230 of SEQ ID NO: 21; or the Z-P1 binding site comprises amino acids corresponding to amino acid positions 4-7, 9, 10, 14, 58, 90, 113, 134-138, 162, 181, 183-185, and 212 of SEQ ID NO: 25; or the Z-P1 binding site comprises amino acids corresponding to amino acid positions 7, 9, 20, 104, 108, 139, 141, 164, 167, 169, 170, 171, 202, 204-210, 242, 245, and 248 of SEQ ID NO: 27; or the Z-P1 binding site comprises amino acids corresponding to amino acid positions 90, 93, 96-99, 132, 133, 136, 142, 152, and 153 of SEQ ID NO: 30; or the Z-P1 binding site comprises amino acids corresponding to amino acid positions 165, 166, 169, 227-230, 231, 235, 248, and 352 of SEQ ID NO: 31; or the Z-P1 binding site comprises amino acids corresponding to amino acid positions 106-109, 141, 142, 145, 151, and 166-168 of SEQ ID NO: 39; or the Z-P1 binding site comprises amino acids corresponding to amino acid positions 106, 107, 313-317, 325, 327, 379, 382-389, 448, 453, 506, and 564-566 of SEQ ID NO: 43.
18. The method of any one of embodiments 14-17, wherein the N-terminal modifier agent is a compound of the following formula:

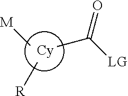

wherein:
M is a metal binding group that comprises sulfonamide, hydroxamic acid, sulfamate, or sulfamide;
the group

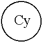

is a 5 or 6 membered aromatic ring which may contain up to three heteroatoms selected from N, O and S as ring members, and is optionally substituted by R;
R represents one or two optional substituents selected from the group consisting of F, Cl, CH3, CF2H, CF3, OH, OCH3, OCF3, NH2, N(CH3)2, NO2, SCH3, SO2CH3, CH2OH, B(OH)2, CN, CONH2, and CONHCH3; and
LG is a leaving group.
19. The method of embodiment 18, wherein LG is selected from the group consisting of N-succinimidyloxy, sulfo-N-succinimidyloxy, pentafluorophenoxy, tetrafluorophenoxy, 4-sulfo-phenoxy, and pyridinyl-2-oxy N-oxide.
20. The method of any one of embodiments 14-19, wherein the engineered metalloprotein binder binds to the N-terminally modified target peptide with a thermodynamic dissociation constant (Kd) of 500 nM or less.
21. The method of any one of embodiments 14-20, further comprising step (c): removing the modified NTAA residue from the N-terminally modified target peptide, thereby exposing a new NTAA residue.
22. The method of embodiment 21, wherein steps (a), (b) and (c) are repeated sequentially at least one time.
23. The method of any one of embodiments 14-22, further comprising immobilizing the target peptide on a solid support before step (a).
24. The method of embodiment 23, wherein the target peptide immobilized on a solid support is associated with a nucleic acid recording tag.
25. The method of any one of embodiments 14-24, wherein the engineered binder comprises a detectable label or a nucleic acid tag or a nucleic acid coding tag.
26. The method of any one of embodiments 14-25, wherein the N-terminal modifier agent further comprises a peptide coupling reagent.
27. The method of embodiment 26, wherein the peptide coupling reagent is a compound of Formula (1) or (2), wherein:
Formula (1) is

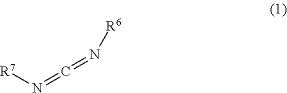

or a salt or conjugate thereof, wherein
R6 and R7 are each independently C1-6 alkyl, —CO2C1-4 alkyl, —ORk, aryl, heteroaryl, cycloalkyl or heterocyclyl, wherein the C1-6 alkyl, —CO2C1-4 alkyl, —ORk, aryl, and cycloalkyl are each unsubstituted or substituted; and
Rk is H, C1-6 alkyl, or heterocyclyl, wherein the C1-6 alkyl and heterocyclyl are each unsubstituted or substituted; wherein heterocyclyl can be 5-8 membered ring comprising one or two heteroatoms selected from N, O and S as ring members, where the heteroaryl can be a 5-6 membered single ring or 8-10 membered bicyclic ring, each of which comprises one to three heteroatoms selected from N, O and S as ring members; and
Formula (2) is:

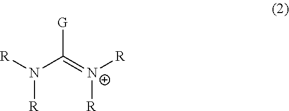

wherein:
each R is independently C1-4 alkyl, optionally substituted with up to three groups selected from halo, C1-2 alkoxy, C1-2 haloalkyl, and C1-2 haloalkoxy; and
two R groups on the same N can optionally cyclize to form a 5-7 membered ring optionally containing an additional heteroatom selected from N, O and S as a ring member, and optionally substituted with one or two groups selected from oxo, C1-2 alkyl, C1-2 alkoxy, C1-2 haloalkyl, and C1-2 haloalkoxy; and
G is selected from the group consisting of halo, benzotriazolyloxy, halobenzotriazolyloxy, pyridinotriazolyloxy, benzotriazolyl-N-oxide, pyridinotriazolyl-N-oxide, —O—(N-succinimide), 1-cyano-2-ethoxy-2-oxoethylideneaminooxy, and —O—(N-phthalimide).
28. The method of embodiment 26, wherein the peptide coupling reagent is selected from the group consisting of dicyclohexyl carbodiimide (DCC), diisopropyl carbodiimide (DIPC), 1-ethyl-3-(3-dimethylaminopropyl)carbodiimide (EDC), 1-cyclohexyl-(2-morpholinoethyl)carbodiimide tosylate (CMCT), COMU, HATU, HBTU, TBTU, HCTU, and TSTU, PyBOP, PyAOP, PyOxim, and BOP, and (3-(diethoxyphosphoryloxy)-1,2,3-benzotriazin-4(3H)-one) (DEPBT).
29. An engineered metalloprotein binder that specifically binds to an N-terminally modified target peptide modified by an N-terminal modifier agent, wherein:
a) the N-terminally modified target peptide has a formula: Z-P1-P2-peptide, wherein Z is an N-terminal modification capable of coordinating or chelating a zinc metal cation M, P1-P2-peptide is a target peptide before modification with the N-terminal modifier agent, Z-P1 is a modified N-terminal amino acid (NTAA) residue of the target peptide, and P2 is a penultimate terminal amino acid residue of the target peptide;
b) the engineered metalloprotein binder specifically binds to the N-terminally modified target peptide through interaction between the engineered metalloprotein binder and the Z-P1 of the N-terminally modified target peptide; and
c) the engineered metalloprotein binder comprises an amino acid sequence X1-C/H/D/E-X2-C/H/D/E-X3-C/H/D/E-X4, wherein C/H/D/E is any single amino acid residue independently selected from the group consisting of amino acid residues C (Cys), H (His),
D (Asp) and E (Glu); X1, X2, X3 and X4 are each any amino acid sequence comprising between 0 and 200 amino acid residues in length, and wherein the amino acid sequence X1-C/H/D/E-X2-C/H/D/E-X3-C/H/D/E-X4 chelates a zinc metal cation M with a thermodynamic dissociation constant of 0.5 nM or less. In preferred embodiments, X1, X2, X3 and X4 together comprise at least 30 amino acid residues in length.
30. The binder of embodiment 29, which binds to the N-terminally modified target peptide with at least a 100-fold greater binding affinity than a model peptide that has at least 90% homology to the engineered binder over the entire sequence length, wherein the model peptide does not comprise the amino acid sequence X1-C/H/D/E-X2-C/H/D/E-X3-C/H/D/E-X4.
31. The binder of embodiment 29 or embodiment 30, which comprises an amino acid sequence having at least about 90% sequence homology to any one of the amino acid sequences selected from the group consisting of SEQ ID NO: 7-SEQ ID NO: 59.
32. The binder of any one of embodiments 29-31, which comprises an amino acid sequence, which differs from one of the amino acid sequences set forth in SEQ ID NO: 7, SEQ ID NO: 8, SEQ ID NO: 9, SEQ ID NO: 14, SEQ ID NO: 15, SEQ ID NO: 16, SEQ ID NO: 17, SEQ ID NO: 21, SEQ ID NO: 25, SEQ ID NO: 27, SEQ ID NO: 30, SEQ ID NO: 31, SEQ ID NO: 39 or SEQ ID NO: 43 by at least one amino acid residue in a Z-P1 binding site, or within 6 Å of the Z-P1 binding site of the engineered metalloprotein binder, wherein the Z-P1 binding site comprises amino acids corresponding to amino acid positions 59, 64, 66, 88, 89, 91, 93, 103, 116, 118, 127, 128, 131, 137, 139, 193, 194, 195, 196, 198, 203, and 205 of SEQ ID NO: 7; or the Z-P1 binding site comprises amino acids corresponding to amino acid positions 60, 63, 65, 68, 87, 88, 90, 92, 102, 115, 117, 127, 137, 139, 193, 194, 195, 196, 197, 198, 203, and 205 of SEQ ID NO: 8; or the Z-P1 binding site comprises amino acids corresponding to amino acid positions 1, 2, 17, 59, 61, 64, 89, 91, 93, 103, 116, 118, 127, 131, 139, 193, 194, 195, 196, 197, 198, 199, 203, and 205 of SEQ ID NO: 9; or the Z-P1 binding site comprises amino acids corresponding to amino acid positions 99, 100, 132-136, 190, 191, 194, 200, and 222 of SEQ ID NO: 14; or the Z-P1 binding site comprises amino acids corresponding to amino acid positions 63-66, 83, 84, 86, 89, 92, 93, 96, 102, 149, 152-155, 158, and 175-177 of SEQ ID NO: 15; or the Z-P1 binding site comprises amino acids corresponding to amino acid positions 117, 255, 256, 257, 258-260, 268, 270, 271, 290, 293, 294, 297, 315, 316, 377, 779, and 821 of SEQ ID NO: 16; or the Z-P1 binding site comprises amino acids corresponding to amino acid positions 41, 58, 59, 60, 61, 62, 65, 77, 105, 107, 108, 109, 110-112, 147, 150, 151, 154, 155, 158, and 185 of SEQ ID NO: 17; or the Z-P1 binding site comprises amino acids corresponding to amino acid positions 133-137, 153, 158, 160, 161, 169, 173, 176, 177, 180, 186, 191, 192, 209, 216, and 230 of SEQ ID NO: 21; or the Z-P1 binding site comprises amino acids corresponding to amino acid positions 4-7, 9, 10, 14, 58, 90, 113, 134-138, 162, 181, 183-185, and 212 of SEQ ID NO: 25; or the Z-P1 binding site comprises amino acids corresponding to amino acid positions 7, 9, 20, 104, 108, 139, 141, 164, 167, 169, 170, 171, 202, 204-210, 242, 245, and 248 of SEQ ID NO: 27; or the Z-P1 binding site comprises amino acids corresponding to amino acid positions 90, 93, 96-99, 132, 133, 136, 142, 152, and 153 of SEQ ID NO: 30; or the Z-P1 binding site comprises amino acids corresponding to amino acid positions 165, 166, 169, 227-230, 231, 235, 248, and 352 of SEQ ID NO: 31; or the Z-P1 binding site comprises amino acids corresponding to amino acid positions 106-109, 141, 142, 145, 151, and 166-168 of SEQ ID NO: 39; or the Z-P1 binding site comprises amino acids corresponding to amino acid positions 106, 107, 313-317, 325, 327, 379, 382-389, 448, 453, 506, and 564-566 of SEQ ID NO: 43.
33. The binder of any one of embodiments 29-32, which binds to the N-terminally modified target peptide with a thermodynamic dissociation constant (Kd) of 500 nM or less.
34. The binder of any one of embodiments 29-33, which comprises a detectable label or a nucleic acid tag.
35. A kit for treating a target peptide, the kit comprises:
(a) an engineered metalloprotein binder of any of embodiments 29-34;
(b) one of more of the following:
1) an N-terminal modifier agent to form an N-terminally modified peptide having a formula: Z-P1-P2-peptide, wherein Z is an N-terminal modification capable of coordinating or chelating a zinc metal cation M, P1-P2-peptide is a target peptide before modification with the N-terminal modifier agent, Z-P1 is a modified N-terminal amino acid (NTAA) residue of the target peptide, and P2 is a penultimate terminal amino acid residue of the target peptide;
2) an agent configured for removing the modified NTAA residue from the N-terminally modified target peptide, thereby exposing a new NTAA residue;
3) an agent configured for immobilizing the target peptide on a solid support;
4) a solid support;
5) a nucleic acid recording tag;
6) a nucleic acid tag or a nucleic acid coding tag;
7) a detectable label; and/or
8) a peptide coupling reagent.

### EXAMPLES

The following examples are offered to illustrate but not to limit the methods, compositions, and uses provided herein. Certain aspects of the present invention, including, but not limited to, embodiments for the Proteocode™ peptide sequencing assay, information transfer between coding tags and recording tags, methods of making nucleotide-peptide conjugates, methods for attachment of nucleotide-peptide conjugates to a support, methods of generating barcodes, methods of generating specific binding agents recognizing an N-terminal amino acid of a peptide, reagents and methods for modifying and/or removing an N-terminal amino acid from a peptide, methods for analyzing extended recording tags were disclosed in the earlier published application US 2019/0145982 A1, US 2020/0348308 A1, US 2020/0348307 A1, US 2021/0208150 A1, WO 2020/223000 A1, the contents of which are incorporated herein by reference in its entirety.

### Example 1. Establishing Human Carbonic Anhydrase 2 (CA II) as Model Metalloprotein Binder for Modified Peptides

Carbonic anhydrase is well known as one of the most efficient enzymes in nature (nearly diffusion limited). This zinc binding protein is expressed in nearly all forms of life, with numerous variants/isozymes that are distinct in protein sequence and structure, depending on the species of origin. The active site zinc ion is catalytic for the conversion of carbon dioxide and water to bicarbonate and is bound at the bottom of the 15 Å deep substrate binding pocket (for human carbonic anhydrase 2, SEQ ID NO: 7) characterized by hydrophobic walls and a hydrophilic cleft. Carbonic anhydrases have been pursued as drug targets for multiple indications, and numerous metal binding small molecule inhibitors have been identified along with corresponding crystal structures and SAR. Carbonic anhydrase is a small (˜30 kD) monomeric protein (although some variants form dimers) with no appreciable post-translational modifications and a single cysteine (for human carbonic anhydrase 2, hCAII, SEQ ID NO: 7). It exhibits high structural stability, binding pocket evolvability using phage display and can be produced on a large scale. Genetic manipulation of carbonic anhydrase is well documented and many natural variants exist across organisms to provide a range of initial scaffolds for computational or practical evaluation. Further, phenylsulfonamide-modified peptides have been shown to bind to carbonic anhydrase with high affinity (Sigal and Whitesides, Benzenesulfonamide-peptide conjugates as probes for secondary binding sites near the active site of carbonic anhydrase, Bioorganic & Medicinal Chemistry letters, Vol. 6. No. 5, pp. 559-564, 1996). Thus, carbonic anhydrase is a promising candidate (binder scaffold) for a specific metalloprotein binder.

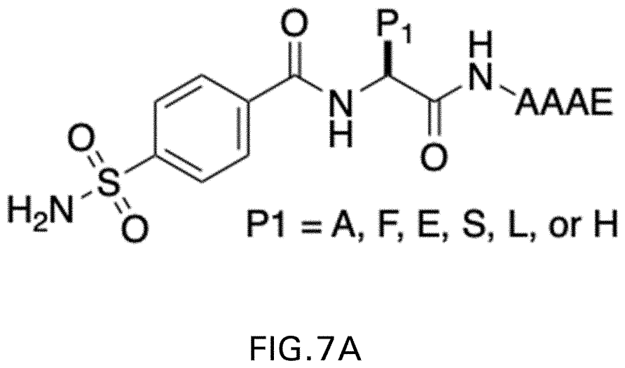

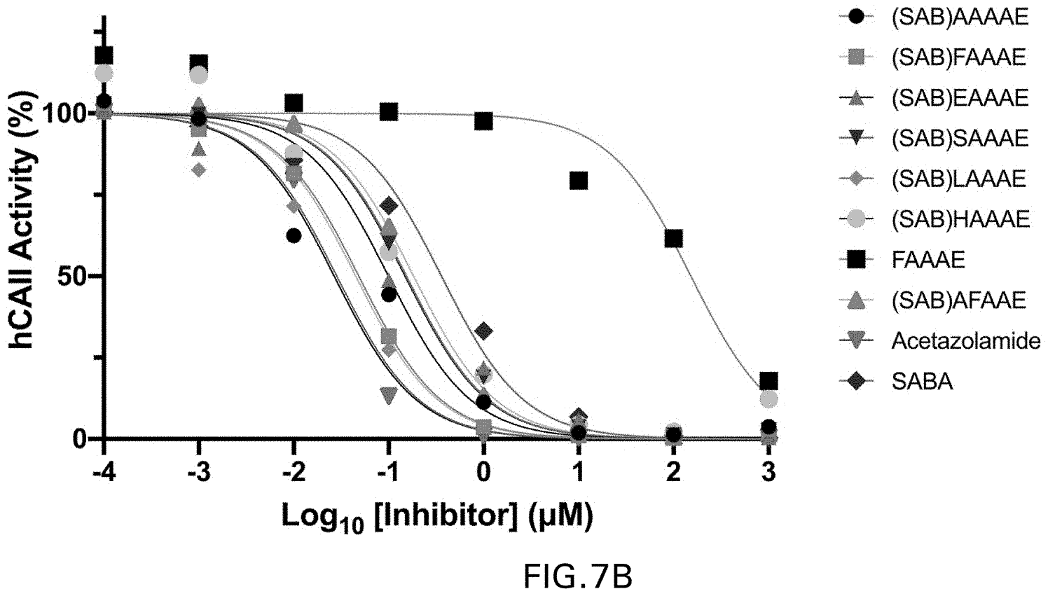

A functional assay for hCAII enzyme was set up. Carbonic anhydrase catalyzes the hydrolysis of 4-nitrophenyl acetate (4-NPA) to nitrophenol, which can be monitored by absorbance at 400 nm. The enzymatic carbonic anhydrase assay generally includes 0.2-1.0 μM enzyme and 10-500 μM 4-NPA in 20-200 μL in assay buffer. Assay buffer compositions can vary in buffer identity (Tris, phosphate, HEPES, etc.) and preferably do not precipitate required metal ions. Metal chelating agents (EDTA or EGTA), salt (NaCl, sulfate), detergent (Tween or Triton), and organic additives (acetonitrile, DMSO) may be employed to facilitate enzyme stability and reagent solubility. For the assay, 1 μM human carbonic anhydrase II, 50 mM MOPS (pH 7.6), 33 mM disodium sulfate, and 1 mM EDTA. To generate NTM-modified peptides, azide-derivatized peptides (via azide-PEG-amine and carbodiimide coupling to C-terminus of peptide) were conjugated to DBCO-coupled beads. As a metal-binding NTM that would possess high affinity binding to hCAII, 4-sulfamoylbenzoic acid (SABA) was employed as a metal binding pharmacophore to modify peptides at the N-terminus. To evaluate P1 dependence of the binding reaction, multiple P1 residues has been tested. SABA-XAAAE-NH₂ and SABA-AFAAE-NH₂ were obtained (FIG. 7A) by treating peptides immobilized on beads with SABA-NHS (X is a random amino acid residue). Binding affinities of the SABA-modified peptides to hCAII were estimated by calculating half-maximal inhibitory concentrations (IC50) of the peptides in the hCAII functional assay. 1 μM of hCAII in 50 mM MOPS (pH 7.6), 33 mM Na₂SO₄, 1 mM EDTA were mixed with different concentrations of the following compounds: SABA-XAAAE-NH₂, SABA-AFAAE-NH₂, FAAAE, SABA and acetazolamide (a control hCAII inhibitor). Effective dilution of each compound was from 1 mM to 0.1 nM, and DMSO was used as a vehicle; the mixtures were incubated for 10 minutes at 37° C. Then p-Nitrophenylacetate (pNP-OAc) in DMSO was added to each well for an effective concentration of 500 μM, and inhibitory rates for each compound were determined by pNP release over 180 s (FIG. 7B). No significant binding/inhibition was observed with the native peptide, whereas SABA-modified peptides show efficient binding/inhibition with nM K_{d} (assuming IC₅₀≈K_{d}). Efficiencies of binding/inhibition vary depending on the P1 residue of the peptide (SABA-P1 binding efficiency: S<H<E<A<F<L) with IC₅₀ ranging from 28 nM to 153 nM (Table 1). P2 dependence show ˜4-fold difference, since IC₅₀ are shown to be 48 nM, 183 nM, 45 nM for SABA-AA v. SABA-AF v. SABA-FA (SABA-P1-P2 groups).

TABLE 1
Half-maximal inhibitory concentrations (IC₅₀) of the
SABA-modified peptides in the hCAII functional assay.

| Compound | IC₅₀ (μM) |
|---|---|
| (SAB)AAAAE | 0.0484 |
| (SAB)FAAAE | 0.04498 |
| (SAB)EAAAE | 0.0993 |
| (SAB)SAAAE | 0.1534 |
| (SAB)LAAAE | 0.02839 |
| (SAB)HAAAE | 0.1485 |
| FAAAE | 151.8 |
| (SAB)AFAAE | 0.1827 |
| Acetazolamide | 0.02665 |
| SABA | 0.3594 |

### Example 2. Selection and Design of Engineered Metalloprotein Binders Suitable for a Protein Binding (Such as NGPS) Assay and Capable of Binding NTM-P1 with Minimal P2 Bias

Part I. Initial binder selection. To identify metalloproteins with potential utility as binders for the NGPS assay, zinc binding proteins with available crystal structures were reviewed from the literature in the Research Collaboratory for Structural Bioinformatics (RCSB) Protein Data Bank (PDB), and those with at least one accessible zinc ion, also referred to as Zn(II), binding site were identified as candidates for computational modeling studies. Accessible Zn(II) binding sites were defined as having trivalent Zn(II) coordination in PDB accession codes (also referred to as PDB IDs), in order to permit NTM-peptide coordination in the fourth Zn(II) coordination site, and binding pockets with either a conical or groove-shaped architecture near the Zn(II) binding site. Where Zn(II) binding sites had tetravalent Zn(II) coordination, one Zn(II)-chelating residue was mutated to either glycine or alanine to permit the fourth Zn(II) coordination site to be occupied by the NTM-peptide. Additionally, noncanonical amino acids were mutated to their canonical counterparts (e.g., cysteinesulfinic acid was mutated to cysteine). In effect, protein scaffolds where the Zn(II) ion is weakly bound and/or largely buried in the protein scaffold were excluded. Excessively large proteins (i.e., ≥100 kDa), those with numerous post-translational modifications (e.g., glycosylation), and oligomeric protein assemblies were also excluded. Crystal structures with small-molecule ligands coordinating the Zn(II) ion were given preference. A non-exhaustive list of PDB accession codes (PDB IDs) used for computational simulations is as follows: 5FCW, 2FV5, 2J83, 5E3C, 4Q4E, 5ELY, 1HEE, 4DJL, 1IAG, 1JAN, 1KAP, 1LML, 1OBR, 2CKI, 3P1V, 3UJZ, 4L63, 4LP6, 5K7J, 4DLM, 4YYT, 2CAB, 3P24, 5KZJ, 1JD0, 1Z97, 3ML5, 4KNM, 5JN8, 1AST, 1C7K, 2X7M, 3U7M, and 5OD1.

Some scaffolds can be further optimized, such as reduced in size, by removing part(s) that form(s) a separate structure distant from a metal-binding portion of the scaffold. For example, 4Q4E scaffold (SEQ ID NO: 16) can be truncated to remove a separate domain which is structurally distinct from the metal-binding domain; the resulting truncated scaffold (SEQ ID NO: 59) has similar metal-binding properties to the original 4Q4E scaffold and similar relative Kd towards NTM-modified dipeptides.

Human carbonic anhydrases (hCA) were used as starting scaffolds for directed evolution toward binding modified NTAA residues of peptides.

Part II. NTM identification for the selected binders. Numerous small-molecule inhibitors of metalloproteins with enzymatic activity have precedence in the literature. In particular, arylsulfonamides and hydroxamic acids are well established Zn(II)-coordinating inhibitors of carbonic anhydrases and other metalloproteases. Additional established Zn(II)-coordinating ligand moieties include imidazoles, thiazoles, pyrazoles, thiols, hydrazides, N-hydroxyureas, squaric acids, carbamoylphosphonates, oxazolines, sulfamides, sulfamates, and quinolines. We designed N-terminal modifications (NTMs) to harbor these Zn(II)-coordinating moieties for high-affinity NTM binding to the Zn(II) ion in its respective metalloprotein. The NTMs were installed on a model dipeptide Ala-Ala (A-A) and used for in silico binding experiments and computational macromolecular modeling.

Based on internal data and computational modeling, metal-binding NTMs were designed such that when combined with the P1 amino acid residue (i.e., the N-terminal amino acid residue of the peptide), the NTM-P1 moiety occupies the hCA substrate pocket, with the P1 sidechain oriented closer to the molecular surface of the pocket. This design forces the P2 residue (penultimate residue) of the peptide to be located just outside the pocket or affinity determining region and contribute less Gibbs free energy to peptide binding. In particular, sulfamoyl benzene, pyrazolemethanimine (PMI), aminoguanidine and their chemical derivatives were evaluated.

Based on the data from Example 1, M64 NTM (FIG. 6A) has been initially selected. A 1.9 Å crystal structure of wild-type human carbonic anhydrase II (hCAII) protein (SEQ ID NO: 7) co-crystallized with M64-F group was obtained. Then, a set of Zn(II)-coordinating NTMs (i.e., derivatives of M64) was designed to provide enhanced binding affinity to the hCAII protein using structure—function analysis and crystal structure-based approach. The resulting NTMs (M64-M91 and M93-M98 (see FIG. 6A-6C)) were evaluated using a colorimetric IC50 assay to determine relative binding affinity of NTMs to the wild-type hCAII protein based on NTM inhibition capacity.

The colorimetric assay used 300 nM of wild-type carbonic anhydrase incubated in 45 μL of 50 mM MOPS (pH 7.5), 33 mM Na₂SO₄, and 1 mM EDTA aliquoted into a 96-well, clear, flat-bottom plate. To each column of the plate, a 1/10 dilution series from 1 mM to 0.1 nM of each NTM was added and incubated at 25° C. for 10 minutes to reach binding equilibrium. To this, 1 mM p-nitrophenylacetate (pNPA) was added to each well and screened on a plate reader at 405 nm wavelength. The initial rate of hydrolysis was observed over the first 60 seconds. The slopes versus the concentration of NTM were put into a non-linear regression equation to determine the IC50 of the NTM to the carbonic anhydrase.

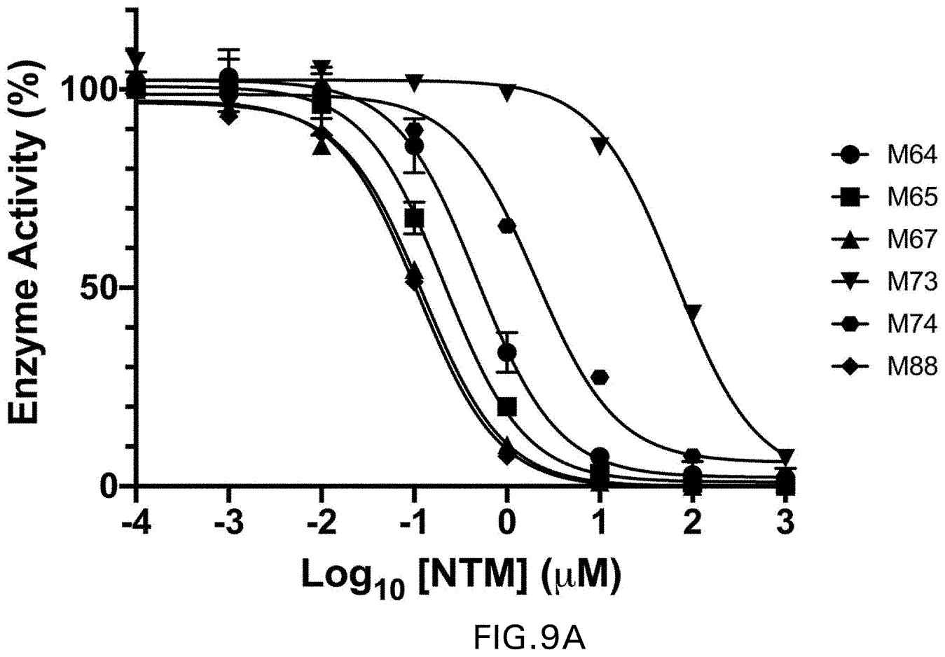

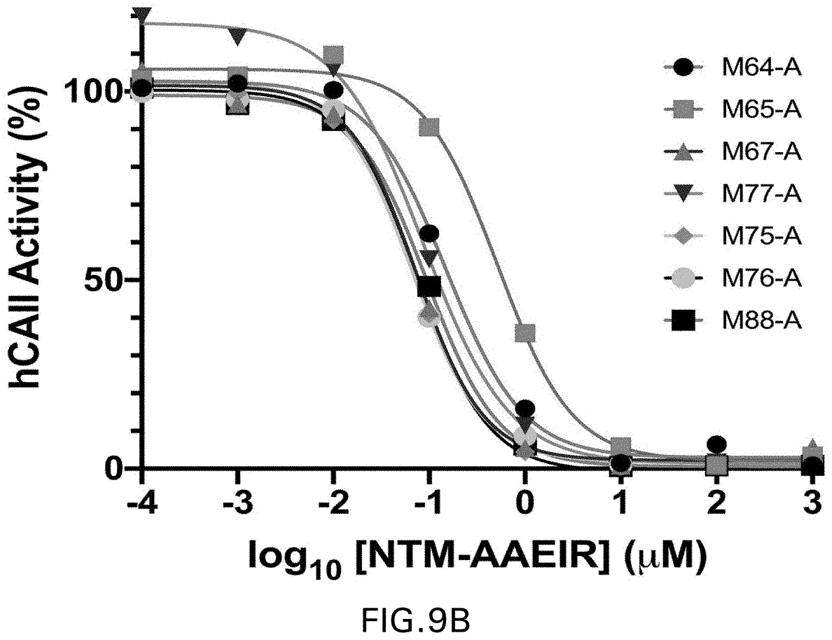

12 M64 derivatives have been screened (Table 2; NTM structures are shown in FIG. 6A-6C). 6 tested NTMs provided ≥2-fold enhanced affinity over M64, as shown in FIG. 9A and Table 2.

Next, the selected NTMs (derivatives of NTM M64) were installed on the N-terminus of a model peptide AAEIR by methods disclosed below. The N-terminally modified peptides were then evaluated using colorimetric IC50 assay to determine relative binding affinity of NTM-AAEIR to wild-type hCAII protein based on NTM-AAEIR inhibition capacity. The slopes versus the concentration of NTM-AAEIR were put into a non-linear regression equation to determine the IC50 of the selected NTM-AAEIR peptides to the wild-type hCAII. The results are shown in FIG. 9B and Table 2.

TABLE 2
Inhibition capacity of NTM M64 derivatives towards
wild-type hCAII protein (relative to M64).

| | | | Fold | NTM-AAEIR | Fold |
|---|---|---|---|---|---|
| NTM | NTM | IC50 | Enhance- | IC50 | Enhance- |
| Substituent | ID | (μM) | ment | (μM) | ment |
| H | M64 | 0.473 | 1.0 | 0.150 | 1.0 |
| 3-endoN | M65 | 0.211 | 2.2 | 0.515 | 0.3 |
| 2-endoN | M66 | 0.992 | 0.5 | n.d. | n.d. |
| 2-NO2 | M67 | 0.124 | 3.8 | 0.073 | 2.0 |
| 3-OCH3 | M68 | n.d. | n.d. | n.d. | n.d. |
| 2-CH3 | M74 | 2.04 | 0.2 | n.d. | n.d. |
| 2-CF3 | M77 | 0.192 | 2.5 | 0.087 | 1.7 |
| 2-NH2 | — | 1.378 | 0.3 | n.d. | n.d. |
| 2-OCH3 | M73 | 65.43 | 0.0 | n.d. | n.d. |
| 2-F | M75 | 0.224 | 2.1 | 0.067 | 2.2 |
| 2-Cl | M76 | 0.180 | 2.6 | 0.077 | 1.9 |
| 3-F | M88 | 0.110 | 4.3 | 0.095 | 1.6 |

n.d.—not determined.
Fold enhancement is calculated relative to NTM M64.

Part III. NTM Parameterization. N-terminal modifications (NTMs), designated M64-M91 and M93-M97 (see FIG. 6A-6C), were parameterized for the Rosetta macromolecular modeling software suite by first generating conformer ensembles of each NTM using open-source RDKit software. For each NTM, each rotatable torsion angle sampled during conformer generation was clustered every 15°, from 0° to 360°, and the average torsion angle and standard deviation of the torsion angle was input to an Rosetta N-terminal modification parameterization patch file for sampling during NTM repacking in PyRosetta, a python-based interface to the Rosetta macromolecular modeling suite. For each atom of each NTM, partial charges were computed using the Amber antechamber software in a semi-empirical (AM1) with bond charge correction (BCC) model, AM1-BCC, which utilizes parameterization against the HF/6-31G* electrostatic potential of a training set of compounds with relevant functional groups. The partial charges of each atom type were also input into the Rosetta N-terminal modification parameterization patch file. NTMs were modeled onto the N-terminus of a dipeptide with amino acid sequence Ala-Ala (i.e., AA). The canonical C-terminus of each N-terminally modified dipeptide was computationally modeled with a dimethylamide moiety on the C-terminus.

Part IV. Computational modeling of potential binders. The three-dimensional coordinates of the metalloprotein binding residues and Zn(II) ion from PDB ID 4YYT, an X-ray diffraction crystal structure solved to 1.07 Å resolution of human carbonic anhydrase II in complex with a compound with a benzenesulfonamide moiety, 4-(2-hydroxyethyl)benzenesulfonamide, was used as a reference template for docking each NTM to the Zn(II) ion binding site in each PDB accession code (PDB ID) selected as binders (see “Part I. Initial binder selection” above). For each PDB ID, the residue number of the Zn(II) ion atom to be computationally modeled as binding the NTM-peptide was manually selected and cataloged in an input file. For each NTM (i.e. M64-M91 and M93-M97), an atom name map was manually generated between the atom names from the 4-(2-hydroxyethyl)benzenesulfonamide compound in PDB ID 4YYT to the structurally similar atom names in each NTM. Additionally, the interatomic distances between the Zn(II) ion atom and each of the three heavy polar atoms in the 4-(2-hydroxyethyl)benzenesulfonamide compound (i.e., the nitrogen and oxygen atoms of the sulfonamide moiety) in PDB ID 4YYT that are in close contact with the Zn(II) ion atom, respectively, were cataloged in an input file. The interatomic distances were applied as distance constraints (using a harmonic potential with 0.1 Å standard deviation) to the structurally similar polar heavy atoms in NTMs M64-M70, M73-M91, and M93-M97. Similarly, the interatomic distances between the Zn(II) ion atom and each of the three heavy polar atoms in the 4-naphthalen-1-yl-˜{N}-oxidanyl-benzamide compound (i.e., the nitrogen and oxygen atoms of the hydroxamate moiety) in PDB ID 5FCW that are in close contact with the Zn(II) ion atom, respectively, were cataloged in an input file. Again, the interatomic distances were applied as applied as distance constraints (using a harmonic potential with 0.1 Å standard deviation) to the structurally similar polar heavy atoms in NTMs M71 and M72 (see below). Prior to computational simulations using the PyRosetta macromolecular design and modeling software suite, the following PDB IDs were prepared in Molecular Operating Environment (MOE) software to close bonds, correct hybridization and partial charges, and model loops that were missing in the protein scaffold deposited to the Protein Data Bank: 5K7J, 1LML, 5FCW, 3UJZ, 4L63, 4LP6, 5ELY, 1HEE, 2X7M, 1JAN, 3U7M, 2J83, 3P24, 5KZJ, 5JN8, and 4YYT. For each PDB ID and each NTM, one PyRosetta simulation was run to model the native protein scaffold (i.e., “native” binder), and one PyRosetta simulation was run to redesign the P1 pocket residues of the protein scaffold (i.e., “designed” binder).

For each PDB ID and NTM simulated using PyRosetta, the metal-chelating residues were algorithmically determined by finding the closest three residues in the protein scaffold containing the Zn(II) ion atom of interest, and for each of the metal-chelating residues algorithmically locating the closest polar heavy atom (of either nitrogen, oxygen, or sulfur atom types) on each metal-chelating residue to the Zn(II) ion atom of interest. Once the Zn(II) ion atom and the metal-binding atoms of each of the three metal-chelating residues were determined, the ordering of the metal-chelating atoms was permuted exhaustively, allowing for six different orderings of heavy polar atoms (i.e., 3!=6 combinations). For each of the six different metal-chelating atom orderings, the three metal-chelating atoms along with the Zn(II) ion were superimposed onto the three metal-chelating atoms and Zn(II) ion atom in PDB ID 4YYT. In each of these six different superimpositions onto PDB ID 4YYT, the 4-(2-hydroxyethyl)benzenesulfonamide compound from PDB ID 4YYT was transferred to the binder using the PDB ID 4YYT crystal structure coordinates, and the protein scaffold from PDB ID 4YYT was deleted. Effectively at this stage, the 4-(2-hydroxyethyl)benzenesulfonamide compound acted as temporary surrogate for the NTM-dipeptide in the binder pocket. A clash score was calculated between the 4-(2-hydroxyethyl)benzenesulfonamide compound and the binder. The superimposition with the lowest clash score (i.e., fewest clashes) was selected as the most appropriate superimposition for further simulation. Subsequently, the NTM of the NTM-dipeptide was superimposed onto the 4-(2-hydroxyethyl)benzenesulfonamide compound in the binder using the aforementioned atom name map, and the 4-(2-hydroxyethyl)benzenesulfonamide compound was deleted. The torsion angle between the metal-binding atoms and the NTM aromatic ring was sampled at a torsion angle equal to the corresponding torsion angle in the 4-(2-hydroxyethyl)benzenesulfonamide compound, with and without adding 180°, and the NTM-dipeptide backbone torsion angles were randomized with bias toward Ramachandran torsion bins for the dipeptide amino acid identities (i.e., AA) a total of 100 times. For each NTM-dipeptide conformation, a clash score between the NTM-dipeptide and the binder was computed. The NTM-dipeptide conformation with the lowest clash score was selected for further simulation. Effectively at this stage, the NTM-dipeptide was docked into the binder and modeled as chelating the Zn(II) ion atom.

Subsequently, the metal-chelating residues and Zn(II) ion atomic 3-dimensional coordinates were constrained in place using a harmonic potential with 0.1 Å standard deviation. Furthermore, the aforementioned distance constraints between the Zn(II) ion atom and each of the three polar heavy atoms in close contact with the Zn(II) ion atom (i.e., as described above using interatomic distances derived from PDB ID 4YYT and PDB ID 5FCW) were applied using a harmonic potential with 0.1 Å standard deviation. Subsequently, P1 pocket residues were algorithmically determined as those on the binder within ≤4.5 Å from any atom in the NTM-dipeptide or those with Ca atoms within ≤6.0 Å of the P1 Cα atom, discounting the metal-chelating residues. For PyRosetta simulations maintaining the native amino acid sequence (discounting mutations to glycine or alanine of up to one residue in the Zn(II) ion binding site, as well as discounting mutations of noncanonical amino acids to their canonical counterparts [see “Binder Selection” above]; termed “native” binders), side-chain rotamers were permitted to repack with a fixed amino acid identity. For the PyRosetta simulations mutating the native amino acid sequence (again discounting mutations to glycine or alanine of up to one residue in the Zn(II) ion binding site, as well as discounting mutations of noncanonical amino acids to their canonical counterparts [see “Part I. Initial binder selection” above]; termed “designed” binders), side-chain rotamers were permitted to repack and/or design to the same or different amino acid identity. Side-chain rotamers and/or amino acid identities were sampled using a Monte Carlo Metropolis criterion algorithm, followed by minimization of protein backbone and side-chains in the full-atom Rosetta energy function “ref2015_cart”. Side-chain repacking and backbone and side-chain minimization steps were iteratively processed in the PyRosetta algorithm FastRelax for the native binders, and the PyRosetta algorithm FastDesign for the designed binders. As such, new conformations of NTM-dipeptide in complex with the binders were algorithmically generated. Finally, biophysical metrics were computationally calculated as given in Tables 3-8.

The purpose of algorithmically mutating the native binders to generate the designed binders was to increase the binding affinity (i.e., decrease the thermodynamic dissociation constant) of the NTM-dipeptide for the native binder (i.e., equivalent to decreasing the change in Gibbs free energy upon NTM-dipeptide binding to the protein scaffold for the designed binder compared to the native binder). PyRosetta software employs a pseudorandom number generator (RNG) to generate a seed (i.e., an integer value) to initialize each PyRosetta simulation. As such, the input RNG-generated seed to the PyRosetta simulation results in a deterministic trajectory. Generally, each design simulation within PyRosetta software was expected to increase the affinity of the NTM-dipeptide for the native binder. As only one design simulation was run per PDB ID per NTM, it was expected that not all design simulations would result in increased affinity of the NTM-dipeptide for the native binder, as the FastDesign algorithm in the PyRosetta simulation could arrive in a local energetic minimum in sequence-structure space, rather than always arriving in the global energetic minimum in sequence-structure space. Therefore, by running the FastDesign algorithm in the PyRosetta simulation using a multitude of different RNG-generated seeds (on the order of using 10³ to 10⁶ unique RNG-generated seeds, with an upper-bound limited only by the practicality of procuring compute resources), it is expected that design simulations in PyRosetta software would result in designed binders with even higher ΔKd (native to designed) (i.e., overall a lower thermodynamic dissociation constant) of the NTM-dipeptide for the designed binder. Future computational protein modeling campaigns will employ multitudinous RNG-generated seeds to arrive in the global energetic minimum in sequence-structure space for each PDB ID and NTM combination. For each PDB ID and NTM combination, the designed binders with the highest ΔKd (native to designed) will be selected for experimental validation.

For each PDB ID, NTM, and either the native binder or designed binder, the following labels and/or biophysical metrics and their descriptions below were computed.

“PDB ID”: the Protein Data Bank accession code for the selected binder.

“NTM”: the N-terminal modification identifier.

“Metal Ion”: the metal ion name and Roman numeral in parentheses representing the ionic charge or oxidation state of the metal ion.

“Metal-chelating Residues”: a comma-separated list where each element represents the residue number followed by the one-letter amino acid identity. There are three metal-chelating residues per binder, and the NTM occupies the fourth metal ion coordination site.

“Native Binder ΔKd (Metal-chelating Residues to Gly)”: for the native binder, the fold-change in the thermodynamic dissociation constant (Kd) of NTM-dipeptide binding upon mutation of the metal-chelating residues to glycine and translation of the metal ion from the interface, as given by the formula

$$Δ {K}_{d} = {e}^{- \frac{Δ Δ G}{R T}} ,$$

where ΔΔG is the value given by “Native Binder ΔΔG (Metal-chelating Residues to Gly) (kcal/mol)” described below, R is the universal gas constant, and T is the temperature at 25° C.

“Designed Binder ΔKd (Metal-chelating Residues to Gly)”: for the designed binder, the fold-change in the thermodynamic dissociation constant (Kd) of NTM-dipeptide binding upon mutation of the metal-chelating residues to glycine and translation of the metal ion from the interface, as given by the formula

$$Δ {K}_{d} = {e}^{- \frac{Δ Δ G}{R T}} ,$$

where ΔΔG is the value given by “Designed Binder ΔΔG (Metal-chelating Residues to Gly) (kcal/mol)” described below, R is the universal gas constant, and T is the temperature at 25° C.

“ΔKd (Native to Designed)”: the fold-change improvement in the thermodynamic dissociation constant (Kd) of NTM-dipeptide binding upon designing the binder from the native binder sequence to the designed binder sequence, as given by the formula

$$Δ {K}_{d} = {e}^{- \frac{ΔΔΔ G}{R T}} ,$$

where ΔΔΔG=ΔΔG^{designed}−ΔΔG^{native}, ΔΔG^{designed} is the value given by “Designed Binder ΔΔG (Metal-chelating Residues to Gly) (kcal/mol)”, ΔΔG^{native} is the value given by “Native Binder ΔΔG (Metal-chelating Residues to Gly) (kcal/mol)”, R is the universal gas constant, and T is the temperature at 25° C. The value is tantamount to

$$Δ {K}_{d} = \frac{Δ {K}_{d}^{designed}}{Δ {K}_{d}^{native}}$$

where ΔK_{d}^{designed} is the value given by “Designed Binder ΔKd (Metal-chelating Residues to Gly)” and ΔK_{d}^{native} is the value given by “Native Binder ΔKd (Metal-chelating Residues to Gly)”.

“P1 Pocket Residues”: a comma-separated list where each element represents a residue number from the binder within ≤4.5 Å from any atom in the NTM-dipeptide or a residue with Ca atom within ≤6.0 Å of the P1 Cα atom, discounting metal-chelating residues as given in “Metal-chelating Residues”. Each of these residues was permitted to repack to different rotamers in the native binder, and repack to different rotamers while updating amino acid identity in the designed binder.

“Mutations (Native to Designed)”: a comma-separated list of mutations from the native binder to the designed binder, where each element represents the native binder amino acid identity followed by the residue number followed by the designed binder amino acid identity.

“Native Binder Sequence”: the amino acid sequence of the native binder used in the computational simulation. “Designed Binder Sequence”: the resulting amino acid sequence of the designed binder after the computational simulation.

Tables comprising data that evaluate relative binding affinities for metalloprotein binder scaffolds and exemplary designed binders are shown below.

TABLE 3
Estimated relative binding affinities for metalloprotein binder
scaffolds using modeled M64-modified AA dipeptide as a substrate.

| PDB | Metal-chelating | Native | Designed | ΔKd (Native to |
|---|---|---|---|---|
| ID | Residues | Binder ΔKd | Binder ΔKd | Designed) |
| 5ELY | 333D,371E,499H | 122.56 | 58.80 | 0.48 |
| 1C7K | 83H,87H,93D | 169.95 | 39933.41 | 234.97 |
| 2CAB | 90H,92H,115H | 1260.37 | 3025.70 | 2.40 |
| 3P24 | 162D,316H,320H | 1530.55 | 568.53 | 0.37 |
| 2X7M | 132H,136H,142H | 1917.26 | 83739.91 | 43.68 |
| 4DJL | 69H,72E,204H | 2064.17 | 3507.48 | 1.70 |
| 1AST | 92H,96H,102H | 3646.08 | 24966.00 | 6.85 |
| 2CKI | 168H,172H,178H | 3759.17 | 9048.85 | 2.41 |
| 5OD1 | 35H,61H,65H | 3802.74 | 936.01 | 0.25 |
| 1OBR | 69H,72E,204H | 4034.94 | 2502.52 | 0.62 |
| 1HEE | 69H,72E,196H | 4324.49 | 1649.98 | 0.38 |
| 4KNM | 92H,94H,117H | 4793.21 | 7027.04 | 1.47 |
| 4L63 | 160H,164H,170H | 5348.71 | 4895.40 | 0.92 |
| 4LP6 | 91H,93H,116H | 6154.33 | 5537.36 | 0.90 |
| 5JN8 | 97H,99H,122H | 6388.24 | 11297.01 | 1.77 |
| 1JAN | 119H,123H,129H | 7690.22 | 7237.69 | 0.94 |
| 3ML5 | 94H,96H,119H | 7730.89 | 2652.53 | 0.34 |
| 4Q4E | 293H,297H,316E | 8287.36 | 26716.71 | 3.22 |
| 2J83 | 166H,170H,176H | 9299.58 | 18565.42 | 2.00 |
| 2FV5 | 190H,194H,200H | 9776.33 | 21453.68 | 2.19 |
| 1LML | 165H,169H,235H | 9923.66 | 73906.30 | 7.45 |
| 4YYT | 91H,93H,116H | 10611.77 | 6598.19 | 0.62 |
| 1Z97 | 91H,93H,116H | 11600.71 | 18565.77 | 1.60 |
| 3UJZ | 408H,412H,418H | 12546.59 | 6165.14 | 0.49 |
| 1JD0 | 89H,91H,115H | 13497.78 | 5259.50 | 0.39 |
| 5E3C | 448H,453H,506E | 20466.38 | 26662.41 | 1.30 |
| 1KAP | 176H,180H,186H | 29950.61 | 176002.03 | 5.88 |
| 5K7J | 181H,185H,212H | 36645.34 | 45366.57 | 1.24 |
| 1IAG | 141H,145H,151H | 70228.70 | 30691.31 | 0.44 |
| 4DLM | 7H,9H,242D | 142141.47 | 818.42 | 0.01 |
| 3U7M | 111C,154H,158H | 541295.44 | 414363.56 | 0.77 |

Some of the tested scaffolds (e.g., 3U7M, 4DLM, 1IAG, 5K7J, 1KAP) show elevated “Native Binder ΔKd” parameter (calculated upon mutation of metal-chelating residues to Gly), indicating that metal-chelating residues and thus the metal ion significantly contribute to binding affinity for binding between the native binder and the M64-modified AA model peptide. Other tested scaffolds show significantly elevated “ΔKd (Native to Designed)” parameter (i.e., the fold-change improvement in Kd upon mutating the binder from the native binder sequence), indicating that the designed (modified) binders have improved binding affinities for binding between the designed binder and the M64-modified AA model peptide.

Examples of such designed binders having improved binding affinities include SEQ ID NOs: 28-31 based on the following scaffolds: 3U7M (having corresponding mutations: G58A, G60V, L61I, A62V, Q65M, I77L, T107D, E109L, G110Q, Y147L, V151L, E155A, and E185L); 1KAP (having corresponding mutations: A134L, A135V, A137V, Y158W, A160I, N161V, Y169R, T173L, E177M, N191H, A192P, R209L, and Y216L); 2X7M (having corresponding mutations: A90I, L93V, G98L, Q99I, E133A, F152P, and S153A); and 1LML (having corresponding mutations: E166V, A229E, S231Y, and F352L).

TABLE 4
Estimated relative binding affinities for metalloprotein binder
scaffolds using modeled M65-modified AA dipeptide as a substrate.

| PDB | Metal-chelating | Native | Designed | ΔKd (Native to |
|---|---|---|---|---|
| ID | Residues | Binder ΔKd | Binder ΔKd | Designed) |
| 1AST | 92H,96H,102H | 624.46 | 8647.93 | 13.85 |
| 1C7K | 83H,87H,93D | 32.38 | 3854.48 | 119.04 |
| 1HEE | 69H,72E,196H | 548.15 | 1705.60 | 3.11 |
| HAG | 141H,145H,151H | 38483.43 | 11906.58 | 0.31 |
| 1JAN | 119H,123H,129H | 12267.20 | 16932.23 | 1.38 |
| 1JD0 | 89H,91H,115H | 2074.28 | 2028.63 | 0.98 |
| 1KAP | 176H,180H,186H | 383.02 | 1905.54 | 4.97 |
| 1LML | 165H,169H,235H | 9543.07 | 11293.15 | 1.18 |
| 1OBR | 69H,72E,204H | 1737.31 | 1493.11 | 0.86 |
| 1Z97 | 91H,93H,116H | 5064.90 | 20860.11 | 4.12 |
| 2CAB | 90H,92H,115H | 504.50 | 985.06 | 1.95 |
| 2CKI | 168H,172H,178H | 10674.58 | 11152.29 | 1.04 |
| 2FV5 | 190H,194H,200H | 7606.85 | 4936.36 | 0.65 |
| 2J83 | 166H,170H,176H | 13645.70 | 26139.29 | 1.92 |
| 2X7M | 132H,136H,142H | 75987.80 | 33487.11 | 0.44 |
| 3ML5 | 94H,96H,119H | 1536.03 | 3717.35 | 2.42 |
| 3P24 | 162D,316H,320H | 20.48 | 4.07 | 0.20 |
| 3U7M | 111C,154H,158H | 114409.43 | 352385.06 | 3.08 |
| 3UJZ | 408H,412H,418H | 18043.30 | 13734.32 | 0.76 |
| 4DJL | 69H,72E,204H | 2083.30 | 1521.04 | 0.73 |
| 4DLM | 7H,9H,242D | 16141.45 | 2301.79 | 0.14 |
| 4KNM | 92H,94H,117H | 7450.23 | 9396.54 | 1.26 |
| 4L63 | 160H,164H,170H | 1775.95 | 23991.78 | 13.51 |
| 4LP6 | 91H,93H,116H | 6288.96 | 10964.18 | 1.74 |
| 4Q4E | 293H,297H,316E | 47969.63 | 650053.88 | 13.55 |
| 4YYT | 91H,93H,116H | 3347.82 | 10851.54 | 3.24 |
| 5E3C | 448H,453H,506E | 298.39 | 12151.96 | 40.73 |
| 5ELY | 333D,371E,499H | 14.12 | 27.22 | 1.93 |
| 5JN8 | 97H,99H,122H | 2959.72 | 6101.65 | 2.06 |
| 5K7J | 181H,185H,212H | 53027.31 | 46548.05 | 0.88 |
| 5OD1 | 35H,61H,65H | 43.77 | 135.18 | 3.09 |

Some of the tested scaffolds (e.g., 3U7M, 2X7M, 5K7J, and 4Q4E) show elevated “Native Binder ΔKd” parameter (calculated upon mutation of metal-chelating residue to Gly), indicating that metal-chelating residues significantly contribute to binding affinity for binding between the unmodified binder and the M65-modified AA model peptide. Other tested scaffolds show significantly elevated “ΔKd (Native to Designed)” parameter (the fold-change improvement in Kd upon mutating the binder from the native binder sequence), indicating that the designed (modified) binders have improved binding affinities for binding between the designed binder and the M65-modified AA model peptide.

Examples of such designed binders having improved binding affinities include SEQ ID NOs: 32-34 based on the following scaffolds: 4Q4E (having corresponding mutations: E117L, F254L, M256T, A258V, M259V, E260P, K270A, Y271L, D286E, R289A, E294V, K315I, V320L, D323I, Y372F, Y377L, and E378W); 3U7M (having corresponding mutations: G58L, V59A, G60M, A62V, Q65L, A74V, E109F, G110Q, L112I, S113A, R124L, Y147L, I150L, V151I, E155A, and E185L); 1Z97 (having corresponding mutations: N59D, K61L, R64L, R88L, Q89F, E103L, F127Y, L131Q, V139I, S193A, L194M, T195A, T196V, C199L, and I203V).

TABLE 5
Estimated relative binding affinities for metalloprotein binder
scaffolds using modeled M72-modified AA dipeptide as a substrate.

| | Metal-chelating | Native | Designed | ΔKd (Native to |
|---|---|---|---|---|
| PDB ID | Residues | Binder ΔKd | Binder ΔKd | Designed) |
| 1AST | 92H,96H,102H | 63722.81 | 153194.55 | 2.40 |
| 1C7K | 83H,87H,93D | 3619.15 | 55440.38 | 15.32 |
| 1HEE | 69H,72E,196H | 4872.80 | 10558.65 | 2.17 |
| 1IAG | 141H,145H,151H | 272387.13 | 117757.55 | 0.43 |
| 1JAN | 119H,123H,129H | 86651.15 | 60249.68 | 0.70 |
| 1JD0 | 89H,91H,115H | 57701.91 | 74639.14 | 1.29 |
| 1KAP | 176H,180H,186H | 607558.25 | 24239.16 | 0.04 |
| 1LML | 165H,169H,235H | 373307.00 | 883379.63 | 2.37 |
| 1OBR | 69H,72E,204H | 5899.94 | 63562.03 | 10.77 |
| 1Z97 | 91H,93H,116H | 137532.61 | 56436.28 | 0.41 |
| 2CAB | 90H,92H,115H | 25980.07 | 26741.91 | 1.03 |
| 2CKI | 168H,172H,178H | 49689.99 | 325644.06 | 6.55 |
| 2FV5 | 190H,194H,200H | 84020.88 | 121252.92 | 1.44 |
| 2J83 | 166H,170H,176H | 393620.22 | 764526.31 | 1.94 |
| 2X7M | 132H,136H,142H | 25480.32 | 221792.09 | 8.70 |
| 3ML5 | 94H,96H,119H | 24181.60 | 75366.99 | 3.12 |
| 3P1V | 292H,296H,303D | 1044.22 | 14978.47 | 14.34 |
| 3P24 | 162D,316H,320H | 1033.65 | 321.75 | 0.31 |
| 3U7M | 111C,154H,158H | 1734168.13 | 3053128.50 | 1.76 |
| 3UJZ | 408H,412H,418H | 21819.33 | 649907.44 | 29.79 |
| 4DJL | 69H,72E,204H | 4125.50 | 6537.24 | 1.58 |
| 4DLM | 7H,9H,242D | 6191.26 | 8855.59 | 1.43 |
| 4KNM | 92H,94H,117H | 16386.69 | 39221.91 | 2.39 |
| 4L63 | 160H,164H,170H | 414865.09 | 706820.19 | 1.70 |
| 4LP6 | 91H,93H,116H | 41791.11 | 127011.07 | 3.04 |
| 4Q4E | 293H,297H,316E | 550570.81 | 1051209.63 | 1.91 |
| 4YYT | 91H,93H,116H | 5946.47 | 119100.20 | 20.03 |
| 5E3C | 448H,453H,506E | 36406.44 | 321.92 | 0.01 |
| 5ELY | 333D,371E,499H | 3.40 | 193.01 | 56.78 |
| 5JN8 | 97H,99H,122H | 105169.23 | 158107.19 | 1.50 |
| 5K7J | 181H,185H,212H | 531165.69 | 275995.47 | 0.52 |
| 5OD1 | 35H,61H,65H | 57407.41 | 105279.06 | 1.83 |

Some of the tested scaffolds (e.g., 3U7M, 1KAP, 4Q4E, 5K7J) show elevated “Native Binder ΔKd” parameter (calculated upon mutation of metal-chelating residue to Gly), indicating that metal-chelating residues significantly contribute to binding affinity for binding between the unmodified binder and the M72-modified AA model peptide. Other tested scaffolds show significantly elevated “ΔKd (Native to Designed)” parameter (the fold-change improvement in Kd upon mutating the binder from the native binder sequence), indicating that the designed (modified) binders have improved binding affinities for binding between the designed binder and the M72-modified AA model peptide.

Examples of such designed binders having improved binding affinities include SEQ ID NOs: 35-38 based on the following scaffolds: 4Q4E (having corresponding mutations: E117V, F254L, M256T, A258M, E260V, F267V, N268H, K270I, Y271A, V272I, V290L, E294A, K315I, Y377L, N776I, and R779L); 3U7M (having corresponding mutations: Q45L, G58A, V59G, G60M, A62V, Q65M, A74V, V75M, Y86W, G110E, Y147L, P148A, V151A, F152M, E155A, and E185L); 1LML (having corresponding mutations: E121H, V124I, E166A, G230K, S231I, A249K, and F352L); 3UJZ (having corresponding mutations: W328E, H329T, G389K, and D409A).

TABLE 6
Estimated relative binding affinities for metalloprotein binder
scaffolds using modeled M83-modified AA dipeptide as a substrate.

| | Metal-chelating | Native | Designed | ΔKd (Native to |
|---|---|---|---|---|
| PDB ID | Residues | Binder ΔKd | Binder ΔKd | Designed) |
| 1AST | 92H,96H,102H | 2157.53 | 643.78 | 0.30 |
| 1C7K | 83H,87H,93D | 3.87 | 757.14 | 195.71 |
| 1HEE | 69H,72E,196H | 101.78 | 447.93 | 4.40 |
| 1IAG | 141H,145H,151H | 18156.32 | 32811.39 | 1.81 |
| 1JAN | 119H,123H,129H | 3457.45 | 4324.86 | 1.25 |
| 1JD0 | 89H,91H,115H | 366.58 | 7387.84 | 20.15 |
| 1KAP | 176H,180H,186H | 210.29 | 1934.99 | 9.20 |
| 1LML | 165H,169H,235H | 7425.52 | 16438.11 | 2.21 |
| 1OBR | 69H,72E,204H | 400.06 | 178.40 | 0.45 |
| 1Z97 | 91H,93H,116H | 1553.89 | 3533.71 | 2.27 |
| 2CAB | 90H,92H,115H | 218.38 | 155.09 | 0.71 |
| 2CKI | 168H,172H,178H | 2304.49 | 6260.11 | 2.72 |
| 2FV5 | 190H,194H,200H | 7887.99 | 6212.09 | 0.79 |
| 2J83 | 166H,170H,176H | 3303.57 | 5819.76 | 1.76 |
| 2X7M | 132H,136H,142H | 2178.02 | 2675.89 | 1.23 |
| 3ML5 | 94H,96H,119H | 437.21 | 854.57 | 1.95 |
| 3P24 | 162D,316H,320H | 90.89 | 116.58 | 1.28 |
| 3UJZ | 408H,412H,418H | 1463.70 | 3880.85 | 2.65 |
| 4DJL | 69H,72E,204H | 148.21 | 317.72 | 2.14 |
| 4DLM | 7H,9H,242D | 1581.41 | 826.23 | 0.52 |
| 4KNM | 92H,94H,117H | 227.25 | 397.20 | 1.75 |
| 4L63 | 160H,164H,170H | 4040.53 | 2388.26 | 0.59 |
| 4LP6 | 91H,93H,116H | 496.85 | 622.44 | 1.25 |
| 4Q4E | 293H,297H,316E | 17175.40 | 29270.72 | 1.70 |
| 4YYT | 91H,93H,116H | 2614.89 | 1839.03 | 0.70 |
| 5E3C | 448H,453H,506E | 551.44 | 2259.82 | 4.10 |
| 5JN8 | 97H,99H,122H | 917.74 | 1065.74 | 1.16 |
| 5K7J | 181H,185H,212H | 21430.29 | 45.75 | 0.00 |
| 5OD1 | 35H,61H,65H | 1673.40 | 3711.28 | 2.22 |

Some of the tested scaffolds (e.g., 5K7J, 1IAG, 4Q4E, 2FV5) show elevated “Native Binder ΔKd” parameter (calculated upon mutation of metal-chelating residue to Gly), indicating that metal-chelating residues significantly contribute to binding affinity for binding between the unmodified binder and the M83-modified AA model peptide. Other tested scaffolds show significantly elevated “ΔKd (Native to Designed)” parameter (the fold-change improvement in Kd upon mutating the binder from the native binder sequence), indicating that the designed (modified) binders have improved binding affinities for binding between the designed binder and the M83-modified AA model peptide.

Examples of such designed binders having improved binding affinities include SEQ ID NOs: 39-41 based on the following scaffolds: HAG (having corresponding mutations: G108L, K109L, E142L, K154P, R166L, and G168E); 4Q4E (having corresponding mutations: E117M, M256T, G257L, A258L, M259I, E260P, Y271L, K282E, D286R, R289A, V290A, E294I, T343I, Y377L, and E378Y); 1LML (having corresponding mutations: E166A, G227A, G230K, S231A, and F352R).

TABLE 7
Estimated relative binding affinities for metalloprotein binder
scaffolds using modeled M86-modified AA dipeptide as a substrate.

| | Metal-chelating | Native | Designed | ΔKd (Native to |
|---|---|---|---|---|
| PDB ID | Residues | Binder ΔKd | Binder ΔKd | Designed) |
| 4Q4E | 293H,297H,316E | 94.46 | 9912.31 | 104.94 |
| 3P24 | 162D,316H,320H | 5.96 | 546.35 | 91.74 |
| 1LML | 165H,169H,235H | 2377.73 | 56506.00 | 23.76 |
| 2J83 | 166H,170H,176H | 1399.24 | 17083.00 | 12.21 |
| 2X7M | 132H,136H,142H | 400.13 | 4735.54 | 11.84 |
| 5E3C | 448H,453H,506E | 3361.75 | 24206.04 | 7.20 |
| 2FV5 | 190H,194H,200H | 5330.16 | 22997.28 | 4.31 |
| 1AST | 92H,96H,102H | 599.93 | 2454.49 | 4.09 |
| 1HEE | 69H,72E,196H | 175.26 | 520.44 | 2.97 |
| 1C7K | 83H,87H,93D | 9608.85 | 21262.48 | 2.21 |
| 2CAB | 90H,92H,115H | 1604.91 | 3465.38 | 2.16 |
| 3UJZ | 408H,412H,418H | 2939.88 | 5962.35 | 2.03 |
| 5K7J | 181H,185H,212H | 3518.29 | 6266.39 | 1.78 |
| 1IAG | 141H,145H,151H | 11713.26 | 18621.20 | 1.59 |
| 1Z97 | 91H,93H,116H | 10557.23 | 11990.08 | 1.14 |
| 1JAN | 119H,123H,129H | 1591.16 | 1676.96 | 1.05 |
| 1OBR | 69H,72E,204H | 1811.64 | 1899.29 | 1.05 |
| 4DJL | 69H,72E,204H | 2206.03 | 2311.16 | 1.05 |
| 4YYT | 91H,93H,116H | 3987.37 | 3931.71 | 0.99 |
| 1KAP | 176H,180H,186H | 1030.36 | 988.12 | 0.96 |
| 2CKI | 168H,172H,178H | 2761.10 | 2629.84 | 0.95 |
| 4L63 | 160H,164H,170H | 5922.17 | 4017.96 | 0.68 |
| 4KNM | 92H,94H,117H | 1591.47 | 979.08 | 0.62 |
| 3P1V | 292H,296H,303D | 74.18 | 43.77 | 0.59 |
| 1JD0 | 89H,91H,115H | 2021.82 | 1153.22 | 0.57 |
| 5JN8 | 97H,99H,122H | 5808.72 | 2999.19 | 0.52 |
| 4LP6 | 91H,93H,116H | 4784.62 | 2140.14 | 0.45 |
| 3ML5 | 94H,96H,119H | 5293.72 | 1478.20 | 0.28 |
| 5OD1 | 35H,61H,65H | 4392.91 | 357.91 | 0.08 |
| 5ELY | 333D,371E,499H | 28.58 | 0.22 | 0.01 |
| 4DLM | 7H,9H,242D | 49820.61 | 324.14 | 0.01 |
| 3U7M | 111C,154H,158H | 47436.00 | 28.83 | 0.00 |

Some of the tested scaffolds (e.g., 4DLM, 3U7M, 1IAG) show elevated “Native Binder ΔKd” parameter (calculated upon mutation of metal-chelating residue to Gly), indicating that metal-chelating residues significantly contribute to binding affinity for binding between the unmodified binder and the M86-modified AA model peptide. Other tested scaffolds show significantly elevated “ΔKd (Native to Designed)” parameter (the fold-change improvement in Kd upon mutating the binder from the native binder sequence), indicating that the designed (modified) binders have improved binding affinities for binding between the designed binder and the M86-modified AA model peptide.

Examples of such designed binders having improved binding affinities include SEQ ID NOs: 42-44 based on the following scaffolds: 1LML (having corresponding mutations: E121H, E166V, A229R, S231V, A249D, and F352L); 5E3C (having corresponding mutations: S106A, F107W, E314W, Y316V, R317F, E325M, E327F, F379M, S382L, A386L, G387M, I388M, N389V, Q564D, A565V, and H566L); 2FV5 (having corresponding mutations: V99L, T132R, G134L, L135I, A136V, R142H, and E191A).

TABLE 8
Estimated relative binding affinities for metalloprotein binder
scaffolds using modeled M93-modified AA dipeptide as a substrate.

| | Metal-chelating | Native | Designed | ΔKd (Native to |
|---|---|---|---|---|
| PDB ID | Residues | Binder ΔKd | Binder ΔKd | Designed) |
| 1AST | 92H,96H,102H | 88.28 | 604.55 | 6.85 |
| 1C7K | 83H,87H,93D | 36.94 | 135.29 | 3.66 |
| 1HEE | 69H,72E,196H | 42.01 | 13279.05 | 316.11 |
| 1IAG | 141H,145H,151H | 449733.69 | 761383.69 | 1.69 |
| 1JAN | 119H,123H,129H | 5577.24 | 2358.56 | 0.42 |
| 1JD0 | 89H,91H,115H | 13848.33 | 8220.91 | 0.59 |
| 1KAP | 176H,180H,186H | 16227.89 | 9729.70 | 0.60 |
| 1LML | 165H,169H,235H | 462.30 | 1269.72 | 2.75 |
| 1OBR | 69H,72E,204H | 292.25 | 457.65 | 1.57 |
| 1Z97 | 91H,93H,116H | 7690.77 | 9501.19 | 1.24 |
| 2CAB | 90H,92H,115H | 1985.88 | 3837.03 | 1.93 |
| 2CKI | 168H,172H,178H | 214.61 | 627.69 | 2.92 |
| 2FV5 | 190H,194H,200H | 74857.21 | 63626.83 | 0.85 |
| 2J83 | 166H,170H,176H | 2659.17 | 6651.53 | 2.50 |
| 2X7M | 132H,136H,142H | 1640.68 | 7633.48 | 4.65 |
| 3ML5 | 94H,96H,119H | 2038.30 | 3126.74 | 1.53 |
| 3P1V | 292H,296H,303D | 0.79 | 6813.98 | 8619.65 |
| 3P24 | 162D,316H,320H | 216.76 | 100.03 | 0.46 |
| 3U7M | 111C,154H,158H | 9019.96 | 38102.75 | 4.22 |
| 3UJZ | 408H,412H,418H | 3886.24 | 1106.90 | 0.28 |
| 4DJL | 69H,72E,204H | 476.38 | 412.89 | 0.87 |
| 4DLM | 7H,9H,242D | 1196381.63 | 236.72 | 0.00 |
| 4KNM | 92H,94H,117H | 327.86 | 2651.61 | 8.09 |
| 4L63 | 160H,164H,170H | 268.50 | 4128.77 | 15.38 |
| 4LP6 | 91H,93H,116H | 2863.43 | 2057.83 | 0.72 |
| 4Q4E | 293H,297H,316E | 71632.62 | 201534.89 | 2.81 |
| 4YYT | 91H,93H,116H | 2612.60 | 5526.18 | 2.12 |
| 5JN8 | 97H,99H,122H | 6403.97 | 15281.48 | 2.39 |
| 5K7J | 181H,185H,212H | 186244.88 | 5069823.00 | 27.22 |
| 5OD1 | 35H,61H,65H | 217.69 | 1749.32 | 8.04 |

Some of the tested scaffolds (e.g., 4DLM, 1IAG, 5K7J, 2FV5, 4Q4E) show elevated “Native Binder ΔKd” parameter (calculated upon mutation of metal-chelating residue to Gly), indicating that metal-chelating residues significantly contribute to binding affinity for binding between the unmodified binder and the M93-modified AA model peptide. Other tested scaffolds show significantly elevated “ΔKd (Native to Designed)” parameter (the fold-change improvement in Kd upon mutating the binder from the native binder sequence), indicating that the designed (modified) binders have improved binding affinities for binding between the designed binder and the M93-modified AA model peptide.

Examples of such designed binders having improved binding affinities include SEQ ID NOs: 45-47 based on the following scaffolds: 5K7J (having corresponding mutations: K7D, P58V, I134L, K136M, L158I, G160M, I161V, N162L, D166M, G179V, I180L, A183K, D184S, A210M, A211V, and L232I); HAG (having corresponding mutations: G108M, K109L, E142L, R166L, and G168D); 4Q4E (having corresponding mutations: E117L, G257I, A258V, M259I, E260P, K282E, D286K, R289Q, V290I, E294A, K315I, V320L, D323V, T343I, Y372F, Y377M, and E378W).

Further, in silico tested scaffolds selected based on high Native Binder ΔKd or high Designed Binder ΔKd (e.g., 3U7M, 4Q4E and 1AST) were further evaluated against a panel of NTMs (M64-M91 and M93-M98). All three scaffolds shared M72 as one of the two NTMs that provide highest relative binding affinity (see Tables 9 and 10).

TABLE 9
Estimated relative binding affinities for PDB ID 3U7M
metalloprotein binder scaffold tested against different
modeled NTM-modified AA dipeptides as substrates.

| | Metal-chelating | Native | Designed | ΔKd (Native to |
|---|---|---|---|---|
| NTM | Residues | Binder ΔKd | Binder ΔKd | Designed) |
| M71 | 111C,154H,158H | 3501028.50 | 2207561.25 | 0.63 |
| M72 | 111C,154H,158H | 1734168.13 | 3053128.50 | 1.76 |
| M89 | 111C,154H,158H | 1031853.69 | 40826.33 | 0.04 |
| M96 | 111C,154H,158H | 976407.19 | 18512.25 | 0.02 |
| M64 | 111C,154H,158H | 541295.44 | 414363.56 | 0.77 |
| M69 | 111C,154H,158H | 482314.28 | 230.61 | 0.00 |
| M79 | 111C,154H,158H | 351483.22 | 164567.31 | 0.47 |
| M74 | 111C,154H,158H | 320104.91 | 154488.63 | 0.48 |
| M77 | 111C,154H,158H | 211693.53 | 8780.37 | 0.04 |
| M87 | 111C,154H,158H | 188750.83 | 33779.50 | 0.18 |
| M73 | 111C,154H,158H | 164210.64 | 1355697.25 | 8.26 |
| M65 | 111C,154H,158H | 114409.43 | 352385.06 | 3.08 |
| M68 | 111C,154H,158H | 112102.16 | 125073.30 | 1.12 |
| M85 | 111C,154H,158H | 103877.56 | 14651.04 | 0.14 |
| M95 | 111C,154H,158H | 60689.87 | 77287.09 | 1.27 |
| M70 | 111C,154H,158H | 47725.49 | 54078.62 | 1.13 |
| M86 | 111C,154H,158H | 47436.00 | 28.83 | 0.00 |
| M76 | 111C,154H,158H | 46162.30 | 42348.16 | 0.92 |
| M75 | 111C,154H,158H | 29492.38 | 161397.19 | 5.47 |
| M88 | 111C,154H,158H | 28316.23 | 83574.75 | 2.95 |
| M66 | 111C,154H,158H | 26813.67 | 41052.95 | 1.53 |
| M94 | 111C,154H,158H | 21822.65 | 7226.57 | 0.33 |
| M93 | 111C,154H,158H | 9019.96 | 38102.75 | 4.22 |
| M67 | 111C,154H,158H | 8874.03 | 2196.70 | 0.25 |
| M82 | 111C,154H,158H | 5851.96 | 70393.65 | 12.03 |
| M84 | 111C,154H,158H | 5279.65 | 13634.45 | 2.58 |
| M97 | 111C,154H,158H | 4746.72 | 233747.84 | 49.24 |
| M91 | 111C,154H,158H | 4423.19 | 170669.83 | 38.59 |
| M80 | 111C,154H,158H | 3808.76 | 6704.17 | 1.76 |
| M81 | 111C,154H,158H | 3596.61 | 2829.83 | 0.79 |
| M78 | 111C,154H,158H | 2998.50 | 8125.12 | 2.71 |
| M83 | 111C,154H,158H | 2515.06 | 0.00 | 0.00 |
| M90 | 111C,154H,158H | 231.28 | 1522.03 | 6.58 |

TABLE 10
Estimated relative binding affinities for 4Q4E metalloprotein
binder scaffold tested against different modeled NTM-
modified AA dipeptides as substrates.

| | Metal-chelating | Native | Designed | ΔKd (Native to |
|---|---|---|---|---|
| NTM | Residues | Binder ΔKd | Binder ΔKd | Designed) |
| M72 | 293H,297H,316E | 550570.81 | 1051209.63 | 1.91 |
| M93 | 293H,297H,316E | 71632.62 | 201534.89 | 2.81 |
| M75 | 293H,297H,316E | 63327.81 | 1008.73 | 0.02 |
| M69 | 293H,297H,316E | 57445.40 | 44320.43 | 0.77 |
| M65 | 293H,297H,316E | 47969.63 | 650053.88 | 13.55 |
| M71 | 293H,297H,316E | 40402.22 | 31.60 | 0.00 |
| M70 | 293H,297H,316E | 25470.41 | 30806.31 | 1.21 |
| M68 | 293H,297H,316E | 21628.25 | 4221563.00 | 195.19 |
| M96 | 293H,297H,316E | 20559.98 | 11119.35 | 0.54 |
| M73 | 293H,297H,316E | 20422.63 | 139066.25 | 6.81 |
| M83 | 293H,297H,316E | 17175.40 | 29270.72 | 1.70 |
| M78 | 293H,297H,316E | 14846.99 | 401.02 | 0.03 |
| M95 | 293H,297H,316E | 13913.03 | 152.32 | 0.01 |
| M66 | 293H,297H,316E | 12065.32 | 17295.57 | 1.43 |
| M85 | 293H,297H,316E | 10439.38 | 70105.59 | 6.72 |
| M76 | 293H,297H,316E | 8862.41 | 19224.22 | 2.17 |
| M74 | 293H,297H,316E | 8742.56 | 4372.22 | 0.50 |
| M64 | 293H,297H,316E | 8287.36 | 26716.71 | 3.22 |
| M81 | 293H,297H,316E | 7297.53 | 186.28 | 0.03 |
| M87 | 293H,297H,316E | 4935.47 | 1178.78 | 0.24 |
| M97 | 293H,297H,316E | 4349.30 | 404.36 | 0.09 |
| M67 | 293H,297H,316E | 3731.95 | 2670.95 | 0.72 |
| M77 | 293H,297H,316E | 3045.48 | 31041.90 | 10.19 |
| M79 | 293H,297H,316E | 2489.93 | 2661.60 | 1.07 |
| M88 | 293H,297H,316E | 2231.58 | 26357.38 | 11.81 |
| M89 | 293H,297H,316E | 1534.39 | 805.60 | 0.53 |
| M91 | 293H,297H,316E | 1369.91 | 4103.11 | 3.00 |
| M80 | 293H,297H.316E | 916.31 | 684.38 | 0.75 |
| M90 | 293H,297H,316E | 547.45 | 497.67 | 0.91 |
| M82 | 293H,297H,316E | 401.61 | 3249.67 | 8.09 |
| M94 | 293H,297H,316E | 217.74 | 391455.53 | 1797.83 |
| M84 | 293H,297H,316E | 217.11 | 274.43 | 1.26 |
| M86 | 293H,297H,316E | 94.46 | 9912.31 | 104.94 |

Engineered (designed) binders presented in the Sequence Listing (SEQ ID NOs: 28-47 show binding diversity across different tested NTMs. By using the described modeling methods, metalloprotein binders can be engineered to recognize a diverse set of Z-P1s on target peptides. Sequences of engineered binders differ significantly from corresponding starting metalloenzyme scaffolds, and each of the engineered binders designed to have an improve binding affinity and having sequences as set forth in SEQ ID NOs: 28-47 contains 5-20 amino acid substitutions from the corresponding starting scaffold. Since most amino acid substitutions were designed to be on the substrate-interaction region of the binders, geometry of substrate-binding pockets of the scaffolds and atomic interactions within them were significantly changed during modelling process.

Engineered binders set forth in SEQ ID NOs: 28-47 typically have about 91-98% sequence identity with corresponding starting scaffolds. Additionally, these binders may be further processed for improving their characteristics, such as Z-P1 affinity, P1 selectivity and/or P2 tolerance. Moreover, conservative amino acid substitutions can be made in the binder's sequence that would improve its characteristics unrelated to the Z-P1 binding, such as improve binder's stability or increase expression level of the binder in bacterial cells. Such conservative amino acid substitutions are known to skilled in the art, and the updated binder's sequence may have less than about 90% sequence identity with the corresponding starting scaffolds (for example, may have about 80% or 85% sequence identity with the corresponding starting scaffolds).

### Example 3. Exemplary Origin, Synthesis and Installation of NTMs on NTAA Residues of Peptides

Structures, origin and installation methods for exemplary N-terminal modifier agents used for modification of NTAA residues of peptides are shown below.

N-terminal modifier agent for M=M64 (in the ester form).

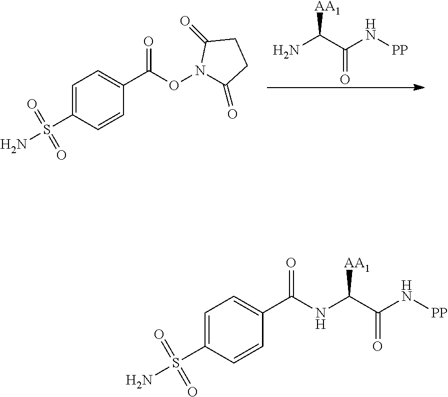

Exemplary method of installing M64 onto N-terminal amino acid of a peptide, shown as NTAA-PP. Peptides, in solution or on solid-support, were dissolved in 25 uL of 0.4 M MOPS buffer, pH=7.6 and 25 uL of acetonitrile (ACN). Separately, the active ester reagent was prepared from M64 and dissolved in 25 uL DMA and 25 uL ACN to a concentration of 0.05 M stock solution. Then, 50 uL of the active ester stock solution was added to the peptide-ACN:MOPS solution and incubated at 65° C. for 60 minutes. Upon completion, the peptides were functionalized with the respective modification as shown in the above schemes.

Alternatively, a surfactant-aqueous coupled system can be employed to install NTM (M64) onto the N-terminal amino acid of peptides. Using a 10 mM solution of 5% DMSO in 2% TGPS-750-M in water containing 1% 2,6-lutidine, the peptides are modified to completion in 20 minutes at 40° C.

M65-M91 and M93-M97 NTMs have been similarly installed on N-terminal amino acids of peptides.

Exemplary NTM Materials and Syntheses.

A. Commercial sources of 4-carboxybenzenesulfonamide and substituted 4-carboxybenzenesulfonamides:

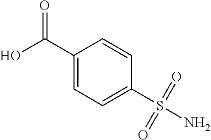 [SMILES: NS(=O)(=O)c1ccc(C(=O)O)cc1]

4-Carboxybenzenesulfonamide; vendor: Combi-Blocks; Item #: QA-8702; MW 201.2; CAS 138-41-0

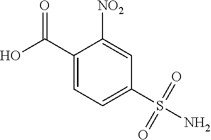 [SMILES: NS(=O)(=O)c1ccc(C(=O)O)c([N+](=O)[O-])c1]

2-Nitro-4-sulfamoylbenzoic acid; vendor: Combi-Blocks; Item #: WZ-9277; MW 246.2; CAS 29092-31-7

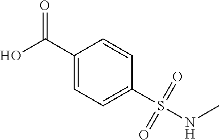

4-[(Methylamino)sulfonyl]benzoic acid; vendor: Combi-Blocks; Item #: ST-1977; MW 215.2; CAS 10252-63-8

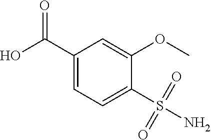

3-Methoxy-4-sulfamoylbenzoic acid; vendor: Enamine; Item #: EN300-189718; MW 231.2; CAS 860562-94-3

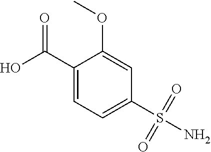

2-Methoxy-4-sulfamoylbenzoic acid; vendor: Fisher Scientific; Item #: BB016296; MW 231.2; CAS 4816-28-8

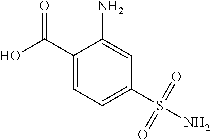 [SMILES: Nc1cc(S(N)(=O)=O)ccc1C(=O)O]

2-Amino-4-sulfamoylbenzoic acid; vendor: Enamine; Item #: EN300-263098; MW 216.2; CAS 25096-72-4

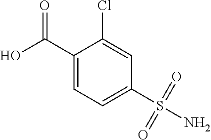 [SMILES: NS(=O)(=O)c1ccc(C(=O)O)c(Cl)c1]

2-Chloro-4-sulfamoylbenzoic acid; vendor: Enamine; Item #: EN300-147322; MW 235.6; CAS 53250-84-3

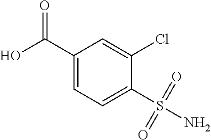 [SMILES: NS(=O)(=O)c1ccc(C(=O)O)cc1Cl]

3-Chloro-4-sulfamoylbenzoic acid; vendor: Enamine; Item #: EN300-64348; MW 235.6; CAS 34263-53-1

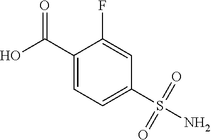 [SMILES: NS(=O)(=O)c1ccc(C(=O)O)c(F)c1]

2-Fluoro-4-sulfamoylbenzoic acid; vendor: Enamine; Item #: EN300-123320; MW 219.2; CAS 714968-42-0

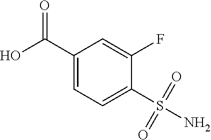 [SMILES: NS(=O)(=O)c1ccc(C(=O)O)cc1F]

3-Fluoro-4-sulfamoylbenzoic acid; vendor: Enamine; Item #: EN300-97835; MW 219.2; CAS 244606-37-9

B. Commercial sources of sulfamoylpyridine carboxylic acids:

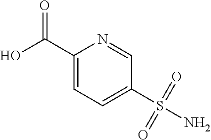 [SMILES: NS(=O)(=O)c1ccc(C(=O)O)nc1]

5-Sulfamoylpyridine-3-carboxylic acid; vendor: Enamine; Item #: EN300-124374; MW 202.2; CAS 1308677-67-9

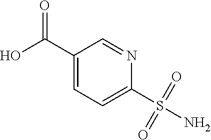 [SMILES: NS(=O)(=O)c1ccc(C(=O)O)cn1]

6-Sulfamoylpyridine-3-carboxylic acid; vendor: Enamine; Item #: EN300-120480; MW 202.2; CAS 285135-56-0

C. Substituted 4-Carboxybenzenesulfonamides prepared in step(s) from commercial materials:

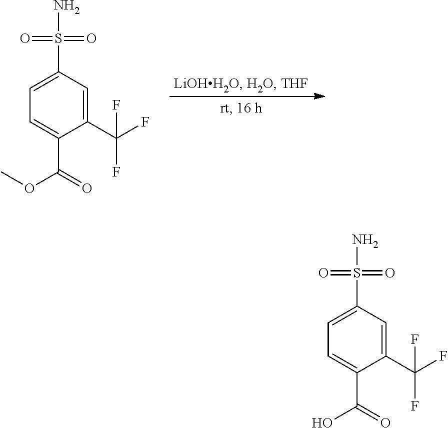 [SMILES: COC(=O)c1ccc(S(N)(=O)=O)cc1C(F)(F)F ; NS(=O)(=O)c1ccc(C(=O)O)c(C(F)(F)F)c1]

To a solution of commercially available methyl 4-sulfamoyl-2-(trifluoromethyl)benzoate (250 mg, 0.88 mmol) in THF (5 mL) a premixture of LiOH.H2O (111 mg, 2.65 mmol) in H₂O (2 mL) was added, and the resulting solution was stirred for 16 h at room temperature. The reaction mixture was quenched with 4 eq. of conc. HCl and the solvents were removed in vacuo. The white residue was suspended in H₂O, sonicated, stirred for 2 h, and the solids were collected by filtration to give the desired 2-trifluoro-4-sulfamoylbenzoic acid as a pure white solid. MS (ESI) 267 (M⁻−H).

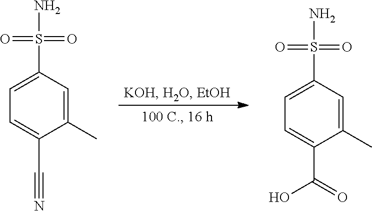

To a solution of commercially available 4-cyano-3-methylbenzene-1-sulfonamide (250 mg, 1.27 mmol) in EtOH (6 mL) H₂O (6 mL) and pulverized KOH (572 mg, 10.19 mmol) were added. The resulting solution was stirred for 16 h at 100° C. The reaction mixture was quenched with 9 eq. of conc. HCl and the solvents were removed in vacuo. The white residue was purified by flash column chromatography (SiO₂) eluting with EtOAc (spiked with 5% AcOH) and heptane using a 10% to 100% gradient to give the desired 2-methyl-4-sulfamoylbenzoic acid as a pure white solid. MS (ESI) 214 (M⁻−H).

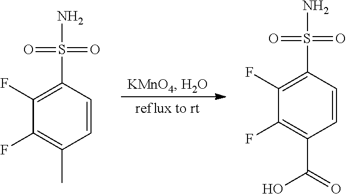

Commercially available 2,3-difluoro-4-methylbenzene-1-sulfonamide was suspended in H₂O (13 mL) and stirred to reflux after which KMnO₄ (858 mg, 5.43 mmol) was added in portions over 50 min. The resulting solution was stirred for an additional 30 min at reflux, then stirred at room temperature for 16 h. The reaction mixture was filtered through a frit and the filtrate was adjusted to pH=1 with conc. HCl. The low pH filtrate was then extracted twice with EtOAc, and the organics were dried (Na₂SO₄), filtered, and evaporated in vacuo. The mostly pure white residue was further triturated in a 1% MeOH/DCM solvent system and filtered to give the desired 2,3-difluoro-4-sulfamoylbenzoic acid as a white solid. MS (ESI) 236 (M⁻−H).

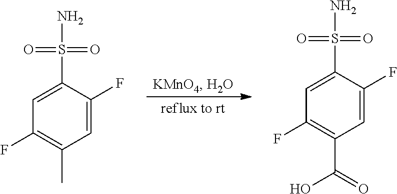

Commercially available 2,5-difluoro-4-methylbenzene-1-sulfonamide was suspended in H₂O (13 mL) and stirred to reflux after which KMnO₄ (858 mg, 5.43 mmol) was added in portions over 50 min. The resulting solution was stirred for an additional 30 min at reflux, then stirred at room temperature for 16 h. The reaction mixture was filtered through a frit and the filtrate was adjusted to pH=1 with conc. HCl. The low pH filtrate was then extracted twice with EtOAc and the organics were dried (Na₂SO₄), filtered, and evaporated in vacuo. The mostly pure white residue was further triturated in a 1% MeOH/DCM solvent system and filtered to give the desired 2,5-difluoro-4-sulfamoylbenzoic acid as a white solid. MS (ESI) 236 (M⁻−H).

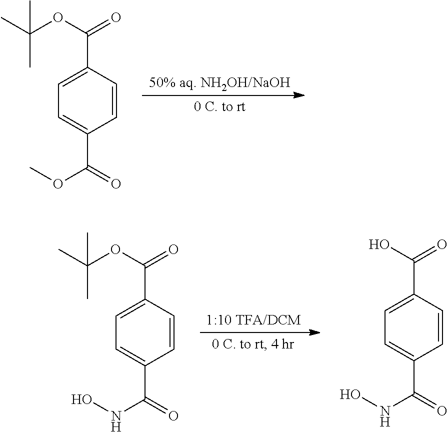 [SMILES: COC(=O)c1ccc(C(=O)OC(C)(C)C)cc1 ; CC(C)(C)OC(=O)c1ccc(C(=O)NO)cc1 ; O=C(O)c1ccc(C(=O)NO)cc1]

Step 1:
Pulverized NaOH (424 mg, 10.60 mmol) was dissolved in a 0° C. solution of hydroxylamine in water (50% wt, 2.50 mL, 42.40 mmol) which was followed by dropwise addition of commercially available tert-butyl methyl terephthalate (250 mg, 1.06 mmol) premixed with THF/MeOH (15/15 mL). The resulting reaction mixture was allowed to warm to room temperature and stirred for 45 min before acetic acid (0.66 mL, 11.66 mmol) was added to quench. The solvents were removed by evaporation under reduced pressure. The resulting crude was treated with saturated aqueous NaHCO₃ (pH adjusted to ˜9) and diluted with ethyl acetate. The organic phase was washed with brine, dried over anhydrous Na₂SO₄, filtered, and concentrated under vacuum to afford the 4-tert-butylcarboxy-benzenehydroxamic acid as a white, crystalline powder. MS (ESI) 238 (M⁻−H).

Step 2:
To a 0° C. solution of 4-tert-butylcarboxy-benzenehydroxamic acid (100 mg, 0.42 mmol) in DCM (5 mL) was added TFA (0.5 mL). The resulting reaction mixture was allowed to warm to room temperature and stirred for 4 h at which time the product was a thick suspension in the reaction mixture. The solids were filtered off and rinsed with DCM to afford 4-carboxybenzenehydroxamic acid as a white powder. MS (ESI) 181 (M⁻−H).

D. Alternative synthesis of sulfamoylpyridine carboxylic acid prepared from commercial materials:

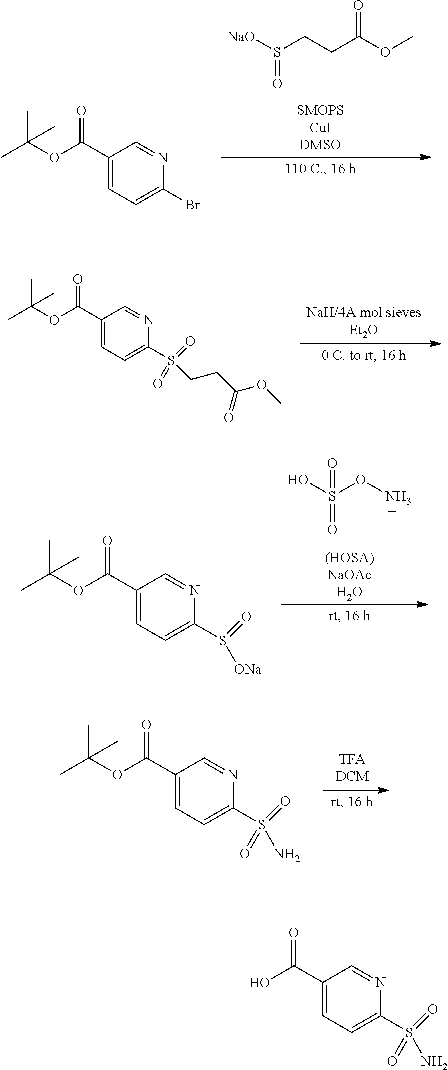

Step 1:
To tert-butyl 6-bromonicotinate (0.5 g, 1.94 mmol) in DMSO (10 mL) SMOPS (1.01 g, 5.82 mmol) and CuI (1.11 g, 5.82 mmol) were added. The reaction was stirred under a natural atmosphere at 110° C. for 16 hours. The mixture was cooled to room temperature, diluted with excess ethyl acetate and filtered through a pad of celite. The filtrate was washed 2× with water, 2× with brine, dried (Na₂SO₄), filtered, and evaporated in vacuo. The residue was purified by flash column chromatography (SiO₂) eluting with EtOAc and heptane using a 20% to 100% gradient to give the desired methyl 3-((5-tert-butylcarboxypyridin-2-yl)sulfonyl)propanoate. MS (ESI) 330 (M⁺+H).

Step 2:
Under an argon atmosphere at 0° C. sodium hydride (22 mg, 0.55 mmol) and activated 4 A molecular sieves (1.42 g, 2.58 g per mmol of starting material) were combined. To the stirring solids methyl 3-((5-tert-butylcarboxypyridin-2-yl)sulfonyl)propanoate (0.18 g, 0.55 mmol) premixed with dry Et₂O (15 mL) was slowly added. After 5 minutes, the ice bath was removed and the reaction was sealed and stirred at room temperature for 16 hours. The mixture was cooled to 0 C, diluted with excess MeOH, and filtered through a pad of celite. The filtrate was evaporated in vacuo, dissolved in water and washed 3× with DCM. The aqueous layer was evaporated in vacuo and coevaporated once with heptane and once with CH₃CN. The white solid residue was the desired and pure 5-tert-butylcarboxypyridin-2-yl-sodium sulfinate. MS (ESI) 245 (M⁺+H).

Step 3:
To 5-tert-butylcarboxypyridin-2-yl)sodium sulfinate (0.15 g, 0.57 mmol) in H₂O (5 mL) sodium acetate (0.057 g, 0.68 mmol) and hydroxylamine-O-sulfonic acid
(0.077 g, 0.68 mmol) were added. The reaction was stirred at room temperature for 16 hours and filtered to give pure 5-tert-butylcarboxypyridin-2-yl-sulfonamide. MS (ESI) 259 (M⁺+H).

Step 4:
To a 0° C. solution of 5-tert-butylcarboxypyridin-2-yl-sulfonamide (0.15 g, 0.58 mmol) in DCM (6 mL) was added TFA (0.6 mL). The resulting reaction mixture was allowed to warm to room temperature and stirred for 16 h. The solvents were evaporated to dryness and covevaporated 2× with heptane to afford 6-sulfamoylpyridine-3-carboxylic acid as a white powder. MS (ESI) 201 (M⁻−H).

### Example 4. Binder Engineering from the Metalloenzyme Scaffolds

Binder engineering involves improving affinities of potential binding sites through rational, structure-based approaches on a parental scaffold and generating libraries that contain degenerate NNK codons at multiple, defined positions using Kunkel mutagenesis and phage display selection. Kunkel mutagenesis is a known site-directed mutagenesis strategy that introduces point mutations by annealing mutation-containing oligonucleotides to single-stranded uracil-containing single strand DNA (dU-ssDNA) templates. Exemplary Kunkel mutagenesis and phage display selection methods are described in U.S. Pat. No. 9,102,711 B2; U.S. Ser. No. 10/906,968 B2; and Kunkel, Proc. Natl. Acad. Sci. USA, 1985, 83(2):488-492.

In this example, high diversity (˜10¹⁰) phage libraries using NNK variant site encoding were constructed targeting residues positions within the substrate-binding pockets of the selected metalloenzymes. Phosphorylated primers were obtained that possess degenerate codons at intended positions and were annealed to uracilated ssDNA containing the parental sequence of the same binder of interest with introduced SacII sites. After polymerase extension and ligation, the heteroduplex DNA was transformed into custom TG1RM cells (Lucigen TG1 Electrocompetent Cells containing a pCDF-1b plasmid expressing SacII enzyme), which removed undesired template DNA with SacII sites resulting in 10⁹-10¹⁰ libraries. Monovalent phage libraries were packaged using standard helper phage and precipitated using PEG/NaCl solution. Using standard protocols, phage libraries were panned against different N-terminally modified target peptides. NTAA modification was applied to target peptides during binder screening and maturation to increase substrate surface available for interaction with the binder, which would result in selection of binders with higher affinity and P1 specificity.

For each round of phage display selection, precipitated phage in the presence of peptide and protein competitors were first depleted against beads coated with off-target peptides for 1 hour at 24° C. and then panned against beads coated with target peptides for 1 hour at 24° C. After washing 6 times with PBST, beads-bound phages were eluted using 0.2 M pH=2.2 glycine for 10 min at 24° C. and then subsequently used to infect mid-log phase TG1 cells. Once the final round of selection was complete, the output was profiled in a phage-based, multiplexed binding assay (Luminex, DiaSorin, USA) against a panel of N-terminally modified target peptides and underwent next-generation sequencing to obtain clone enrichment sequence information. Luminex enables analysis of binding of phage libraries against multiple peptide targets immobilized on beads in a single assay well. This is accomplished by spatially separating immunoassays performed on beads that contain unique fluorophore cores that exhibit distinct excitation/emission profiles. Multiple target peptide-specific beads are combined in a single well of a multi-well microplate to detect and quantify multiple targets simultaneously. Specific binders were isolated against a variety of N-terminally modified target peptides. Based on the sequence identities after enrichment, consensus mutations or mutational hotspots were identified and binders were expressed and purified for testing in the encoding assay.

### Example 5. Binder Maturation

Binder maturation for affinity and specificity involved multiple cycles of error prone PCR prior to library construction via Kunkel mutagenesis and phage display selection, performed essentially as described in Example 4. Briefly, 60-90 cycles of error prone PCR on a parental binder generated PCR amplicons with an average of 4-6 random amino acid mutations per 100 amino acids. The dsDNA amplicon was digested by lambda exonuclease into “megaprimer” ssDNA, which was used to generate heteroduplex DNA by annealing to uracilated ssDNA of the vector containing the parental sequence of the same binder of interest with introduced SacII sites. After polymerase extension and ligation, the heteroduplex DNA was transformed into custom TG1RM cells (Lucigen TG1 Electrocompetent Cells containing a pCDF-1b plasmid expressing SacII enzyme), which removed undesired template DNA with SacII sites resulting in 109-1010 libraries. Monovalent phage libraries were packaged using standard helper phage and precipitated using PEG/NaCl solution. For each round of phage display selection, precipitated phage in the presence of peptide and protein competitors were first depleted against beads coated with off-target peptides for 1 hour at 24° C. and then panned against beads coated with target peptides for 1 hour at 24° C. After washing 6 times with PBST, beads-bound phages were eluted using 0.2 M pH=2.2 glycine for 10 min at 24° C. and then subsequently used to infect mid-log phase TG1 cells. Once the final round of selection was completed, the output was profiled in a phage-based, multiplexed binding assay (Luminex, DiaSorin, USA) against a panel of N-terminally modified target peptides and underwent next-generation sequencing to obtain clone enrichment sequence information. Based on the sequence identities after enrichment, consensus mutations or mutational hotspots were identified and binders were expressed and purified for testing in the encoding assay.

### Example 6. Binder Expression and Purification

Plasmid DNA was received from a vendor generated source containing the identified engineered binder conjugated with an N-terminal hexa-histidine tag and a C-terminal SpyCatcher domain. Plasmids were transformed into chemically competent E. coli cells using standard methods. Recovery was done by adding 150 ul of warm SOC and incubation for 1 hour at 30° C. After recovery, 80 ul of transformed culture was added to 1 ml 2YT containing corresponding antibiotic. The culture was grown overnight and then used to generate stock in glycerol. The stock was then used to inoculate an overnight culture of 2YT containing corresponding antibiotic, and the culture was grown overnight for ˜20 hours at 37° C. This culture was subsequently used to inoculate another larger volume culture of 2YT containing corresponding antibiotic at a 100-fold dilution. The culture was then left at 37° C. for 3-4 hours until an optical density of 0.6 was reached. Temperature was then lowered to 15° C. and protein expression was induced with a final concentration of 0.5 mM IPTG. The cultures were grown for an additional 16-20 hours and the cells were harvested by centrifugation at 4,000 rpm for 20 min. The cellular pellets were stored at −80° C. until ready for use.

Stored cellular pellets were resuspend in 25 mM Tris pH=7.9, 500 mM NaCl, and 10 mM imidazole with included protease inhibitor and were lysed by sonication. The clarified lysate was loaded onto an AKTA FPLC using a tandem purification method of nickel affinity and size-exclusion chromatography. The retained protein was eluted from the nickel affinity column using 25 mM Tris pH 7.9, 500 mM NaCl, 300 mM imidazole directly onto the size-exclusion column. The size-exclusion buffer was 25 mM PO4 pH 7.4 with 150 mM NaCl, and after elution and concentration, glycerol was added to final concentration of 10%. Proteins were aliquoted, frozen, and stored at −80° C.

### Example 7. Evaluation of Binding Efficiencies of Binders Via the Multiplex Encoding Assay

To evaluate binding efficiencies of selected purified binders, a previously developed ProteoCode™ assay (disclosed in detail in US 20190145982 A1, incorporated herein) was used. This variant of the ProteoCode™ assay comprises contacting binder-coding tag conjugates with the N-terminally modified immobilized peptides associated with the recording tags. If affinity of the binder to the modified NTAA of the immobilized peptides is strong enough (typically, Kd should be less than 500 nM, and preferably, less than 200 nM), the coding tag and the recording tag form hybridization complex via hybridization of the corresponding spacer regions to allow transfer of barcode information from the coding tag to the recording tag via a primer extension reaction (the encoding reaction), generating extended recording tag. Sequencing of extended recording tags after the encoding cycle may be used to identify binder(s) that was (were) bound to the immobilized peptide. At the same time, estimating fractions of the recording tags being extended (encoded) during primer extension reaction provides estimate of efficiency of the encoding reaction, which directly correlates with binding affinity of the binder to the particular modified NTAA.

The described encoding assay was used to generate binding profiles for the selected binders across a set of 288 peptides (17X17 combination of different P1 and P2 residues) modified with a specific N-terminal modifier agent. For the encoding assay, selected binding agents engineered from metalloenzyme scaffolds as described in the previous Examples 4-6 were used. Each binding agent was conjugated to a corresponding nucleic acid coding tag comprising barcode with identifying information regarding the binding agent. The coding tag specific for the binding agent was attached to SpyTag via a PEG linker, and the resulting fusions were reacted with binding agent-SpyCatcher fusion protein via SpyTag-SpyCatcher interaction, essentially as described in US 2021/0208150 A1. Briefly, amine-functionalized oligonucleotide coding tags were conjugated to a heterobifunctional linker containing an NHS ester, PEG24 linker and maleimide. Excess linker was removed by acetone purification, and excess linker in solution was removed by centrifugation. Purified oligonucleotide-PEG24-maleimide was incubated overnight with SpyTag peptide forming a conjugate via a cysteine residue. The sample was spun down to remove precipitate and the supernatant was transferred to a 10 k molecular weight filter to remove excess SpyTag peptide. After multiple washes, the final bioconjugate of SpyTag peptide containing a PEG24 linker and coding tag oligonucleotide was obtained and subsequently combined with the binder/SpyCatcher fusion protein spontaneously forming the final binder-fused coding tag conjugate.

An array of target peptide-recording tag conjugates having a variety of different NTAAs was generated (17×17 combination of different P1 and P2 residues). The peptides containing C-terminally attached 6-Azido-L-lysine were reacted with DBCO-C2-modified 17 nt oligonucleotides in 100 mM HEPES, pH=7.0 at 60° C. for 1 hour. Each NTAA peptide-oligonucleotide conjugate was ligated to two different 15 nt DNA fragments containing a 7 nt barcode and an 8 nt spacer sequence using splint DNA and T4 DNA ligase to generate a peptide-recording tag conjugate with two different barcodes. A total of 576 peptide-recording tag conjugates were prepared and pooled for ligation and immobilization on short hairpin capture DNAs attached to the beads (NHS-Activated Sepharose High Performance, Cytiva, USA).

The capture DNAs were attached to the beads using trans-cyclooctene (TCO) and methyltetrazine (mTet)-based click chemistry. TCO-modified short hairpin capture DNAs (16 basepair stem, 4 base loop, 17 base 5′ overhang) were reacted with mTet-coated beads. The peptide-recording tag pools (20 nM) were annealed to the hairpin capture DNAs attached to the beads in 0.5 M NaCl, 50 mM sodium citrate, 0.02% SDS, pH 7.0, and incubated for 30 minutes at 37° C. The beads were washed once with 1× phosphate buffer, 0.1% Tween 20 and resuspended in 1× Quick ligation solution (New England Biolabs, USA) with T4 DNA ligase. After a 30 min incubation at 25° C., the beads were washed once with 1× phosphate buffer, 0.1% Tween 20, three times with 0.1 M NaOH, 0.1% Tween 20, three times with 1× phosphate buffer, 0.1% Tween 20, and resuspended in 50 μL of PBST.

Before the encoding assay, the beads with immobilized target peptide-recording tag conjugates were treated with an N-terminal modifier agent by methods disclosed in Example 3 above to modify the N-terminal of the immobilized peptides. The modified beads with peptide conjugates were washed once with 70% Ethanol, washed once with water and resuspended in PBST. The coding tags attached to the binding agents form a loop with 12 bp duplex and 9 nt spacer at the 3′, which is complementary to the 3′ spacer of the recording tag on the beads.

The cycle of the encoding assay described in this example consists of contacting the immobilized peptides with a metalloenzyme binding agent-coding tag conjugate. For this, each binding agent (50 nM) was incubated with the recording tag-peptide conjugates immobilized on the beads for 30 min at 25° C., followed by washing twice with 1× phosphate buffer, pH 7.3, 500 mM NaCl, 0.1% Tween 20. This was followed by transferring information of the coding tag to the recording tags associated with the target peptides by a primer extension reaction after partial hybridization between the coding tag and the recording tag through a shared spacer region using a DNA polymerase having 5′-to-3′ polymerization activity and having substantially reduced 3′-to-5′ exonuclease activity. Extension was performed by addition of 50 mM Tris-HCl, pH 7.5, 2 mM MgSO₄, 50 mM NaCl, 1 mM DTT, 0.1 mg/mL BSA, 0.1% Tween 20, dNTP mixture (125 uM of each) and 0.125 U/uL of Klenow fragment (3′→5′ exo-) (MCLAB, USA) at 25° C. for 15 min, followed by one wash of 1× phosphate buffer, 0.1% Tween 20, twice with 0.1 M NaOH+0.1% Tween 20, and twice with 1× phosphate buffer, 0.1% Tween 20. After the recording tag extension, the binding agent-coding tag conjugate was washed away, and the sample was capped by introducing with primer binding site for PCR and NGS with incubation of 400 nM of an end capping oligo with 0.125 U/uL of WT Klenow fragment (3′→5′ exo-), dNTPs (each at 125 uM), 50 mM Tris-HCl (pH, 7.5), 2 mM MgSO₄, 50 mM NaCl, 1 mM DTT, 0.1% Tween 20, and 0.1 mg/mL BSA at 25° C. for 10 min. The beads were washed once with 1× phosphate buffer, 0.1% Tween 20, twice with 0.1 M NaOH+0.1% Tween 20, and twice with 1× phosphate buffer, 0.1% Tween 20. Then, the extended recording tags were amplified and analyzed by nucleic acid sequencing.

Sequencing of recording tags after the encoding cycle was used to estimate fractions of the recording tags being extended (encoded) during primer extension reactions. The efficiencies of the encoding reactions were evaluated based on yield (based on fractions of recording tag reads contained barcode information of the coding tag (encoded)) and background signal (fractions of recording tag reads contained barcode information that are associated with a non-cognate peptide).

### Example 8. High Specificity Binders Against Modified N-Terminal Amino Acid (NTAA) Residues of Target Peptides Generated from the Metalloenzyme Scaffold

An exemplary metalloenzyme scaffold (sequence set forth in SEQ ID NO: 7) was used to generate a panel of binders specific for selected modified N-terminal amino acid (NTAA) residues (Z-P1) of target peptides.

Binder engineering and maturation from the metalloenzyme scaffold were performed essentially as described in Examples 4 and 5. The crystal structures of the scaffold were retrieved from the PDB database (4LP6, 4YYT), and used to guide selection of key residues in the structure for modification during engineering and maturation. M64 N-terminal modification (NTM) that coordinates zinc (ZnII) ion was installed on target peptides to provide more binding surface and achieve better specificity during engineering. Specific binders were successfully selected against M64-modified D, F, H, E, T, A, G, V, S, I NTAA residues (e.g., SEQ ID NO: 48-SEQ ID NO: 57).

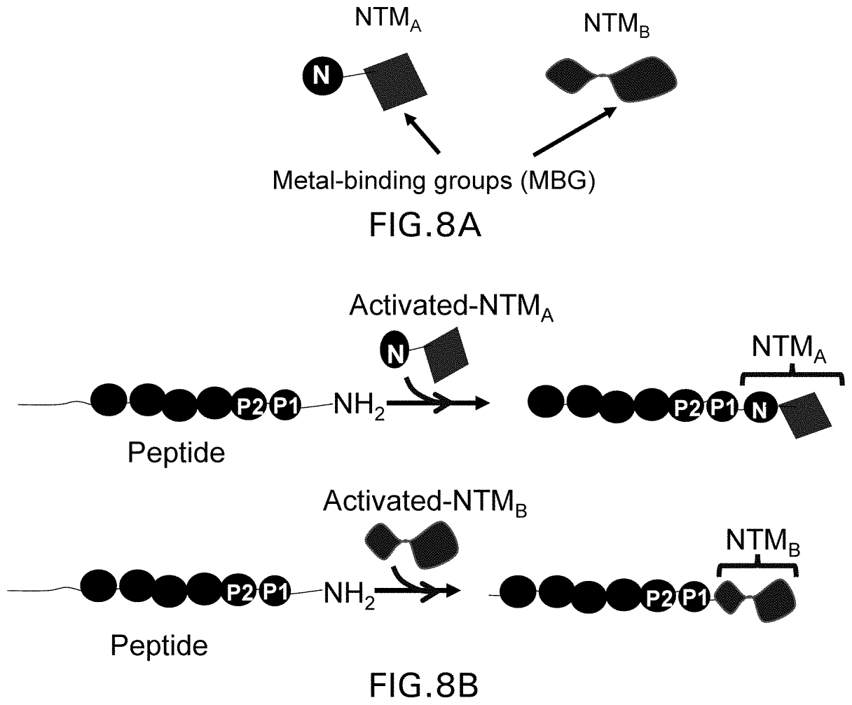

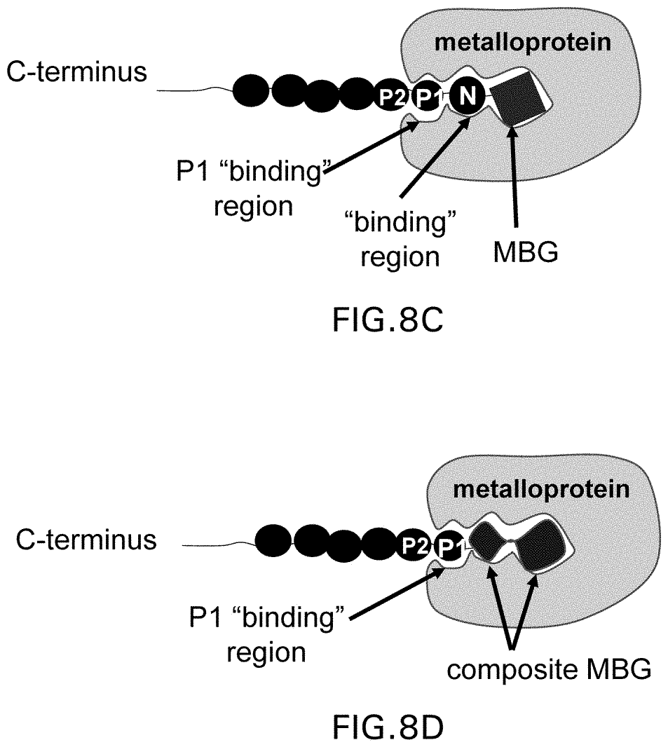

The N-terminal modifications were chosen based on size (having a volume, preferably, from about 100 Å³ to about 500 Å³), and also based on ability to coordinate Zn(II) ion and also to interact with substrate binding pockets of metalloenzyme scaffolds, forming hydrogen bond-based, hydrophobic or other non-covalent interactions. The aim for an engineered metalloenzyme-based binder is to specifically bind to the N-terminally modified target peptide through interaction between the engineered binder and the Z-P1 of the N-terminally modified target peptide, so that, preferably, binding specificity between the engineered binder and the N-terminally modified target peptide is predominantly or substantially determined by interaction between the engineered binder and the Z-P1 of the N-terminally modified target peptide. It can be achieved with a proper geometry of substrate binding pocket of the engineered metalloprotein binder, when there is minimal or no interaction between the binder and the P2 residue of the target peptide. When P1-P2 part occupies a volume encompassing the substrate binding pocket of the engineered binder, and P1 residues is modified with an NTM having a volume similar to a volume of an amino acid residue, it would effectively preclude the P2 residue from entering into or interacting with an affinity determining region of the engineered binder interacting with the N-terminally modified target peptide (FIG. 8A-8D).

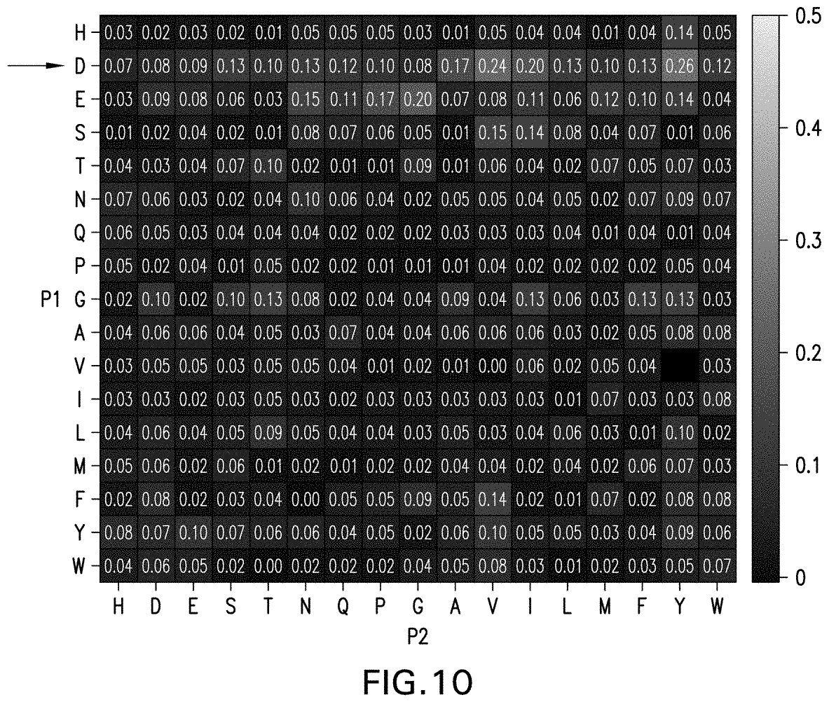

Thus, an engineered binder should have relatively high selectivity towards a modified P1 (Z-P1) residue and broad tolerance for different P2 residues. To evaluate whether the engineered binders selected from different metalloenzyme-based scaffolds possess these features, heatmap arrays were generated, where each cell of the array represents an encoding efficiency of the given binder that binds to a specific combination of P1-P2 residues of the target peptide. To generate such heatmap arrays, encoding data (fractions of the recording tags being encoded) were collected in parallel as described in Example 7 for an immobilized set of 288 peptides (17×17 combination of different P1 and P2 residues) and plotted as two-dimensional matrix for diverse P1-P2 combinations (see e.g., FIG. 10-FIG. 13). Encoding efficiencies are shown as black/white gradient, wherein the more intense white color represents higher encoding efficiency (FIG. 10-FIG. 13).

An example of heatmap data for a representative M64-D-specific binder is shown on FIG. 10. FIG. 10 shows results of multiple parallel encoding reactions for the binder engineered from the scaffold having sequence as set forth in SEQ ID NO: 7 to specifically recognize M64-modified D NTAA residue of target peptides, and the binder's sequence is as set forth in SEQ ID NO: 48.

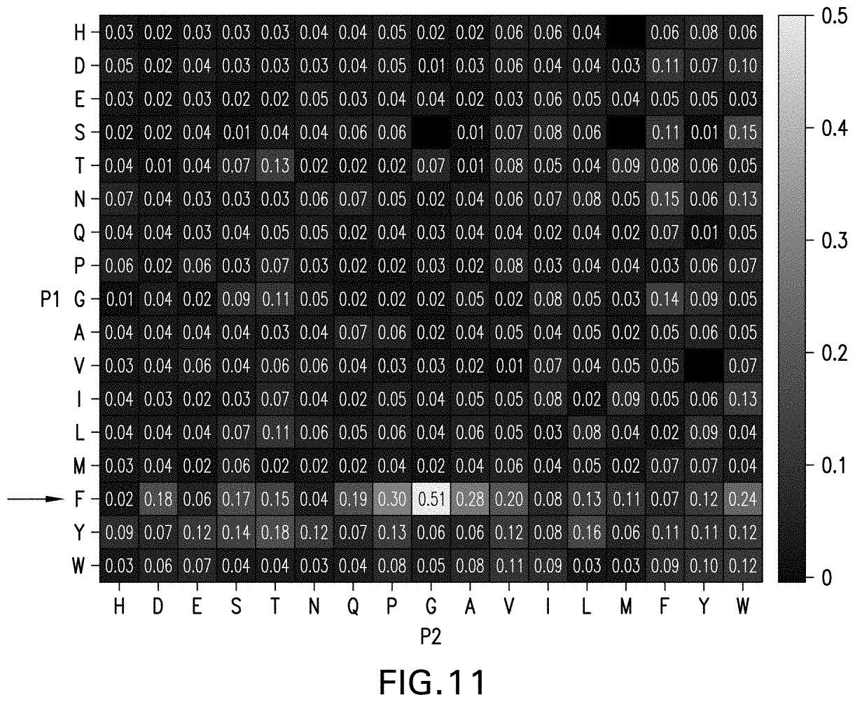

Another example of heatmap data for a representative M64-F-specific binder is shown on FIG. 11. FIG. 11 shows results of multiple parallel encoding reactions for the binder engineered from the scaffold having sequence as set forth in SEQ ID NO: 7 to specifically recognize M64-modified F NTAA residue of target peptides, and the binder's sequence is as set forth in SEQ ID NO: 51.

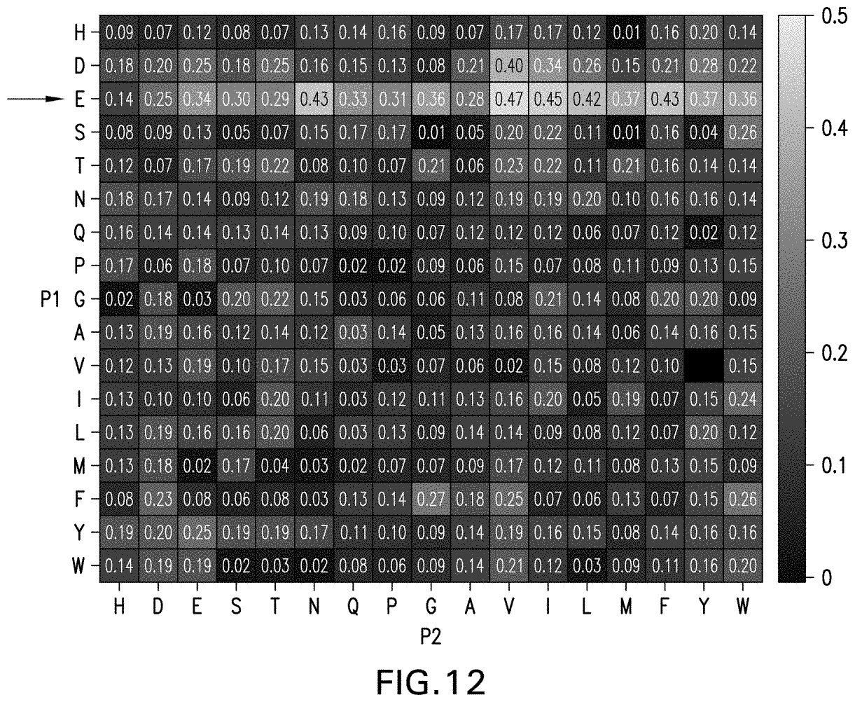

Another example of heatmap data for a representative M64-E-specific binder is shown on FIG. 12. FIG. 12 shows results of multiple parallel encoding reactions for the binder engineered from the scaffold having sequence as set forth in SEQ ID NO: 7 to specifically recognize M64-modified E NTAA residue of target peptides, and the binder's sequence is as set forth in SEQ ID NO: 55.

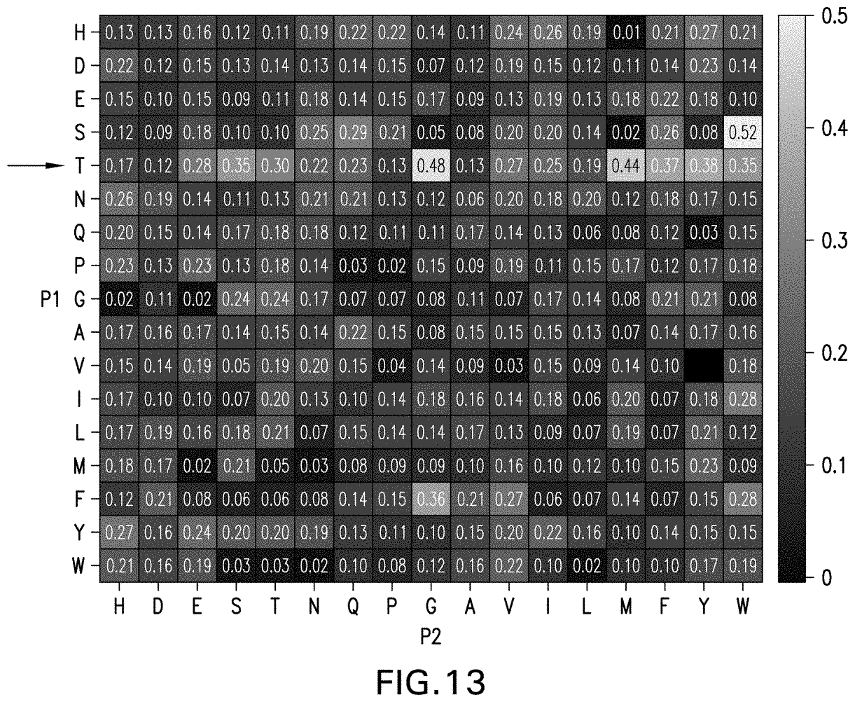

Another example of heatmap data for a representative M64-T-specific binder is shown on FIG. 13. FIG. 13 shows results of multiple parallel encoding reactions for the binder engineered from the scaffold having sequence as set forth in SEQ ID NO: 7 to specifically recognize M64-modified T NTAA residue of target peptides, and the binder's sequence is as set forth in SEQ ID NO: 57.

Such binders can be used in combination with each other and other metalloprotein binders to identify different modified NTAA residues of target peptides.

Kd values for the selected engineered hCAII binders were obtained using the colorimetric assay similar to described in Example 2.

In the colorimetric assay, 300 nM of wild-type carbonic anhydrase or engineered hCAII binders were aliquoted into a 96-well, clear, flat-bottom plate in 45 uL of 50 mM MOPS (pH7.5), 33 mM Na₂SO₄, 1 mM EDTA and 0.1% Tween 20. To each column of the plate, a 1/10 dilution series from 1 mM to 0.1 nM of each NTM-derivatized peptide was added and incubated at 25 C for 30 minutes to reach binding equilibrium. To this, 1 mM p-nitrophenylacetate (pNPA) was added to each well and screened on a plate reader at 405 nm. The initial rate of hydrolysis was observed over the first 60 seconds. The slopes versus the concentration of NTM-derivatized peptide were put into a non-linear regression equation to determine the IC50 (50% inhibitory concentration) of the NTM-derivatized peptide to the wild-type hCAII or engineered hCAII binders. The IC50 value measured in this experiment (see Table 11) provided relative binding affinities of the binders (Kd values).

TABLE 11
IC50 values (μM) for wild-type hCAII, wild-type hCAI and two engineered
hCAII binders tested against five M64-derivatized model peptides.

| SEQ ID NO: | Specificity | M64-DAEIR | M64-EAEIR | M64-AAEIR | M64-FAEIR | M64-LAEIR |
|---|---|---|---|---|---|---|
| 7 | N/A | 0.089 ± 0.020 | 0.630 ± 0.098 | 0.020 ± 0.004 | 0.018 ± 0.003 | 0.028 ± 0.008 |
| 58 | Hydrophobics | 0.136 ± 0.038 | 1.088 ± 0.360 | 0.109 ± 0.034 | 0.012 ± 0.001 | 0.016 ± 0.004 |
| 48 | D | 0.101 ± 0.021 | 9.476 ± 2.676 | 8.334 ± 1.767 | 10.04 ± 5.519 | 162.8 ± 124.0 |
| 51 | F | 7.191 ± 2.740 | 193.3 ± 96.32 | 3.239 ± 1.134 | 0.310 ± 0.112 | 6.907 ± 2.184 |

### Example 9. Quantification of Engineered Binder's P1 Selectivity and P2 Tolerability Based on Calculating Corresponding P1 and P2 Gini Scores

To quantify engineered binder's P1 selectivity and P2 tolerance, relative P1 selectivity towards a modified P1 (Z-P1) residue and relative P2 tolerance for different P2 residues were calculated as corresponding Gini coefficients. The Gini coefficient is a single number that demonstrates a degree of inequality in a distribution (a measure of inequality). It is used to estimate how far a given distribution deviates from a totally equal distribution. The Gini coefficient is defined as follows.

For a population uniform on the values yᵢ, i=1 to n, indexed in non-decreasing order (yᵢ≤yᵢ₊₁):

$$G = \frac{1}{n} ( n + 1 - 2 ( \frac{{{\sum }^{n}}_{i = 1} ( n + 1 - i ) {y}_{i}}{{{\sum }^{n}}_{i = 1} {y}_{i}} ) )$$

This may be simplified to:

$$G = \frac{2 {{\sum }^{n}}_{i = 1} i {y}_{i}}{n {{\sum }^{n}}_{i = 1} {y}_{i}} - \frac{n + 1}{n} . \qquad ( Equation 1 )$$

This formula applies to any population, since each member can be assigned its own yᵢ (Damgaard, Christian. “Gini Coefficient.” From MathWorld—A Wolfram Web Resource). To calculate Gini coefficient for engineered binder's P1 selectivity based on heatmap data, the above formula was used, where n represents number of P1 residues (n=17), and yᵢ represent fractions of recording tags encoded on the ith most encoding P1. Similarly, to calculate Gini coefficient for engineered binder's P2 tolerance based on heatmap data, the above formula was used, where n represents number of P2 residues (n=17), and yᵢ represent fractions of recording tags encoded on the ith most encoding P2. Higher P1 indicates more selectivity towards the particular Z-P1 residue the binders specifically binds to, whereas lower P2 score indicates less selectivity towards particular P2 residue (and higher tolerance). These scores provide only relative estimation of selectivity, and they were arbitrary set to be: P1 score more than 0.15 for a binder to be considered as specific; and P2 score less than 0.4 for a binder to be considered P2-independent. It should be noted that the scores may be further improved through further binder selection and maturation process.

For preferred engineered binders to be used in the ProteoCode™ assay or in another high throughput peptide analysis assay, binding specificity between the engineered binder and the N-terminally modified target peptide is predominantly or substantially determined by interaction between the engineered binder and the Z-P1 of the N-terminally modified target peptide. It implies or indicates that such engineered binder has a high P1 score (for example, more than 0.25) and will have a low P2 score (for example, less than 0.3). Depending on particular assay, more or less specific binders can be employed. Alternative measurements of binder's P1 selectivity and P2 tolerance can be utilized, and different threshold values for P1 selectivity and P2 tolerance can be set.

To evaluate Z-P1 specificity (via P1 selectivity and P2 tolerance) of selected binders engineered by the methods described in Example 4 and 5, P1 and P2 scores were calculated for the binders based on multiplex encoding data (heatmap data) and shown in Table 12. Corresponding binder sequences (based on SEQ ID NOs) are as set forth in the Sequence Listing Starting scaffolds for the binders are shown in the second column of Table 1 (based on SEQ ID NOs) together with the NTM used to modify P1 residue.

TABLE 12
Z-P1 specificity, P1 selectivity and P2
tolerance of selected engineered binders.

| | SEQ ID NO of | | | |
|---|---|---|---|---|
| SEQ ID NO | the scaffold | Specificity | | |
| of the binder | and NTM | towards P1 | P1 score | P2 score |
| 48 | 7, M = M64 | D | 0.293762153 | 0.194449 |
| 49 | 7, M = M64 | D | 0.369155476 | 0.19823689 |
| 50 | 7, M = M64 | D | 0.248650734 | 0.25259301 |
| 51 | 7, M = M64 | F | 0.321647176 | 0.35559918 |
| 53 | 7, M = M64 | F | 0.232907355 | 0.242115 |
| 55 | 7, M = M64 | E | 0.292695353 | 0.19862444 |
| 56 | 7, M = M64 | E | 0.278916867 | 0.24028269 |
| 57 | 7, M = M64 | T | 0.278718119 | 0.21382942 |
| 7 | 7, M = M64 | — | 0.941176471 | 0.94117647 |
| 58 | 58, M = M64 | Small | 0.337689944 | 0.23579931 |
| | | hydrophobic | | |

Engineered binders presented in the Sequence Listing and in Table 12 show diversity across Z-P1 specificity, since D, E, T and F represent amino acid residues having different biochemical properties (charged, polar uncharged and hydrophobic). Thus, by using the described methods, metalloprotein binders can be engineered to recognize a diverse set of Z-P1s on target peptides.

Sequences of engineered binders differ significantly from corresponding starting metalloenzyme scaffolds, and each of the engineered binders with sequences as set forth in SEQ ID NOs: 48-57 contains 3-10 amino acid substitutions from the corresponding starting scaffold. Since most amino acid substitutions were designed to be on the substrate-interaction region of the binders, geometry of substrate-binding pockets of the scaffolds and atomic interactions within them were significantly changed during engineering and maturation process.

Engineered binders shown in Table 12 typically have about 97-98% sequence identity with corresponding starting scaffold. Additionally, these binders may be further processed through another maturation round for improving their characteristics, such as Z-P1 affinity, P1 selectivity and/or P2 tolerance. In the next maturation round new amino acid substitutions will likely be introduced, and the updated binder's sequence may be further away from the sequence of the corresponding starting scaffold, such that it will have about 90 or 95% sequence identity with the corresponding starting scaffold. Moreover, conservative amino acid substitutions can be made in the binder's sequence that would improve its characteristics unrelated to the Z-P1 binding, such as improve binder's stability or increase expression level of the binder in bacterial cells. Such conservative amino acid substitutions are known to skilled in the art, and the updated binder's sequence may have less than about 90% sequence identity with the corresponding starting scaffold (for example, may have about 80% or 85% sequence identity with the corresponding starting scaffold).

The present disclosure is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method of treating a target peptide, the method comprises the following steps:
(a) contacting the target peptide with an N-terminal modifier agent to form an N-terminally modified peptide having a formula: Z-P1-P2-peptide, wherein Z is an N-terminal modification capable of coordinating or chelating a zinc metal cation M, P1-P2-peptide is a target peptide before modification with the N-terminal modifier agent, Z-P1 is a modified N-terminal amino acid (NTAA) residue of the target peptide, and P2 is a penultimate terminal amino acid residue of the target peptide; and
(b) contacting an engineered metalloprotein binder with the N-terminally modified target peptide to allow the engineered binder to specifically bind to the N-terminally modified target peptide through interaction between the engineered binder and the modified NTAA residue of the N-terminally modified target peptide, wherein the engineered binder comprises an amino acid sequence X1-C/H/D/E-X2-C/H/D/E-X3-C/H/D/E-X4, wherein C/H/D/E is any single amino acid residue independently selected from the group consisting of amino acid residues C (Cys), H (His), D (Asp) and E (Glu); X1, X2, X3 and X4 are each any amino acid sequence comprising between 0 and 200 amino acid residues in length, and wherein the amino acid sequence X1-C/H/D/E-X2-C/H/D/E-X3-C/H/D/E-X4 chelates a zinc metal cation M with a thermodynamic dissociation constant of 0.5 nM or less.

2. The method of claim 1, wherein the engineered metalloprotein binder binds to the N-terminally modified target peptide with at least a 100-fold greater binding affinity than a model polypeptide that has at least 90% homology to the engineered binder over the entire sequence length, wherein the model polypeptide does not comprise the amino acid sequence X1-C/H/D/E-X2-C/H/D/E-X3-C/H/D/E-X4.

3. The method of claim 1, wherein the engineered metalloprotein binder comprises an amino acid sequence having at least about 90% sequence homology to any one of the amino acid sequences selected from the group consisting of SEQ ID NO: 7-SEQ ID NO: 59.

4. The method of claim 1, wherein the engineered metalloprotein binder comprises an amino acid sequence, which differs from one of the amino acid sequences set forth in SEQ ID NO: 7, SEQ ID NO: 8, SEQ ID NO: 9, SEQ ID NO: 14, SEQ ID NO: 15, SEQ ID NO: 16, SEQ ID NO: 17, SEQ ID NO: 21, SEQ ID NO: 25, SEQ ID NO: 27, SEQ ID NO: 30, SEQ ID NO: 31, SEQ ID NO: 39 or SEQ ID NO: 43 by at least one amino acid residue within 6 Å of a Z-P1 binding site of the engineered metalloprotein binder, wherein the Z-P1 binding site comprises amino acids corresponding to amino acid positions 59, 64, 66, 88, 89, 91, 93, 103, 116, 118, 127, 128, 131, 137, 139, 193, 194, 195, 196, 198, 203, and 205 of SEQ ID NO: 7; or the Z-P1 binding site comprises amino acids corresponding to amino acid positions 60, 63, 65, 68, 87, 88, 90, 92, 102, 115, 117, 127, 137, 139, 193, 194, 195, 196, 197, 198, 203, and 205 of SEQ ID NO: 8; or the Z-P1 binding site comprises amino acids corresponding to amino acid positions 1, 2, 17, 59, 61, 64, 89, 91, 93, 103, 116, 118, 127, 131, 139, 193, 194, 195, 196, 197, 198, 199, 203, and 205 of SEQ ID NO: 9; or the Z-P1 binding site comprises amino acids corresponding to amino acid positions 99, 100, 132-136, 190, 191, 194, 200, and 222 of SEQ ID NO: 14; or the Z-P1 binding site comprises amino acids corresponding to amino acid positions 63-66, 83, 84, 86, 89, 92, 93, 96, 102, 149, 152-155, 158, and 175-177 of SEQ ID NO: 15; or the Z-P1 binding site comprises amino acids corresponding to amino acid positions 117, 255, 256, 257, 258-260, 268, 270, 271, 290, 293, 294, 297, 315, 316, 377, 779, and 821 of SEQ ID NO: 16; or the Z-P1 binding site comprises amino acids corresponding to amino acid positions 41, 58, 59, 60, 61, 62, 65, 77, 105, 107, 108, 109, 110-112, 147, 150, 151, 154, 155, 158, and 185 of SEQ ID NO: 17; or the Z-P1 binding site comprises amino acids corresponding to amino acid positions 133-137, 153, 158, 160, 161, 169, 173, 176, 177, 180, 186, 191, 192, 209, 216, and 230 of SEQ ID NO: 21; or the Z-P1 binding site comprises amino acids corresponding to amino acid positions 4-7, 9, 10, 14, 58, 90, 113, 134-138, 162, 181, 183-185, and 212 of SEQ ID NO: 25; or the Z-P1 binding site comprises amino acids corresponding to amino acid positions 7, 9, 20, 104, 108, 139, 141, 164, 167, 169, 170, 171, 202, 204-210, 242, 245, and 248 of SEQ ID NO: 27; or the Z-P1 binding site comprises amino acids corresponding to amino acid positions 90, 93, 96-99, 132, 133, 136, 142, 152, and 153 of SEQ ID NO: 30; or the Z-P1 binding site comprises amino acids corresponding to amino acid positions 165, 166, 169, 227-230, 231, 235, 248, and 352 of SEQ ID NO: 31; or the Z-P1 binding site comprises amino acids corresponding to amino acid positions 106-109, 141, 142, 145, 151, and 166-168 of SEQ ID NO: 39; or the Z-P1 binding site comprises amino acids corresponding to amino acid positions 106, 107, 313-317, 325, 327, 379, 382-389, 448, 453, 506, and 564-566 of SEQ ID NO: 43.

5. The method of claim 1, wherein the N-terminal modifier agent is a compound of the following formula: 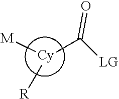
wherein:
M is a metal binding group that comprises sulfonamide, hydroxamic acid, sulfamate, or sulfamide;
the group 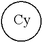
is a 5 or 6 membered aromatic ring which may contain up to three heteroatoms selected from N, O and S as ring members, and is optionally substituted by R;
R represents one or two optional substituents selected from the group consisting of F, Cl, CH3, CF2H, CF3, OH, OCH3, OCF3, NH2, N(CH3)2, NO2, SCH3, SO2CH3, CH2OH, B(OH)2, CN, CONH2, and CONHCH3; and
LG is a leaving group.

6. The method of claim 5, wherein LG is selected from the group consisting of N-succinimidyloxy, sulfo-N-succinimidyloxy, pentafluorophenoxy, tetrafluorophenoxy, 4-sulfo-phenoxy, and pyridinyl-2-oxy N-oxide.

7. The method of claim 1, wherein the engineered metalloprotein binder binds to the N-terminally modified target peptide with a thermodynamic dissociation constant (Kd) of 500 nM or less.

8. The method of claim 1, further comprising step (c): removing the modified NTAA residue from the N-terminally modified target peptide, thereby exposing a new NTAA residue.

9. The method of claim 8, wherein steps (a), (b) and (c) are repeated sequentially at least one time.

10. The method of claim 1, further comprising immobilizing the target peptide on a solid support before step (a).

11. The method of claim 10, wherein the target peptide immobilized on a solid support is associated with a nucleic acid recording tag.

12. The method of claim 1, wherein the engineered binder comprises a detectable label, or a nucleic acid tag, or a nucleic acid coding tag.

13. The method of claim 1, wherein the N-terminal modifier agent further comprises a peptide coupling reagent.

14. The method of claim 13, wherein the peptide coupling reagent is a compound of Formula (1) or (2), wherein:
Formula (1) is 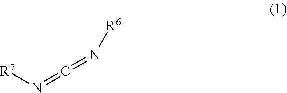
or a salt or conjugate thereof, wherein
R6 and R7 are each independently C1-6 alkyl, —CO2C1-4 alkyl, —ORk, aryl, heteroaryl, cycloalkyl or heterocyclyl, wherein the C1-6 alkyl, —CO2C1-4 alkyl, —ORk, aryl, and cycloalkyl are each unsubstituted or substituted; and
Rk is H, C1-6 alkyl, or heterocyclyl, wherein the C1-6 alkyl and heterocyclyl are each unsubstituted or substituted; wherein heterocyclyl can be 5-8 membered ring comprising one or two heteroatoms selected from N, O and S as ring members, where the heteroaryl can be a 5-6 membered single ring or 8-10 membered bicyclic ring, each of which comprises one to three heteroatoms selected from N, O and S as ring members; and
Formula (2) is: 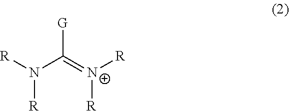
wherein:
each R is independently C1-4 alkyl, optionally substituted with up to three groups selected from halo, C1-2 alkoxy, C1-2 haloalkyl, and C1-2 haloalkoxy; and
two R groups on the same N can optionally cyclize to form a 5-7 membered ring optionally containing an additional heteroatom selected from N, O and S as a ring member, and optionally substituted with one or two groups selected from oxo, C1-2 alkyl, C1-2 alkoxy, C1-2 haloalkyl, and C1-2 haloalkoxy; and
G is selected from the group consisting of halo, benzotriazolyloxy, halobenzotriazolyloxy, pyridinotriazolyloxy, benzotriazolyl-N-oxide, pyridinotriazolyl-N-oxide, —O—(N-succinimide), 1-cyano-2-ethoxy-2-oxoethylideneaminooxy, and —O—(N-phthalimide).

15. The method of claim 13, wherein the peptide coupling reagent is selected from the group consisting of dicyclohexyl carbodiimide (DCC), diisopropyl carbodiimide (DIPC), 1-ethyl-3-(3-dimethylaminopropyl)carbodiimide (EDC), 1-cyclohexyl-(2-morpholinoethyl)carbodiimide tosylate (CMCT), COMU, HATU, HBTU, TBTU, HCTU, and TSTU, PyBOP, PyAOP, PyOxim, and BOP, and (3-(diethoxyphosphoryloxy)-1,2,3-benzotriazin-4(3H)-one) (DEPBT).

16. An engineered metalloprotein binder that specifically binds to an N-terminally modified target peptide modified by an N-terminal modifier agent, wherein:
a) the N-terminally modified target peptide has a formula: Z-P1-P2-peptide, wherein Z is an N-terminal modification capable of coordinating or chelating a zinc metal cation M, P1-P2-peptide is a target peptide before modification with the N-terminal modifier agent, Z-P1 is a modified N-terminal amino acid (NTAA) residue of the target peptide, and P2 is a penultimate terminal amino acid residue of the target peptide;
b) the engineered metalloprotein binder specifically binds to the N-terminally modified target peptide through interaction between the engineered metalloprotein binder and the Z-P1 of the N-terminally modified target peptide; and
c) the engineered metalloprotein binder comprises an amino acid sequence X1-C/H/D/E-X2-C/H/D/E-X3-C/H/D/E-X4, wherein C/H/D/E is any single amino acid residue independently selected from the group consisting of amino acid residues C (Cys), H (His), D (Asp) and E (Glu); X1, X2, X3 and X4 are each any amino acid sequence comprising between 0 and 200 amino acid residues in length, and wherein the amino acid sequence X1-C/H/D/E-X2-C/H/D/E-X3-C/H/D/E-X4 chelates a zinc metal cation M with a thermodynamic dissociation constant of 0.5 nM or less.

17. The binder of claim 16, which binds to the N-terminally modified target peptide with at least a 100-fold greater binding affinity than a model peptide that has at least 90% homology to the engineered binder over the entire sequence length, wherein the model peptide does not comprise the amino acid sequence X1-C/H/D/E-X2-C/H/D/E-X3-C/H/D/E-X4.

18. The binder of claim 16, which comprises an amino acid sequence having at least about 90% sequence homology to any one of the amino acid sequences selected from the group consisting of SEQ ID NO: 7-SEQ ID NO: 59.

19. The binder of claim 16, which comprises an amino acid sequence, which differs from one of the amino acid sequences set forth in SEQ ID NO: 7, SEQ ID NO: 8, SEQ ID NO: 9, SEQ ID NO: 14, SEQ ID NO: 15, SEQ ID NO: 16, SEQ ID NO: 17, SEQ ID NO: 21, SEQ ID NO: 25, SEQ ID NO: 27, SEQ ID NO: 30, SEQ ID NO: 31, SEQ ID NO: 39 or SEQ ID NO: 43 by at least one amino acid residue within 6 Å of a Z-P1 binding site of the engineered metalloprotein binder, wherein the Z-P1 binding site comprises amino acids corresponding to amino acid positions 59, 64, 66, 88, 89, 91, 93, 103, 116, 118, 127, 128, 131, 137, 139, 193, 194, 195, 196, 198, 203, and 205 of SEQ ID NO: 7; or the Z-P1 binding site comprises amino acids corresponding to amino acid positions 60, 63, 65, 68, 87, 88, 90, 92, 102, 115, 117, 127, 137, 139, 193, 194, 195, 196, 197, 198, 203, and 205 of SEQ ID NO: 8; or the Z-P1 binding site comprises amino acids corresponding to amino acid positions 1, 2, 17, 59, 61, 64, 89, 91, 93, 103, 116, 118, 127, 131, 139, 193, 194, 195, 196, 197, 198, 199, 203, and 205 of SEQ ID NO: 9; or the Z-P1 binding site comprises amino acids corresponding to amino acid positions 99, 100, 132-136, 190, 191, 194, 200, and 222 of SEQ ID NO: 14; or the Z-P1 binding site comprises amino acids corresponding to amino acid positions 63-66, 83, 84, 86, 89, 92, 93, 96, 102, 149, 152-155, 158, and 175-177 of SEQ ID NO: 15; or the Z-P1 binding site comprises amino acids corresponding to amino acid positions 117, 255, 256, 257, 258-260, 268, 270, 271, 290, 293, 294, 297, 315, 316, 377, 779, and 821 of SEQ ID NO: 16; or the Z-P1 binding site comprises amino acids corresponding to amino acid positions 41, 58, 59, 60, 61, 62, 65, 77, 105, 107, 108, 109, 110-112, 147, 150, 151, 154, 155, 158, and 185 of SEQ ID NO: 17; or the Z-P1 binding site comprises amino acids corresponding to amino acid positions 133-137, 153, 158, 160, 161, 169, 173, 176, 177, 180, 186, 191, 192, 209, 216, and 230 of SEQ ID NO: 21; or the Z-P1 binding site comprises amino acids corresponding to amino acid positions 4-7, 9, 10, 14, 58, 90, 113, 134-138, 162, 181, 183-185, and 212 of SEQ ID NO: 25; or the Z-P1 binding site comprises amino acids corresponding to amino acid positions 7, 9, 20, 104, 108, 139, 141, 164, 167, 169, 170, 171, 202, 204-210, 242, 245, and 248 of SEQ ID NO: 27; or the Z-P1 binding site comprises amino acids corresponding to amino acid positions 90, 93, 96-99, 132, 133, 136, 142, 152, and 153 of SEQ ID NO: 30; or the Z-P1 binding site comprises amino acids corresponding to amino acid positions 165, 166, 169, 227-230, 231, 235, 248, and 352 of SEQ ID NO: 31; or the Z-P1 binding site comprises amino acids corresponding to amino acid positions 106-109, 141, 142, 145, 151, and 166-168 of SEQ ID NO: 39; or the Z-P1 binding site comprises amino acids corresponding to amino acid positions 106, 107, 313-317, 325, 327, 379, 382-389, 448, 453, 506, and 564-566 of SEQ ID NO: 43.

20. The binder of claim 16, which binds to the N-terminally modified target peptide with a thermodynamic dissociation constant (Kd) of 500 nM or less.

21. The binder of claim 16, which comprises a detectable label or a nucleic acid tag.

22. A kit for treating a target peptide, the kit comprises:
(a) an engineered metalloprotein binder of claim 16;
(b) one of more of the following:
1) an N-terminal modifier agent to form an N-terminally modified peptide having a formula: Z-P1-P2-peptide, wherein Z is an N-terminal modification capable of coordinating or chelating a zinc metal cation M, P1-P2-peptide is a target peptide before modification with the N-terminal modifier agent, Z-P1 is a modified N-terminal amino acid (NTAA) residue of the target peptide, and P2 is a penultimate terminal amino acid residue of the target peptide;
2) an agent configured for removing the modified NTAA residue from the N-terminally modified target peptide, thereby exposing a new NTAA residue;
3) an agent configured for immobilizing the target peptide on a solid support;
4) a solid support;
5) a nucleic acid recording tag;
6) a nucleic acid tag or a nucleic acid coding tag;
7) a detectable label; and/or
8) a peptide coupling reagent.

23. The kit of claim 22, wherein the kit comprises:
an N-terminal modifier agent to form an N-terminally modified peptide having a formula: Z-P1-P2-peptide, wherein Z is an N-terminal modification capable of coordinating or chelating a zinc metal cation M, P1-P2-peptide is a target peptide before modification with the N-terminal modifier agent, Z-P1 is a modified N-terminal amino acid (NTAA) residue of the target peptide, and P2 is a penultimate terminal amino acid residue of the target peptide;
an agent configured for immobilizing the target peptide on a solid support; and
a nucleic acid recording tag.


SEQUENCE LISTING (66 sequences)

SEQ ID NO: 1 - DNA, 20 bp, Artificial Sequence
  P5 primer
aatgatacggcgaccaccga

SEQ ID NO: 2 - DNA, 24 bp, Artificial Sequence
  P7 primer
caagcagaagacggcatacgagat

SEQ ID NO: 3 - protein, 723 aa, Thermomonas hydrothermalis
  Dipeptidyl peptidase from Thermomonas hydrothermalis (with the signal peptide)
MHKTRLVAALAAALATLAPAAWADEGMWVPQQLPEIAGALKKAGLKLDPKQLSDLTGDPMGAVVSLGGCTGSFVSPQGLVATNHHCAYGAIQLNSTPEKNLIKDGFNAPTQADELSAGPNARIYVLEGITDVTAQAKAAMAAAGNDPVARANALEAFEKKITSDCEAEPGYRCRVYSFMGGITYRLFKNLEIKDVRLVYAPPSSVGKFGGDIDNWMWPRHTGDFSFYRAYVGKDGKPAPYSKDNVPYRPKHWLKIADTPLGEGDFVMVAGYPGRTDRYALVAEFENTQNWLYPAISKAYKDQIALVEAAAKDNPEIAVKYAAALAGWNNTSKNFDGQLEGFKRNDVLAIKRREEAAVLEWLRARGKAGTPALEAHAALVKLVADTARTQERDLVLGSFNRTGIIGVAVNLYRLAIERQKPDAEREPGYQQRDLPVIEGSLKQMERRYVPAMDRQLRAYWLDRYVALPAAQHVAAVDAWLGGSDKAAAEAALARLDQSRLGSLEERLKWFNADRAAFEASTDPAIQYAVAVMPTLLAMEQQAKTRYGVALEARPRYLQAVVDYKKSKGQAVYPDANSTLRITYGHVKGYTGLNGKVYTPFTTLEEVAAKETGVEPFDNPKALLEAVAAKRYAGLADARLGTVPVNFLADLDITGGNSGSPVLDANGRLVGLAFDGTLESVASNWVFDPVLTRMISVDQRYMRWIMQEVMPAPQLLEELGVPPRQ

SEQ ID NO: 4 - protein, 700 aa, Thermomonas hydrothermalis
  Dipeptidyl peptidase from Thermomonas hydrothermalis (without the signal peptide)
DEGMWVPQQLPEIAGALKKAGLKLDPKQLSDLTGDPMGAVVSLGGCTGSFVSPQGLVATNHHCAYGAIQLNSTPEKNLIKDGFNAPTQADELSAGPNARIYVLEGITDVTAQAKAAMAAAGNDPVARANALEAFEKKITSDCEAEPGYRCRVYSFMGGITYRLFKNLEIKDVRLVYAPPSSVGKFGGDIDNWMWPRHTGDFSFYRAYVGKDGKPAPYSKDNVPYRPKHWLKIADTPLGEGDFVMVAGYPGRTDRYALVAEFENTQNWLYPAISKAYKDQIALVEAAAKDNPEIAVKYAAALAGWNNTSKNFDGQLEGFKRNDVLAIKRREEAAVLEWLRARGKAGTPALEAHAALVKLVADTARTQERDLVLGSFNRTGIIGVAVNLYRLAIERQKPDAEREPGYQQRDLPVIEGSLKQMERRYVPAMDRQLRAYWLDRYVALPAAQHVAAVDAWLGGSDKAAAEAALARLDQSRLGSLEERLKWFNADRAAFEASTDPAIQYAVAVMPTLLAMEQQAKTRYGVALEARPRYLQAVVDYKKSKGQAVYPDANSTLRITYGHVKGYTGLNGKVYTPFTTLEEVAAKETGVEPFDNPKALLEAVAAKRYAGLADARLGTVPVNFLADLDITGGNSGSPVLDANGRLVGLAFDGTLESVASNWVFDPVLTRMISVDQRYMRWIMQEVMPAPQLLEELGVPPRQ

SEQ ID NO: 5 - protein, 710 aa, Caldithrix abyssi
  Dipeptidyl peptidase from Caldithrix abyssi (with the signal peptide)
MKIRLFGVLLLFTFSLFAEEGMYPITEIHKLNLKKLGIELSADQIFSENEVSLSDAIVQIGGCTGSFISPEGLILTNHHCAFRAIQNISSTENDYLTNGFVAHTLQEERPAKGYTVRITERVEDVSQRVLNAVKHIEDPIEREKAIEKITKQIVKEQEQKHPGKRAAVSEMFPGKTYYLFIYTYLKDVRLVYAPPRSIGEFGGEFDNWEWPRHTGDFTLMRAYVAPDGSPSDYSEENVPYRPKSYLKVAAKGVEEGDRVFILGYPGRTYRHRTSAFLAFEYEFRMPFVVDWYQWQIDLLTTLGKDDADRSLKFSSWIKGLANTEKNYRGKLQGIRRIGLLEQKKNEEEKIQVFIAENNLKKYQHVLTEIKQIYHTYRQSAVREMLLSYFGRSPVLPAVARTLVLAAEERQKEDLERERAFMDRNFKRTQTYTLLRLKNFDSQADQLILQELLKKAAALPEDQRISALRSIFKLDDAAETRQVISEAYRKTRLSDPEFVKTCFAKTPDELKALNDPLINWMLALKEDYETLKNIRKERNGKLRRLRALWLEAKQAYLKTDFIPDANGTYRMTFGFIEGYAPADAVYKAPITTGRGILEKHTGKSPFDTPEKLLALLKAKQFGPFVSKTVGTLPVGILYSCDTTGGNSGSPVLNARGQLVGLNFDRAFEATINDYAWNHQYSRSIGVDIRYILFLLKYFSGAEHLLEEMGVQ

SEQ ID NO: 6 - protein, 692 aa, Caldithrix abyssi
  Dipeptidyl peptidase from Caldithrix abyssi (without the signal peptide)
EEGMYPITEIHKLNLKKLGIELSADQIFSENEVSLSDAIVQIGGCTGSFISPEGLILTNHHCAFRAIQNISSTENDYLTNGFVAHTLQEERPAKGYTVRITERVEDVSQRVLNAVKHIEDPIEREKAIEKITKQIVKEQEQKHPGKRAAVSEMFPGKTYYLFIYTYLKDVRLVYAPPRSIGEFGGEFDNWEWPRHTGDFTLMRAYVAPDGSPSDYSEENVPYRPKSYLKVAAKGVEEGDRVFILGYPGRTYRHRTSAFLAFEYEFRMPFVVDWYQWQIDLLTTLGKDDADRSLKFSSWIKGLANTEKNYRGKLQGIRRIGLLEQKKNEEEKIQVFIAENNLKKYQHVLTEIKQIYHTYRQSAVREMLLSYFGRSPVLPAVARTLVLAAEERQKEDLERERAFMDRNFKRTQTYTLLRLKNFDSQADQLILQELLKKAAALPEDQRISALRSIFKLDDAAETRQVISEAYRKTRLSDPEFVKTCFAKTPDELKALNDPLINWMLALKEDYETLKNIRKERNGKLRRLRALWLEAKQAYLKTDFIPDANGTYRMTFGFIEGYAPADAVYKAPITTGRGILEKHTGKSPFDTPEKLLALLKAKQFGPFVSKTVGTLPVGILYSCDTTGGNSGSPVLNARGQLVGLNFDRAFEATINDYAWNHQYSRSIGVDIRYILFLLKYFSGAEHLLEEMGVQ

SEQ ID NO: 7 - protein, 256 aa, Homo sapiens
  Carbonic anhydrase II scaffold
HWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASF

SEQ ID NO: 8 - protein, 256 aa, Homo sapiens
  Carbonic anhydrase I scaffold
WGYDDKNGPEQWSKLYPIANGNNQSPVDIKTSETKHDTSLKPISVSYNPATAKEIINVGHSFHVNFEDNQDRSVLKGGPFSDSYRLFQFHFHWGSTNEHGSEHTVDGVKYSAELHVAHWNSAKYSSLAEAASKADGLAVIGVLMKVGEANPKLQKVLDALQAIKTKGKRAPFTNFDPSTLLPSSLDFWTYPGSLTHPPLYESVTWIICKESISVSSEQLAQFRSLLSNVEGDNAVPMQHNNRPTQPLKGRTVRASF

SEQ ID NO: 9 - protein, 256 aa, Homo sapiens
  Carbonic anhydrase III scaffold
EWGYASHNGPDHWHELFPNAKGENQSPIELHTKDIRHDPSLQPWSVSYDGGSAKTILNNGKTCRVVFDDTYDRSMLRGGPLPGPYRLRQFHLHWGSSDDHGSEHTVDGVKYAAELHLVHWNPKYNTFKEALKQRDGIAVIGIFLKIGHENGEFQIFLDALDKIKTKGKEAPFTKFDPSSLFPASRDYWTYQGSLTTPPCEECIVWLLLKEPMTVSSDQMAKLRSLLSSAENEPPVPLVSNWRPPQPINNRVVRASF

SEQ ID NO: 10 - protein, 266 aa, Homo sapiens
  Carbonic anhydrase IV scaffold
AESHWCYEVQAESSNYPCLVPVKWGGNCQKDRQSPINIVTTKAKVDKKLGRFFFSGYDKKQTWTVQNNGHSVMMLLENKASISGGGLPAPYQAKQLHLHWSDLPYKGSEHSLDGEHFAMEMHIVHEKEKGTSRNVKEAQDPEDEIAVLAFLVEAGTQVNEGFQPLVEALSNIPKPEMSTTMAESSLLDLLPKEEKLRHYFRYLGSLTTPTCDEKVVWTVFREPIQLHREQILAFSQKLYYDKEQTVSMKDNVRPLQQLGQRTVIKS

SEQ ID NO: 11 - protein, 262 aa, Homo sapiens
  Carbonic anhydrase VII scaffold
GHHGWGYGQDDGPSHWHKLYPIAQGDRQSPINIISSQAVYSPSLQPLELSYEACMSLSITNNGHSVQVDFNDSDDRTVVTGGPLEGPYRLKQFHFHWGKKHDVGSEHTVDGKSFPSELHLVHWNAKKYSTFGEAASAPDGLAVVGVFLETGDEHPSMNRLTDALYMVRFKGTKAQFSCFNPKSLLPASRHYWTYPGSLTTPPLSESVTWIVLREPISISERQMGKFRSLLFTSEDDERIHMVNNFRPPQPLKGRVVKASFRA

SEQ ID NO: 12 - protein, 260 aa, Homo sapiens
  Carbonic anhydrase XII scaffold
KWTYFGPDGENSWSKKYPSCGGLLQSPIDLHSDILQYDASLTPLEFQGYNLSANKQFLLTNNGHSVKLNLPSDMHIQGLQSRYSATQLHLHWGNPNDPHGSEHTVSGQHFAAELHIVHYNSDLYPDASTASNKSEGLAVLAVLIEMGSFNPSYDKIFSHLQHVKYKGQEAFVPGFNIEELLPERTAEYYRYRGSLTTPPCNPTVLWTVFRNPVQISQEQLLALETALYCTHMDDPSPREMINNFRQVQKFDERLVYTSFS

SEQ ID NO: 13 - protein, 259 aa, Homo sapiens
  Carbonic anhydrase XIII scaffold
LSWGYREHNGPIHWKEFFPIADGDQQSPIEIKTKEVKYDSSLRPLSIKYDPSSAKIISNSGHSFNVDFDDTENKSVLRGGPLTGSYRLRQVHLHWGSADDHGSEHIVDGVSYAAELHVVHWNSDKYPSFVEAAHEPDGLAVLGVFLQIGEPNSQLQKITDTLDSIKEKGKQTRFTNFDLLSLLPPSWDYWTYPGSLTVPPLLESVTWIVLKQPINISSQQLAKFRSLLCTAEGEAAAFLVSNHRPPQPLKGRKVRASFH

SEQ ID NO: 14 - protein, 261 aa, Homo sapiens
  ADAM17 (aka TACE) scaffold
ADPDPMKNTCKLLVVADHRFYRYMGRGEESTTTNYLIELIDRVDDIYRNTAWDNAGFKGYGIQIEQIRILKSPQEVKPGEKHYNMAKSYPNEEKDAWDVKMLLEQFSFDIAEEASKVCLAHLFTYQDFDMGTLGLAYGGSPRANSHGGVCPKAYYSPVGKKNIYLNSGLTSTKNYGKTILTKEADLVTTHELGHNFGAEHDPDGLAECAPNEDQGGKYVMYPIAVSGDHENNKMFSQCSKQSIYKTIESKAQECFQERSNA

SEQ ID NO: 15 - protein, 200 aa, Astacus astacus
  Astacin, scaffold
AAILGDEYLWSGGVIPYTFAGVSGADQSAILSGMQELEEKTCIRFVPRTTESDYVEIFTSGSGCWSYVGRISGAQQVSLQANGCVYHGTIIHELMHAIGFYHEHTRMDRDNYVTINYQNVDPSMTSNFDIDTYSRYVGEDYQYYSIMHYGKYSFSIQWGVLETIVPLQNGIDLTDPYDKAHMLQTDANQINNLYTNECSL

SEQ ID NO: 16 - protein, 866 aa, Escherichia coli
  Aminopeptidase N, scaffold
PQAKYRHDYRAPDYQITDIDLTFDLDAQKTVVTAVSQAVRHGASDAPLRLNGEDLKLVSVHINDEPWTAWKEEEGALVISNLPERFTLKIINEISPAANTALEGLYQSGDALCTQCEAEGFRHITYYLDRPDVLARFTTKIIADKIKYPFLLSNGNRVAQGELENGRHWVQWQDPFPKPCYLFALVAGDFDVLRDTFTTRSGREVALELYVDRGNLDRAPWAMTSLKNSMKWDEERFGLEYDLDIYMIVAVDFFNMGAMENKGLNIFNSKYVLARTDTATDKDYLDIERVIGHEYFHNWTGNRVTCRDWFQLSLKEGLTVFRDQEFSSDLGSRAVNRINNVRTMRGLQFAEDASPMAHPIRPDMVIEMNNFYTLTVYEKGAEVIRMIHTLLGEENFQKGMQLYFERHDGSAATCDDFVQAMEDASNVDLSHFRRWYSQSGTPIVTVKDDYNPETEQYTLTISQRTPATPDQAEKQPLHIPFAIELYDNEGKVIPLQKGGHPVNSVLNVTQAEQTFVFDNVYFQPVPALLCEFSAPVKLEYKWSDQQLTFLMRHARNDFSRWDAAQSLLATYIKLNVARHQQGQPLSLPVHVADAFRAVLLDEKIDPALAAEILTLPSVNEMAELFDIIDPIAIAEVREALTRTLATELADELLAIYNANYQSEYRVEHEDIAKRTLRNACLRFLAFGETHLADVLVSKQFHEANNMTDALAALSAAVAAQLPCRDALMQEYDDKWHQNGLVMDKWFILQATSPAANVLETVRGLLQHRSFTMSNPNRIRSLIGAFAGSNPAAFHAEDGSGYLFLVEMLTDLNSRNPQVASRLIEPLIRLKRYDAKRQEKMRAALEQLKGLENLSGDLYEKITKALA

SEQ ID NO: 17 - protein, 185 aa, Staphylococcus aureus
  Peptide deformylase, scaffold
MLTMKDIIRDGHPTLRQKAAELELPLTKEEKETLIAMREFLVNSQDEEIAKRYGLRSGVGLAAPQINISKRMIAVLIPDDGSGKSYDYMLVNPKIVSHSVQEAYLPTGEGCLSVDDNVAGLVHRHNRITIKAKDIEGNDIQLRLKGYPAIVFQHEIDHLNGVMFYDHIDKDHPLQPHTDAVEVLE

SEQ ID NO: 18 - protein, 696 aa, Homo sapiens
  Glutamate carboxypeptidase II, scaffold
KHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 19 - protein, 307 aa, Bos taurus
  Carboxypeptidase A, scaffold
ARSTNTFNYATYHTLDEIYDFMDLLVAQHPELVSKLQIGRSYEGRPIYVLKFSTGGSNRPAIWIDLGIHSREWITQATGVWFAKKFTENYGQNPSFTAILDSMDIFLEIVTNPNGFAFTHSENRLWRKTRSVTSSSLCVGVDANRNWDAGFGKAGASSSPCSETYHGKYANSEVEVKSIVDFVKNHGNFKAFLSIHSYSQLLLYPYGYTTQSIPDKTELNQVAKSAVAALKSLYGTSYKYGSIITTIYQASGGSIDWSYNQGIKYSFTFELRDTGRYGFLLPASQIIPTAQETWLGVLTIMEHTVNN

SEQ ID NO: 20 - protein, 323 aa, Bos taurus [sequence omitted]

SEQ ID NO: 21 - protein, 470 aa, Pseudomonas aeruginosa
  Alkaline protease, PAO1, scaffold
GRSDAYTQVDNFLHAYARGGDELVNGHPSYTVDQAAEQILREQASWQKAPGDSVLTLSYSFLTKPNDFFNTPWKYVSDIYSLGKFSAFSAQQQAQAKLSLQSWSDVTNIHFVDAGQGDQGDLTFGNFSSSVGGAAFAFLPDVPDALKGQSWYLINSSYSANVNPANGNYGRQTLTHEIGHTLGLSHPGDYNAGEGDPTYADATYAEDTRAYSVMSYWEEQNTGQDFKGAYSSAPLLDDIAAIQKLYGANLTTRTGDTVYGFNSNTERDFYSATSSSSKLVFSVWDAGGNDTLDFSGFSQNQKINLNEKALSDVGGLKGNVSIAAGVTVENAIGGSGSDLLIGNDVANVLKGGAGNDILYGGLGADQLWGGAGADTFVYGDIAESSAAAPDTLRDFVSGQDKIDLSGLDAFVNGGLVLQYVDAFAGKAGQAILSYDAASKAGSLAIDFSGDAHADFAINLIGQATQADIVV

SEQ ID NO: 22 - protein, 323 aa, Thermoactinomyces vulgaris [sequence omitted]

SEQ ID NO: 23 - protein, 860 aa, Escherichia coli
  StcE, scaffold
NSAIYFNTSQPINDLQGSLAAEVKFAQSQILPAHPKEGDSQPHLTSLRKSLLLVRPVKADDKTPVQVEARDDNNKILGTLTLYPPSSLPDTIYHLDGVPEGGIDFTPHNGTKKIINTVAEVNKLSDASGSSIHSHLTNNALVEIHTANGRWVRDIYLPQGPDLEGKMVRFVSSAGYSSTVFYGDRKVTLSVGNTLLFKYVNGQWFRSGELENNRITYAQHIWSAELPAHWIVPGLNLVIKQGNLSGRLNDIKIGAPGELLLHTIDIGMLTTPRDRFDFAADAAAHREYFQTIPVSRMIVNNYAPLHLKEVMLPTGELLTDMDPGNGGWHSGTMRQRIGKELVSHGIDNANYGLNSTAGLGENSHPYVVAQLAAHNSRGNYANGIQVHGGSGGGGIVTLDSTLGNEFSHDVGHNYGLGHYVDGFKGSVHRSAENNNSTWGWDGDKKRFIPNFYPSQTNEKSCLNNQCQEPFDGHKFGFDAMAGGSPFSAANRFTMYTPNSSAIIQRFFENKAVFDSRSSTGFSKWNADTQEMEPYEHTIDRAEQITASVNELSESKMAELMAEYAVVKVHMWNGNWTRNIYIPTASADNRGSILTINHEAGYNSYLFINGDEKVVSQGYKKSFVSDGQFWKERDVVDTREARKPEQFGVPVTTLVGYYDPEGTLSSYIYPAMYGAYGFTYSDDSQNLSDNDCQLQVDTKEGQLRFRLANHRANNTVMNKFHINVPTESQPTQATLVCNNKILDTKSLTPAPEGLTYTVNGQALPAKENEGCIVSVNSGKRYCLPVGQRSGYSLPDWIVGQEVYVDSGAKAKVLLSDWDNLSYNRIGEFVGNVNPADMKKVKAWNGQYLDFSKPRSMRVVYK

SEQ ID NO: 24 - protein, 259 aa, Escherichia coli
  AB5 holotoxin, scaffold
MAERTPNEEKKVIGYADHNGQLYNITSIYGPVINYTVPDENITINTINSTGERTQLTINYSDYVREAFNEWAPSGIRVQQVSSSGAEARVVSFSTTNYADNSLGSTIFDPSGNSRTRIDIGSFNRIVMNNFEKLKSRGAIPANMSPEEYIKLKLRITIKHEIGHILGLLHNNEGGSYFPHGVGLEVARCRLLNQAPSIMLNGSNYDYIDRLSHYLERPVTETDIGPSRNDIEGVRVMRRGGSGNSFTNRFSCLGLGLAF

SEQ ID NO: 25 - protein, 249 aa, Saccharopolyspora
  ZE2 (TIM barrel), scaffold
GSPRYLKGWLKDVVQLSLRRPSFRASRQRPIISLNERILEFNKRNITAIIAEYKRKSPSGLDVERDPIEYSKFMERYAVGLSILTEEKYFNGSYETLRKIASSVSIPILMADFIVKESQIDDAYNLGADTVLLIVKILTERELESLLEYARSYGMEPLIGINDENDLDIALRIGARFIGIHSADHETLEINKENQRKLISMIPSNVVKVAAHGISERNEIEELRKLGVNAFLIGSSLMRNPEKIKEFIL

SEQ ID NO: 26 - protein, 94 aa, Artificial Sequence
  MID1sc10 (de novo helical bundle), scaffold
GSPLAQQIKNTLTFIGQANAAGRMDEVRTLQQNLHPLWAEYFQQTEGSGGSPLAQQIQYGHVLIHQARAAGRMDEVRRLSENTLQLMKEYFQQS

SEQ ID NO: 27 - protein, 287 aa, Burkholderia multivorans ATCC 17616
  Amidohydrolase, scaffold
ALRIDSHQHFWRYRAADYPWIGAGMGVLARDYLPDALHPLMHAQALGASIAVQARAGRDETAFLLELACDEARIAAVVGWEDLRAPQLAERVAEWRGTKLRGFRHQLQDEADVRAFVDDADFARGVAWLQANDYVYDVLVFERQLPDVQAFCARHDAHWLVLDHAGKPALAEFDRDDTALARWRAALRELAALPHVVCKLSGLVTEADWRRGLRASDLRHIEQCLDAALDAFGPQRLMFGSDWPVCLLAASYDEVASLVERWAESRLSAAERSALWGGTAARCYALP

SEQ ID NO: 28 - protein, 185 aa, Artificial Sequence
  PDB ID 3U7M; mutant; M64
MLTMKDIIRDGHPTLRQKAAELELPLTKEEKETLIAMREFLVNSQDEEIAKRYGLRSAVVIVAPMINISKRMIAVLLPDDGSGKSYDYMLVNPKIVSHSVQEAYLPDGLQCLSVDDNVAGLVHRHNRITIKAKDIEGNDIQLRLKGLPAILFQHAIDHLNGVMFYDHIDKDHPLQPHTDAVEVLL

SEQ ID NO: 29 - protein, 470 aa, Artificial Sequence
  PDB ID 1KAP; mutant; M64
GRSDAYTQVDNFLHAYARGGDELVNGHPSYTVDQAAEQILREQASWQKAPGDSVLTLSYSFLTKPNDFFNTPWKYVSDIYSLGKFSAFSAQQQAQAKLSLQSWSDVTNIHFVDAGQGDQGDLTFGNFSSSVGGLVFVFLPDVPDALKGQSWYLINSSWSIVVNPANGNRGRQLLTHMIGHTLGLSHPGDYHPGEGDPTYADATYAEDTLAYSVMSLWEEQNTGQDFKGAYSSAPLLDDIAAIQKLYGANLTTRTGDTVYGFNSNTERDFYSATSSSSKLVFSVWDAGGNDTLDFSGFSQNQKINLNEKALSDVGGLKGNVSIAAGVTVENAIGGSGSDLLIGNDVANVLKGGAGNDILYGGLGADQLWGGAGADTFVYGDIAESSAAAPDTLRDFVSGQDKIDLSGLDAFVNGGLVLQYVDAFAGKAGQAILSYDAASKAGSLAIDFSGDAHADFAINLIGQATQADIVV

SEQ ID NO: 30 - protein, 182 aa, Artificial Sequence
  PDB ID 2X7M; mutant; M64
LVPRGSHMKLCLVAFDGRIPMLSSIVDRFEEHVSEYLGEVKVKKKRAKLPEHAYSKVRGQYLARALLDTLRGMKGEYDRVLGLTSEDLYIPGVNFVFLIARCPGREAVVSVARLLDPDPELYLERVVKELTHALGHTFGLGHCPDRNCVMSPASSLLEVDRKSPNFCRRCTELLQRNLKRGG

SEQ ID NO: 31 - protein, 478 aa, Artificial Sequence
  PDB ID 1LML; mutant; M64
VVRDVNWGALRIAVSTEDLTDPAYHCARVGQHVKDHAGAIVTCTAEDILTNEKRDILVKHLIPQAVQLHTERLKVQQVQGKWKVTDMVGDICGDFKVPQAHITEGFSNTDFVMYVASVPSEEGVLAWATTCQTFSDGHPAVGVINIPAANIASRYDQLVTRVVTHVMAHALGFSGPFFEDARIVANVPNVRGKNFDVPVINSSTAVAKAREQYGCDTLEYLEVEDQGGEGYAGSHIKMRNAQDELMAPAAAAGYYTALTMAIFQDLGFYQADFSKAEVMPWGQNAGCAFLTNKCMEQSVTQWPAMFCNESEDAIRCPTSRLSLGACGVTRHPGLPPYWQYFTDPSLAGVSALMDYCPVVVPYSDGSCTQRASEAHASLLPFNVFSDAARCIDGAFRPKATDGIVKSYAGLCANVQCDTATRTYSVQVHGSNDYTNCTPGLRVELSTVSNAFEGGGYITCPPYVEVCQGNVQAAKDGGN

SEQ ID NO: 32 - protein, 866 aa, Artificial Sequence
  PDB ID 4Q4E; mutant; M65
PQAKYRHDYRAPDYQITDIDLTFDLDAQKTVVTAVSQAVRHGASDAPLRLNGEDLKLVSVHINDEPWTAWKEEEGALVISNLPERFTLKIINEISPAANTALEGLYQSGDALCTQCLAEGFRHITYYLDRPDVLARFTTKIIADKIKYPFLLSNGNRVAQGELENGRHWVQWQDPFPKPCYLFALVAGDFDVLRDTFTTRSGREVALELYVDRGNLDRAPWAMTSLKNSMKWDEERFGLEYDLDIYMIVAVDFLNTGVVPNKGLNIFNSALVLARTDTATDKDYLEIEAVIGHVYFHNWTGNRVTCRDWFQLSLIEGLTLFRIQEFSSDLGSRAVNRINNVRTMRGLQFAEDASPMAHPIRPDMVIEMNNFFTLTVLWKGAEVIRMIHTLLGEENFQKGMQLYFERHDGSAATCDDFVQAMEDASNVDLSHFRRWYSQSGTPIVTVKDDYNPETEQYTLTISQRTPATPDQAEKQPLHIPFAIELYDNEGKVIPLQKGGHPVNSVLNVTQAEQTFVFDNVYFQPVPALLCEFSAPVKLEYKWSDQQLTFLMRHARNDFSRWDAAQSLLATYIKLNVARHQQGQPLSLPVHVADAFRAVLLDEKIDPALAAEILTLPSVNEMAELFDIIDPIAIAEVREALTRTLATELADELLAIYNANYQSEYRVEHEDIAKRTLRNACLRFLAFGETHLADVLVSKQFHEANNMTDALAALSAAVAAQLPCRDALMQEYDDKWHQNGLVMDKWFILQATSPAANVLETVRGLLQHRSFTMSNPNRIRSLIGAFAGSNPAAFHAEDGSGYLFLVEMLTDLNSRNPQVASRLIEPLIRLKRYDAKRQEKMRAALEQLKGLENLSGDLYEKITKALA

SEQ ID NO: 33 - protein, 185 aa, Artificial Sequence
  PDB ID 3U7M; mutant; M65
MLTMKDIIRDGHPTLRQKAAELELPLTKEEKETLIAMREFLVNSQDEEIAKRYGLRSLAMLVAPLINISKRMIVVLIPDDGSGKSYDYMLVNPKIVSHSVQEAYLPTGFQCIAVDDNVAGLVHLHNRITIKAKDIEGNDIQLRLKGLPALIFQHAIDHLNGVMFYDHIDKDHPLQPHTDAVEVLL

SEQ ID NO: 34 - protein, 257 aa, Artificial Sequence
  PDB ID 1Z97; mutant; M65
EWGYASHNGPDHWHELFPNAKGENQSPIELHTKDIRHDPSLQPWSVSYDGGSAKTILNDGLTCLVVFDDTYDRSMLRGGPLPGPYRLLFFHLHWGSSDDHGSLHTVDGVKYAAELHLVHWNPKYNTYKEAQKQRDGIAIIGIFLKIGHENGEFQIFLDALDKIKTKGKEAPFTKFDPSSLFPASRDYWTYQGAMAVPPLEECVVWLLLKEPMTVSSDQMAKLRSLLSSAENEPPVPLVSNWRPPQPINNRVVRASFK

SEQ ID NO: 35 - protein, 185 aa, Artificial Sequence
  PDB ID 3U7M; mutant; M72
MLTMKDIIRDGHPTLRQKAAELELPLTKEEKETLIAMREFLVNSLDEEIAKRYGLRSAGMLVAPMINISKRMIVMLIPDDGSGKSWDYMLVNPKIVSHSVQEAYLPTGEECLSVDDNVAGLVHRHNRITIKAKDIEGNDIQLRLKGLAAIAMQHAIDHLNGVMFYDHIDKDHPLQPHTDAVEVLL

SEQ ID NO: 36 - protein, 866 aa, Artificial Sequence
  PDB ID 4Q4E; mutant; M72
PQAKYRHDYRAPDYQITDIDLTFDLDAQKTVVTAVSQAVRHGASDAPLRLNGEDLKLVSVHINDEPWTAWKEEEGALVISNLPERFTLKIINEISPAANTALEGLYQSGDALCTQCVAEGFRHITYYLDRPDVLARFTTKIIADKIKYPFLLSNGNRVAQGELENGRHWVQWQDPFPKPCYLFALVAGDFDVLRDTFTTRSGREVALELYVDRGNLDRAPWAMTSLKNSMKWDEERFGLEYDLDIYMIVAVDFLNTGMMVNKGLNIVHSIAILARTDTATDKDYLDIERLIGHAYFHNWTGNRVTCRDWFQLSLIEGLTVFRDQEFSSDLGSRAVNRINNVRTMRGLQFAEDASPMAHPIRPDMVIEMNNFYTLTVLEKGAEVIRMIHTLLGEENFQKGMQLYFERHDGSAATCDDFVQAMEDASNVDLSHFRRWYSQSGTPIVTVKDDYNPETEQYTLTISQRTPATPDQAEKQPLHIPFAIELYDNEGKVIPLQKGGHPVNSVLNVTQAEQTFVFDNVYFQPVPALLCEFSAPVKLEYKWSDQQLTFLMRHARNDFSRWDAAQSLLATYIKLNVARHQQGQPLSLPVHVADAFRAVLLDEKIDPALAAEILTLPSVNEMAELFDIIDPIAIAEVREALTRTLATELADELLAIYNANYQSEYRVEHEDIAKRTLRNACLRFLAFGETHLADVLVSKQFHEANNMTDALAALSAAVAAQLPCRDALMQEYDDKWHQNGLVMDKWFILQATSPAANVLETVRGLLQHRSFTMSNPIRILSLIGAFAGSNPAAFHAEDGSGYLFLVEMLTDLNSRNPQVASRLIEPLIRLKRYDAKRQEKMRAALEQLKGLENLSGDLYEKITKALA

SEQ ID NO: 37 - protein, 478 aa, Artificial Sequence
  PDB ID 1LML; mutant; M72
VVRDVNWGALRIAVSTEDLTDPAYHCARVGQHVKDHAGAIVTCTAEDILTNEKRDILVKHLIPQAVQLHTERLKVQQVQGKWKVTDMVGDICGDFKVPQAHITEGFSNTDFVMYVASVPSHEGILAWATTCQTFSDGHPAVGVINIPAANIASRYDQLVTRVVTHAMAHALGFSGPFFEDARIVANVPNVRGKNFDVPVINSSTAVAKAREQYGCDTLEYLEVEDQGGAKIAGSHIKMRNAQDELMAPKAAAGYYTALTMAIFQDLGFYQADFSKAEVMPWGQNAGCAFLTNKCMEQSVTQWPAMFCNESEDAIRCPTSRLSLGACGVTRHPGLPPYWQYFTDPSLAGVSALMDYCPVVVPYSDGSCTQRASEAHASLLPFNVFSDAARCIDGAFRPKATDGIVKSYAGLCANVQCDTATRTYSVQVHGSNDYTNCTPGLRVELSTVSNAFEGGGYITCPPYVEVCQGNVQAAKDGGN

SEQ ID NO: 38 - protein, 860 aa, Artificial Sequence
  3UJZ; mutant, M72
NSAIYFNTSQPINDLQGSLAAEVKFAQSQILPAHPKEGDSQPHLTSLRKSLLLVRPVKADDKTPVQVEARDDNNKILGTLTLYPPSSLPDTIYHLDGVPEGGIDFTPHNGTKKIINTVAEVNKLSDASGSSIHSHLTNNALVEIHTANGRWVRDIYLPQGPDLEGKMVRFVSSAGYSSTVFYGDRKVTLSVGNTLLFKYVNGQWFRSGELENNRITYAQHIWSAELPAHWIVPGLNLVIKQGNLSGRLNDIKIGAPGELLLHTIDIGMLTTPRDRFDFAADAAAHREYFQTIPVSRMIVNNYAPLHLKEVMLPTGELLTDMDPGNGGETSGTMRQRIGKELVSHGIDNANYGLNSTAGLGENSHPYVVAQLAAHNSRGNYANGIQVHGKSGGGGIVTLDSTLGNEFSHAVGHNYGLGHYVDGFKGSVHRSAENNNSTWGWDGDKKRFIPNFYPSQTNEKSCLNNQCQEPFDGHKFGFDAMAGGSPFSAANRFTMYTPNSSAIIQRFFENKAVFDSRSSTGFSKWNADTQEMEPYEHTIDRAEQITASVNELSESKMAELMAEYAVVKVHMWNGNWTRNIYIPTASADNRGSILTINHEAGYNSYLFINGDEKVVSQGYKKSFVSDGQFWKERDVVDTREARKPEQFGVPVTTLVGYYDPEGTLSSYIYPAMYGAYGFTYSDDSQNLSDNDCQLQVDTKEGQLRFRLANHRANNTVMNKFHINVPTESQPTQATLVCNNKILDTKSLTPAPEGLTYTVNGQALPAKENEGCIVSVNSGKRYCLPVGQRSGYSLPDWIVGQEVYVDSGAKAKVLLSDWDNLSYNRIGEFVGNVNPADMKKVKAWNGQYLDFSKPRSMRVVYK

SEQ ID NO: 39 - protein, 201 aa, Artificial Sequence
  PDB ID 1IAG; M83
NLPQRYIELVVVADRRVFMKYNSDLNIIRTRVHEIVNIINKFYRSLNIRVSLTDLEIWSGQDFITIQSSSSNTLNSFGEWRERVLLIWKRHDNAQLLTAINFEGKIILLAYTSSMCNPRSSVGIVKDHSPINLLVAVTMAHLLGHNLGMEHDGPDCLRGASLCIMLPELTPGRSYEFSDDSMGYYQKFLNQYKPQCILNKP

SEQ ID NO: 40 - protein, 866 aa, Artificial Sequence
  PDB ID 4Q4E; mutant; M83
PQAKYRHDYRAPDYQITDIDLTFDLDAQKTVVTAVSQAVRHGASDAPLRLNGEDLKLVSVHINDEPWTAWKEEEGALVISNLPERFTLKIINEISPAANTALEGLYQSGDALCTQCMAEGFRHITYYLDRPDVLARFTTKIIADKIKYPFLLSNGNRVAQGELENGRHWVQWQDPFPKPCYLFALVAGDFDVLRDTFTTRSGREVALELYVDRGNLDRAPWAMTSLKNSMKWDEERFGLEYDLDIYMIVAVDFFNTLLIPNKGLNIFNSKLVLARTDTATDEDYLRIEAAIGHIYFHNWTGNRVTCRDWFQLSLKEGLTVFRDQEFSSDLGSRAVNRINNVRIMRGLQFAEDASPMAHPIRPDMVIEMNNFYTLTVLYKGAEVIRMIHTLLGEENFQKGMQLYFERHDGSAATCDDFVQAMEDASNVDLSHFRRWYSQSGTPIVTVKDDYNPETEQYTLTISQRTPATPDQAEKQPLHIPFAIELYDNEGKVIPLQKGGHPVNSVLNVTQAEQTFVFDNVYFQPVPALLCEFSAPVKLEYKWSDQQLTFLMRHARNDFSRWDAAQSLLATYIKLNVARHQQGQPLSLPVHVADAFRAVLLDEKIDPALAAEILTLPSVNEMAELFDIIDPIAIAEVREALTRTLATELADELLAIYNANYQSEYRVEHEDIAKRTLRNACLRFLAFGETHLADVLVSKQFHEANNMTDALAALSAAVAAQLPCRDALMQEYDDKWHQNGLVMDKWFILQATSPAANVLETVRGLLQHRSFTMSNPNRIRSLIGAFAGSNPAAFHAEDGSGYLFLVEMLTDLNSRNPQVASRLIEPLIRLKRYDAKRQEKMRAALEQLKGLENLSGDLYEKITKALA

SEQ ID NO: 41 - protein, 478 aa, Artificial Sequence
  PDB ID 1LML; mutant; M83
VVRDVNWGALRIAVSTEDLTDPAYHCARVGQHVKDHAGAIVTCTAEDILTNEKRDILVKHLIPQAVQLHTERLKVQQVQGKWKVTDMVGDICGDFKVPQAHITEGFSNTDFVMYVASVPSEEGVLAWATTCQTFSDGHPAVGVINIPAANIASRYDQLVTRVVTHAMAHALGFSGPFFEDARIVANVPNVRGKNFDVPVINSSTAVAKAREQYGCDTLEYLEVEDQAGAKAAGSHIKMRNAQDELMAPAAAAGYYTALTMAIFQDLGFYQADFSKAEVMPWGQNAGCAFLTNKCMEQSVTQWPAMFCNESEDAIRCPTSRLSLGACGVTRHPGLPPYWQYFTDPSLAGVSARMDYCPVVVPYSDGSCTQRASEAHASLLPFNVFSDAARCIDGAFRPKATDGIVKSYAGLCANVQCDTATRTYSVQVHGSNDYTNCTPGLRVELSTVSNAFEGGGYITCPPYVEVCQGNVQAAKDGGN

SEQ ID NO: 42 - protein, 478 aa, Artificial Sequence
  PDB ID 1LML; mutant; M86
VVRDVNWGALRIAVSTEDLTDPAYHCARVGQHVKDHAGAIVTCTAEDILTNEKRDILVKHLIPQAVQLHTERLKVQQVQGKWKVTDMVGDICGDFKVPQAHITEGFSNTDFVMYVASVPSHEGVLAWATTCQTFSDGHPAVGVINIPAANIASRYDQLVTRVVTHVMAHALGFSGPFFEDARIVANVPNVRGKNFDVPVINSSTAVAKAREQYGCDTLEYLEVEDQGGRGVAGSHIKMRNAQDELMAPDAAAGYYTALTMAIFQDLGFYQADFSKAEVMPWGQNAGCAFLTNKCMEQSVTQWPAMFCNESEDAIRCPTSRLSLGACGVTRHPGLPPYWQYFTDPSLAGVSALMDYCPVVVPYSDGSCTQRASEAHASLLPFNVFSDAARCIDGAFRPKATDGIVKSYAGLCANVQCDTATRTYSVQVHGSNDYTNCTPGLRVELSTVSNAFEGGGYITCPPYVEVCQGNVQAAKDGGN

SEQ ID NO: 43 - protein, 724 aa, Artificial Sequence
  PDB ID 5E3C; mutant; M86
DTQYILPNDIGVSSLDSREAFRLLSPTERLYAYHLSRAAWYGGLAVLLQTSPEAPYIYALLSRLFRAQDPDQLRQHALAEGLTEEEYQAFLVYAAGVYSNMGNYKAWGDTKFVPNLPKEKLERVILGSEAAQQHPEEVRGLWQTCGELMFSLEPRLRHLGLGKEGITTYFSGNCTMEDAKLAQDFLDSQNLSAYNTRLFKEVDGCGKPYYEVRLASVLGSEPSLDSEVTSKLKSYEFRGSPFQVTRGDYAPILQKVVEQLEKAKAYAANSHQGQMLAQYIESFTQGSIEAHKRGSRFWIQDKGPIVESYIGFIWSVFDPFGSRGMFFGFVAVVNKAMSAKFERLVASAEQLLKELPWPPTFEKDKFLTPDFTSLDVLTMAGLGIPLMMVIPNYDDLRQTEGFKNVSLGNVLAVAYATQREKLTFLEEDDKDLYILWKGPSFDVQVGLHALLGHGSGKLFVQDEKGAFNFDQETVINPETGEQIQSWYRCGETWDSKFSTIASSYEECRAESVGLYLSLHPQVLEIFGFEGADAEDVIYVNWLNMVRAGLLALEFYTPEAFNWRDVLMQARFVILRVLLEAGEGLVTITPTTGSDGRPDARVRLDRSKIRSVGKPALERFLRRLQVLKSTGDVAGGRALYEGYATVTDAPPESFLTLRDTVLLRKESRKLIVQPNTRLEGSDVQLLEYEASAAGLIRSFSERFPEDGPELEEILTQLATADARFW

SEQ ID NO: 44 - protein, 261 aa, Artificial Sequence
  PDB ID 2FV5; mutant; M86
ADPDPMKNTCKLLVVADHRFYRYMGRGEESTTTNYLIELIDRVDDIYRNTAWDNAGFKGYGIQIEQIRILKSPQEVKPGEKHYNMAKSYPNEEKDAWDLKMLLEQFSFDIAEEASKVCLAHLFTYQDFDMGRLLIVYGGSPHANSHGGVCPKAYYSPVGKKNIYLNSGLTSTKNYGKTILTKEADLVTTHALGHNFGAEHDPDGLAECAPNEDQGGKYVMYPIAVSGDHENNKMFSQCSKQSIYKTIESKAQECFQERSNA

SEQ ID NO: 45 - protein, 249 aa, Artificial Sequence
  PDB ID 5K7J; mutant; M93
GSPRYLDGWLKDVVQLSLRRPSFRASRQRPIISLNERILEFNKRNITAIIAEYKRKSVSGLDVERDPIEYSKFMERYAVGLSILTEEKYFNGSYETLRKIASSVSIPILMADFIVKESQIDDAYNLGADTVLLLVMILTERELESLLEYARSYGMEPIIMVLDENMLDIALRIGARFIVLHSKSHETLEINKENQRKLISMIPSNVVKVMVHGISERNEIEELRKLGVNAFIIGSSLMRNPEKIKEFIL

SEQ ID NO: 46 - protein, 201 aa, Artificial Sequence
  PDB ID 1IAG; mutant; M93
NLPQRYIELVVVADRRVFMKYNSDLNIIRTRVHEIVNIINKFYRSLNIRVSLTDLEIWSGQDFITIQSSSSNTLNSFGEWRERVLLIWKRHDNAQLLTAINFEGKIIMLAYTSSMCNPRSSVGIVKDHSPINLLVAVTMAHLLGHNLGMEHDGKDCLRGASLCIMLPDLTPGRSYEFSDDSMGYYQKFLNQYKPQCILNKP

SEQ ID NO: 47 - protein, 866 aa, Artificial Sequence
  PDB ID 4Q4E; mutant; M93
PQAKYRHDYRAPDYQITDIDLTFDLDAQKTVVTAVSQAVRHGASDAPLRLNGEDLKLVSVHINDEPWTAWKEEEGALVISNLPERFTLKIINEISPAANTALEGLYQSGDALCTQCLAEGFRHITYYLDRPDVLARFTTKIIADKIKYPFLLSNGNRVAQGELENGRHWVQWQDPFPKPCYLFALVAGDFDVLRDTFTTRSGREVALELYVDRGNLDRAPWAMTSLKNSMKWDEERFGLEYDLDIYMIVAVDFFNMIVIPNKGLNIFNSKYVLARTDTATDEDYLKIEQIIGHAYFHNWTGNRVTCRDWFQLSLIEGLTLFRVQEFSSDLGSRAVNRINNVRIMRGLQFAEDASPMAHPIRPDMVIEMNNFFTLTVMWKGAEVIRMIHTLLGEENFQKGMQLYFERHDGSAATCDDFVQAMEDASNVDLSHFRRWYSQSGTPIVTVKDDYNPETEQYTLTISQRTPATPDQAEKQPLHIPFAIELYDNEGKVIPLQKGGHPVNSVLNVTQAEQTFVFDNVYFQPVPALLCEFSAPVKLEYKWSDQQLTFLMRHARNDFSRWDAAQSLLATYIKLNVARHQQGQPLSLPVHVADAFRAVLLDEKIDPALAAEILTLPSVNEMAELFDIIDPIAIAEVREALTRTLATELADELLAIYNANYQSEYRVEHEDIAKRTLRNACLRFLAFGETHLADVLVSKQFHEANNMTDALAALSAAVAAQLPCRDALMQEYDDKWHQNGLVMDKWFILQATSPAANVLETVRGLLQHRSFTMSNPNRIRSLIGAFAGSNPAAFHAEDGSGYLFLVEMLTDLNSRNPQVASRLIEPLIRLKRYDAKRQEKMRAALEQLKGLENLSGDLYEKITKALA

SEQ ID NO: 48 - protein, 256 aa, Artificial Sequence
  Carbonic anhydrase II mutant, M=M64
HWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQHPDGLAVLGIFLKVGSAKPGLQKVVDVLDSIKTKGKSADFTNFDPRGLLPESLDYWTYPGSRTTPPLLESVTWIVLKEPISVSSEQVLKFRKLNFNGEGEPEELMVDNWRPAQPLKNRQIKASF

SEQ ID NO: 49 - protein, 256 aa, Artificial Sequence
  Carbonic anhydrase II mutant, M=M64
HWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFDKAVRQPDGLAVLGIFLKVGSAKPGLQKVVDVLDSIKTKGKSADFTNFDPRGLLPESLDYWTYPGSRTTPPLLESVTWIVLKEPISVSSEQVLKFRKLNFNGEGEPEELMVDNWRPAQPLKNRQIKASF

SEQ ID NO: 50 - protein, 256 aa, Artificial Sequence
  Carbonic anhydrase II mutant, M=M64
HWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFDKAVQQPDGLAVLGIFLNVGSARPGLQKVVDVLDSIKTKGKSAYFTNFDPRGLLPESLDYWTYPGSRTTPPLLESVTWIVLKEPISVSSEQILKFRKLNFNREGEPEELMVDNWRPTQPLKNRQIKASF

SEQ ID NO: 51 - protein, 256 aa, Artificial Sequence
  Carbonic anhydrase II mutant, M=M64
HWGYGKHNGPEHWHKDFPIAKGERQSPVDIDTHTAKYDPSLKPLSVSYDQATSLRILNNGHAFEVEFDDSQDKAVLKGGPLDGTYRLVQFHFHWGSLDGQGSEHTVDKKKYAAELHLVHWNTKYGDYGKAMQQPDGLAVLGIFLKVGSAKPGLQKVVDVLDSIKTKGKSADFTNFDPRGLLPESLDYWTYPGSGTVPPLLESVTWIVLKEPISVSSEQVLKFRKLNFNGEGEPEELMVDNWRPAQPLKNRQIKASF

SEQ ID NO: 52 - protein, 256 aa, Artificial Sequence
  Carbonic anhydrase II mutant, M=M64
HWGYGKHNGPEHWHKDFPIAKGERQSPVDIDTHTAKYDPSLKPLSVSYDQATSLRILNDGHAFQVEFDDSQDKAVLKGGPLDGTYRLVQFHFHWGSLDGQGSEHTVDKKKYAAELHLVHWNTKYGDYGKAMQQPDGLAVLGIFLKVGSAKPGLQKVVDVLDSIKTKGKSADFTNFDPRGLLPESLDYWTYPGSGTVPPLLESVTWIVLKEPISVSSEQVLKFRKLNFNGEGEPEELMVDNWRPAQPLKNRQIKASF

SEQ ID NO: 53 - protein, 256 aa, Artificial Sequence
  Carbonic anhydrase II mutant, M=M64
HWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAMQQPDGLAVLGIFLKVGSAKPGLQKVVDVLDSIKTKGKSADFTNFDPRGLLPESLDYWTYPGSGTVPPLLESVTWIVLKEPISVSSEQVLKFRKLNFNGEGEPEELMVDNWRPAQPLKNRQIKASF

SEQ ID NO: 54 - protein, 256 aa, Artificial Sequence
  Carbonic anhydrase II mutant, M=M64
HWGYGKHNGPEHWHKDFPIAKGERQSPVDIDTHTAKYDPSLKPLSVSYDQATSLRILNDGHAFEVEFDDSQDKAVLKGGPLDGTYRLIQFHFHWGSLDGQGSEHTVDKKKYAAELHLVHWNTKYGDFGKALQQPDGMAILGIFLKVGSAKPGLQKVVDVLDSIKTKGKSADFTNFDPRGLLPESLDYWTYPGSQTIPPLLESVTWIVLKEPISVSSEQVLKFRKLNFNGEGEPEELMVDNWRPAQPLKNRQIKASF

SEQ ID NO: 55 - protein, 256 aa, Artificial Sequence
  Carbonic anhydrase II mutant, M=M64
HWGYGKHNGPEHWHKDFPIAKGERQSPVDIDTHTAKYDPSLKPLSVSYDQATSLRILNNGHAFLVEFDDSQDKAVLKGGPLDGTYRLKQFHFHWGSLDGQGSEHTVDKKKYAAELHLVHWNTKYGDYGKALQQPDGIAVLGIFLKVGSAKPGLQKVVDVLDSIKTKGKSADFTNFDPRGLLPESLDYWTYPGSRTIPPLLESVTWIVLKEPISVSSEQVLKFRKLNFNGEGEPEELMVDNWRPAQPLKNRQIKASF

SEQ ID NO: 56 - protein, 256 aa, Artificial Sequence
  Carbonic anhydrase II mutant, M=M64
HWGYGKHNGPEHWHKDFPIAKGERQSPVDIDTHTAKYDPSLKPLSVSYDQATSLRILNNGHAFLVEFDDSQDKAVLKGGPLDGTYRLIQFHFHWGSLDGQGSEHTVDKKKYAAELHLVHWNTKYGDYGKALQQPDGIAVLGIFLKVGSAKPGLQKVVDVLDSIKTKGKSADFTNFDPRGLLPESLDYWTYPGSRTIPPLLESVTWIVLKEPISVSSEQVLKFRKLNFNGEGEPEELMVDNWRPAQPLKNRQIKASF

SEQ ID NO: 57 - protein, 256 aa, Artificial Sequence
  Carbonic anhydrase II mutant, M=M64
HWGYGKHNGPEHWHKDFPIAKGERQSPVDIDTHTAKYDPSLKPLSVSYDQATSLRILNDGHAFRVEFDDSQDKAVLKGGPLDGTYRLKQFHFHWGSLDGQGSEHTVDKKKYAAELHLVHWNTKYGDFGKAGQQPDGIAILGIFLKVGSAKPGLQKVVDVLDSIKTKGKSADFTNFDPRGLLPESLDYWTYPGSETIPPLLESVTWIVLKEPISVSSEQVLKFRKLNFNGEGEPEELMVDNWRPAQPLKNRQIKASF

SEQ ID NO: 58 - protein, 257 aa, Artificial Sequence
  Carbonic anhydrase I, M=M64
DWGYDDKNGPEQWSKLYPIANGNNQSPVDIKTSETKHDTSLKPISVSYNPATAKEIINVGHSFHVNFEDNDNRSVLKGGPFSDSYRLFQFHFHWGSTNEHGSEHTVDGVKYSAELHVAHWNSAKYSSLAEAASKADGLAVIGVLMKVGEANPKLQKVLDALQAIKTKGKRAPFTNFDPSTLLPSSLDFWTYPGSLTHPPLYESVTWIICKESISVSSEQLAQFRSLLSNVEGDNAVPMQHNNRPTQPLKGRTVRASF

SEQ ID NO: 59 - protein, 722 aa, Artificial Sequence
  truncated 4Q4E scaffold
PQAKYRHDYRAPDYQITDIDLTFDLDAQKTVVTAVSQAVRHGASDAPLRLNGEDLKLVSVHINDEPWTAWKEEEGALVISNLPERFTLKIINEISPAANTALEGLYQSGDALCTQCEAEGFRHITYYLDRPDVLARFTTKIIADKIKYPFLLSNGNRVAQGELENGRHWVQWQDPFPKPCYLFALVAGDFDVLRDTFTTRSGREVALELYVDRGNLDRAPWAMTSLKNSMKWDEERFGLEYDLDIYMIVAVDFFNMGAMENKGLNIFNSKYVLARTDTATDKDYLDIERVIGHEYFHNWTGNRVTCRDWFQLSLKEGLTVFRDQEFSSDLGSRAVNRINNVRTMRGLQFAEDASPMAHPIRPDMVIEMNNFYTLTVYEKGAEVIRMIHTLLGEENFQKGMQLYFERHDGSAATCDDFVQAMEDASNVDLSHFRRWYSQSGTPIVTVKDDYNPETEQYTLTISQRTPATPDQAEKQPLHIPFAIELYDNEGKVIPLQKGGHPVNSVLNVTQAEQTFVFDNVYFQPVPALLCEFSAPVKLEYKWSDQQLTFLMRHARNDFSRWDAAQSLLATYIKLNVARHQQGQPLSLPVHVADAFRAVLLDEKIDPALAAEILTLPSVNEMAELFDIIDPIAIAEVREALTRTLATELADELLAIYNANYQSEYRVEHEDIAKRTLRNACLRFLAFGETHLADVLVSKQFHEANNMTDALAALSAAVAAQLP

SEQ ID NO: 60 - protein, 5 aa, Artificial Sequence
  model peptide
AFAAE

SEQ ID NO: 61 - protein, 5 aa, Artificial Sequence
  model peptide
  misc_feature (1)..(1): Xaa is any standard L-amino acid residue
XAAAE

SEQ ID NO: 62 - protein, 5 aa, Artificial Sequence
  model peptide
DAEIR

SEQ ID NO: 63 - protein, 5 aa, Artificial Sequence
  model peptide
EAEIR

SEQ ID NO: 64 - protein, 5 aa, Artificial Sequence
  model peptide
AAEIR

SEQ ID NO: 65 - protein, 5 aa, Artificial Sequence
  model peptide
FAEIR

SEQ ID NO: 66 - protein, 5 aa, Artificial Sequence
  model peptide
LAEIR